(19) United States
(12) Patent Application Publication
(10) Pub. No.: US 2019/0337946 A1
(43) Pub. Date: Nov. 7, 2019

(54) TRIAZOLOPYRIDINE COMPOUNDS AND METHODS OF USE THEREOF

(71) Applicant: Genentech, Inc., South San Francisco, CA (US)

(72) Inventors: Simon Charles Goodacre, Harlow Essex (GB); Nicholas Charles Ray, Harlow Essex (GB); Mark Edward Zak, San Mateo, CA (US); Yun-Xing Cheng, Beijing (CN); Wei Li, Beijing (CN)

(73) Assignee: Genentech, Inc., South San Francisco, CA (US)

(21) Appl. No.: 16/515,253

(22) Filed: Jul. 18, 2019

Related U.S. Application Data
(63) Continuation of application No. 15/975,462, filed on May 9, 2018, now abandoned.
Application No. 15/975,462 is a continuation of application No. 15/692,907, filed on Aug. 31, 2017, now abandoned.
Application No. 15/692,907 is a continuation of application No. PCT/EP2016/054343, filed on Mar. 2, 2016.
(60) Provisional application No. 62/134,838, filed on Mar. 18, 2015.
(60) Provisional application No. 62/128,234, filed on Mar. 4, 2015.

(30) Foreign Application Priority Data
Jan. 27, 2016 (CN) PCT/CN2016/072287

(51) Int. Cl. C07D 471/04; C07D 471/10

(52) U.S. Cl. CPC C07D 471/04; C07D 471/10

(57) ABSTRACT

Triazolopyridine compounds that are inhibitors of JAK kinase, such as JAK1, compositions containing these compounds and methods for treating diseases mediated by a JAK kinase. In particular, provided are compounds of formula (I), stereoisomers, tautomers, solvates, prodrugs or pharmaceutically acceptable salts thereof, where R^{1a}, R^{1b}, R^{1c}, R², R³, R⁴ and R⁵ are defined herein, pharmaceutical compositions comprising the compound and a pharmaceutically acceptable carrier, adjuvant or vehicle, methods of using the compound or composition in therapy, for example, for treating a disease or condition mediated by a JAK kinase in a patient.

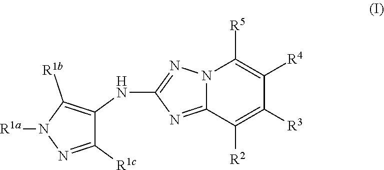

### CROSS-REFERENCE TO RELATED APPLICATIONS

This patent application is a continuation of U.S. application Ser. No. 15/975,462, filed May 9, 2018, which is a continuation of U.S. application Ser. No. 15/692,907, filed Aug. 31, 2017, which claims priority to continuation International Application No. PCT/EP2016/054343, filed Mar. 2, 2016, which claims the benefit of International Application No. PCT/CN2016/072287, filed Jan. 27, 2016, U.S. Provisional Appl. No. 62/134,838, filed Mar. 18, 2015, and U.S. Provisional Appl. No. 62/128,234, filed Mar. 4, 2015, each of which is incorporated herein by reference in its entirety.

The invention relates to organic compounds useful for therapy and/or prophylaxis in a patient, and in particular to inhibitors of JAK kinases useful for diagnosis and treatment of diseases or conditions responsive to the inhibition of a JAK kinase.

### BACKGROUND OF THE INVENTION

Cytokine pathways mediate a broad range of biological functions, including many aspects of inflammation and immunity. Janus kinases (JAK), including JAK1, JAK2, JAK3 and TYK2, are cytoplasmic protein kinases that associate with type I and type II cytokine receptors and regulate cytokine signal transduction. Cytokine engagement with cognate receptors triggers activation of receptor associated JAKs and this leads to JAK-mediated tyrosine phosphorylation of signal transducer and activator of transcription (STAT) proteins and ultimately transcriptional activation of specific gene sets (Schindler et al., 2007, J. Biol. Chem. 282: 20059-63). JAK1, JAK2 and TYK2 exhibit broad patterns of gene expression, while JAK3 expression is limited to leukocytes. Cytokine receptors are typically functional as heterodimers, and as a result, more than one type of JAK kinase is usually associated with cytokine receptor complexes. The specific JAKs associated with different cytokine receptor complexes have been determined in many cases through genetic studies and corroborated by other experimental evidence. Exemplary therapeutic benefits of the inhibition of JAK enzymes are discussed, for example, in International Application No. WO 2013/014567.

JAK1 was initially identified in a screen for novel kinases (Wilks A. F., 1989, Proc. Natl. Acad. Sci. U.S.A. 86:1603-1607). Genetic and biochemical studies have shown that JAK1 is functionally and physically associated with the type I interferon (e.g., IFNalpha), type II interferon (e.g., IFNgamma), and IL-2 and IL-6 cytokine receptor complexes (Kisseleva et al., 2002, Gene 285:1-24; Levy et al., 2005, Nat. Rev. Mol. Cell Biol. 3:651-662; O'Shea et al., 2002, Cell, 109 (suppl.): S121-S131). JAK1 knockout mice die perinatally due to defects in LIF receptor signaling (Kisseleva et al., 2002, Gene 285:1-24; O'Shea et al., 2002, Cell, 109 (suppl.): S121-S131). Characterization of tissues derived from JAK1 knockout mice demonstrated critical roles for this kinase in the IFN, IL-10, IL-2/IL-4 and IL-6 pathways. A humanized monoclonal antibody targeting the IL-6 pathway (Tocilizumab) was recently approved by the European Commission for the treatment of moderate-to-severe rheumatoid arthritis (Scheinecker et al., 2009, Nat. Rev. Drug Discov. 8:273-274).

CD4 T cells play an important role in asthma pathogenesis through the production of TH2 cytokines within the lung, including IL-4, IL-9 and IL-13 (Cohn et al., 2004, Annu. Rev. Immunol. 22:789-815). IL-4 and IL-13 induce increased mucus production, recruitment of eosinophils to the lung, and increased production of IgE (Kasaian et al., 2008, Biochem. Pharmacol. 76(2): 147-155). IL-9 leads to mast cell activation, which exacerbates the asthma symptoms (Kearley et al., 2011, Am. J. Resp. Crit. Care Med., 183(7): 865-875). The IL-4Rα chain activates JAK1 and binds to either IL-4 or IL-13 when combined with the common gamma chain or the IL-13Rα1 chain respectively (Pernis et al., 2002, J. Clin. Invest. 109(10):1279-1283). The common gamma chain can also combine with IL-9Rα to bind to IL-9, and IL-9Rα activates JAK1 as well (Demoulin et al., 1996, Mol. Cell Biol. 16(9):4710-4716). While the common gamma chain activates JAK3, it has been shown that JAK1 is dominant over JAK3, and inhibition of JAK1 is sufficient to inactivate signaling through the common gamma chain despite JAK3 activity (Haan et al., 2011, Chem. Biol. 18(3):314-323). Inhibition of IL-4, IL-13 and IL-9 signaling by blocking the JAK/STAT signaling pathway can alleviate asthmatic symptoms in pre-clinical lung inflammation models (Mathew et al., 2001, J. Exp. Med. 193(9): 1087-1096; Kudlacz et. al., 2008, Eur. J. Pharmacol. 582(1-3): 154-161).

Biochemical and genetic studies have shown an association between JAK2 and single-chain (e.g., EPO), IL-3 and interferon gamma cytokine receptor families (Kisseleva et al., 2002, Gene 285:1-24; Levy et al., 2005, Nat. Rev. Mol. Cell Biol. 3:651-662; O'Shea et al., 2002, Cell, 109 (suppl.): S121-S131). Consistent with this, JAK2 knockout mice die of anemia (O'Shea et al., 2002, Cell, 109 (suppl.): S121-S131). Kinase activating mutations in JAK2 (e.g., JAK2 V617F) are associated with myeloproliferative disorders in humans.

JAK3 associates exclusively with the gamma common cytokine receptor chain, which is present in the IL-2, IL-4, IL-7, IL-9, IL-15 and IL-21 cytokine receptor complexes. JAK3 is critical for lymphoid cell development and proliferation and mutations in JAK3 result in severe combined immunodeficiency (SCID) (O'Shea et al., 2002, Cell, 109 (suppl.): S121-S131). Based on its role in regulating lymphocytes, JAK3 and JAK3-mediated pathways have been targeted for immunosuppressive indications (e.g., transplantation rejection and rheumatoid arthritis) (Baslund et al., 2005, Arthritis & Rheumatism 52:2686-2692; Changelian et al., 2003, Science 302: 875-878).

TYK2 associates with the type I interferon (e.g., IFNalpha), IL-6, IL-10, IL-12 and IL-23 cytokine receptor complexes (Kisseleva et al., 2002, Gene 285:1-24; Watford, W. T. & O'Shea, J. J., 2006, Immunity 25:695-697). Consistent with this, primary cells derived from a TYK2 deficient human are defective in type I interferon, IL-6, IL-10, IL-12 and IL-23 signaling. A fully human monoclonal antibody targeting the shared p40 subunit of the IL-12 and IL-23 cytokines (Ustekinumab) was recently approved by the European Commission for the treatment of moderate-to-severe plaque psoriasis (Krueger et al., 2007, N. Engl. J. Med. 356:580-92; Reich et al., 2009, Nat. Rev. Drug Discov. 8:355-356). In addition, an antibody targeting the IL-12 and IL-23 pathways underwent clinical trials for treating Crohn's Disease (Mannon et al., 2004, N. Engl. J. Med. 351:2069-79).

There exists a need in the art for additional or alternative treatments of conditions mediated by JAK kinases, such as those described above.

### BRIEF SUMMARY OF THE INVENTION

Disclosed are triazolopyridine compounds that are inhibitors of JAK kinases, compositions containing these compounds and methods for treating diseases mediated by JAK kinases.

In one aspect, provided is a compound of formula (I):

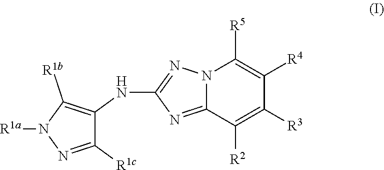

or a stereoisomer, tautomer, solvate, prodrug or salt thereof, wherein where R^{1a}, R^{1b}, R^{1c}, R², R³, R⁴ and R⁵ are defined herein.

In some embodiments, provided is a compound of formula (Ia):

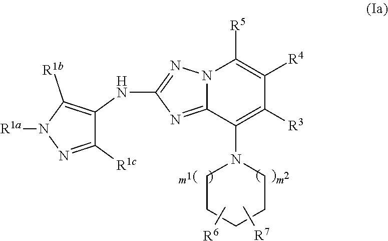

or a stereoisomer, tautomer, solvate, prodrug or salt thereof, where R^{1a}, R^{1b}, R^{1c}, R³, R⁴, R⁵, R⁶, R⁷, m¹ and m² are as defined herein.

In some embodiments, provided is a compound of formula (Ic):

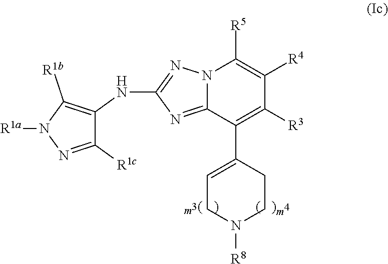

or a stereoisomer, tautomer, solvate, prodrug or salt thereof, where R^{1a}, R^{1b}, R^{1c}, R³, R⁴, R⁵, R⁸, m³ and m⁴ are as defined herein.

In some embodiments, provided is a compound of formula (Ik):

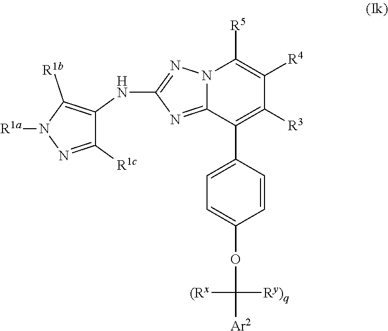

or a stereoisomer, tautomer, solvate, prodrug or salt thereof, where R^{1a}, R^{1b}, R^{1c}, R³, R⁴, R⁵, R^{x}, R^{y}, Ar² and q are as defined herein.

Also provided herein are compounds of formulae (I), (Ia), (Ib), (Ic), (Id), (Ie), (If), (Ig), (Ih), (Ii), (Ij), (Ik) and (II), as described below.

Further provided is a pharmaceutical composition comprising a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof; and optionally further comprising a pharmaceutically acceptable carrier, diluent or excipient.

In another aspect, provided is a method of inhibiting a Janus kinase activity (e.g., a JAK1 kinase activity) in a cell, comprising introducing into said cell an amount effective to inhibit said kinase of a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof. Also provided is a method of inhibiting an JAK kinase activity comprising contacting a Janus kinase (e.g., a JAK1 kinase) with a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof.

Another aspect includes a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, for use in therapy, such as the treatment of an inflammatory disease or cancer.

Another aspect includes a method of preventing, treating or lessening the severity of a disease or condition responsive to the inhibition of a Janus kinase, such as JAK1 kinase, in a patient. The method can comprise administering to the patient a therapeutically effective amount of a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof.

Another aspect includes the use of a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, in the manufacture of a medicament for the treatment of a disease responsive to the inhibition of a Janus kinase, such as JAK1 kinase.

Another aspect includes a kit for treating a disease or disorder responsive to the inhibition of a Janus kinase, such as JAK1 kinase. The kit can comprise a first pharmaceutical composition comprising a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, and instructions for use.

### DETAILED DESCRIPTION OF THE INVENTION

The invention provides, inter alia, triazolopyridine compounds, and stereoisomers, tautomers, salts (e.g., pharmaceutically acceptable salts), solvates and prodrugs thereof. Compositions (e.g., pharmaceutical compositions) comprising the triazolopyridine compounds, and pharmaceutical formulations thereof, are useful in inhibiting a JAK kinase, such as JAK1, in a cell, and in the treatment of diseases, conditions and/or disorders responsive to the inhibition of a JAK kinase activity in a patient.

### Definition

The term “a” or “an” as used herein, unless clearly indicated otherwise, refers to one or more.

Reference to “about” a value or parameter herein includes (and describes) embodiments that are directed to that value or parameter per se. For example, description referring to “about X” includes description of “X”.

“Halogen” or “halo” refers to F, Cl, Br or I. Additionally, terms such as “haloalkyl,” are meant to include monohaloalkyl and polyhaloalkyl.

The term “alkyl” refers to a saturated linear or branched-chain monovalent hydrocarbon radical, wherein the alkyl radical may be optionally substituted. In one example, the alkyl radical is one to eighteen carbon atoms (C₁-C₁₈). In other examples, the alkyl radical is C₀-C₆, C₀-C₅, C₀-C₃, C₁-C₁₂, C₁-C₁₀, C₁-C₈, C₁-C₆, C₁-C₅, C₁-C₄, or C₁-C₃. Co alkyl refers to a bond. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl and 1-octyl.

In some embodiments, substituents for “optionally substituted alkyls” include one to four instances of F, Cl, Br, I, OH, SH, CN, NH₂, NHCH₃, N(CH₃)₂, NO₂, N₃, C(O)CH₃, COOH, CO₂CH₃, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, phenyl and heterocyclic portions thereof may be optionally substituted, such as by one to four instances of substituents selected from this same list.

The term “alkenyl” refers to linear or branched-chain monovalent hydrocarbon radical with at least one site of unsaturation, i.e., a carbon-carbon double bond, wherein the alkenyl radical may be optionally substituted, and includes radicals having “cis” and “trans” orientations, or alternatively, “E” and “Z” orientations. In one example, the alkenyl radical is two to eighteen carbon atoms (C₂-C₁₈). In other examples, the alkenyl radical is C₂-C₁₂, C₂-C₁₀, C₂-C₈, C₂-C₆ or C₂-C₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl. In some embodiments, substituents for “optionally substituted alkenyls” include one to four instances of F, Cl, Br, I, OH, SH, CN, NH₂, NHCH₃, N(CH₃)₂, NO₂, N₃, C(O)CH₃, COOH, CO₂CH₃, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, phenyl and heterocyclic portions thereof may be optionally substituted, such as by one to four instances of substituents selected from this same list.

The term “alkynyl” refers to a linear or branched monovalent hydrocarbon radical with at least one site of unsaturation, i.e., a carbon-carbon, triple bond, wherein the alkynyl radical may be optionally substituted. In one example, the alkynyl radical is two to eighteen carbon atoms (C₂-C₁₈). In other examples, the alkynyl radical is C₂-C₁₂, C₂-C₁₀, C₂-C₈, C₂-C₆ or C₂-C₃. Examples include, but are not limited to, ethynyl (—C≡CH), prop-1-ynyl (—C≡CCH₃), prop-2-ynyl (propargyl, —CH₂C≡CH), but-1-ynyl, but-2-ynyl and but-3-ynyl. In some embodiments, substituents for “optionally substituted alkynyls” include one to four instances of F, Cl, Br, I, OH, SH, CN, NH₂, NHCH₃, N(CH₃)₂, NO₂, N₃, C(O)CH₃, COOH, CO₂CH₃, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, phenyl and heterocyclic portions thereof may be optionally substituted, such as by one to four instances of substituents selected from this same list.

“Alkylene” refers to a saturated, branched or straight chain hydrocarbon group having two monovalent radical centers derived by the removal of two hydrogen atoms from the same or two different carbon atoms of a parent alkane. In one example, the divalent alkylene group is one to eighteen carbon atoms (C₁-C₁₈). In other examples, the divalent alkylene group is C₀-C₆, C₀-C₅, C₀-C₃, C₁-C₁₂, C₁-C₁₀, C₁-C₈, C₁-C₆, C₁-C₅, C₁-C₄, or C₁-C₃. The group Co alkylene refers to a bond. Example alkylene groups include methylene (—CH₂—), 1,1-ethyl (—CH(CH₃)—), (1,2-ethyl (—CH₂CH₂—), 1,1-propyl (—CH(CH₂CH₃)—), 2,2-propyl (—C(CH₃)₂—), 1,2-propyl (—CH(CH₃)CH₂—), 1,3-propyl (—CH₂CH₂CH₂—), 1,1-dimethyleth-1,2-yl (—C(CH₃)₂CH₂—), 1,4-butyl (—CH₂CH₂CH₂CH₂—), and the like.

“Alkenylene” refers to an unsaturated, branched or straight chain hydrocarbon group having two monovalent radical centers derived by the removal of two hydrogen atoms from the same or two different carbon atoms of a parent alkene. In one example, the alkenylene group is two to eighteen carbon atoms (C₂-C₁₈). In other examples, the alkenylene group is C₂-C₁₂, C₂-C₁₀, C₂-C₈, C₂-C₆ or C₂-C₃. An exemplary alkenylene group is 1,2-ethylene (—CH═CH—).

“Alkynylene” refers to an unsaturated, branched or straight chain hydrocarbon group having two monovalent radical centers derived by the removal of two hydrogen atoms from the same or two different carbon atoms of a parent alkyne. In one example, the alkynylene radical is two to eighteen carbon atoms (C₂-C₁₈). In other examples, the alkynylene radical is C₂-C₁₂, C₂-C₁₀, C₂-C₈, C₂-C₆ or C₂-C₃. Example alkynylene radicals include: acetylene (—C≡C—), propargyl (—CH₂C≡C—), and 4-pentynyl (—CH₂CH₂CH₂C≡C—).

The term “heteroalkyl” refers to a straight or branched chain monovalent hydrocarbon radical, consisting of the stated number of carbon atoms, or, if none are stated, up to 18 carbon atoms, and from one to five heteroatoms selected from the group consisting of O, N, Si and S, and wherein the nitrogen and sulfur atoms can optionally be oxidized and the nitrogen heteroatom can optionally be quaternized. In some embodiments, the heteroatom is selected from O, N and S, wherein the nitrogen and sulfur atoms can optionally be oxidized and the nitrogen heteroatom can optionally be quaternized. The heteroatom(s) can be placed at any interior position of the heteroalkyl group, including the position at which the alkyl group is attached to the remainder of the molecule (e.g., —O—CH₂—CH₃). Examples include —CH₂—CH₂—O—CH₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —Si(CH₃)₃ and —CH₂—CH═N—OCH₃. Up to two heteroatoms can be consecutive, such as, for example, —CH₂—NH—OCH₃ and —CH₂—O—Si(CH₃)₃. Heteroalkyl groups can be optionally substituted. In some embodiments, substituents for “optionally substituted heteroalkyls” include one to four instances of F, Cl, Br, I, OH, SH, CN, NH₂, NHCH₃, N(CH₃)₂, NO₂, N₃, C(O)CH₃, COOH, CO₂CH₃, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, phenyl and heterocyclic portions thereof may be optionally substituted, such as by one to four instances of substituents selected from this same list.

“Amidine” means the group —C(NH)—NHR in which R is hydrogen, alkyl, cycloalkyl, aryl or heterocyclyl, wherein the alkyl, cycloalkyl, aryl and heterocyclyl groups are as defined herein. A particular amidine is the group —C(NH)—NH₂.

“Amino” means primary (i.e., —NH₂), secondary (i.e., —NRH) and tertiary (i.e., —NRR) amines, that are optionally substituted, in which R is alkyl, cycloalkyl, aryl, or heterocyclyl, wherein the alkyl, cycloalkyl, aryl and heterocyclyl groups are as defined herein. Particular secondary and tertiary amines are alkylamine, dialkylamine, arylamine, diarylamine, aralkylamine and diaralkylamine, wherein the alkyl and aryl portions can be optionally substituted. Particular secondary and tertiary amines are methylamine, ethylamine, propylamine, isopropylamine, phenylamine, benzylamine, dimethylamine, diethylamine, dipropylamine and diisopropylamine.

“Aryl” refers to a carbocyclic aromatic group, whether or not fused to one or more groups, having the number of carbon atoms designated, or if no number is designated, up to 14 carbon atoms. One example includes aryl groups having 6-14 carbon atoms. Another example includes aryl groups having 6-10 carbon atoms. Examples of aryl groups include phenyl, naphthyl, biphenyl, phenanthrenyl, naphthacenyl, 1,2,3,4-tetrahydronaphthalenyl, 1H-indenyl, 2,3-dihydro-1H-indenyl, and the like (see, e.g., Lang's Handbook of Chemistry (Dean, J. A., ed.) 13^{th} ed. Table 7-2 [1985]). A particular aryl is phenyl. Substituted phenyl or substituted aryl means a phenyl group or aryl group substituted with one, two, three, four or five substituents, for example, 1-2, 1-3 or 1-4 substituents, such as chosen from groups specified herein (see “optionally substituted” definition), such as F, Cl, Br, I, OH, SH, CN, NH₂, NHCH₃, N(CH₃)₂, NO₂, N₃, C(O)CH₃, COOH, CO₂CH₃, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, phenyl and heterocyclic portions thereof may be optionally substituted, such as by one to four instances of substituents selected from this same list. Examples of the term “substituted phenyl” include a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl, 2,4-difluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(alkyl)phenyl group such as 4-methylphenyl, 2,4-dimethylphenyl, 2-methylphenyl, 4-(isopropyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such 4-carboxyphenyl, a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; or a mono- or di(N-(methylsulfonylamino))phenyl such as 3-(N-methylsulfonylamino))phenyl. Also, the term “substituted phenyl” represents disubstituted phenyl groups where the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, 2-chloro-5-difluoromethoxy and the like, as well as trisubstituted phenyl groups where the substituents are different, for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino.

“Cycloalkyl” refers to a non-aromatic, saturated or partially unsaturated hydrocarbon ring group wherein the cycloalkyl group may be optionally substituted independently with one or more substituents described herein. In one example, the cycloalkyl group is 3 to 12 carbon atoms (C₃-C₁₂). In other examples, cycloalkyl is C₃-C₈, C₃-C₁₀ or C₅-C₁₀. In other examples, the cycloalkyl group, as a monocycle, is C₃-C₈, C₃-C₆ or C₅-C₆. In another example, the cycloalkyl group, as a bicycle, is C₇-C₁₂. In another example, the cycloalkyl group, as a spiro system, is C₅-C₁₂. Examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[2.2.2]octane and bicyclo[3.2.2]nonane. Examples of spiro cycloalkyl include, spiro[2.2]pentane, spiro[2.3]hexane, spiro[2.4]heptane, spiro[2.5]octane and spiro[4.5]decane. In some embodiments, substituents for “optionally substituted cycloalkyls” include one to four instances of F, Cl, Br, I, OH, SH, CN, NH₂, NHCH₃, N(CH₃)₂, NO₂, N₃, C(O)CH₃, COOH, CO₂CH₃, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, aryl and heterocyclic portions thereof may be optionally substituted, such as by one to four instances of substituents selected from this same list.

“Guanidine” or “guanidinyl” means the group —NH—C(NH)—NHR in which R is hydrogen, alkyl, cycloalkyl, aryl or heterocyclyl, wherein the alkyl, cycloalkyl, aryl and heterocyclyl groups are as defined herein. A particular guanidine is the group —NH—C(NH)—NH₂.

“Heterocyclic group”, “heterocyclic”, “heterocycle”, “heterocyclyl”, or “heterocyclo” are used interchangeably and refer to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocycle, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 1 to 4 heteroatoms. In one example, heterocyclyl includes 1 to 3 heteroatoms. In another example, heterocyclyl includes 3- to 7-membered monocycles having 1-2, 1-3 or 1-4 heteroatoms selected from nitrogen, sulfur or oxygen. In another example, heterocyclyl includes 4- to 6-membered monocycles having 1-2, 1-3 or 1-4 heteroatoms selected from nitrogen, sulfur or oxygen. In another example, heterocyclyl includes 3-membered monocycles. In another example, heterocyclyl includes 4-membered monocycles. In another example, heterocyclyl includes 5-6 membered monocycles, e.g., 5-6 membered heteroaryl.

In another example, heterocyclyl includes 3-11 membered heterocycloyalkyls, such as 4-11 membered heterocycloalkyls. In some embodiments, a heterocycloalkyl includes at least one nitrogen. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]+OH⁻). Example heterocycles are oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, isoquinolinyl, tetrahydroisoquinolinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoi sothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocycles containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocycles containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Example benzo-fused 5-membered heterocycles are benzoxazol-2-yl, benzthiazol-2-yl and benzimidazol-2-yl. Example 6-membered heterocycles contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are other example heterocycle groups. Heterocycles may be optionally substituted. For example, substituents for “optionally substituted heterocycles” include one to four instances of F, Cl, Br, I, OH, SH, CN, NH₂, NHCH₃, N(CH₃)₂, NO₂, N₃, C(O)CH₃, COOH, CO₂CH₃, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, aryl and heterocyclic portions thereof may be optionally substituted, such as by one to four instances of substituents selected from this same list.

“Heteroaryl” refers to any mono-, bi-, or tricyclic ring system where at least one ring is a 5- or 6-membered aromatic ring containing from 1 to 4 heteroatoms selected from nitrogen, oxygen, and sulfur, and in an example embodiment, at least one heteroatom is nitrogen. See, for example, Lang's Handbook of Chemistry (Dean, J. A., ed.) 13^{th} ed. Table 7-2 [1985]. Included in the definition are any bicyclic groups where any of the above heteroaryl rings are fused to an aryl ring, wherein the aryl ring or the heteroaryl ring is joined to the remainder of the molecule. In one embodiment, heteroaryl includes 5-6 membered monocyclic aromatic groups where one or more ring atoms is nitrogen, sulfur or oxygen. Example heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl and purinyl, as well as benzo-fused derivatives, for example benzoxazolyl, benzofuryl, benzothiazolyl, benzothiadiazolyl, benzotriazolyl, benzoimidazolyl and indolyl. Heteroaryl groups can be optionally substituted. In some embodiments, substituents for “optionally substituted heteroaryls” include one to four instances of F, Cl, Br, I, OH, SH, CN, NH₂, NHCH₃, N(CH₃)₂, NO₂, N₃, C(O)CH₃, COOH, CO₂CH₃, methyl, ethyl, propyl, iso-propyl, butyl, isobutyl, cyclopropyl, methoxy, ethoxy, propoxy, trifluoromethyl, difluoromethyl, sulfonylamino, methanesulfonylamino, SO, SO₂, phenyl, piperidinyl, piperizinyl, and pyrimidinyl, wherein the alkyl, phenyl and heterocyclic portions thereof may be optionally substituted, such as by one to four instances of substituents selected from this same list.

“Heteroarylene” refers to a heteroaryl having two monovalent radical centers derived by the removal of two hydrogen atoms from two different atoms of a parent heteroaryl group.

In particular embodiments, a heterocyclyl group is attached at a carbon atom of the heterocyclyl group. By way of example, carbon bonded heterocyclyl groups include bonding arrangements at position 2, 3, 4, 5, or 6 of a pyridine ring, position 3, 4, 5, or 6 of a pyridazine ring, position 2, 4, 5, or 6 of a pyrimidine ring, position 2, 3, 5, or 6 of a pyrazine ring, position 2, 3, 4, or 5 of a furan, tetrahydrofuran, thiofuran, thiophene, pyrrole or tetrahydropyrrole ring, position 2, 4, or 5 of an oxazole, imidazole or thiazole ring, position 3, 4, or 5 of an isoxazole, pyrazole, or isothiazole ring, position 2 or 3 of an aziridine ring, position 2, 3, or 4 of an azetidine ring, position 2, 3, 4, 5, 6, 7, or 8 of a quinoline ring or position 1, 3, 4, 5, 6, 7, or 8 of an isoquinoline ring.

In certain embodiments, the heterocyclyl group is N-attached. By way of example, nitrogen bonded heterocyclyl or heteroaryl groups include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The term “alkoxy” refers to a linear or branched monovalent radical represented by the formula —OR in which R is alkyl, as defined herein. Alkoxy groups include methoxy, ethoxy, propoxy, isopropoxy, mono-, di- and tri-fluoromethoxy and cyclopropoxy.

“Acyl” means a carbonyl containing substituent represented by the formula —C(O)—R in which R is hydrogen, alkyl, cycloalkyl, aryl or heterocyclyl, wherein the alkyl, cycloalkyl, aryl and heterocyclyl are as defined herein. Acyl groups include alkanoyl (e.g., acetyl), aroyl (e.g., benzoyl), and heteroaroyl (e.g., pyridinoyl).

“Optionally substituted” unless otherwise specified means that a group may be unsubstituted or substituted by one or more (e.g., 1, 2, 3, 4, or 5 or more, or any range derivable therein) of the substituents listed for that group in which said substituents may be the same or different. In an embodiment, an optionally substituted group has 1 substituent. In another embodiment an optionally substituted group has 2 substituents. In another embodiment an optionally substituted group has 3 substituents. In another embodiment an optionally substituted group has 4 substituents. In another embodiment an optionally substituted group has 5 substituents.

Optional substituents for alkyl radicals, alone or as part of another substituent (e.g., alkoxy), as well as alkylenyl, alkenyl, alkynyl, heteroalkyl, heterocycloalkyl, and cycloalkyl, also each alone or as part of another substituent, can be a variety of groups, such as those described herein, as well as selected from the group consisting of halogen; oxo; CN; NO; N₃; —OR′; perfluoro-C₁-C₄ alkoxy; unsubstituted C₃-C₇ cycloalkyl; C₃-C₇ cycloalkyl substituted by halogen, OH, CN, unsubstituted C₁-C₆ alkyl, unsubstituted C₁-C₆ alkoxy, oxo or NR′R″; unsubstituted C₆-C₁₀ aryl (e.g., phenyl); C₆-C₁₀ aryl substituted by halogen, OH, CN, unsubstituted C₁-C₆ alkyl, unsubstituted C₁-C₆ alkoxy, or NR′R″; unsubstituted 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S); 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S) substituted by halogen, OH, CN, unsubstituted C₁-C₆ alkyl, unsubstituted C₁-C₆ alkoxy, oxo or NR′R″; —NR′R″; —SR′; —SiR′R″R′″; —OC(O)R′; —C(O)R′; —CO₂R′; —CONR′R″; —OC(O)NR′R″; —NR″C(O)R′; —NR′″C(O)NR′R″; —NR″C(O)₂R′; —S(O)₂R′; —S(O)₂NR′R″; —NR'S(O)₂R″; —NR″ 'S(O)₂NR′R″; amidinyl; guanidinyl; —(CH₂)₁₋₄—OR′; —(CH₂)₁₋₄—NR′R″; —(CH₂)₁₋₄—SR′; —(CH₂)₁₋₄—SiR′R″R′″; —(CH₂)₁₋₄—OC(O)R′; —(CH₂)₁₋₄—C(O)R′; —(CH₂)₁₋₄—CO₂R′; and —(CH₂)₁₋₄CONR′R″, or combinations thereof, in a number ranging from zero to (2m′+1), where m′ is the total number of carbon atoms in such radical. R′, R″ and R′″each independently refer to groups including, for example, hydrogen; unsubstituted C₁-C₆ alkyl; C₁-C₆ alkyl substituted by halogen, OH, CN, unsubstituted C₁-C₆ alkyl, unsubstituted C₁-C₆ alkoxy, oxo or NR′R″; unsubstituted C₁-C₆ heteroalkyl; C₁-C₆ heteroalkyl substituted by halogen, OH, CN, unsubstituted C₁-C₆ alkyl, unsubstituted C₁-C₆ alkoxy, oxo or NR′R″; unsubstituted C₆-C₁₀ aryl; C₆-C₁₀ aryl substituted by halogen, OH, CN, unsubstituted C₁-C₆ alkyl, unsubstituted C₁-C₆ alkoxy, or NR′R″; unsubstituted 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S); and 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S) substituted by halogen, OH, CN, unsubstituted C₁-C₆ alkyl, unsubstituted C₁-C₆ alkoxy, oxo or NR′R″. When R′ and R″ are attached to the same nitrogen atom, they can be combined with the nitrogen atom to form a 3-, 4-, 5-, 6-, or 7-membered ring wherein a ring atom is optionally substituted with N, O or S and wherein the ring is optionally substituted with halogen, OH, CN, unsubstituted C₁-C₆ alkyl, unsubstituted C₁-C₆ alkoxy, oxo or NR′R″. For example, —NR′R″ is meant to include 1-pyrrolidinyl and 4-morpholinyl.

Similarly, optional substituents for the aryl and heteroaryl groups are varied. In some embodiments, substituents for aryl and heteroaryl groups are selected from the group consisting of halogen; CN; NO; N₃; —OR′; perfluoro-C₁-C₄ alkoxy; unsubstituted C₃-C₇ cycloalkyl; C₃-C₇ cycloalkyl substituted by halogen, OH, CN, unsubstituted C₁-C₆ alkyl, unsubstituted C₁-C₆ alkoxy, oxo or NR′R″; unsubstituted C₆-C₁₀ aryl (e.g., phenyl); C₆-C₁₀ aryl substituted by halogen, OH, CN, unsubstituted C₁-C₆ alkyl, unsubstituted C₁-C₆ alkoxy, or NR′R″; unsubstituted 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S); 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S) substituted by halogen, OH, CN, unsubstituted C₁-C₆ alkyl, unsubstituted C₁-C₆ alkoxy, oxo or

NR′R″; —NR′R″; —SR′; —SiR′R″R′″; —OC(O)R′; —C(O)R′; —CO₂R′; —CONR′R″; —OC(O)NR′R″; —N R″C(O)R′; —NR′″C(O)NR′R″; —NR″C(O)₂R′; —S(O)₂R′; —S(O)₂NR′R″; —NR'S(O)₂R″; —NR′″S(O)₂NR′R″; amidinyl; guanidinyl; —(CH₂)₁₋₄—OR′; —(CH₂)₁₋₄—NR′R″; —(CH₂)₁₋₄—SR′; —(CH₂)₁₋₄-SiR′R″R′″; —(CH₂)₁₋₄—OC(O)R′; —(CH₂)₁₋₄—C(O)R′; —(CH₂)₁₋₄—CO₂R′; and —(CH₂)₁₋₄CONR′R″, or combinations thereof, in a number ranging from zero to (2m′+1), where m′ is the total number of carbon atoms in such radical. R′, R″ and R′″each independently refer to groups including, for example, hydrogen; unsubstituted C₁-C₆ alkyl; C₁-C₆ alkyl substituted by halogen, OH, CN, unsubstituted C₁-C₆ alkyl, unsubstituted C₁-C₆ alkoxy, oxo or NR′R″; unsubstituted C₁-C₆ heteroalkyl; C₁-C₆ heteroalkyl substituted by halogen, OH, CN, unsubstituted C₁-C₆ alkyl, unsubstituted C₁-C₆ alkoxy, oxo or NR′R″; unsubstituted C₆-C₁₀ aryl; C₆-C₁₀ aryl substituted by halogen, OH, CN, unsubstituted C₁-C₆ alkyl, unsubstituted C₁-C₆ alkoxy, or NR′R″; unsubstituted 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S); and 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S) substituted by halogen, OH, CN, unsubstituted C₁-C₆ alkyl, unsubstituted C₁-C₆ alkoxy, oxo or NR′R″. When R′ and R″ are attached to the same nitrogen atom, they can be combined with the nitrogen atom to form a 3-, 4-, 5-, 6-, or 7-membered ring wherein a ring atom is optionally substituted with N, O or S and wherein the ring is optionally substituted with halogen, OH, CN, unsubstituted C₁-C₆ alkyl, unsubstituted C₁-C₆ alkoxy, oxo or NR′R″. For example, —NR′R″ is meant to include 1-pyrrolidinyl and 4-morpholinyl.

The term “oxo” refers to ═O or (═O)₂.

As used herein a wavy line “ [symbol] ” that intersects a bond in a chemical structure indicate the point of attachment of the atom to which the wavy bond is connected in the chemical structure to the remainder of a molecule, or to the remainder of a fragment of a molecule. In some embodiments, an arrow together with an asterisk is used in the manner of a wavy line to indicate a point of attachment.

In certain embodiments, divalent groups are described generically without specific bonding configurations. It is understood that the generic description is meant to include both bonding configurations, unless specified otherwise. For example, in the group R¹—R²—R³, if the group R² is described as —CH₂C(O)—, then it is understood that this group can be bonded both as R¹—CH₂C(O)—R³, and as R¹—C(O)CH₂—R³, unless specified otherwise.

The phrase “pharmaceutically acceptable” refers to molecular entities and compositions that do not produce an adverse, allergic or other untoward reaction when administered to an animal, such as, for example, a human, as appropriate.

Compounds of the present invention may be in the form of a salt, such as a pharmaceutically acceptable salt. “Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, benzenesulfonic acid, p-toluenesulfonic acid, salicyclic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particular base addition salts are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethyl aminoethanol, tromethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particular organic non-toxic bases include isopropylamine, diethylamine, ethanolamine, tromethamine, dicyclohexylamine, choline, and caffeine.

In some embodiments, a salt is selected from a hydrochloride, hydrobromide, trifluoroacetate, sulphate, phosphate, acetate, fumarate, maleate, tartrate, lactate, citrate, pyruvate, succinate, oxalate, methanesulphonate, p-toluenesulphonate, bisulphate, benzenesulphonate, ethanesulphonate, malonate, xinafoate, ascorbate, oleate, nicotinate, saccharinate, adipate, formate, glycolate, palmitate, L-lactate, D-lactate, aspartate, malate, L-tartrate, D-tartrate, stearate, furoate (e.g., 2-furoate or 3-furoate), napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate, 2-naphthalenesulphonate, 2,5-dichlorobenzenesulphonate, D-mandelate, L-mandelate, cinnamate, benzoate, adipate, esylate, malonate, mesitylate (2-mesitylenesulphonate), napsylate (2-naphthalenesulfonate), camsylate (camphor-10-sulphonate, for example (1S)-(+)-10-camphorsulfonic acid salt), glutamate, glutarate, hippurate (2-(benzoylamino)acetate), orotate, xylate (p-xylene-2-sulphonate), and pamoic (2,2′-dihydroxy-1,1′-dinaphthylmethane-3,3′-dicarboxylate).

A “sterile” formulation is aseptic or free from all living microorganisms and their spores.

“Stereoisomers” refer to compounds that have identical chemical constitution, but differ with regard to the arrangement of the atoms or groups in space. Stereoisomers include diastereomers, enantiomers, conformers and the like.

“Chiral” refers to molecules which have the property of non-superimposability of the mirror image partner, while the term “achiral” refers to molecules which are superimposable on their mirror image partner.

“Diastereomer” refers to a stereoisomer with two or more centers of chirality and whose molecules are not mirror images of one another. Diastereomers have different physical properties, e.g., melting points, boiling points, spectral properties or biological activities. Mixtures of diastereomers may separate under high resolution analytical procedures such as electrophoresis and chromatography such as HPLC.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Certain compounds of the present invention can exist in unsolvated forms as well as solvated forms, including hydrated forms. A “solvate” refers to an association or complex of one or more solvent molecules and a compound of the present invention. Examples of solvents that form solvates include water, isopropanol, ethanol, methanol, DMSO, ethyl acetate, acetic acid, and ethanolamine. Certain compounds of the present invention can exist in multiple crystalline or amorphous forms. In general, all physical forms are intended to be within the scope of the present invention. The term “hydrate” refers to the complex where the solvent molecule is water.

A “metabolite” refers to a product produced through metabolism in the body of a specified compound or salt thereof. Such products can result, for example, from the oxidation, reduction, hydrolysis, amidation, deamidation, esterification, deesterification, enzymatic cleavage, and the like, of the administered compound.

Metabolite products typically are identified by preparing a radiolabelled (e.g., ¹⁴C or ³H) isotope of a compound of the invention, administering it in a detectable dose (e.g., greater than about 0.5 mg/kg) to an animal such as rat, mouse, guinea pig, monkey, or to a human, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

“Amino-protecting group” as used herein refers to a derivative of the groups commonly employed to block or protect an amino group while reactions are carried out on other functional groups on the compound. Examples of such protecting groups include carbamates, amides, alkyl and aryl groups, and imines, as well as many N-heteroatom derivatives which can be removed to regenerate the desired amine group. Particular amino protecting groups are Pmb (p-Methoxybenzyl), Boc (tert-Butyloxycarbonyl), Fmoc (9-Fluorenylmethyloxycarbonyl) and Cbz (Carbobenzyloxy). Further examples of these groups are found in T. W. Greene and P. G. M. Wuts, “Protecting Groups in Organic Synthesis, 3^{rd} ed., John Wiley & Sons, Inc., 1999. The term “protected amino” refers to an amino group substituted with one of the above amino-protecting groups.

“Carboxy-protecting group” as used herein refers to those groups that are stable to the conditions of subsequent reaction(s) at other positions of the molecule, which may be removed at the appropriate point without disrupting the remainder of the molecule, to give the unprotected carboxy-group. Examples of carboxy protecting groups include, ester groups and heterocyclyl groups. Ester derivatives of the carboxylic acid group may be employed to block or protect the carboxylic acid group while reactions are carried out on other functional groups on the compound. Examples of such ester groups include substituted arylalkyl, including substituted benzyls, such as 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, alkyl or substituted alkyl esters such as methyl, ethyl, t-butyl allyl or t-amyl, triphenylmethyl (trityl), 4-methoxytrityl, 4,4′-dimethoxytrityl, 4,4′,4″-trimethoxytrityl, 2-phenylprop-2-yl, thioesters such as t-butyl thioester, silyl esters such as trimethylsilyl, t-butyldimethylsilyl esters, phenacyl, 2,2,2-trichloroethyl, beta-(trimethylsilyl)ethyl, beta-(di(n-butyl)methylsilyl)ethyl, p-toluenesulfonyl ethyl, 4-nitrobenzyl sulfonylethyl, allyl, cinnamyl, 1-(trimethylsilylmethyl)prop-1-en-3-yl, and like moieties. Another example of carboxy-protecting groups are heterocyclyl groups such as 1,3-oxazolinyl. Further examples of these groups are found in T. W. Greene and P. G. M. Wuts, “Protecting Groups in Organic Synthesis, 3^{rd} ed., John Wiley & Sons, Inc., 1999. The term “protected carboxy” refers to a carboxy group substituted with one of the above carboxy-protecting groups.

“Hydroxy-protecting group” as used herein refers to a derivative of the hydroxy group commonly employed to block or protect the hydroxy group while reactions are carried out on other functional groups on the compound. Examples of such protecting groups include tetrahydropyranyloxy, benzoyl, acetoxy, carbamoyloxy, benzyl, and silylethers (e.g., TBS, TBDPS) groups. Further examples of these groups are found in T. W. Greene and P. G. M. Wuts, “Protecting Groups in Organic Synthesis, 3^{rd} ed., John Wiley & Sons, Inc., 1999. The term “protected hydroxy” refers to a hydroxy group substituted with one of the above hydroxy-protecting groups.

A “subject,” “individual,” or “patient” is a vertebrate. In certain embodiments, the vertebrate is a mammal. Mammals include, but are not limited to, farm animals (such as cows), sport animals, pets (such as guinea pigs, cats, dogs, rabbits and horses), primates, mice and rats. In certain embodiments, a mammal is a human. In embodiments comprising administration of a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, to a patient, the patient is typically in need thereof.

The term “Janus kinase” refers to JAK1, JAK2, JAK3 and TYK2 protein kinases. In some embodiments, a Janus kinase may be further defined as one of JAK1, JAK2, JAK3 or TYK2. In any embodiment, any one of JAK1, JAK2, JAK3 and TYK2 may be specifically excluded as a Janus kinase. In some embodiments, a Janus kinase is JAK1. In some embodiments, a Janus kinase is a combination of JAK1 and JAK2.

The terms “inhibiting” and “reducing,” or any variation of these terms, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity (e.g., JAK1 activity) compared to normal.

In some embodiments, a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, is selective for inhibition of JAK1 over JAK3 and TYK2. In some embodiments, a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, is selective for inhibition of JAK1 over JAK2, JAK3, or TYK2, or any combination of JAK2, JAK3, or TYK2. In some embodiments, a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, is selective for inhibition of JAK1 and JAK2 over JAK3 and TYK2. In some embodiments, a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, is selective for inhibition of JAK1 over JAK3. By “selective for inhibition” it is meant that the compound is at least a 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, better inhibitor of a particular Janus kinase (e.g., JAK1) activity compared to another particular Janus kinase (e.g., JAK1) activity, or is at least a 2-, 3-, 4-, 5-, 10-, 25-, 50-, 100-, 250-, or 500-fold better inhibitor of a particular Janus kinase (e.g., JAK1) activity compared to another particular Janus kinase (e.g., JAK1) activity.

“Therapeutically effective amount” means an amount of a compound of the present invention, such as a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, that (i) treats or prevents the particular disease, condition or disorder, or (ii) attenuates, ameliorates or eliminates one or more symptoms of the particular disease, condition, or disorder, and optionally (iii) prevents or delays the onset of one or more symptoms of the particular disease, condition or disorder described herein. In some embodiments, the therapeutically effective amount is an amount sufficient to decrease or alleviate the symptoms of an autoimmune or inflammatory disease (e.g., asthma). In some embodiments, a therapeutically effective amount is an amount of a chemical entity described herein sufficient to significantly decrease the activity or number of B-cells. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; or relieve to some extent one or more of the symptoms associated with the cancer. To the extent the drug may prevent growth or kill existing cancer cells, it may be cytostatic or cytotoxic. For cancer therapy, efficacy can, for example, be measured by assessing the time to disease progression (TTP) or determining the response rate (RR).

“Treatment” (and variations such as “treat” or “treating”) refers to clinical intervention in an attempt to alter the natural course of the individual or cell being treated. Desirable effects of treatment include preventing recurrence of disease, alleviation of symptoms, diminishment of any direct or indirect pathological consequences of the disease, stabilized (i.e., not worsening) state of disease, decreasing the rate of disease progression, amelioration or palliation of the disease state, prolonging survival as compared to expected survival if not receiving treatment and remission or improved prognosis. In some embodiments, compounds of the invention are used to slow the progression of a disease or disorder. Those in need of treatment include those already with the condition or disorder as well as those prone to have the condition or disorder, (for example, through a genetic mutation) or those in which the condition or disorder is to be prevented.

“Inflammatory disorder” refers to any disease, disorder or syndrome in which an excessive or unregulated inflammatory response leads to excessive inflammatory symptoms, host tissue damage, or loss of tissue function. “Inflammatory disorder” also refers to a pathological state mediated by influx of leukocytes or neutrophil chemotaxis.

“Inflammation” refers to a localized, protective response elicited by injury or destruction of tissues, which serves to destroy, dilute, or wall off (sequester) both the injurious agent and the injured tissue. Inflammation is notably associated with influx of leukocytes or neutrophil chemotaxis. Inflammation can result from infection with pathogenic organisms and viruses and from noninfectious means such as trauma or reperfusion following myocardial infarction or stroke, immune responses to foreign antigens, and autoimmune responses. Accordingly, inflammatory disorders amenable to treatment with a compound of the present invention, such as a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, encompass disorders associated with reactions of the specific defense system as well as with reactions of the nonspecific defense system.

“Specific defense system” refers to the component of the immune system that reacts to the presence of specific antigens. Examples of inflammation resulting from a response of the specific defense system include the classical response to foreign antigens, autoimmune diseases, and delayed type hypersensitivity responses mediated by T-cells. Chronic inflammatory diseases, the rejection of solid transplanted tissue and organs, e.g., kidney and bone marrow transplants, and graft versus host disease (GVHD), are further examples of inflammatory reactions of the specific defense system.

The term “nonspecific defense system” refers to inflammatory disorders that are mediated by leukocytes that are incapable of immunological memory (e.g., granulocytes, and macrophages). Examples of inflammation that result, at least in part, from a reaction of the nonspecific defense system include inflammation associated with conditions such as adult (acute) respiratory distress syndrome (ARDS) or multiple organ injury syndromes; reperfusion injury; acute glomerulonephritis; reactive arthritis; dermatoses with acute inflammatory components; acute purulent meningitis or other central nervous system inflammatory disorders such as stroke; thermal injury; inflammatory bowel disease; granulocyte transfusion associated syndromes; and cytokine-induced toxicity.

“Autoimmune disease” refers to any group of disorders in which tissue injury is associated with humoral or cell-mediated responses to the body's own constituents. Non-limiting examples of autoimmune diseases include rheumatoid arthritis, lupus and multiple sclerosis.

“Allergic disease” as used herein refers to any symptoms, tissue damage, or loss of tissue function resulting from allergy. “Arthritic disease” as used herein refers to any disease that is characterized by inflammatory lesions of the joints attributable to a variety of etiologies. “Dermatitis” as used herein refers to any of a large family of diseases of the skin that are characterized by inflammation of the skin attributable to a variety of etiologies. “Transplant rejection” as used herein refers to any immune reaction directed against grafted tissue, such as organs or cells (e.g., bone marrow), characterized by a loss of function of the grafted and surrounding tissues, pain, swelling, leukocytosis, and thrombocytopenia. The therapeutic methods of the present invention include methods for the treatment of disorders associated with inflammatory cell activation.

“Inflammatory cell activation” refers to the induction by a stimulus (including, but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including, but not limited to, major histocompatability antigens or cell adhesion molecules) in inflammatory cells (including but not limited to monocytes, macrophages, T lymphocytes, B lymphocytes, granulocytes (i.e., polymorphonuclear leukocytes such as neutrophils, basophils, and eosinophils), mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory disorder.

In some embodiments, inflammatory disorders which can be treated according to the methods of this invention include, but are not limited to, asthma, rhinitis (e.g., allergic rhinitis), allergic airway syndrome, atopic dermatitis, bronchitis, rheumatoid arthritis, psoriasis, contact dermatitis, chronic obstructive pulmonary disease and delayed hypersensitivity reactions.

The terms “cancer” and “cancerous”, “neoplasm”, and “tumor” and related terms refer to or describe the physiological condition in mammals that is typically characterized by unregulated cell growth. A “tumor” comprises one or more cancerous cells. Examples of cancer include carcinoma, blastoma, sarcoma, seminoma, glioblastoma, melanoma, leukemia, and myeloid or lymphoid malignancies. More particular examples of such cancers include squamous cell cancer (e.g., epithelial squamous cell cancer) and lung cancer including small-cell lung cancer, non-small cell lung cancer (“NSCLC”), adenocarcinoma of the lung and squamous carcinoma of the lung. Other cancers include skin, keratoacanthoma, follicular carcinoma, hairy cell leukemia, buccal cavity, pharynx (oral), lip, tongue, mouth, salivary gland, esophageal, larynx, hepatocellular, gastric, stomach, gastrointestinal, small intestine, large intestine, pancreatic, cervical, ovarian, liver, bladder, hepatoma, breast, colon, rectal, colorectal, genitourinary, biliary passage, thyroid, papillary, hepatic, endometrial, uterine, salivary gland, kidney or renal, prostate, testis, vulval, peritoneum, anal, penile, bone, multiple myeloma, B-cell lymphoma, central nervous system, brain, head and neck, Hodgkin's, and associated metastases. Examples of neoplastic disorders include myeloproliferative disorders, such as polycythemia vera, essential thrombocytosis, myelofibrosis, such as primary myelofibrosis, and chronic myelogenous leukemia (CML).

A “chemotherapeutic agent” is an agent useful in the treatment of a given disorder, for example, cancer or inflammatory disorders. Examples of chemotherapeutic agents are well-known in the art and include examples such as those disclosed in U.S. Publ. Appl. No. 2010/0048557, incorporated herein by reference. Additionally, chemotherapeutic agents include pharmaceutically acceptable salts, acids or derivatives of any of chemotherapeutic agents, as well as combinations of two or more of them.

“Package insert” is used to refer to instructions customarily included in commercial packages of therapeutic products that contain information about the indications, usage, dosage, administration, contraindications or warnings concerning the use of such therapeutic products.

The terms “compound(s) of this invention,” and “compound(s) of the present invention” and the like, unless otherwise indicated, include compounds of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), and stereoisomers (including atropisomers), geometric isomers, tautomers, solvates, metabolites, isotopes, salts (e.g., pharmaceutically acceptable salts), and prodrugs thereof. In some embodiments, solvates, metabolites, isotopes or prodrugs are excluded, or any combination thereof.

Unless otherwise stated, structures depicted herein are also meant to include compounds that differ only in the presence of one or more isotopically enriched atoms. Exemplary isotopes that can be incorporated into compounds of the present invention, such as a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5, and Letters A-S), or a stereoisomer, tautomer, solvate or prodrug thereof, or a pharmaceutically acceptable salt thereof, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

The use of the term “or” is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device or method being employed to determine the value.

As used herein, “a” or “an” means one or more, unless clearly indicated otherwise. As used herein, “another” means at least a second or more.

Headings used herein are intended only for organizational purposes.

### Inhibitors of Janus Kinases

Compounds according to the invention are detailed herein, including in the Brief Summary of the Invention and the appended claims. The invention includes the use of all of the compounds described herein, including any and all stereoisomers, including geometric isomers (cis/trans), salts (including pharmaceutically acceptable salts) and solvates of the compounds described herein, as well as methods of making such compounds.

In one aspect, provided is a compound of formula (I):

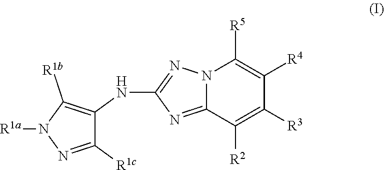

or a stereoisomer, tautomer, solvate, prodrug or salt thereof, wherein:

R^{1a} is hydrogen, C₁-C₆ alkyl, C₃-C₈ cycloalkyl, phenyl, or 3-11 membered heterocyclyl and R^{1a} is optionally substituted by R⁹;

R^{1b} and R^{1c} are each independently hydrogen, C₁-C₆ alkyl, or C₃-C₈ cycloalkyl;

R² is a 3-11 membered heterocyclyl containing at least 1 nitrogen, selected from groups (a)-(e) and (h)-(j); a C₅-C₈ cycloalkenyl ring (f); a —O—(CR^{x}R^{y})_{q}-Ar² group (g); or a Ar¹—O—(CR^{x}R^{y})_{q}-Ar² group (k), where each R^{x} and R^{y} are independently hydrogen or C₁-C₆ alkyl, each q is independently 0 to 3, Ar¹ is 1,4-phenylene and Ar² is optionally substituted C₆-C₁₀ aryl or optionally substituted 5-11 membered heteroaryl:

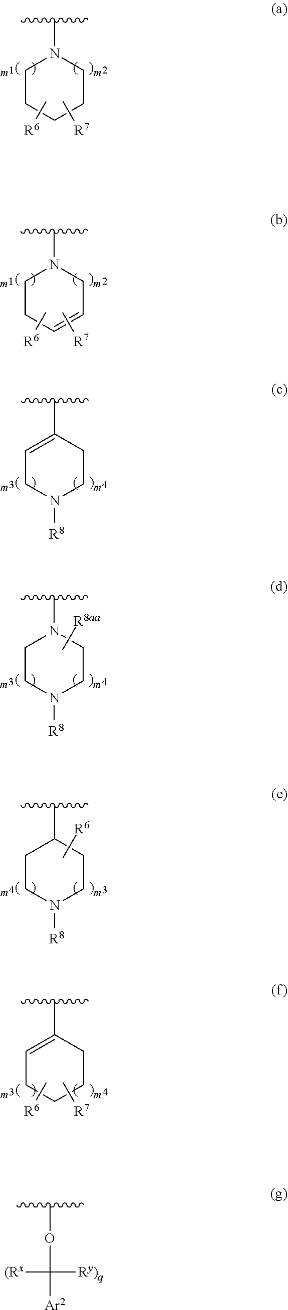

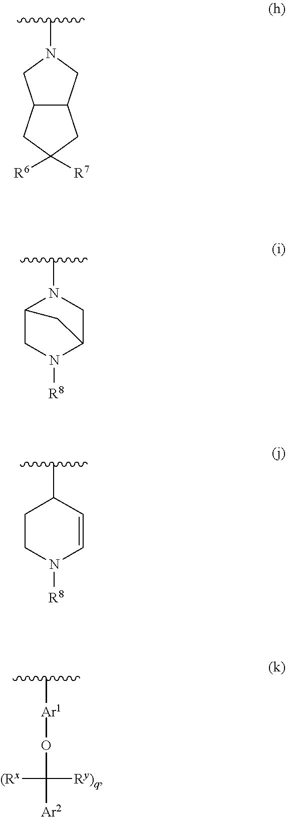

wherein the wavy line represents the point of attachment of R² in formula (I);

R³, R⁴ and R⁵ are each independently selected from the group consisting of hydrogen, CH₃, CH₂CH₃, OCH₃, CF₃, F and C₁;

R⁶ and R⁷ are independently selected from the group consisting of hydrogen, halogen, OH, CN, phenyl, C₁-C₆ alkyl, (C₀-C₆ alkylene)C₃-C₈ cycloalkyl, (C₀-C₆ alkylene)3-11 membered heterocyclyl, (C₀-C₆ alkylene)C(O)NR^{a}R^{b}, (C₀-C₆ alkylene)NR^{a}C(O)(C₁-C₆ alkyl), (C₀-C₆ alkylene)NR^{a}C(O)(phenyl), (C₀-C₆ alkylene)C(O)R^{8a}, (C₀-C₆ alkylene)C(O)OR^{8a}, C₁-C₆ alkoxy, —O—(C₃-C₆ cycloalkyl), —O—(C₀-C₆ alkylene)C(O)NR^{a}R^{b}, —C═N—O—(C₁-C₆ alkyl), —O—(C₁-C₆ alkyl)3-11 membered heterocyclyl, (C₀-C₆ alkylene)NR^{a}SO₂(C₁-C₆ alkyl), (C₀-C₆ alkylene)NR^{a}SO₂(phenyl), and —O-(3-11 membered heterocyclyl); wherein said alkyl, alkylene, alkoxy, cycloalkyl, phenyl and heterocyclyl are each independently optionally substituted,
or R⁶ and R⁷ together form an optionally substituted phenyl or optionally substituted 3-11 membered heterocyclyl;

R⁸ is H, C₁-C₆ alkyl, (C₀-C₆ alkylene)phenyl, (C₀-C₆ alkylene)C₃-C₈ cycloalkyl, (C₀-C₆ alkylene)3-11 membered heterocyclyl, C(O)NR^{a}R^{b}, SO₂NR^{a}R^{b}, (C₁-C₆ alkylene)C(O)OR^{8a} or C(O)R^{8a}, wherein said alkyl, alkylene, heterocyclyl and phenyl are each independently optionally substituted;

R^{8a} is H, NR^{a}R^{b}, C₁-C₆ alkyl, (C₀-C₆ alkylene)C₃-C₈ cycloalkyl, (C₀-C₆ alkylene)phenyl, or (C₀-C₆ alkylene)3-11 membered heterocyclyl, wherein said alkyl, alkylene, cycloalkyl, phenyl and heterocyclyl are each independently optionally substituted;

R^{8aa} is H, C₁-C₆ alkyl optionally substituted by OH, or C(O)NR^{a}R^{b}; or

or R⁸ and R^{8aa} together form an optionally substituted 3-11 membered heterocyclyl;

R⁹, independently at each occurrence, is OH, halogen, CN, C₁-C₆ alkyl, C₃-C₈ cycloalkyl, phenyl, 3-11 membered heterocyclyl, 5-11 membered heteroaryl, —C(O)NR^{a}R^{b}, —NR^{a}R^{b}, (C₁-C₆ alkylene)C₃-C₈ cycloalkyl, (C₁-C₆ alkylene)phenyl, (C₁-C₆ alkylene)3-11 membered heterocyclyl, (C₁-C₆ alkylene)5-11 membered heteroaryl, (C₁-C₆ alkylene)C(O)NR^{a}R^{b}, (C₁-C₆ alkylene)NR^{a}R^{b}, or C(O)(C₁-C₆ alkyl), wherein said alkyl, alkylene, cycloalkyl, phenyl, heterocyclyl and heteroaryl are each independently optionally substituted;

R^{a} and R^{b}, independently at each occurrence, are selected from the group consisting of hydrogen, C₁-C₆ alkyl optionally substituted by halogen or CN, (C₀-C₆ alkylene)C₃-C₈ cycloalkyl, or (C₀-C₆ alkylene)phenyl, and wherein one or more alkylene units of any alkyl group is independently optionally substituted by —O—, or alternatively R^{a} and R^{b} may be joined together with the nitrogen atom to which they are attached to form an optionally substituted 3-11 membered heterocyclyl; and

m¹, m², m³ and m⁴ are each independently 0, 1 or 2.

In some embodiments, the compound is of the formula (I) as defined herein, provided that the compounds is other than a compound selected from the group consisting of Compound Nos. 1x to 7x and salts thereof.

| Compound No. | Chemical Name |
|---|---|
| 1x | N-(1-isobutylpiperidin-4-yl)-N-methyl-2-(4-((8-(1-(4,4,4-trifluorobutanoyl)- |
| | 1,2,3,6-tetrahydropyridin-4-yl)-[1,2,4]triazolo[1,5-a]pyridin-2-yl)amino)- |
| | 1H-pyrazol-1-yl)acetamide |
| 2x | 2-(4-((8-(4-(4-chlorophenyl)-4-(hydroxymethyl)piperidin-1-yl)- |
| | [1,2,4]triazolo[1,5-a]pyridin-2-yl)amino)-1H-pyrazol-1-yl)-1-(4- |
| | (morpholinomethyl)piperidin-1-yl)ethanone |
| 3x | methyl 3-(4-(2-(4-((8-(4-(4-chlorophenyl)-4-(hydroxymethyl)piperidin-1- |
| | yl)-[1,2,4]triazolo[1,5-a]pyridin-2-yl)amino)-1H-pyrazol-1- |
| | yl)acetyl)piperazin-1-yl)propanoate formate |
| 4x | 2-(4-((8-(4-(4-chlorophenyl)-4-(hydroxymethyl)piperidin-1-yl)- |
| | [1,2,4]triazolo[1,5-a]pyridin-2-yl)amino)-1H-pyrazol-1-yl)-1-(4- |
| | (methylamino)piperidin-1-yl)ethanone |
| 5x | 3-((1-(2-(4-((8-(4-(4-chlorophenyl)-4-(hydroxymethyl)piperidin-1-yl)- |
| | [1,2,4]triazolo[1, 5-a]pyridin-2-yl)amino)-1H-pyrazol-1-yl)acetyl)piperidin- |
| | 4-yl)(methyl)amino)propanenitrile |
| 6x | (1-(2-((1H-pyrazol-4-yl)amino)-[1,2,4]triazolo[1,5-a]pyridin-8-yl)-4-(4- |
| | chlorophenyl)piperidin-4-yl)methanol |
| 7x | 8-(4-(methylsulfonyl)phenoxy)-N-(1-(2-(pyrrolidin-1-yl)ethyl)-1H-pyrazol- |
| | 4-yl)-[1,2,4]triazolo[1,5-a]pyridin-2-amine |

In some embodiments, provided is a compound of formula (Ia):

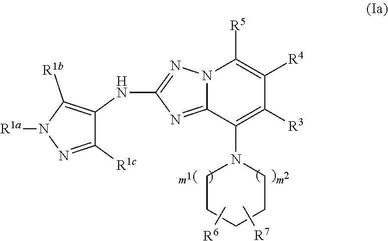

or a stereoisomer, tautomer, solvate, prodrug or salt thereof, where R^{1a}, R^{1b}, R^{1c}, R³, R⁴, R⁵, R⁶, R⁷, m¹ and m² are as defined herein.

In some embodiments, the compound is of the formula (Ia) as defined herein, provided that the compounds is other than a compound selected from the group consisting of Compound Nos. 2x to 6x and salts thereof.

In some embodiments, in a compound of the present invention, such as a compound of formula (I) or (Ia), m¹ is 1 and m² is 1, or m¹ is 2 and m² is 1. In some embodiments, m¹ is 1 and m² is 1. In some of these embodiments, R⁶ and R⁷ are attached to the ring at the same carbon atom. In some of these embodiments, R⁶ is optionally substituted C₁-C₆ alkyl (e.g., C₁-C₆ alkyl optionally substituted with OH). In some of these embodiments, R⁷ is optionally substituted phenyl (e.g., phenyl optionally substituted with halo). In some of these embodiments, R⁶ is hydroxymethyl and R⁷ is 4-chlorophenyl.

In some embodiments of compounds of the present invention, such as a compound of formula (I) or (Ia), the moiety

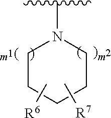

is selected from:

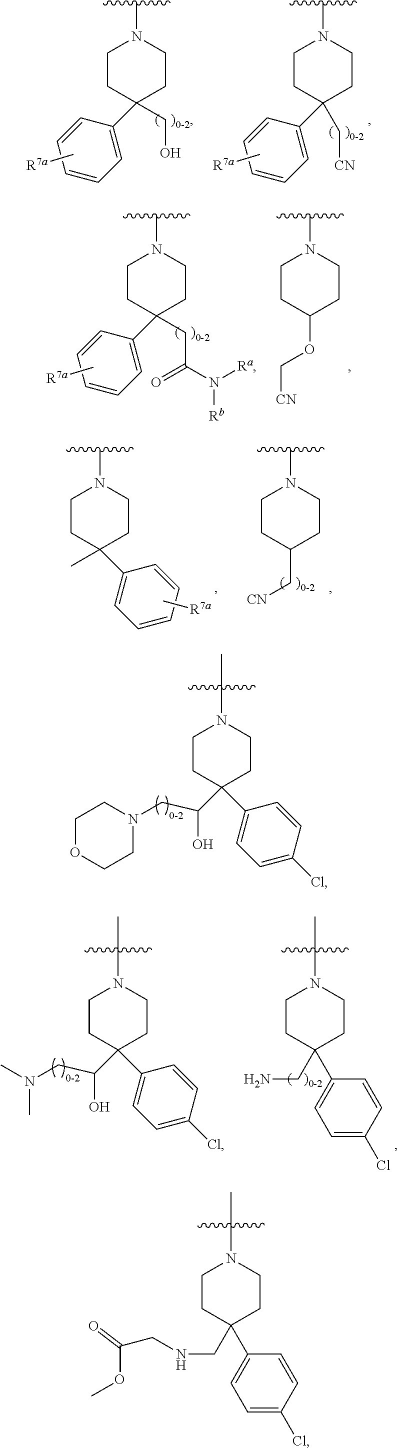

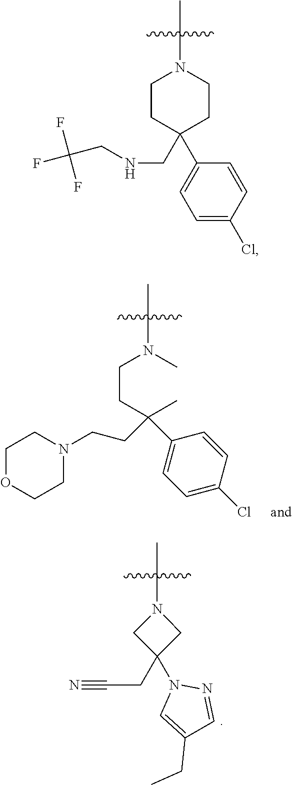

wherein R^{7a} is selected from hydrogen, halogen, OH, C₁-C₆ alkyl, C₁-C₆ alkoxy, —S—(C₁-C₆ alkyl), C₁-C₆ haloalkyl and CN.

In some embodiments, the moiety N

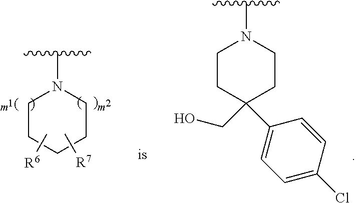

In some embodiments, a compound of formula (I) is further defined as a compound of formula (Ib):

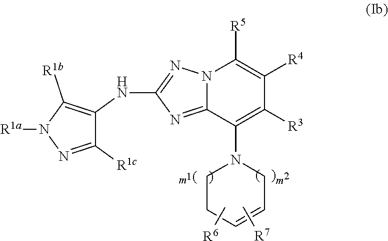

or a stereoisomer, tautomer, solvate, prodrug or salt thereof, where R^{1a}, R^{1b}, R^{1c}, R³, R⁴, R⁵, R⁶, R⁷, m¹ and m² are as defined herein.

In some embodiments of a compound of the present invention, such as a compound of formula (I) or (Ib), m¹ is 1 and m² is 2, or m¹ is 2 and m² is 1, or m¹ is 1 and m² is 1.

In some embodiments of compounds of the present invention, such as a compound of formula (I) or (Ib), the moiety

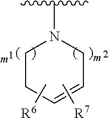

is selected from:

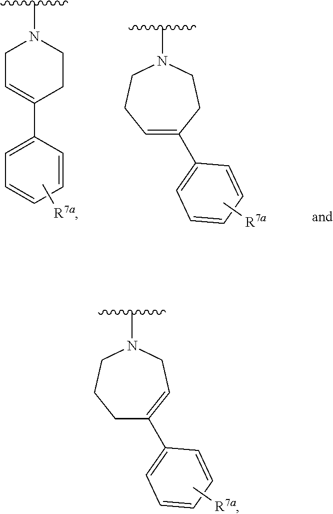

wherein R^{7a} is selected from hydrogen, halogen, C₁-C₆ alkyl, C₁-C₆ haloalkyl and CN.

In some embodiments, a compound of formula (I) is further defined as a compound of formula (Ic):

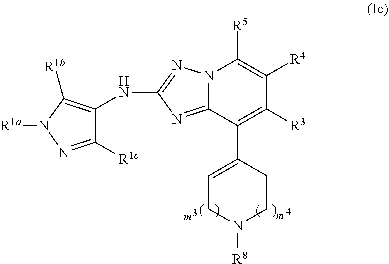

or a stereoisomer, tautomer, solvate, prodrug or salt thereof, where R^{1a}, R^{1b}, R^{1c}, R³, R⁴, R⁵, R⁸, m³ and m⁴ are as defined herein.

In some embodiments, the compound is of the formula (Ic) as defined herein, provided that the compounds is other than Compound No. 1x and salts thereof.

In some embodiments of a compound of the present invention, such as a compound of formula (I) or (Ic), m³ is 1 and m⁴ is 1, or m³ is 1 and m⁴ is 2, or m³ is 1 and m⁴ is 0. In some embodiments, m³ is 1 and m⁴ is 1. In some embodiments, R⁸ is C(O)R^{8a}. In some of these embodiments, R^{8a} is C₁-C₆ alkyl optionally substituted with halo. In some embodiments, R⁸ is C(O)CH₂CH₂CF₃.

In some embodiments of compounds of the present invention, such as a compound of formula (I) or (Ic), the moiety

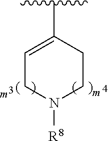

is selected from:

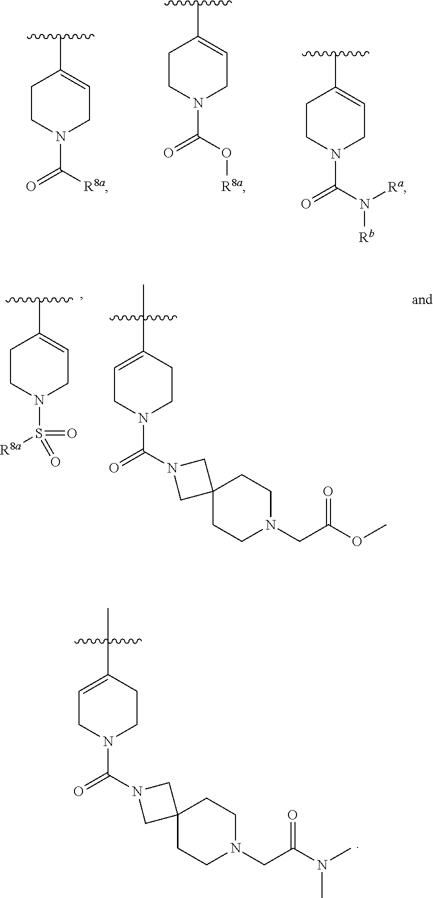

In some embodiments, the moiety

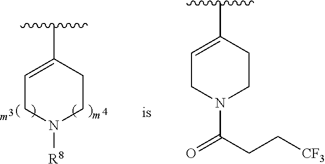

In some embodiments, a compound of formula (I) is further defined as a compound of formula (Id):

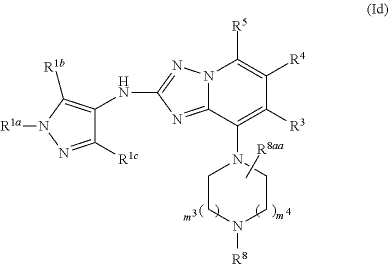

or a stereoisomer, tautomer, solvate, prodrug or salt thereof, where R^{1a}, R^{1b}, R^{1c}, R³, R⁴, R⁵, R⁸, R^{8aa}, m³ and m⁴ are as defined herein.

In some embodiments of a compound of the present invention, such as a compound of formula (I) or (Id), m³ is 1 and m⁴ is 1, m³ is 1 and m⁴ is 1, or m³ is 1 and m⁴ is 2.

In some embodiments, a compound of formula (I) is further defined as a compound of formula (Ie):

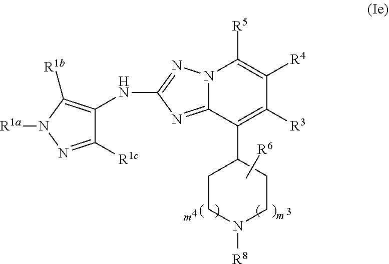

or a stereoisomer, tautomer, solvate, prodrug or salt thereof, where R^{1a}, R^{1b}, R^{1c}, R³, R⁴, R⁵, R⁶, R⁸, m³ and m⁴ are as defined herein.

In some embodiments of a compound of the present invention, such as a compound of formula (I) or (Ie), m³ is 0 and m⁴ is 1 or m³ is 1 and m⁴ is 1.

In some embodiments, a compound of formula (I) is further defined as a compound of formula (If):

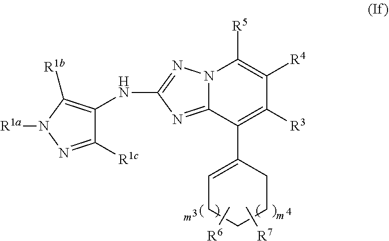

or a stereoisomer, tautomer, solvate, prodrug or salt thereof, where R^{1a}, R^{1b}, R^{1c}, R³, R⁴, R⁵, R⁶, R⁷, m³ and m⁴ are as defined herein.

In some embodiments of a compound of the present invention, such as a compound of formula (I) or (If), m³ is 1 and m⁴ is 1.

In some embodiments, a compound of formula (I) is further defined as a compound of formula (Ig):

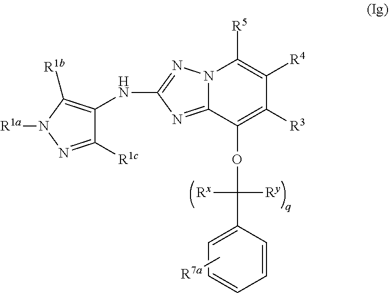

or a stereoisomer, tautomer, solvate, prodrug or salt thereof, where R^{1a}, R^{1b}, R^{1c}, R³, R⁴, R⁵, R^{x}, R^{y} and q are as defined herein and R^{7a} is selected from hydrogen, OH, halogen, C₁-C₆ alkyl, C₁-C₆ alkoxy, C₁-C₆ haloalkyl and CN. In some embodiments, q is either 0 or 1, and when q is 1, then R^{x} and R^{y} are hydrogen.

In some embodiments, the compound is of the formula (Ig) as defined herein, provided that the compounds is other than Compound No. 7x and salts thereof.

In some embodiments, a compound of formula (I) is further defined as a compound of formula (Ih):

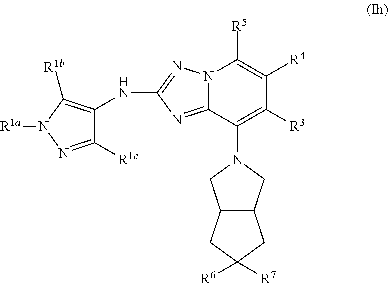

or a stereoisomer, tautomer, solvate, prodrug or salt thereof, where R^{1a}, R^{1b}, R^{1c}, R³, R⁴, R⁵, R⁶ and R⁷ are as defined herein.

In some embodiments, a compound of formula (I) is further defined as a compound of formula (Ii)

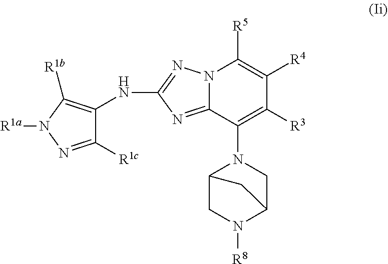

or a stereoisomer, tautomer, solvate, prodrug or salt thereof, where R^{1a}, R^{1b}, R^{1c}, R³, R⁴, R⁵ and R⁸ are as defined herein.

In some embodiments, a compound of formula (I) is further defined as a compound of formula (Ij)

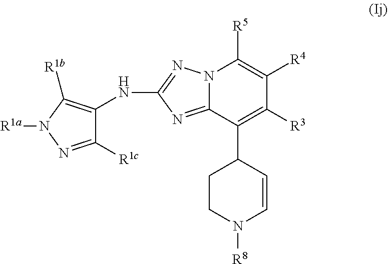

or a stereoisomer, tautomer, solvate, prodrug or salt thereof, where R^{1a}, R^{1b}, R^{1c}, R³, R⁴, R⁵ and R⁸ are as defined herein.

In some embodiments, a compound of formula (I) is further defined as a compound of formula (Ik):

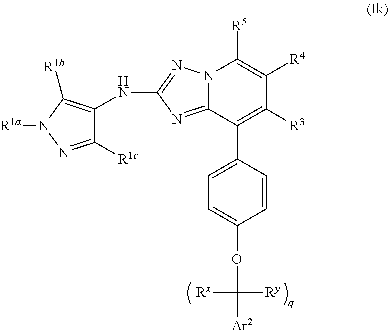

or a stereoisomer, tautomer, solvate, prodrug or salt thereof, where R^{1a}, R^{1b}, R^{1c}, R³, R⁴, R⁵, R^{x}, R^{y}, Ar² and q are as defined herein.

In some embodiments of a compound of the present invention, such as a compound of formula (I) or (Ik), q is 1. In some embodiments, q is 1 and each of R^{x} and R^{y} is hydrogen. In some embodiments, Ar² is optionally substituted 5-11 membered heteroaryl. In some embodiments, Ar² is 6-membered heteroaryl optionally substituted with OR′ where R′ is C₁-C₆ alkyl optionally substituted with C₁-C₆ alkoxy. In some embodiments, Ar² is 6-(2-methoxyethoxy)-3-pyridyl. In some embodiments, q is 1, each of R^{x} and R^{y} is hydrogen, and Ar² is 6-(2-methoxyethoxy)-3-pyridyl.

In some embodiments of compounds of the present invention, such as a compound of formula (I) or (Ik), the moiety

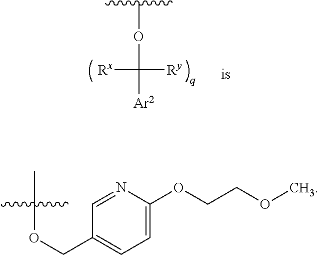

In some embodiments of a compound of the present invention, such as a compound of formula (I), (Ia), (Ib), (If) or (Ih), R⁶ is hydrogen. In some embodiments of a compound of the present invention, such as a compound of formula (I), (Ia), (Ib), (If) or (Ih), R⁷ is OH or C₁-C₆-alkoxy. In some embodiments of a compound of the present invention, such as a compound of formula (I), (Ia), (Ib), (If) or (Ih), R⁶ is H and R⁷ and is substituted phenyl.

In some embodiments of a compound of the present invention, such as a compound of formula (I), (Ia), (Ib) or (If), R⁶ and R⁷ are attached to the ring at the same carbon atom. In some embodiments of compounds of the present invention, such as a compound of formula (I), (Ia), (Ib) or (If), one or both of R⁶ and R⁷ is located at the para position of the ring. In some embodiments, R⁶ is hydroxymethyl and R⁷ is 4-chlorophenyl. In some embodiments of compounds of the present invention, such as a compound of formula (I), (Ia), (Ib) or (If), R⁶ and R⁷ are attached to different ring atoms.

In some embodiments of a compound of the present invention, such as a compound of formula (I), (Ia), (Ib) or (If), R⁶ is C₁-C₆ alkyl or C₁-C₆-alkoxy, and R⁷ is optionally substituted phenyl, such as phenyl substituted by halogen, CN, C₁-C₆ alkyl or C₁-C₆ alkoxy. In some embodiments in a compound of the present invention, such as a compound of formula (I), (Ia), (Ib) or (If), R⁶ is C₁-C₆ alkyl, C₃-C₆ cycloalkyl or optionally substituted phenyl, such as phenyl substituted by halogen, CN, C₁-C₆ alkyl or C₁-C₆ alkoxy, and R⁷ is OH, (C₀-C₆ alkylene)C(O)NR^{a}R^{b}, (C₀-C₆ alkylene)CN or —O—(C₀-C₆ alkyl)CN. In some embodiments in a compound of the present invention, such as a compound of formula (I), (Ia), (Ib) or (If), R⁶ is hydrogen and R⁷ is selected from (C₀-C₆ alkylene)C(O)NR^{a}R^{b}, (C₀-C₆ alkylene)CN, C₁-C₆-alkoxy, —O—(C₃-C₆ cycloalkyl), —O—(C₀-C₆ alkylene)C(O)NR^{a}R^{b}, and —O—(C₁-C₆ alkylene)CN. In some embodiments in a compound of the present invention, such as a compound of formula (I), (Ia), (Ib) or (If), R⁶ and R⁷ together form a 3-11 membered heterocycloalkyl (such as a heterocycloalkyl containing at least one nitrogen) optionally substituted by oxo.

In some embodiments of a compound of the present invention, such as a compound of formula (I), (Ic), (Id), (Ie), (Ii) or (Ij), R⁸ is substituted phenyl, such as mono- or disubstituted phenyl, C(O)NR^{a}R^{b} or C(O)R^{8a}. In some embodiments, R⁸ is C(O)NR^{a}R^{b}. In some embodiments, R⁸ is C(O)R^{8a}. In some embodiments, R⁸ is C(O)R^{8a} where R^{8a} is 4,4,4-trifluorobutanoyl.

In some embodiments of a compound of the present invention, such as a compound of formula (I), (Ia), (Ib), (Ic), (Id), (Ie), (If), (Ig), (Ih), (Ii), (Ij) or (Ik), R⁹ is optionally substituted C₁-C₆ alkyl or optionally substituted 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S). For example, in some embodiments of compounds of the present invention, such as a compound of formula (I), (Ia), (Ib), (Ic), (Id), (Ie), (If), (Ig), (Ih), (Ii), (Ij) or (Ik), the optional substituents of optionally substituted C₁-C₆ alkyl of R⁹ or optionally substituted 3-11 membered heterocyclyl of R⁹ are selected from OH; CN; NR^{a}R^{b}; C(O)NR^{a}R^{b}; C₁-C₆ alkyl; C₃-C₈ cycloalkyl; C₁-C₆ alkoxy; oxo; phenyl; 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S) optionally substituted by C₁-C₆ alkyl, NR^{a}R^{b}, or 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S); C(O)C₁-C₆ alkyl; and C(O)-3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S) optionally substituted by C₁-C₆ alkyl.

In some embodiments of the compound of formula (I), (Ia), (Ib), (Ic), (Id), (Ie), (If), (Ig), (Ih), (Ii), (Ij) or (Ik), or a stereoisomer, tautomer, solvate, prodrug or salt thereof, where ^{1a} is C₁-C₆ alkyl optionally substituted by R⁹ or 3-11 membered heterocyclyl optionally substituted by R⁹. In some embodiments, R^{1a} is C₁-C₆ alkyl optionally substituted by OH, halogen, CN, optionally substituted phenyl, optionally substituted 3-11 membered heterocyclyl, optionally substituted 5-11 membered heteroaryl, or —NR^{a}R^{b}. In some embodiments, R^{1a} is C₁-C₆ alkyl optionally substituted by OH, halogen, CN, 3-11 membered heterocyclyl optionally substituted with C₁-C₆ alkyl or C₁-C₆ haloalkyl, 5-11 membered heteroaryl optionally substituted with C₁-C₆ alkyl or C₁-C₆ haloalkyl, or —NR^{a}R^{b}. In some embodiments, R^{1a} is C₁-C₆ alkyl substituted by 1 to 5 substituents independently selected from OH, halogen and CN. In some embodiments, R^{1a} is C₁-C₆ alkyl substituted by phenyl. In some embodiments, R^{1a} is C₁-C₆ alkyl substituted by 3-11 membered heterocyclyl optionally substituted by C₁-C₆ alkyl. In some embodiments, R^{1a} is C₁-C₆ alkyl substituted by piperidin-4-yl, piperazin-1-yl, 4-methylpiperazin-1-yl, morpholin-1-yl or pyrrolidin-2-yl. In some embodiments, R^{1a} is C₁-C₆ alkyl substituted by 5-11 membered heteroaryl. In some embodiments, R^{1a} is C₁-C₆ alkyl substituted by —NR^{a}R^{b}, wherein R^{a} and R^{b} are independently hydrogen or methyl. In some embodiments, R^{1a} is 3-11 membered heterocyclyl optionally substituted by C₁-C₆ alkyl. In some embodiments, R^{1a} is 3-11 membered heterocyclyl optionally substituted by C₁-C₆ alkyl which is optionally substituted with phenyl or cyano.

In some embodiments, the compound is of formula (I), (Ia), (Ib), (Ic), (Id), (Ie), (If), (Ig), (Ih), (Ii), (Ij) or (Ik), or a stereoisomer, tautomer, solvate, prodrug or salt thereof, where R^{1a} is other than C₁-C₆ alkyl substituted by —C(O)NR^{a}R^{b}.

In some embodiments, the compound is of formula (I), (Ia), (Ib), (Ic), (Id), (Ie), (If), (Ig), (Ih), (Ii), (Ij) or (Ik), or a stereoisomer, tautomer, solvate, prodrug or salt thereof, where R^{1a} is selected from the group consisting of:

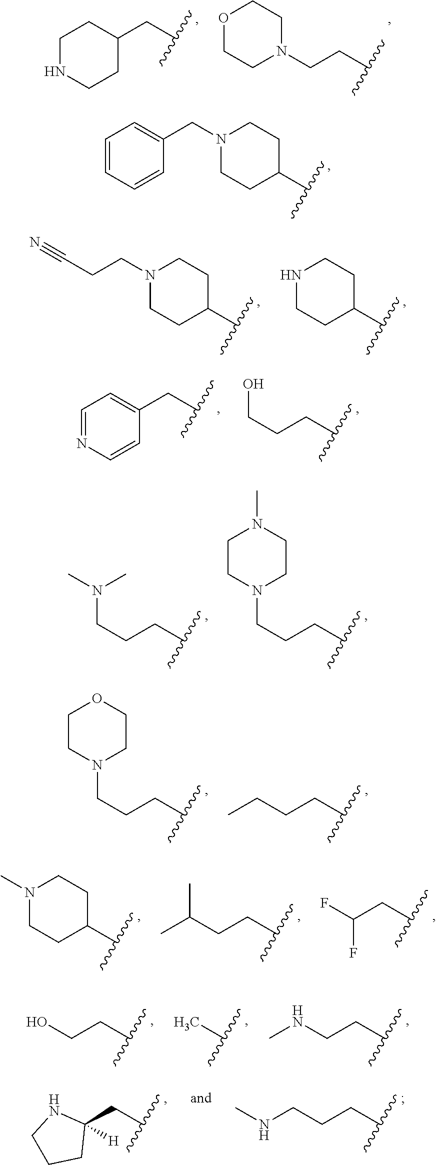

wherein the wavy line represents the point of attachment of R^{1a} in formula (I), (Ia), (Ib), (Ic), (Id), (Ie), (If), (Ig), (Ih), (Ii), (Ij) or (Ik).

In some embodiments, the compound is of formula (I), (Ia), (Ib), (Ic), (Id), (Ie), (If), (Ig), (Ih), (Ii), (Ij) or (Ik), or a stereoisomer, tautomer, solvate, prodrug or salt thereof, where R^{1a} is selected from the group consisting of:

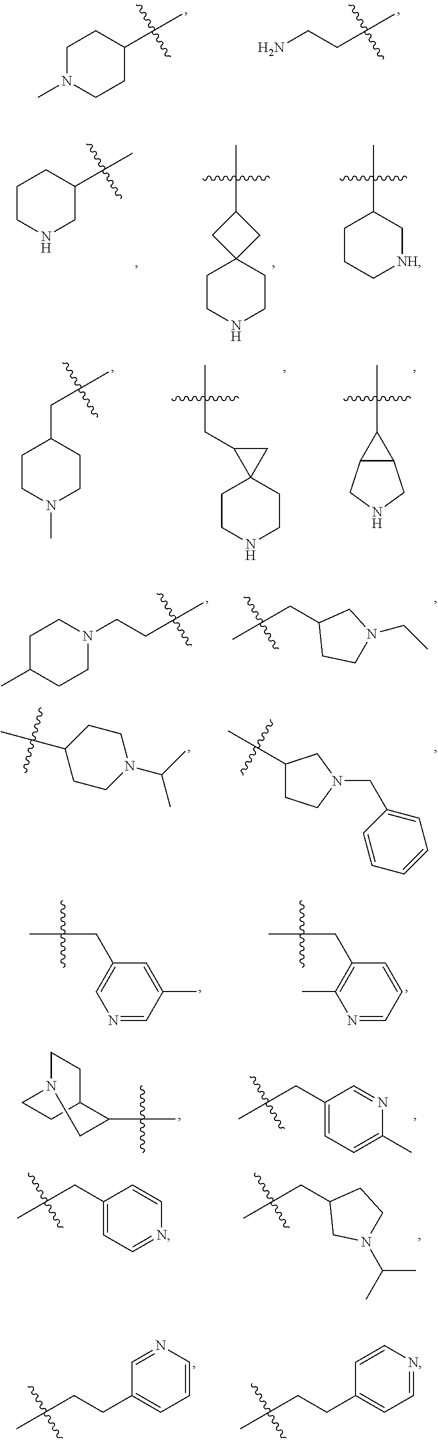

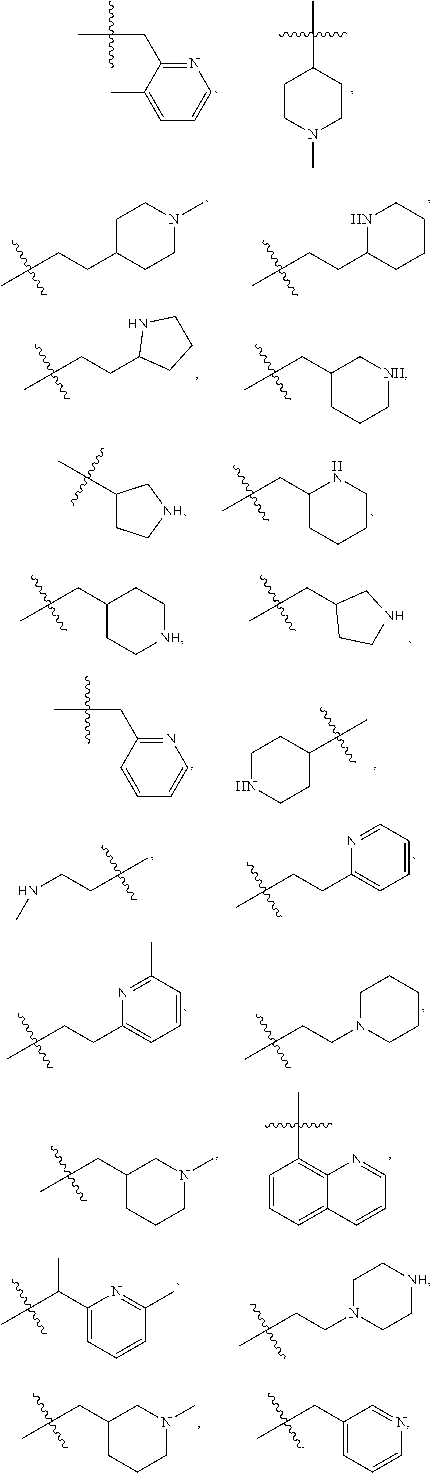

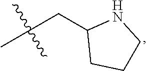 [SMILES: *CC1CCCN1]

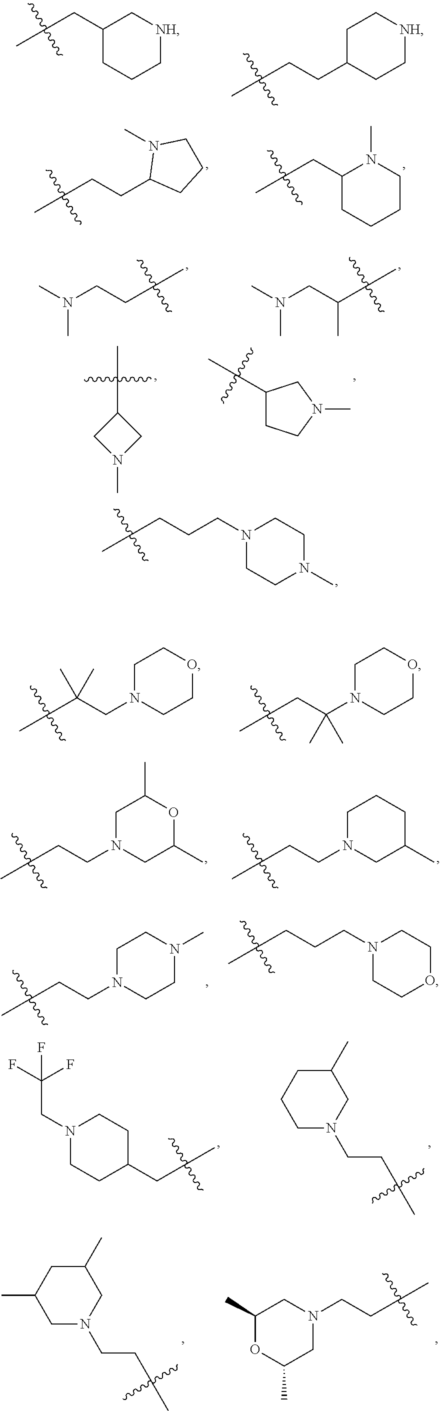

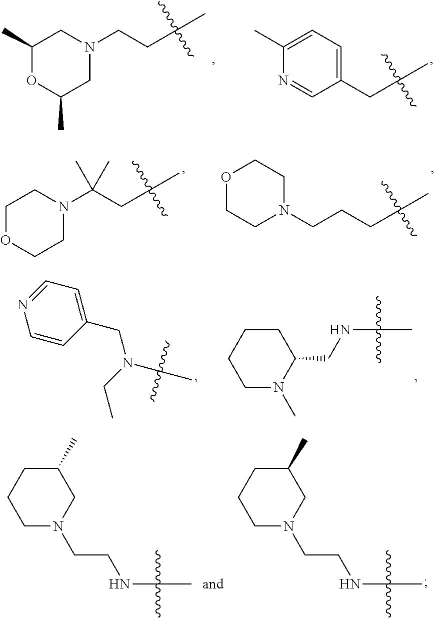

wherein the wavy line represents the point of attachment of R^{1a} in formula (I), (Ia), (Ib), (Ic), (Id), (Ie), (If), (Ig), (Ih), (Ii), (Ij) or (Ik).

In some embodiments, the compound is of formula (I), (Ia), (Ib), (Ic), (Id), (Ie), (If), (Ig), (Ih), (Ii), (Ij) or (Ik), or a stereoisomer, tautomer, solvate, prodrug or salt thereof, where R^{1b} and R^{1c} are independently hydrogen or C₁-C₆ alkyl (e.g., methyl). In some embodiments, R^{1b} and R^{1c} are each hydrogen.

In some embodiments, the compound is of formula (I), (Ia), (Ib), (Ic), (Id), (Ie), (If), (Ig), (Ih), (Ii), (Ij) or (Ik), or a stereoisomer, tautomer, solvate, prodrug or salt thereof, where R³, R⁴ and R⁵ are each independently selected from the group consisting of hydrogen, CH₃, CH₂CH₃, OCH₃, CF₃, F and C₁. In some embodiments, R³, R⁴ and R⁵ are each independently selected from the group consisting of hydrogen, CH₃, CH₂CH₃, CF₃, F and C₁. In some embodiments, R³ is hydrogen. In some embodiments, R⁴ is hydrogen. In some embodiments, R⁵ is hydrogen. In some embodiments, R³, R⁴ and R⁵ are each independently hydrogen. In some embodiments, none of R³, R⁴ and R⁵ are OCH₃.

In some embodiments, the compound is of formula (I), or a stereoisomer, tautomer, solvate, prodrug or salt thereof, where R² is selected from the group consisting of:

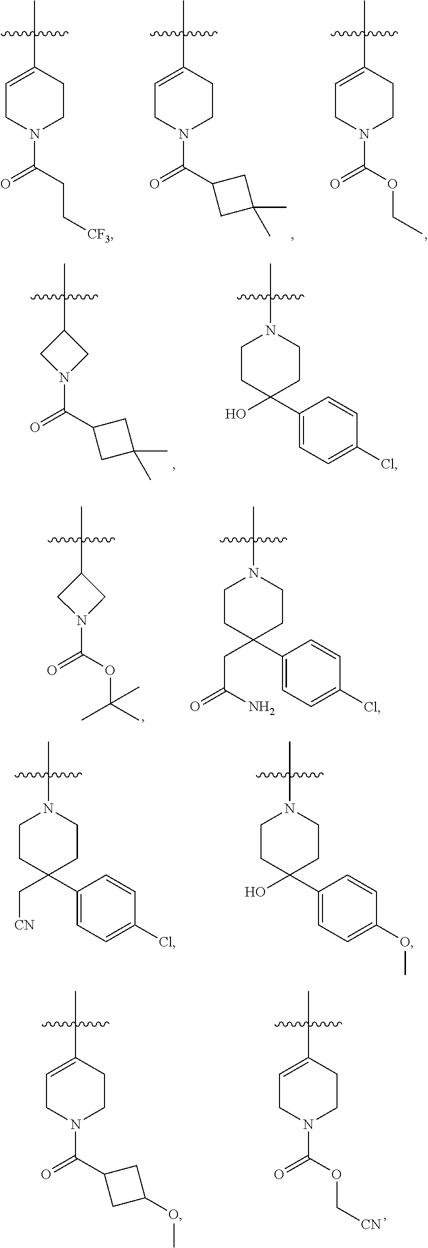

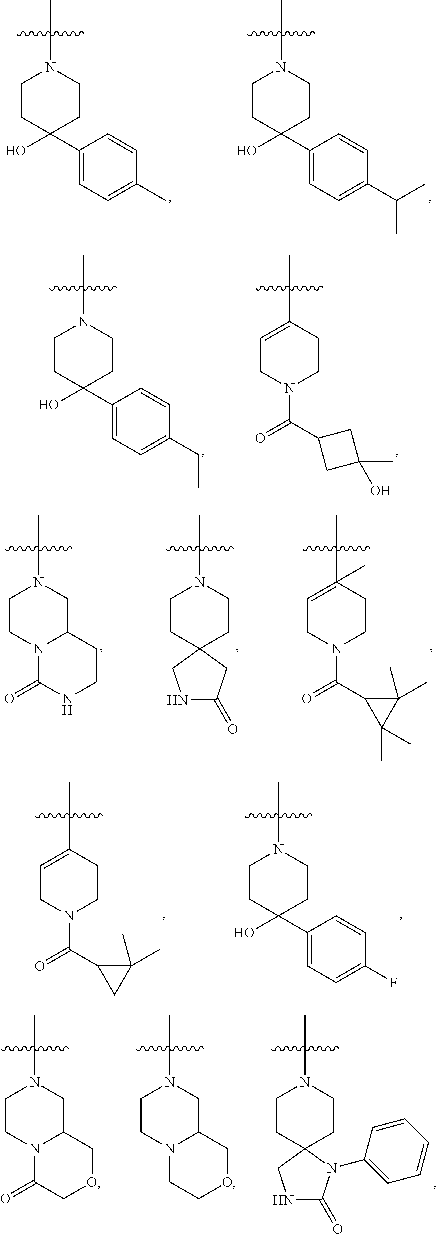

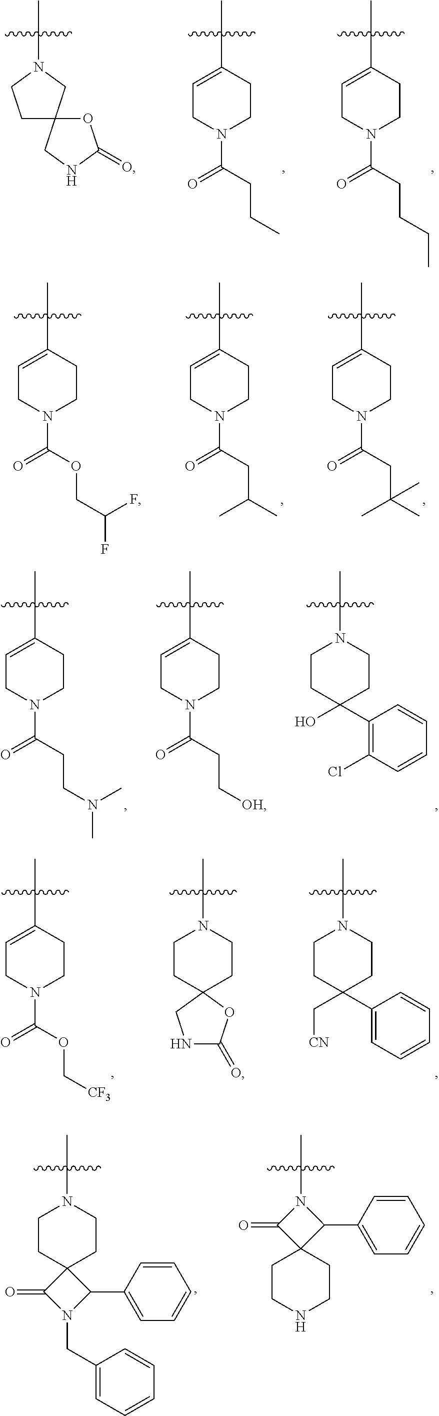

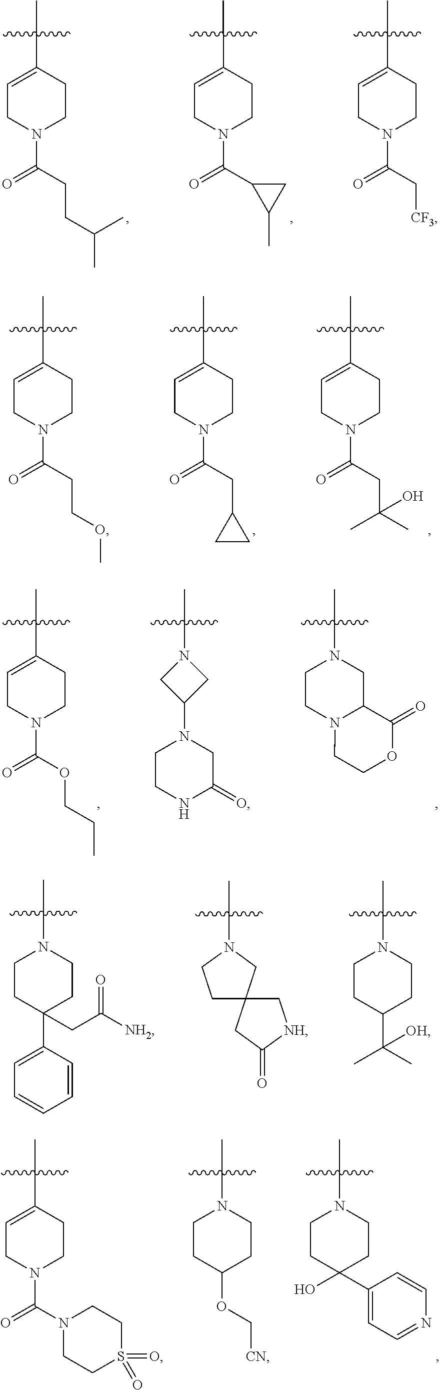

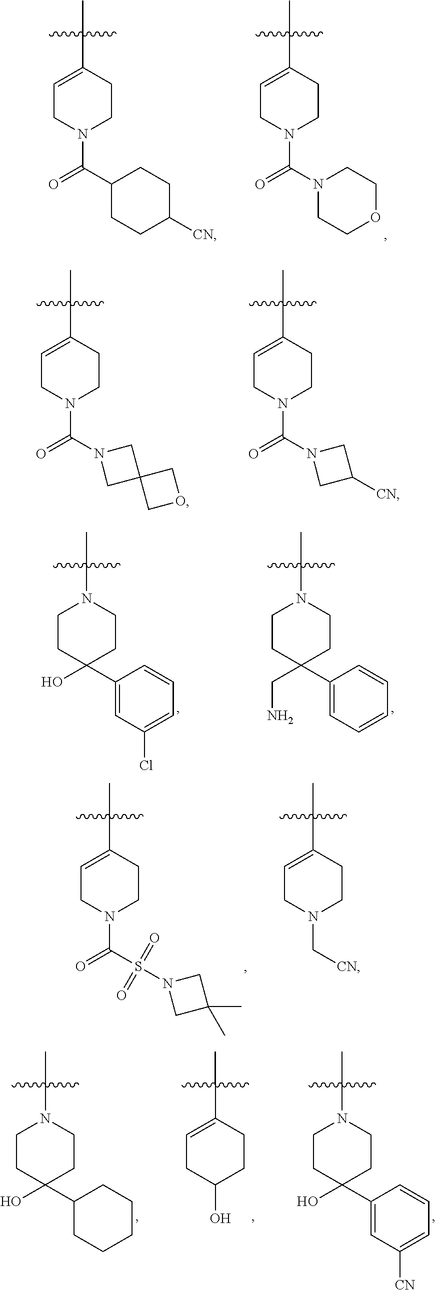

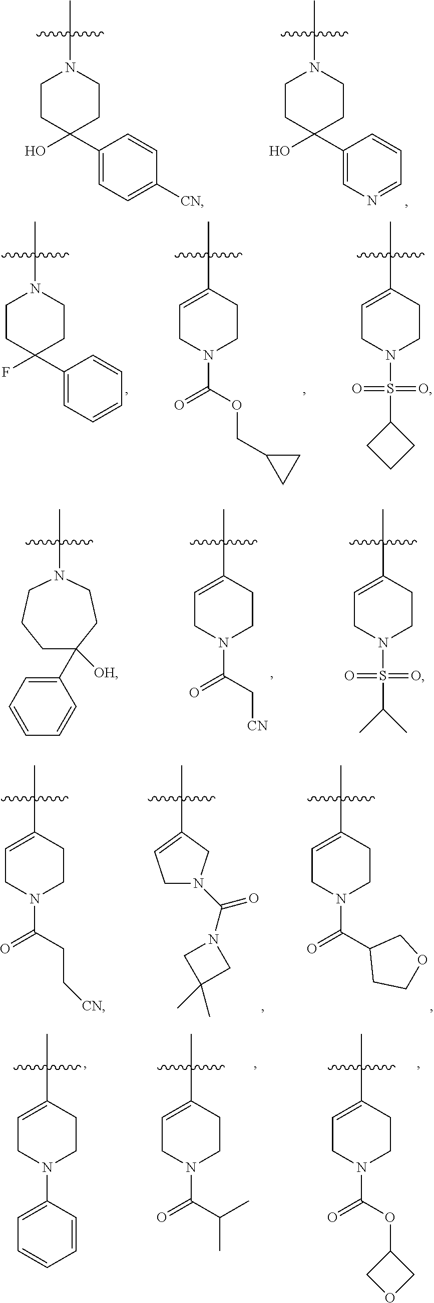

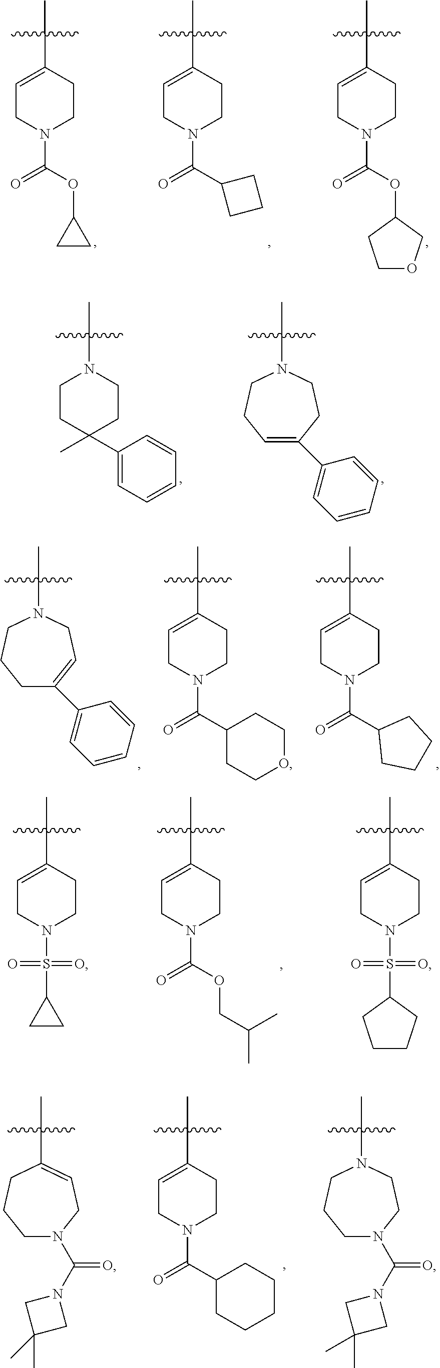

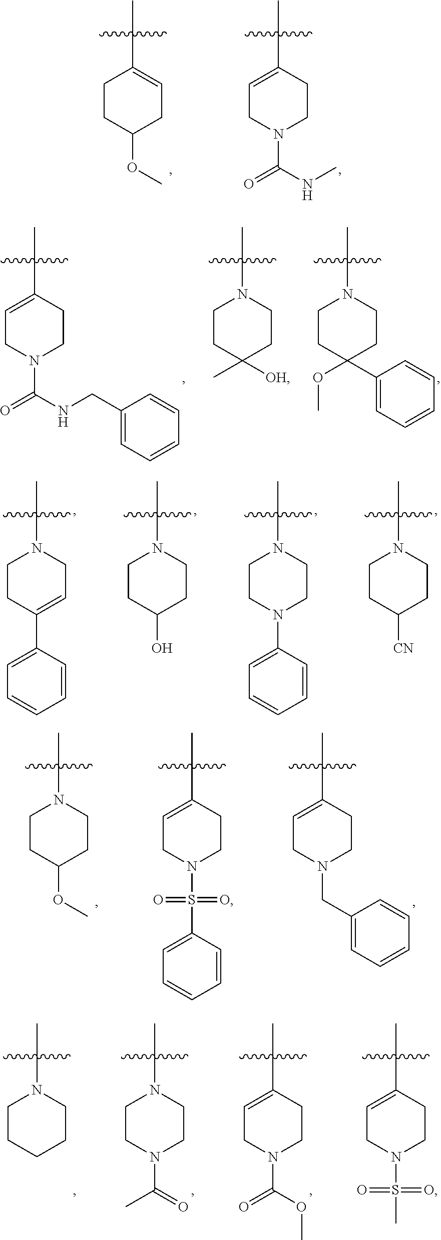

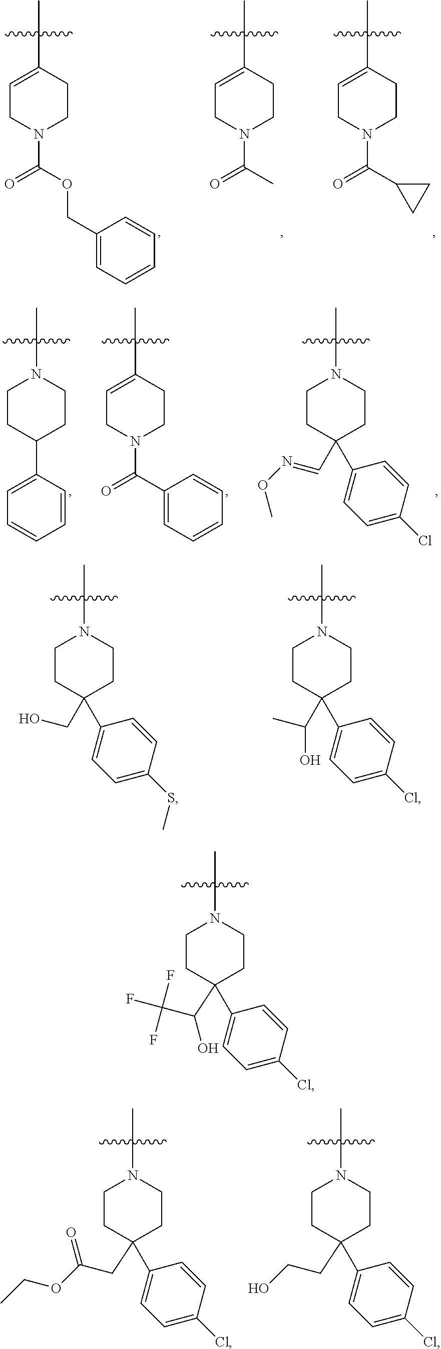

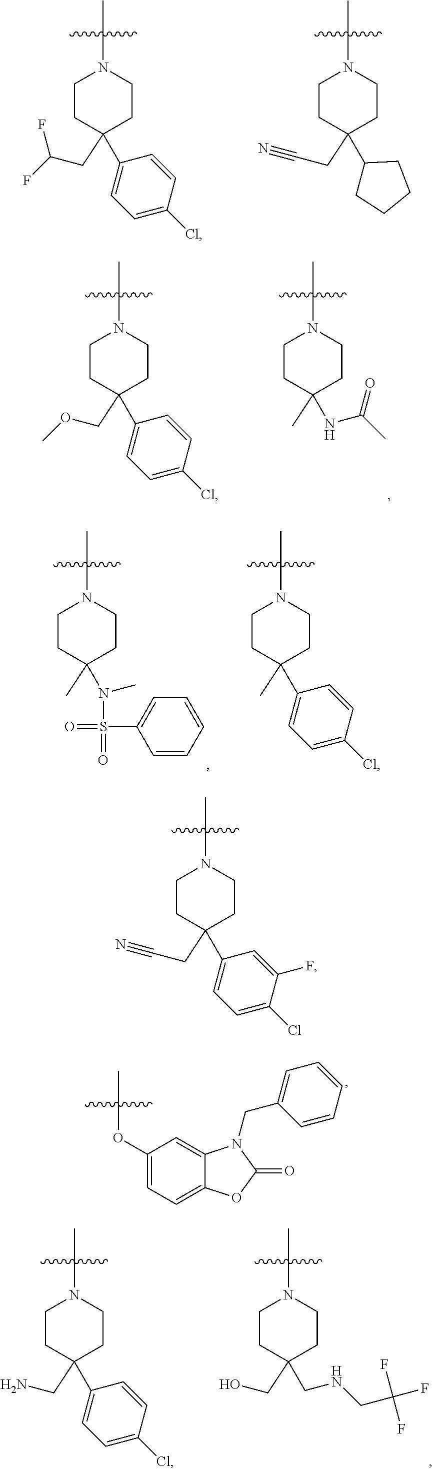

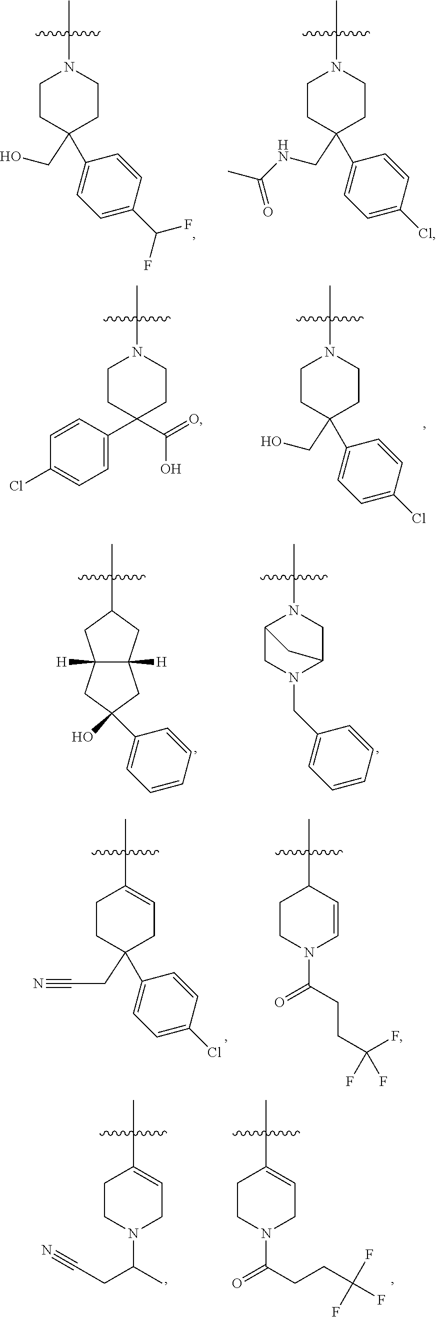

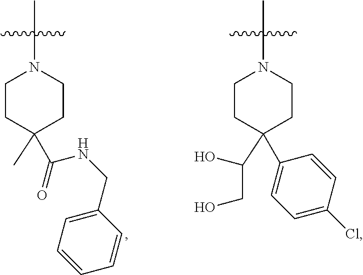 [SMILES: *N1CCC(C)(C(=O)NCc2ccccc2)CC1 ; *N1CCC(c2ccc(Cl)cc2)(C(O)CO)CC1]

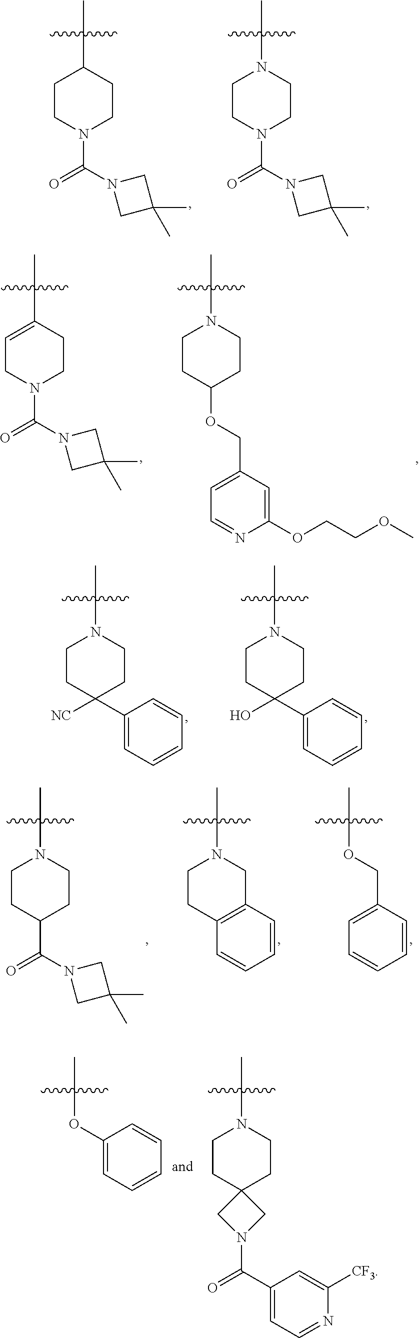

In some embodiments of the compound of formula (I), R² is 4-(4-chlorophenyl)-4-(hydroxymethyl)piperidin-1-yl, 1-(4,4,4-trifluorobutanoyl)-1,2,3,6-tetrahydropyridin-4-yl or 4-((6-(2-methoxyethoxy)pyridin-3-yl)methoxy)phenyl.

It is intended and understood that each and every variation of R², R³, R⁴ and R⁵ described for formula (I) may be combined with each and every variation of R^{1a}, R^{1b} and R^{1c} described for formula (I) as if each and every combination is individually described. For example, in some embodiments, each R^{1b}, R^{1c}, R³, R⁴ and R⁵ is hydrogen, R² is 4-(4-chlorophenyl)-4-(hydroxymethyl)piperidin-1-yl, 1-(4,4,4-trifluorobutanoyl)-1,2,3,6-tetrahydropyridin-4-yl or 4-((6-(2-methoxyethoxy)pyridin-3-yl)methoxy)phenyl, and R^{1a} is selected from the group consisting of:

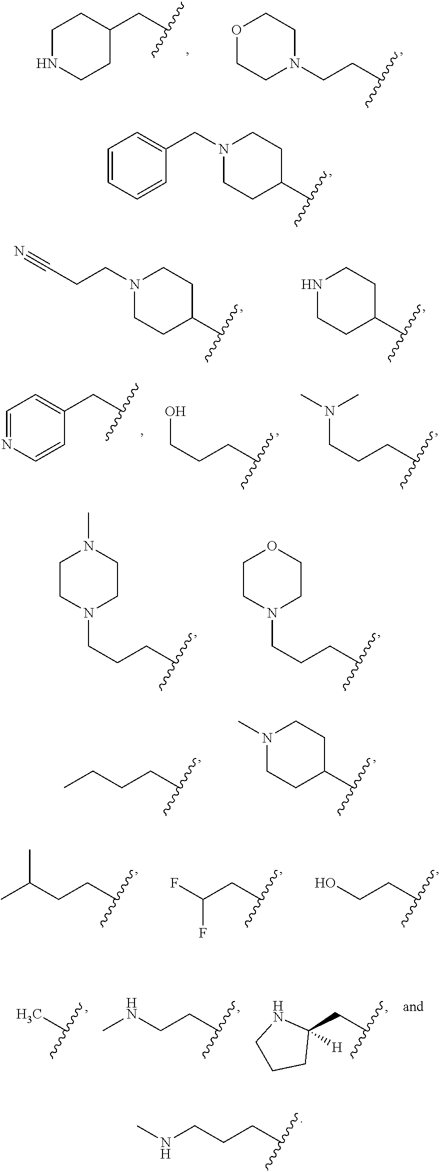

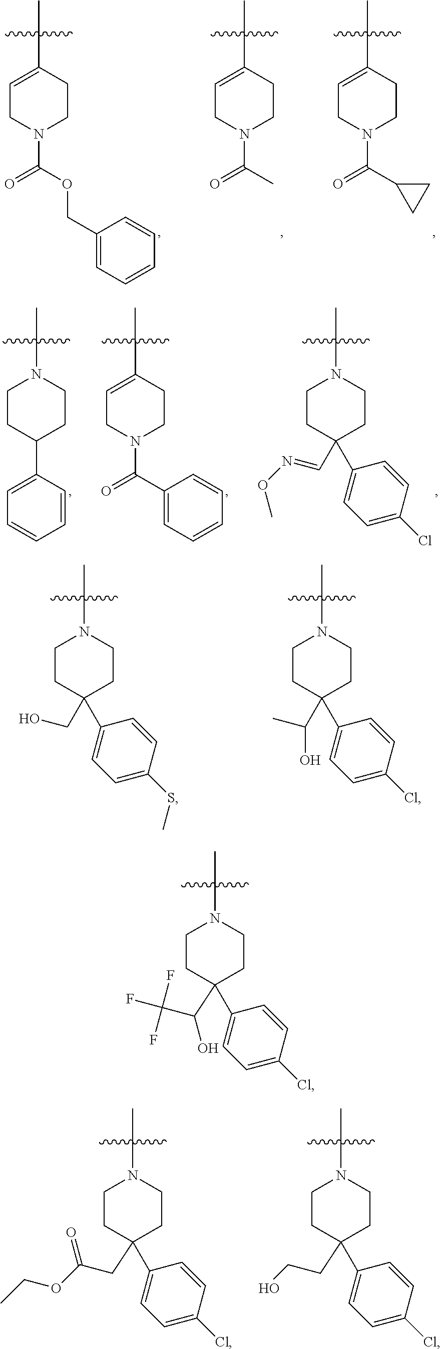

Another aspect of the invention provides compounds of formula (II):

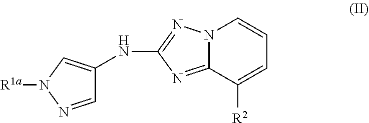

or a stereoisomer, tautomer, solvate, prodrug or salt thereof, wherein:

R^{1a} is hydrogen, C₁-C₆ alkyl, C₃-C₈ cycloalkyl, phenyl, or 3-11 membered heterocyclyl and R^{1a} is optionally substituted by R⁹;

R² is a 3-11 membered heterocyclyl containing at least 1 nitrogen, selected from groups (a)-(e) and (h)-(j); a C₅-C₈ cycloalkenyl ring (f); a —O—(CR^{x}R^{y})_{q}-Ar² group (g); or a Ar¹—O—(CR^{x}R^{y})_{q}-Ar² group (k), where each R^{x} and R^{y} are independently hydrogen or C₁-C₆ alkyl, each q is independently 0 to 3, Ar¹ is 1,4-phenylene and Ar² is optionally substituted C₆-C₁₀ aryl or optionally substituted 5-11 membered heteroaryl:

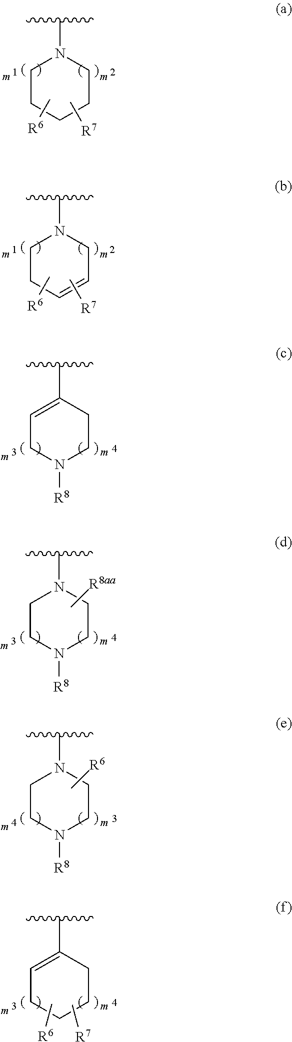

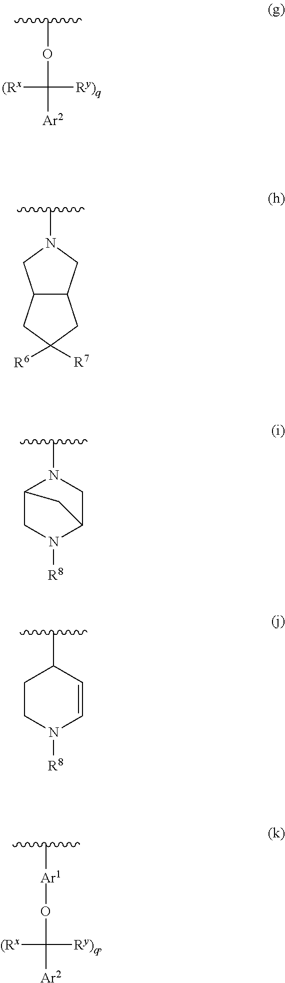

wherein the wavy line represents the point of attachment of R² in formula (II);

R⁶ and R⁷ are independently selected from the group consisting of hydrogen, halogen, OH, CN, phenyl, C₁-C₆ alkyl, (C₀-C₆ alkylene)C₃-C₈ cycloalkyl, (C₀-C₆ alkylene)3-11 membered heterocyclyl, (C₀-C₆ alkylene)C(O)NR^{a}R^{b}, (C₀-C₆ alkylene)NR^{a}C(O)(C₁-C₆ alkyl), (C₀-C₆ alkylene)NR^{a}C(O)(phenyl), (C₀-C₆ alkylene)C(O)R^{8a}, (C₀-C₆ alkylene)C(O)OR^{8a}, C₁-C₆ alkoxy, —O—(C₃-C₆ cycloalkyl), —O—(C₀-C₆ alkylene)C(O)NR^{a}R^{b}, —C═N—O—(C₁-C₆ alkyl), —O—(C₁-C₆ alkyl)3-11 membered heterocyclyl, (C₀-C₆ alkylene)NR^{a}SO₂(C₁-C₆ alkyl), (C₀-C₆ alkylene)NR^{a}SO₂(phenyl), and —O-(3-11 membered heterocyclyl); wherein said alkyl, alkylene, alkoxy, cycloalkyl, phenyl and heterocyclyl are each independently optionally substituted,
or R⁶ and R⁷ together form an optionally substituted phenyl or optionally substituted 3-11 membered heterocyclyl;

R⁸ is H, C₁-C₆ alkyl, (C₀-C₆ alkylene)phenyl, (C₀-C₆ alkylene)C₃-C₈ cycloalkyl, (C₀-C₆ alkylene)3-11 membered heterocyclyl, C(O)NR^{a}R^{b}, SO₂NR^{a}R^{b}, (C₁-C₆ alkylene)C(O)OR^{8a} or C(O)R^{8a}, wherein said alkyl, alkylene, heterocyclyl and phenyl are each independently optionally substituted;

R^{8a} is H, NR^{a}R^{b}, C₁-C₆ alkyl, (C₀-C₆ alkylene)C₃-C₈ cycloalkyl, (C₀-C₆ alkylene)phenyl, or (C₀-C₆ alkylene)3-11 membered heterocyclyl, wherein said alkyl, alkylene, cycloalkyl, phenyl and heterocyclyl are each independently optionally substituted;

R^{8aa} is H, C₁-C₆ alkyl optionally substituted by OH, or C(O)NR^{a}R^{b}; or

or R⁸ and R^{8aa} together form an optionally substituted 3-11 membered heterocyclyl;

R⁹, independently at each occurrence, is OH, halogen, CN, C₁-C₆ alkyl, C₃-C₈ cycloalkyl, phenyl, 3-11 membered heterocyclyl, 5-11 membered heteroaryl, —C(O)NR^{a}R^{b}, —NR^{a}R^{b}, (C₁-C₆ alkylene)C₃-C₈ cycloalkyl, (C₁-C₆ alkylene)phenyl, (C₁-C₆ alkylene)3-11 membered heterocyclyl, (C₁-C₆ alkylene)5-11 membered heteroaryl, (C₁-C₆ alkylene)C(O)NR^{a}R^{b}, (C₁-C₆ alkylene)NR^{a}R^{b}, or C(O)(C₁-C₆ alkyl), wherein said alkyl, alkylene, cycloalkyl, phenyl, heterocyclyl and heteroaryl are each independently optionally substituted;

R^{a} and R^{b}, independently at each occurrence, are selected from the group consisting of hydrogen, C₁-C₆ alkyl optionally substituted by halogen or CN, (C₀-C₆ alkylene)C₃-C₈ cycloalkyl, or (C₀-C₆ alkylene)phenyl, and wherein one or more alkylene units of any alkyl group is independently optionally substituted by —O—, or alternatively R^{a} and R^{b} may be joined together with the nitrogen atom to which they are attached to form an optionally substituted 3-11 membered heterocyclyl; and

m¹, m², m³ and m⁴ are each independently 0, 1 or 2.

In some embodiments, the compound is of the formula (II) as defined herein, provided that the compounds is other than a compound selected from the group consisting of Compound Nos. 1x to 7x and salts thereof.

In some embodiments of the compound of formula (II), R^{1a} is C₁-C₆ alkyl optionally substituted by R⁹ or 3-11 membered heterocyclyl optionally substituted by R⁹. In some embodiments, R^{1a} is C₁-C₆ alkyl optionally substituted by OH, halogen, CN, 3-11 membered heterocyclyl optionally substituted with C₁-C₆ alkyl or C₁-C₆ haloalkyl, 5-11 membered heteroaryl optionally substituted with C₁-C₆ alkyl or C₁-C₆ haloalkyl, or —NR^{a}R^{b}. In some embodiments, R^{1a} is 3-11 membered heterocyclyl optionally substituted by C₁-C₆ alkyl which is optionally substituted with phenyl or cyano.

In some embodiments, the compound is of formula (II), or a stereoisomer, tautomer, solvate, prodrug or salt thereof, where R^{1a} is selected from the group consisting of:

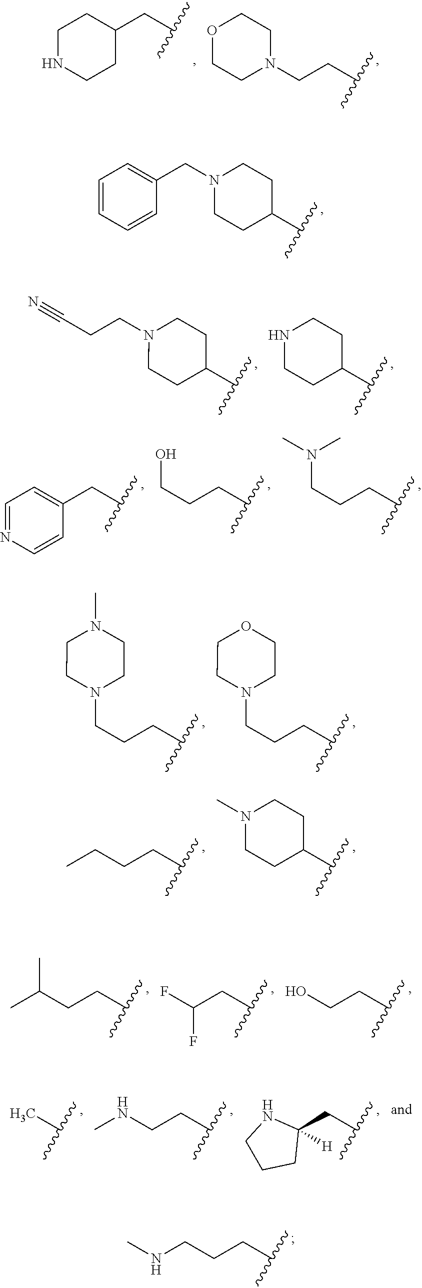

wherein the wavy line represents the point of attachment of R^{1a} in formula (II).

In some embodiments of the compound of formula (II), R² is 4-(4-chlorophenyl)-4-(hydroxymethyl)piperidin-1-yl, 1-(4,4,4-trifluorobutanoyl)-1,2,3,6-tetrahydropyridin-4-yl or 4-((6-(2-methoxyethoxy)pyridin-3-yl)methoxy)phenyl.

It is intended and understood that each and every variation of R^{1a} and R² or combinations thereof may be applicable to formula (II) as if each and every combination is individually described. It is further understood and intended that each and every variation of m¹, m², m³, m⁴, R⁶, R⁷, R⁸, R^{8a}, R^{8aa}, R⁹, R^{a} and R^{b} described herein, where applicable, may be combined with each other, and may be applied to formula (II) as if each and every combination is individually described.

In certain embodiments, R² is ring (a). In certain embodiments, R² is ring (b). In certain embodiments, R² is ring (c). In certain embodiments, R² is ring (d). In certain embodiments, R² is ring (e). In certain embodiments, R² is ring (f). In certain embodiments, R² is of the formula (g). In certain embodiments, R² is ring (h). In certain embodiments, R² is ring (i). In certain embodiments, R² is ring (j). In certain embodiments, R² is ring (k).

In some embodiments of a compound of the present invention, such as a compound of formula (I), (Ia), (Ib), (If), (Ih), or (II), where applicable, R⁶ and R⁷ are attached to the ring at the same carbon atom. In some embodiments of a compound of the present invention, such as a compound of formula (I), (Ia), (Ib), (If), (Ih), or (II), where applicable, R⁶ and R⁷ are independently selected from the group consisting of hydrogen; halogen; OH; CN; phenyl; phenyl substituted by halogen, CN, C₁-C₆ alkyl or C₁-C₆ alkoxy; C₁-C₆ alkyl; C₁-C₆ alkyl substituted by OH or CN; (C₀-C₆ alkylene)C₃-C₈ cycloalkyl; (C₀-C₆ alkylene)3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S), such as piperidinyl; (C₀-C₆ alkylene)C(O)NR^{a}R^{b}; (C₀-C₆ alkylene)NR^{a}C(O)(C₁-C₆ alkyl); (C₀-C₆ alkylene)C(O)R^{8a}; (C₀-C₆ alkylene)C(O)OR^{8a}; C₁-C₆ alkoxy; C₁-C₆ alkoxy substituted by CN; —O—(C₃-C₆ cycloalkyl), —O—(C₀-C₆ alkylene)C(O)NR^{a}R^{b}, and —O-(3-11 membered heterocyclyl) (e.g., 5-6 membered heteroaryl containing 1 to 4 heteroatoms selected from O, N and S or 4-11 membered heterocycloalkyl containing 1 to 4 heteroatoms selected from O, N and S).

In some embodiments of a compound of the present invention, such as a compound of formula (I), (Ia), (Ib), (If), (Ih), or (II), where applicable, R⁶ is C₁-C₆ alkyl or C₁-C₆-alkoxy, and R⁷ is optionally substituted phenyl, such as phenyl substituted by halogen, CN, C₁-C₆ alkyl or C₁-C₆ alkoxy. In some embodiments in a compound of the present invention, such as a compound of formula (I), (Ia), (Ib), (If), (Ih), or (II), where applicable, R⁶ is C₁-C₆ alkyl, C₃-C₆ cycloalkyl or optionally substituted phenyl, such as phenyl substituted by halogen, CN, C₁-C₆ alkyl or C₁-C₆ alkoxy, and R⁷ is OH, (C₀-C₆ alkylene)C(O)NR^{a}R^{b}, (C₀-C₆ alkylene)CN or —O—(C₀-C₆ alkyl)CN. In some embodiments in a compound of the present invention, such as a compound of formula (I), (Ia), (Ib), (If), (Ih), or (II), where applicable, R⁶ is hydrogen and R⁷ is selected from (C₀-C₆ alkylene)C(O)NR^{a}R^{b}, (C₀-C₆ alkylene)CN, C₁-C₆-alkoxy, —O—(C₃-C₆ cycloalkyl), —O—(C₀-C₆ alkylene)C(O)NR^{a}R^{b}, and —O—(C₁-C₆ alkylene)CN. In some embodiments in a compound of the present invention, such as a compound of formula (I), (Ia), (Ib), (If), (Ih), or (II), where applicable, R⁶ and R⁷ together form a 3-11 membered heterocycloalkyl, such as containing 1-4 heteroatoms selected from O, N and S, optionally substituted by oxo.

In some embodiments of a compound of the present invention, such as a compound of formula (I), (Ia), (Ib), (If), (Ih), or (II), where applicable, optional substituents of R⁶ and R⁷, or R⁶ taken together with R⁷, are selected from the group consisting of halogen, CN, OH, oxo, C₁-C₆ alkyl, C₁-C₆ alkoxy, and C₁-C₆ alkoxy-C₁-C₆ alkyl-C₁-C₆ alkoxy.

In some embodiments of compounds of the present invention, such as a compound of formula (I), (Ic), (Id), (Ie), (Ii), (Ij), or (II), where applicable, R⁸ is selected from the group consisting of C₁-C₆ alkyl optionally substituted with halogen, CN, C₁-C₆ alkoxy, or OH; (C₀-C₆ alkylene)phenyl, such as (C₀-C₁ alkylene)phenyl, where the alkylene is unsubstituted, where the phenyl may be optionally substituted with halogen, CN, oxo or OH; C(O)NR^{a}R^{b}, wherein R^{a} and R^{b} are each independently hydrogen or C₁-C₆ alkyl optionally substituted by halogen, OH or CN, or R^{a} and R^{b} together form a 3-11 membered heterocycloalkyl group, such as containing 1-4 heteroatoms selected from O, N and S, optionally substituted with C₁-C₆ alkyl, oxo, CN or OH; SO₂NR^{a}R^{b}, wherein R^{a} and R^{b} are each independently hydrogen or C₁-C₆ alkyl optionally substituted by halogen, OH or CN, or R^{a} and R^{b} together form a 3-11 membered heterocycloalkyl group, such as containing 1-4 heteroatoms selected from O, N and S, optionally substituted by C₁-C₆ alkyl, halogen, oxo, CN or OH; C(O)OR^{8a} or C(O)R^{8a}, wherein R^{8a} is C₁-C₆ alkyl optionally substituted by halogen, C₁-C₆ alkoxy, oxo, CN or OH, or R^{8a} is a C₃-C₈ cycloalkyl group optionally substituted by C₁-C₆ alkyl, or R^{8a} is a 3-11 membered heterocycloyalkyl, such as containing 1-4 heteroatoms selected from O, N and S, optionally substituted by C₁-C₆ alkyl, halogen, oxo, CN or OH.

In some embodiments of a compound of the present invention, such as a compound of formula (I), (Ic), (Id), (Ie), (Ii), (Ij), or (II), where applicable, optional substituents of R⁸ are selected from the group consisting of halogen, oxo, CN, OH, C₁-C₆ alkyl, NH₂, NH(C₁-C₆ alkyl), and N(C₁-C₆ alkyl)₂.

In some embodiments of a compound of the present invention, such as a compound of formula (I), (Id), or (II), where applicable, R⁸ and R^{8aa} together form a 3-11 membered heterocyclyl (e.g., a 5-6 membered heteroaryl containing 1-4 heteroatoms selected from O, N and S or 4-11 membered heterocycloalkyl containing 1-4 heteroatoms selected from O, N and S) optionally substituted by halogen, oxo, CN, OH, C₁-C₆ alkyl or C₁-C₆ alkoxy.

In some embodiments of a compound of the present invention, such as a compound of formula (I), (Ia), (Ib), (Ic), (Id), (Ie), (If), (Ig), (Ih), (Ii), (Ij), (Ik) or (II), where applicable, R⁶, R⁷ and R⁸ are each independently selected from C(O)NR^{a}R^{b}, C(O)R^{8a}, and C(O)OR⁸a. In some embodiments, R^{a} and R^{b} are independently selected from hydrogen, C₁-C₆ alkyl, or (C₁-C₆ alkylene)phenyl, or R^{a} and R^{b} are taken together to form a 3-11 membered heterocyclyl (e.g., a 5-6 membered heteroaryl containing 1-4 heteroatoms selected from O, N and S or 4-11 membered heterocycloalkyl containing 1-4 heteroatoms selected from O, N and S) optionally substituted by halogen, C₁-C₆ alkyl, oxo, OH, CN, NH₂, NHCH₃, or N(CH₃)₂. In some embodiments, R^{8a} is selected from C₁-C₆ alkyl optionally substituted by halogen, CN, OH, NH₂, NHCH₃, or N(CH₃)₂; C₃-C₈ cycloalkyl optionally substituted by C₁-C₆ alkyl or C₁-C₆ alkoxy; 3-11 membered heterocyclyl (e.g., a 5-6 membered heteroaryl containing 1-4 heteroatoms selected from O, N and S or 4-11 membered heterocycloalkyl containing 1-4 heteroatoms selected from O, N and S) optionally substituted by halogen, CN, OH, oxo, NH₂, NHCH₃, N(CH₃)₂, or C₁-C₆ alkyl.

In some embodiments of a compound of the present invention, such as a compound of formula (I), (Ia), (Ib), (Ic), (Id), (Ie), (If), (Ig), (Ih), (Ii), (Ij), (Ik) or (II), where applicable, R⁹, independently at each occurrence, is OH; halogen; C₁-C₆ alkyl optionally substituted with halogen, OH, CN, C₁-C₆ alkoxy, 5-6 membered heteroaryl (e.g., containing 1-4 heteroatoms selected from O, N and S), 3-11 membered heterocycloalkyl (e.g., containing 1-4 heteroatoms selected from O, N and S), NH₂, NHCH₃, or N(CH₃)₂; NH₂, NHCH₃, or N(CH₃)₂; (C₀-C₆ alkylene)C₃-C₈ cycloalkyl wherein the cycloalkyl is optionally substituted by halogen, C₁-C₆ alkyl, CN, OH, oxo or NR^{a}R^{b}; (C₀-C₆ alkylene)phenyl wherein the phenyl is optionally substituted by halogen, CN, OH, C₁-C₆ alkyl, or NR^{a}R^{b}; (C₀-C₆ alkylene)3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S), wherein the heterocyclyl is optionally substituted by halogen, CN, OH, oxo, C₁-C₆ alkyl, C(O)C₁-C₆ alkyl, NR^{a}R^{b} or 5-6 membered heteroaryl optionally substituted by C₁-C₆ alkyl; (C₀-C₆ alkylene)C(O)NR^{a}R^{b}; (C₀-C₆ alkylene)NR^{a}R^{b}; or C(O)(C₁-C₆ alkyl); wherein unless otherwise specified, R^{a} and R^{b} are independently at each occurrence hydrogen, C₁-C₆ alkyl, (C₀-C₆ alkylene)C₃-C₈ cycloalkyl, or (C₀-C₆ alkylene)phenyl, and wherein one or more alkylene units of any alkyl group is independently optionally substituted by —O—, or alternatively R^{a} and R^{b} may be joined together with the nitrogen atom to which they are attached to form an optionally substituted 3-11 membered heterocyclyl, e.g., 5-6 membered heteroaryl containing 1 to 4 heteroatoms selected from O, N and S or 4-11 membered heterocycloalkyl containing 1 to 4 heteroatoms selected from O, N and S, and wherein said optional substituents of said 3-11 membered heterocyclyl group are selected from CN, halogen, OH, C(O)CH₃, 5-6 membered heteroaryl optionally substituted by C₁-C₆ alkyl or halogen, and C₁-C₆ alkyl optionally substituted by halogen, OH, CN, oxo, or C₁-C₆ alkoxy. In some embodiments, R^{a} and R^{b} are selected from NH₂, NHCH₃, and N(CH₃)₂.

In some embodiments of a compound of the present invention, such as a compound of formula (I), (Ia), (Ib), (Ic), (Id), (Ie), (If), (Ig), (Ih), (Ii), (Ij), (Ik) or (II), where applicable, an optional substituent of R⁹ is selected from the group consisting of halogen, CN, OH, C₁-C₆ alkyl, C₁-C₆ alkoxy, or NR^{a}R^{b}, wherein NR^{a}R^{b} is selected from the group consisting of NH₂, NHCH₃, and N(CH₃)₂, NH-(3-11 membered heterocyclyl, e.g., 5-6 membered heteroaryl containing 1 to 4 heteroatoms selected from O, N and S or 4-11 membered heterocycloalkyl containing 1 to 4 heteroatoms selected from O, N and S), or R^{a} and R^{b} may be joined together with the nitrogen atom to which they are attached to form an optionally substituted 3-11 membered heterocyclyl, e.g., 5-6 membered heteroaryl containing 1 to 4 heteroatoms selected from O, N and S or 4-11 membered heterocycloalkyl containing 1 to 4 heteroatoms selected from O, N and S, and wherein said optional substituents of said 3-11 membered heterocyclyl group are selected from CN, halogen, OH, C(O)(C₁-C₆ alkyl) (e.g., C(O)CH₃), 5-6 membered heteroaryl optionally substituted with C₁-C₆ alkyl or halogen, and C₁-C₆ alkyl optionally substituted by halogen, OH, CN, oxo, OH or C₁-C₆ alkoxy.

In some embodiments of a compound of the present invention, such as a compound of formula (I), (Ia), (Ib), (Ic), (Id), (Ie), (If), (Ig), (Ih), (Ii), (Ij), (Ik) or (II), where applicable, R^{a} and R^{b}, independently at each occurrence, are selected from the group consisting of NH₂, NHCH₃, and N(CH₃)₂, or R^{a} and R^{b} may be joined together with the nitrogen atom to which they are attached to form an optionally substituted 3-11 membered heterocyclyl, e.g., 5-6 membered heteroaryl containing 1 to 4 heteroatoms selected from O, N and S or 4-11 membered heterocycloalkyl containing 1 to 4 heteroatoms selected from O, N and S, and wherein said optional substituents of said 3-11 membered heterocyclyl group are selected from CN, halogen, OH, C(O)(C₁-C₆ alkyl) (e.g., C(O)CH₃), 5-6 membered heteroaryl containing 1 to 4 heteroatoms selected from O, N and S optionally substituted with halogen, OH, CN or C₁-C₆ alkyl, and C₁-C₆ alkyl optionally substituted by halogen, OH, CN, oxo, OH or C₁-C₆ alkoxy.

In some embodiments of a compound of the present invention, such as a compound of formula (I), (Ig), (Ik), or (II), Ar² is optionally substituted phenyl or optionally substituted 5-11 membered heteroaryl. In some embodiments in a compound of the present invention, such as a compound of formula (I), (Ig), (Ik), or (II), Ar² is substituted pyridyl substituted with a heteroalkyl (e.g., 2-methoxyethoxy)pyridin-3-yl), q is 1, and R^{x} and R^{y} are each independently hydrogen.

In some embodiments, a compound of formula (I) excludes a compound of formula (Ia). In some embodiments, a compound of formula (I) excludes a compound of formula (Ib). In some embodiments, a compound of formula (I) excludes a compound of formula (Ic). In some embodiments, a compound of formula (I) excludes a compound of formula (Id). In some embodiments, a compound of formula (I) excludes a compound of formula (Ie). In some embodiments, a compound of formula (I) excludes a compound of formula (If). In some embodiments, a compound of formula (I) excludes a compound of formula (Ig). In some embodiments, a compound of formula (I) excludes a compound of formula (Ih). In some embodiments, a compound of formula (I) excludes a compound of formula (Ii). In some embodiments, a compound of formula (I) excludes a compound of formula (Ij). In some embodiments, a compound of formula (I) excludes a compound of formula (Ik). In some embodiments, a compound of formula (I) excludes two or more compounds of formula (I), (Ia), (Ib), (Ic), (Id), (Ie), (If), (Ig), (Ih), (Ii), (Ij) and (Ik).

In any compound of the present invention, including a compound of formula (I), (Ia), (Ib), (Ic), (Id), (Ie), (If), (Ig), (Ih), (Ii), (Ij), (Ik) or (II), any substituent indicated as “optionally substituted”, such as portions of R², R⁶, R⁷, R⁶ together with R⁷, R⁸, R^{8a}, R⁸ together with R^{8aa}, or R⁹, may be optionally substituted by, e.g., halogen; oxo; CN; NO; N₃; —OR′; perfluoro-C₁-C₄ alkoxy; unsubstituted C₃-C₇ cycloalkyl; C₃-C₇ cycloalkyl substituted by halogen, CN, OH, unsubstituted C₁-C₆ alkyl, unsubstituted C₁-C₆ alkoxy, oxo or NR′R″; unsubstituted C₆-C₁₀ aryl (e.g., phenyl); C₆-C₁₀ aryl substituted by halogen, CN, OH, unsubstituted C₁-C₆ alkyl, unsubstituted C₁-C₆ alkoxy, or NR′R″; unsubstituted 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S); 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S) substituted by halogen, CN, OH, unsubstituted C₁-C₆ alkyl, unsubstituted C₁-C₆ alkoxy, oxo or NR′R″; —NR′R″; —SR′; —SiR′R″R′″; —OC(O)R′; —C(O)R′; —CO₂R′; —CONR′R″; —OC(O)NR′R″; —NR″C(O)R′; —NR′″C(O)NR′R″; —NR″C(O)₂R′; —S(O)₂R′; —S(O)₂NR′R″; —NR'S(O)₂R″; —NR″ 'S(O)₂NR′R″; amidinyl; guanidinyl; —(CH₂)₁₋₄—OR′; —(CH₂)₁₋₄—NR′R″; —(CH₂)₁₋₄—SR′; —(CH₂)₁₋₄-SiR′R″R′″; —(CH₂)₁₋₄—OC(O)R′; —(CH₂)₁₋₄—C(O)R′; —(CH₂)₁₋₄—CO₂R′; and —(CH₂)₁₋₄CONR′R″, or combinations thereof, in a number ranging from zero to (2m′+1), where m′ is the total number of carbon atoms in such radical. R′, R″ and R′″each independently refer to groups including, for example, hydrogen; unsubstituted C₁-C₆ alkyl; C₁-C₆ alkyl substituted by halogen, CN, OH, unsubstituted C₁-C₆ alkyl, unsubstituted C₁-C₆ alkoxy, oxo or NR^{a}R^{b}; unsubstituted C₁-C₆ heteroalkyl; C₁-C₆ heteroalkyl substituted by halogen, CN, OH, unsubstituted C₁-C₆ alkyl, unsubstituted C₁-C₆ alkoxy, oxo or NR^{a}R^{b}; unsubstituted C₆-C₁₀ aryl; C₆-C₁₀ aryl substituted by halogen, CN, OH, unsubstituted C₁-C₆ alkyl, unsubstituted C₁-C₆ alkoxy, or NR^{a}R^{b}; unsubstituted 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S); and 3-11 membered heterocyclyl (e.g., 5-6 membered heteroaryl containing 1 to 4 heteroatoms selected from O, N and S or 4-11 membered heterocycloalkyl containing 1 to 4 heteroatoms selected from O, N and S) substituted by halogen, CN, OH, unsubstituted C₁-C₆ alkyl, unsubstituted C₁-C₆ alkoxy, oxo or NR^{a}R^{b}. When R′ and R″ are attached to the same nitrogen atom, they can be combined with the nitrogen atom to form a 3-, 4-, 5-, 6-, or 7-membered ring wherein a ring atom is optionally substituted with N, O or S and wherein the ring is optionally substituted with halogen, CN, OH, unsubstituted C₁-C₆ alkyl, unsubstituted C₁-C₆ alkoxy, oxo or NR′R″.

Representative compounds of the invention, and salts, are listed in Table 1. Throughout the application, if there is a discrepancy between the structure and its associated name, the structure prevails.

TABLE 1

| No. or | | |
|---|---|---|
| Letter | Structure | Name |
| 1-1 | 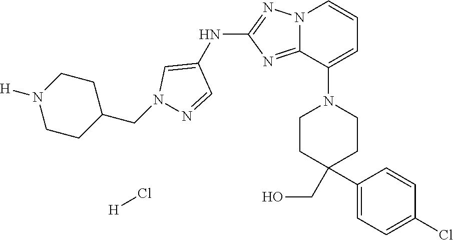 [SMILES: OCC1(c2ccc(Cl)cc2)CCN(c2cccn3nc(Nc4cnn(CC5CCNCC5)c4)nc23)CC1 ; Cl] | [4-(4-chlorophenyl)-1-[2-[[1-(4- piperidylmethyl)pyrazol-4- yl]amino]-[1,2,4]triazolo[1,5- a]pyridin-8-yl]-4- piperidyl]methanol; hydrochloride |
| 1-2 | 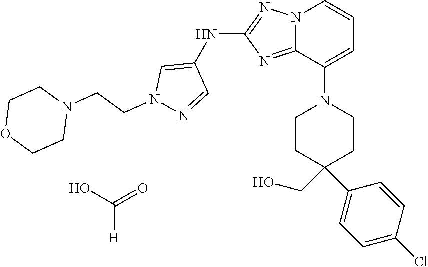 [SMILES: OCC1(c2ccc(Cl)cc2)CCN(c2cccn3nc(Nc4cnn(CCN5CCOCC5)c4)nc23)CC1 ; O=CO] | [4-(4-chlorophenyl)-1-[2-[[1-(2- morpholinoethyl)pyrazol-4- yl]amino]-[1,2,4]triazolo[1,5- a]pyridin-8-yl]-4- piperidyl]methanol; formic acid |
| 1-3 | 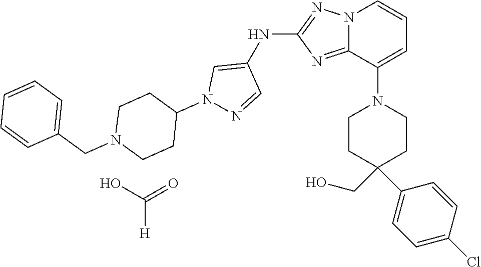 | [1-[2-[[1-(1-benzyl-4- piperidyl)pyrazol-4-yl]amino]- [1,2,4]triazolo[1,5-a]pyridin-8-yl]-4- (4-chlorophenyl)-4- piperidyl]methanol; formic acid |
| 1-4 | 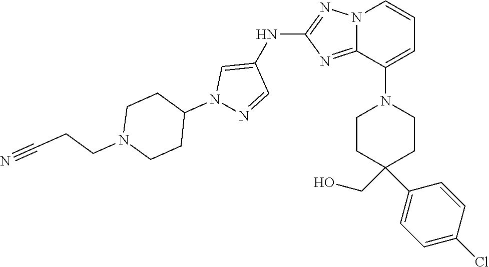 [SMILES: N#CCCN1CCC(n2cc(Nc3nc4c(N5CCC(CO)(c6ccc(Cl)cc6)CC5)cccn4n3)cn2)CC1] | 3-[4-[4-[[8-[4-(4-chlorophenyl)-4- (hydroxymethyl)-1-piperidyl]- [1,2,4]triazolo[1,5-a]pyridin-2- yl]amino]pyrazol-1-yl]-1- piperidyl]propanenitrile |
| 1-5 | 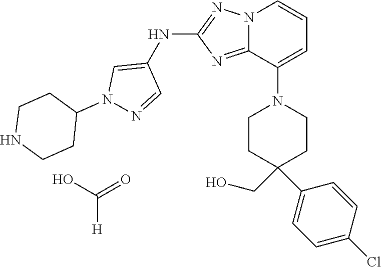 | [4-(4-chlorophenyl)-1-[2-[[1-(4- piperidyl)pyrazol-4-yl]amino]- [1,2,4]triazolo[1,5-a]pyridin-8-yl]-4- piperidyl]methanol; formic acid |
| 1-6 | 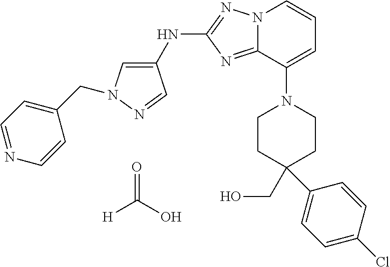 [SMILES: OCC1(c2ccc(Cl)cc2)CCN(c2cccn3nc(Nc4cnn(Cc5ccncc5)c4)nc23)CC1 ; O=CO] | [4-(4-chlorophenyl)-1-[2-[[1-(4- pyridylmethyl)pyrazol-4-yl]amino]- [1,2,4]triazolo[1,5-a]pyridin-8-yl]-4- piperidyl]methanol; formic acid |
| 1-7 | 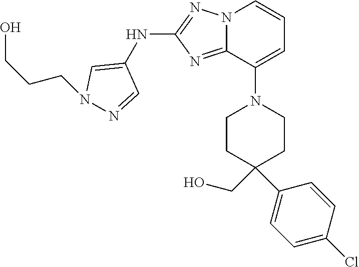 [SMILES: OCCCn1cc(Nc2nc3c(N4CCC(CO)(c5ccc(Cl)cc5)CC4)cccn3n2)cn1] | 3-[4-[[8-[4-(4-chlorophenyl)-4- (hydroxymethyl)-1-piperidyl]- [1,2,4]triazolo[1,5-a]pyridin-2- yl]amino]pyrazol-1-yl]propan-1-ol |
| 1-8 | 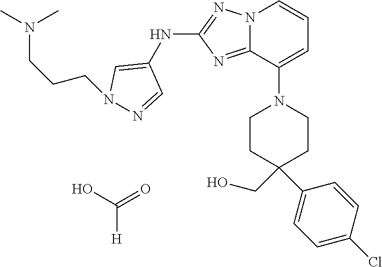 [SMILES: CN(C)CCCn1cc(Nc2nc3c(N4CCC(CO)(c5ccc(Cl)cc5)CC4)cccn3n2)cn1 ; O=CO] | [4-(4-chlorophenyl)-1-[2-[[1-[3- (dimethylamino)propyl]pyrazol-4- yl]amino]-[1,2,4]triazolo[1,5- a]pyridin-8-yl]-4- piperidyl]methanol; formic acid |
| 1-9 | 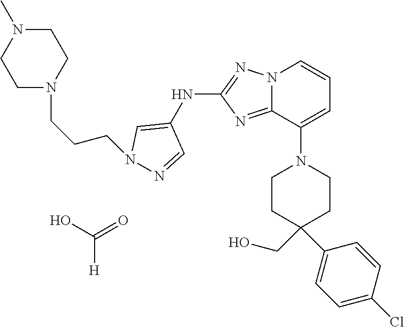 [SMILES: CN1CCN(CCCn2cc(Nc3nc4c(N5CCC(CO)(c6ccc(Cl)cc6)CC5)cccn4n3)cn2)CC1 ; O=CO] | [4-(4-chlorophenyl)-1-[2-[[1-[3-(4- methylpiperazin-1- yl)propyl]pyrazol-4-yl]amino]- [1,2,4]triazolo[1,5-a]pyridin-8-yl]-4- piperidyl]methanol; formic acid |
| 1-10 | 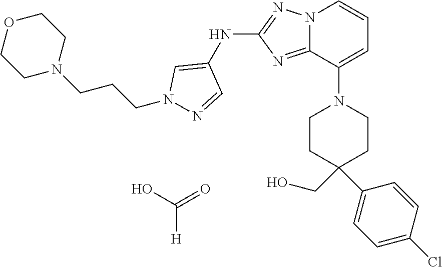 [SMILES: OCC1(c2ccc(Cl)cc2)CCN(c2cccn3nc(Nc4cnn(CCCN5CCOCC5)c4)nc23)CC1 ; O=CO] | [4-(4-chlorophenyl)-1-[2-[[1-(3- morpholinopropyl)pyrazol-4- yl]amino]-[1,2,4]triazolo[1,5- a]pyridin-8-yl]-4- piperidyl]methanol; formic acid |
| 1-11 | 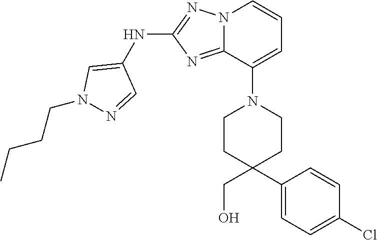 [SMILES: CCCCn1cc(Nc2nc3c(N4CCC(CO)(c5ccc(Cl)cc5)CC4)cccn3n2)cn1] | [1-[2-[(1-butylpyrazol-4-yl)amino]- [1,2,4]triazolo[1,5-a]pyridin-8-yl]-4- (4-chlorophenyl)-4- piperidyl]methanol |
| 1-12 | 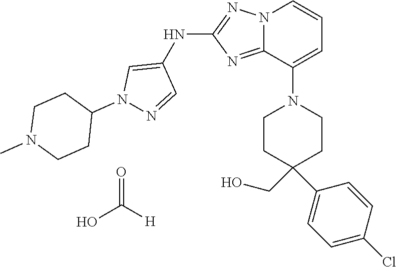 | [4-(4-chlorophenyl)-1-[2-[[1-(1- methyl-4-piperidyl)pyrazol-4- yl]amino]-[1,2,4]triazolo[1,5- a]pyridin-8-yl]-4- piperidyl]methanol; formic acid |
| 1-13 | 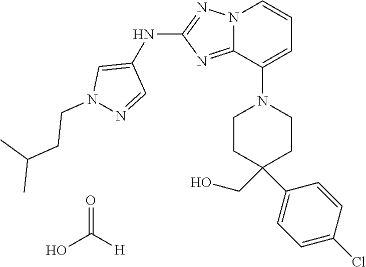 [SMILES: CC(C)CCn1cc(Nc2nc3c(N4CCC(CO)(c5ccc(Cl)cc5)CC4)cccn3n2)cn1 ; O=CO] | [4-(4-chlorophenyl)-1-[2-[(1- isopentylpyrazol-4-yl)amino]- [1,2,4]triazolo[1,5-a]pyridin-8-yl]-4- piperidyl]methanol; formic acid |
| 1-14 | 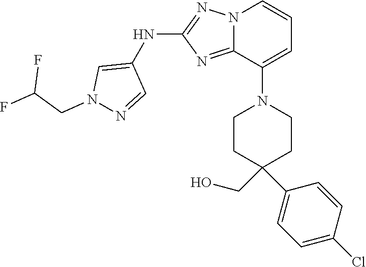 [SMILES: OCC1(c2ccc(Cl)cc2)CCN(c2cccn3nc(Nc4cnn(CC(F)F)c4)nc23)CC1] | [4-(4-chlorophenyl)-1-[2-[[1-(2,2- difluoroethyl)pyrazol-4-yl]amino]- [1,2,4]triazolo[1,5-a]pyridin-8-yl]-4- piperidyl]methanol |
| 1-15 | 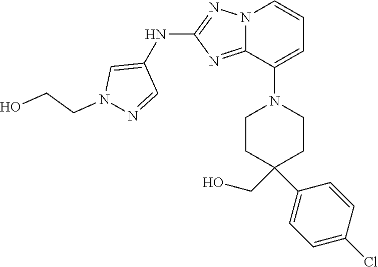 [SMILES: OCCn1cc(Nc2nc3c(N4CCC(CO)(c5ccc(Cl)cc5)CC4)cccn3n2)cn1] | 2-[4-[[8-[4-(4-chlorophenyl)-4- (hydroxymethyl)-1-piperidyl]- [1,2,4]triazolo[1,5-a]pyridin-2- yl]amino]pyrazol-1-yl]ethanol |
| 2-1 | 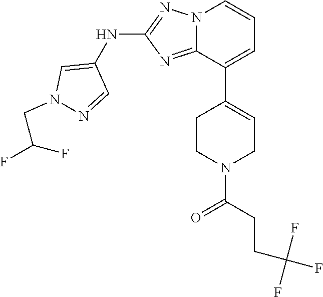 [SMILES: O=C(CCC(F)(F)F)N1CC=C(c2cccn3nc(Nc4cnn(CC(F)F)c4)nc23)CC1] | 1-[4-[2-[[1-(2,2- difluoroethyl)pyrazol-4-yl]amino]- [1,2,4]triazolo[1,5-a]pyridin-8-yl]- 3,6-dihydro-2H-pyridin-1-yl]-4,4,4- trifluoro-butan-1-one |
| 2-2 | 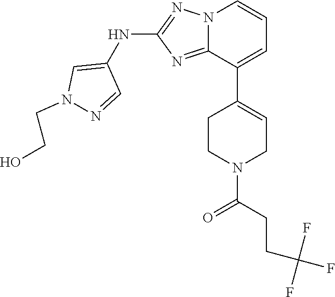 [SMILES: O=C(CCC(F)(F)F)N1CC=C(c2cccn3nc(Nc4cnn(CCO)c4)nc23)CC1] | 4,4,4-trifluoro-1-[4-[2-[[1-(2- hydroxyethyl)pyrazol-4-yl]amino]- [1,2,4]triazolo[1,5-a]pyridin-8-yl]- 3,6-dihydro-2H-pyridin-1-yl]butan- 1-one |
| 3-1 | 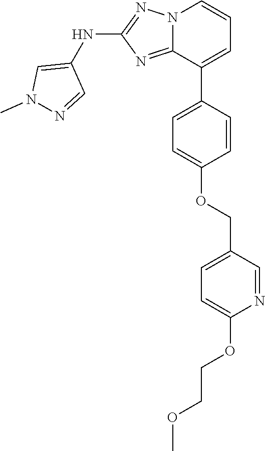 [SMILES: COCCOc1ccc(COc2ccc(-c3cccn4nc(Nc5cnn(C)c5)nc34)cc2)cn1] | 8-[4-[[6-(2-methoxyethoxy)-3- pyridyl]methoxy]phenyl]-N-(1- methylpyrazol-4-yl)- [1,2,4]triazolo[1,5-a]pyridin-2-amine |
| 3-2 | 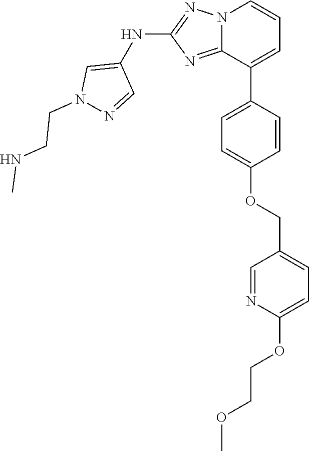 [SMILES: CNCCn1cc(Nc2nc3c(-c4ccc(OCc5ccc(OCCOC)nc5)cc4)cccn3n2)cn1] | 8-[4-[[6-(2-methoxyethoxy)-3- pyridyl]methoxy]phenyl]-N-[1-[2- (methylamino)ethyl]pyrazol-4-yl]- [1,2,4]triazolo[1,5-a]pyridin-2-amine |
| 3-3 | 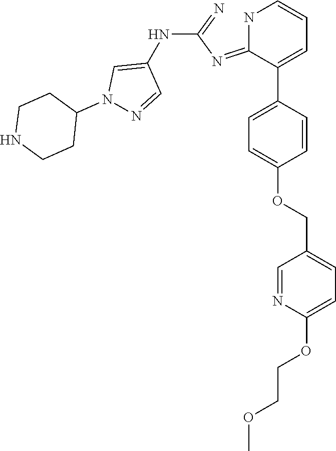 [SMILES: COCCOc1ccc(COc2ccc(-c3ccc[nH]/c3=N\C(=N)Nc3cnn(C4CCNCC4)c3)cc2)cn1] | 8-[4-[[6-(2-methoxyethoxy)-3- pyridyl]methoxy]phenyl]-N-[1-(4- piperidyl)pyrazol-4-yl]- [1,2,4]triazolo[1,5-a]pyridin-2-amine |
| 3-4 | 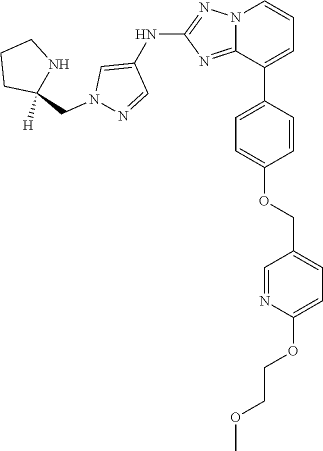 [SMILES: COCCOc1ccc(COc2ccc(-c3cccn4nc(Nc5cnn(C[C@H]6CCCN6)c5)nc34)cc2)cn1] | 8-[4-[[6-(2-methoxyethoxy)-3- pyridyl]methoxy]phenyl]-N-[1- [[(2R)-pyrrolidin-2- yl]methyl]pyrazol-4-yl]- [1,2,4]triazolo[1,5-a]pyridin-2-amine |
| 3-5 | 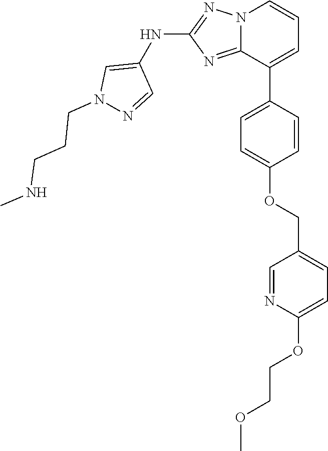 [SMILES: CNCCCn1cc(Nc2nc3c(-c4ccc(OCc5ccc(OCCOC)nc5)cc4)cccn3n2)cn1] | 8-[4-[[6-(2-methoxyethoxy)-3- pyridyl]methoxy]phenyl]-N-[1-[3- (methylamino)propyl]pyrazol-4-yl]- [1,2,4]triazolo[1,5-a]pyridin-2-amine |
| A | 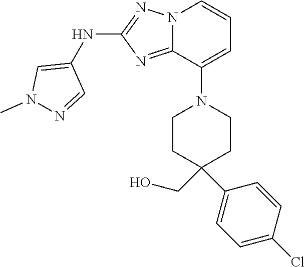 [SMILES: Cn1cc(Nc2nc3c(N4CCC(CO)(c5ccc(Cl)cc5)CC4)cccn3n2)cn1] | [4-(4-chlorophenyl)-1-[2-[(1- methylpyrazol-4-yl)amino]- [1,2,4]triazolo[1,5-a]pyridin-8-yl]-4- piperidyl]methanol |
| B | 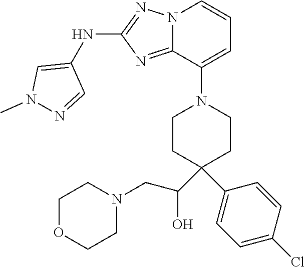 [SMILES: Cn1cc(Nc2nc3c(N4CCC(c5ccc(Cl)cc5)(C(O)CN5CCOCC5)CC4)cccn3n2)cn1] (mixture of enantiomers) | 1-[4-(4-chlorophenyl)-1-[2-[(1- methylpyrazol-4-yl)amino]- [1,2,4]triazolo[1,5-a]pyridin-8-yl]-4- piperidyl]-2-morpholino-ethanol Or 1-[4-(4-chlorophenyl)-1-[2-[(1- methyl-1H-pyrazol-4-yl)amino]- [1,2,4]triazolo[1,5-a]pyridin-8- yl]piperidin-4-yl]-2-(morpholin-4- yl)ethan-1-ol |
| C | 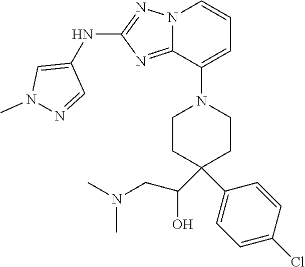 [SMILES: CN(C)CC(O)C1(c2ccc(Cl)cc2)CCN(c2cccn3nc(Nc4cnn(C)c4)nc23)CC1] (mixture of enantiomers) | 1-[4-(4-chlorophenyl)-1-[2-[(1- methylpyrazol-4-yl)amino]- [1,2,4]triazolo[1,5-a]pyridin-8-yl]-4- piperidyl]-2-(dimethylamino)ethanol |
| D | 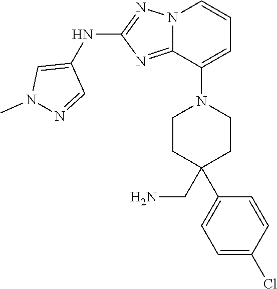 [SMILES: Cn1cc(Nc2nc3c(N4CCC(CN)(c5ccc(Cl)cc5)CC4)cccn3n2)cn1] | 8-[4-(aminomethyl)-4-(4- chlorophenyl)-1-piperidyl]-N-(1- methylpyrazol-4-yl)- [1,2,4]triazolo[1,5-a]pyridin-2-amine |
| E | 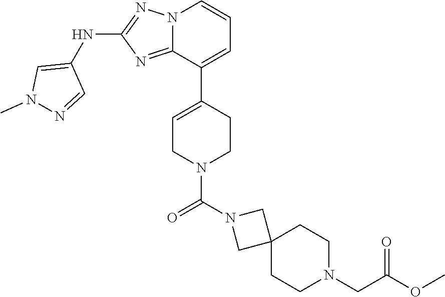 | methyl 2-[2-[4-[2-[(1-methylpyrazol- 4-yl)amino]-[1,2,4]triazolo[1,5- a]pyridin-8-yl]-3,6-dihydro-2H- pyridine-1-carbonyl]-2,7- diazaspiro[3.5]nonan-7-yl]acetate |
| F | 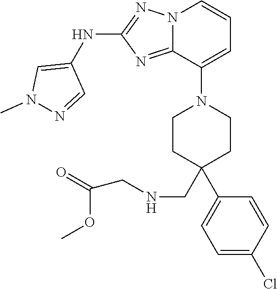 [SMILES: COC(=O)CNCC1(c2ccc(Cl)cc2)CCN(c2cccn3nc(Nc4cnn(C)c4)nc23)CC1] | formic acid; methyl 2-[[4-(4- chlorophenyl)-1-[2-[(1- methylpyrazol-4-yl)amino]- [1,2,4]triazolo[1,5-a]pyridin-8-yl]-4- piperidyl]methylamino] |
| G | 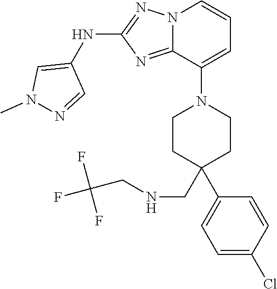 [SMILES: Cn1cc(Nc2nc3c(N4CCC(CNCC(F)(F)F)(c5ccc(Cl)cc5)CC4)cccn3n2)cn1] | 8-[4-(4-chlorophenyl)-4-[(2,2,2- trifluoroethylamino)methyl]-1- piperidyl]-N-(1-methylpyrazol-4-yl)- [1,2,4]triazolo[1,5-a]pyridin-2-amine |
| H | 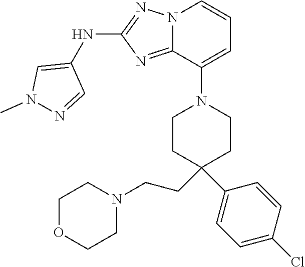 [SMILES: Cn1cc(Nc2nc3c(N4CCC(CCN5CCOCC5)(c5ccc(Cl)cc5)CC4)cccn3n2)cn1] | 8-[4-(4-chlorophenyl)-4-(2- morpholinoethyl)-1-piperidyl]-N-(1- methylpyrazol-4-yl)- [1,2,4]triazolo[1,5-a]pyridin-2-amine |
| I | 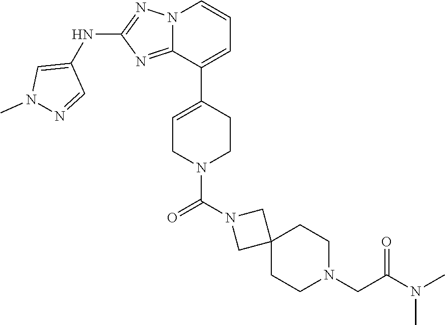 | N,N-dimethyl-2-[2-[4-[2-[(1- methylpyrazol-4-yl)amino]- [1,2,4]triazolo[1,5-a]pyridin-8-yl]- 3,6-dihydro-2H-pyridine-1-carbonyl]- 2,7-diazaspiro[3.5]nonan-7- yl]acetamide |
| J | 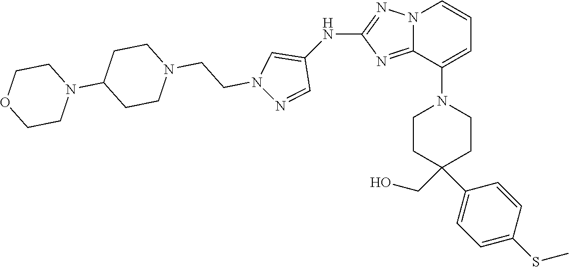 [SMILES: CSc1ccc(C2(CO)CCN(c3cccn4nc(Nc5cnn(CCN6CCC(N7CCOCC7)CC6)c5)nc34)CC2)cc1] | [4-(4-methylsulfanylphenyl)-1-[2-[[1- [2-(4-morpholino-1- piperidyl)ethyl]pyrazol-4-yl]amino]- [1,2,4]triazolo[1,5-a]pyridin-8-yl]-4- piperidyl]methanol |
| K | 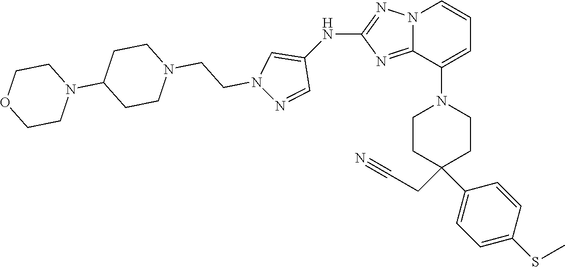 [SMILES: CSc1ccc(C2(CC#N)CCN(c3cccn4nc(Nc5cnn(CCN6CCC(N7CCOCC7)CC6)c5)nc34)CC2)cc1] | 2-[4-(4-methylsulfanylphenyl)-1-[2- [[1-[2-(4-morpholino-1- piperidyl)ethyl]pyrazol-4-yl]amino]- [1,2,4]triazolo[1,5-a]pyridin-8-yl]-4- piperidyl]acetonitrile |
| L | 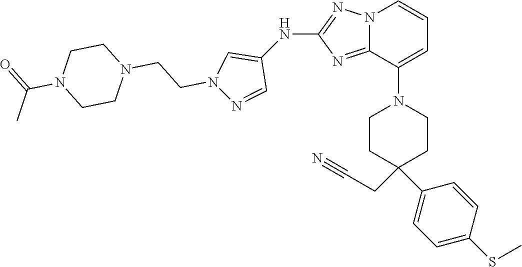 [SMILES: CSc1ccc(C2(CC#N)CCN(c3cccn4nc(Nc5cnn(CCN6CCN(C(C)=O)CC6)c5)nc34)CC2)cc1] | 2-[1-[2-[[1-[2-(4-acetylpiperazin-1- yl)ethyl]pyrazol-4-yl]amino]- [1,2,4]triazolo[1,5-a]pyridin-8-yl]-4- (4-methylsulfanylphenyl)-4- piperidyl]acetonitrile |
| M | 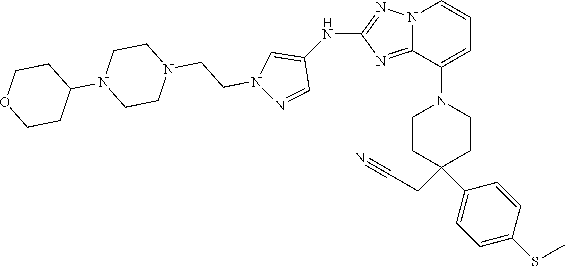 [SMILES: CSc1ccc(C2(CC#N)CCN(c3cccn4nc(Nc5cnn(CCN6CCN(C7CCOCC7)CC6)c5)nc34)CC2)cc1] | 2-[4-(4-methylsulfanylphenyl)-1-[2- [[1-[2-(4-tetrahydropyran-4- ylpiperazin-1-yl)ethyl]pyrazol-4- yl]amino]-[1,2,4]triazolo[1,5- a]pyridin-8-yl]-4- piperidyl]acetonitrile |
| N | 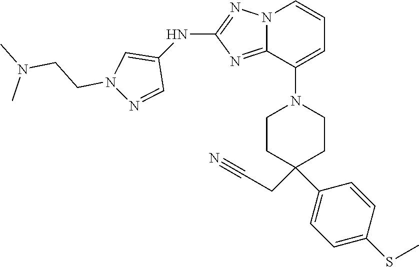 | 2-[1-[2-[[l-[2- (dimethylamino)ethyl]pyrazol-4- yl]amino]-[1,2,4]triazolo[1,5- a]pyridin-8-yl]-4-(4- methylsulfanylphenyl)-4- piperidyl]acetonitrile |
| O | 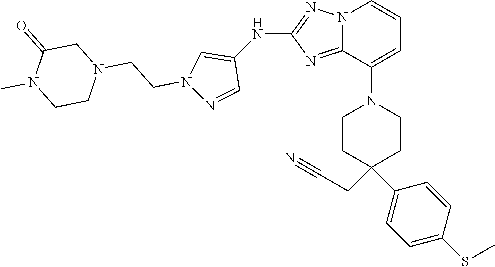 | 2-[1-[2-[[1-[2-(4-methyl-3-oxo- piperazin-1-yl)ethyl]pyrazol-4- yl]amino]-[1,2,4]triazolo[1,5- a]pyridin-8-yl]-4-(4- methylsulfanylphenyl)-4- piperidyl]acetonitrile |
| P | 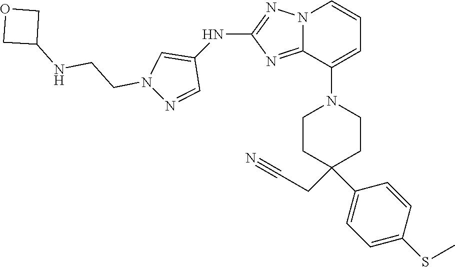 [SMILES: CSc1ccc(C2(CC#N)CCN(c3cccn4nc(Nc5cnn(CCNC6COC6)c5)nc34)CC2)cc1] | 2-[4-(4-methylsulfanylphenyl)-1-[2- [[1-[2-(oxetan-3- ylamino)ethyl]pyrazol-4-yl]amino]- [1,2,4]triazolo[1,5-a]pyridin-8-yl]-4- piperidyl]acetonitrile |
| Q | 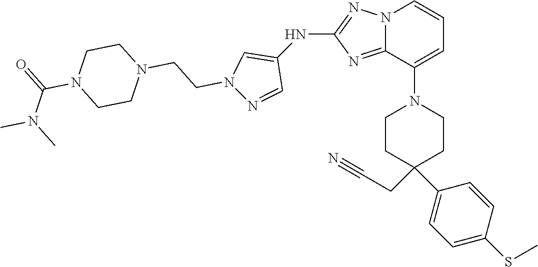 | 4-[2-[4-[[8-[4-(cyanomethyl)-4-(4- methylsulfanylphenyl)-1-piperidyl]- [1,2,4]triazolo[1,5-a]pyridin-2- yl]amino]pyrazol-1-yl]ethyl]-N,N- dimethyl-piperazine-1-carboxamide |
| R | 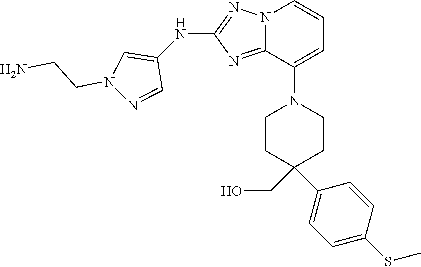 | [1-[2-[[1-(2-aminoethyl)pyrazol-4- yl]amino]-[1,2,4]triazolo[1,5- a]pyridin-8-yl]-4-(4- methylsulfanylphenyl)-4- piperidyl]methanol |
| S | 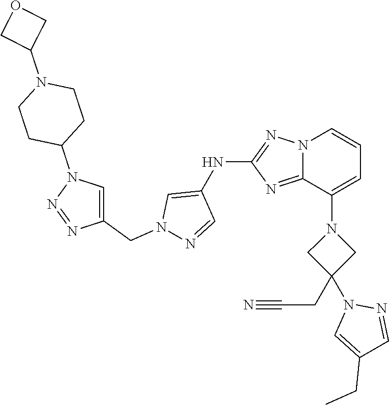 [SMILES: CCc1cnn(C2(CC#N)CN(c3cccn4nc(Nc5cnn(Cc6cn(C7CCN(C8COC8)CC7)nn6)c5)nc34)C2)c1] | 2-[3-(4-ethylpyrazol-1-yl)-1-[2-[[1- [[1-[1-(oxetan-3-yl)-4- piperidyl]triazol-4-yl]methyl]pyrazol- 4-yl]amino]-[1,2,4]triazolo[1,5- a]pyridin-8-yl]azetidin-3- yl]acetonitrile Or 2-[3-(4-ethyl-1H-pyrazol-1-yl)-1-(2- [[1-([1-[1-(oxetan-3-yl)piperidin-4- yl]-1H-1,2,3-triazol-4-yl]methyl)-1H- pyrazol-4-yl]amino]- [1,2,4]triazolo[1,5-a]pyridin-8- yl)azetidin-3-yl]acetonitrile |

In some embodiments, the invention relates to one or more of the compounds depicted in Table 1 (e.g., a compound selected from Compound Nos. 1-1 to 1-15, 2-1, 2-2, 3-1 to 3-5, and Letters A-S), and uses thereof. In some embodiments, the invention relates to one or more stereoisomers (e.g. diastereomers or enantiomers) of a compound depicted in Table 1 (e.g., a compounds selected from Compound Nos. 1-1 to 1-15, 2-1, 2-2, 3-1 to 3-5 and Letters A-S), and uses thereof.

The compounds provided herein may contain asymmetric or chiral centers, and, therefore, exist in different stereoisomeric forms. It is intended that all stereoisomeric forms of the compounds provided herein, including but not limited to: diastereomers, enantiomers, and atropisomers as well as mixtures thereof such as racemic mixtures, form part of the present invention. In addition, the present invention embraces all geometric and positional isomers. For example, if a compound incorporates a double bond or a fused ring, both the cis- and trans-forms, as well as mixtures, are embraced within the scope of the invention. Both the single positional isomers and mixture of positional isomers, e.g., resulting from the N-oxidation of the purine and pyrazolyl rings, or the E and Z forms of the compound (for example vinyl moieties), are also within the scope of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention, as defined by the claims, embrace both solvated and unsolvated forms.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In some embodiments, substantially pure” intends a composition that contains no more than 35%, 30%, 25%, 20%, 15%, 10%, 5%, 2% or 1% impurity, wherein the impurity denotes a compound other than the compound comprising the majority of the composition or a salt thereof.

In one variation, the compounds herein are synthetic compounds prepared for administration to an individual. In another variation, compositions are provided containing a compound in substantially pure form. In another variation, the invention embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

### General Synthetic Methods

The invention includes methods of making the compounds (as well as compositions comprising the compounds) described herein. The compounds of the invention may be prepared by a number of processes as generally described below and more specifically in the Examples hereinafter. In the following process descriptions, the symbols when used in the formulae depicted are to be understood to represent those groups described above in relation to the formulae herein.

Compounds described herein (e.g., Formulae I, II and variations thereof) may be synthesized by synthetic routes described herein. In certain embodiments, processes well-known in the chemical arts can be used, in addition to, or in light of, the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), Beilsteins Handbuch der organischen Chemie, 4, Aufl. ed. Springer-Verlag, Berlin, including supplements (also available via the Beilstein online database)), or Comprehensive Heterocyclic Chemistry, Editors Katrizky and Rees, Pergamon Press, 1984.

Compounds described herein (e.g., Formulae I, II and variations thereof) may be prepared singly or as compound libraries comprising at least 2, for example 5 to 1,000 compounds, or 10 to 100 compounds described herein (e.g., Formulae I, II and variations thereof). Libraries of compounds described herein may be prepared by a combinatorial ‘split and mix’ approach or by multiple parallel syntheses using either solution phase or solid phase chemistry, by procedures known to those skilled in the art. Thus according to a further aspect of the invention there is provided a compound library comprising at least 2 compounds described herein, a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S.

For illustrative purposes, reaction Schemes 1-24 depicted below provide routes for synthesizing the compounds of the present invention as well as key intermediates. For a more detailed description of the individual reaction steps, see the Examples section below. Those skilled in the art will appreciate that other synthetic routes may be used. Although some specific starting materials and reagents are depicted in the Schemes and discussed below, other starting materials and reagents can be substituted to provide a variety of derivatives or reaction conditions. In addition, many of the compounds prepared by the methods described below can be further modified in light of this disclosure using conventional chemistry well known to those skilled in the art.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups include acetyl, trifluoroacetyl, benzyl, phenylsulfonyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Other conversions commonly used in the synthesis of compounds of the present invention, and which can be carried out using a variety of reagents and conditions, include the following:
(1) Reaction of a carboxylic acid with an amine to form an amide. Such a transformation can be achieved using various reagents known to those skilled in the art but a comprehensive review can be found in Tetrahedron, 2005, 61, 10827-10852.
(2) Reaction of a primary or secondary amine with an aryl halide or pseudo halide, e.g., a triflate, commonly known as a “Buchwald-Hartwig cross-coupling,” can be achieved using a variety of catalysts, ligands and bases. A review of these methods is provided in Comprehensive Organic Name Reactions and Reagents, 2010, 575-581.
(3) A palladium cross-coupling reaction between an aryl halide and a vinyl boronic acid or boronate ester. This transformation is a type of “Suzuki-Miyaura cross-coupling,” a class of reaction that has been thoroughly reviewed in Chemical Reviews, 1995, 95(7), 2457-2483.
(4) The hydrolysis of an ester to give the corresponding carboxylic acid is well known to those skilled in the art and conditions include: for methyl and ethyl esters, the use of a strong aqueous base such as lithium, sodium or potassium hydroxide or a strong aqueous mineral acid such as HCl; for a tert-butyl ester, hydrolysis would be carried out using acid, for example, HCl in dioxane or trifluoroacetic acid (TFA) in dichloromethane (DCM).

Schemes 1 to 24 detail the reactions available for the preparation of compounds of the invention wherein R² of Formula (I) is of type (a) to (k). Scheme 1 outlines a method for the preparation of intermediates of formula (1-5).

Intermediate (1-1), prepared according to a procedure contained in WO2009/155551, may be converted via a diazotization reaction to a compound of Formula (1-2). Compounds of Formula (1-1) and (1-2) may be coupled to compounds of Formula (1-3) and (1-4) using a palladium catalysed Buchwald-Hartwig cross-coupling reaction, with a catalyst such as tris(dibenzylideneacetone)dipalladium (0) (Pd₂(dba)₃) or palladium (II) acetate (Pd(OAc)₂), a phosphine ligand such as 4,5-bis(diphenylphosphino)-9,9-dimethylxanthene (Xantphos) and a base such as caesium carbonate to afford compounds of Formula (1-5).

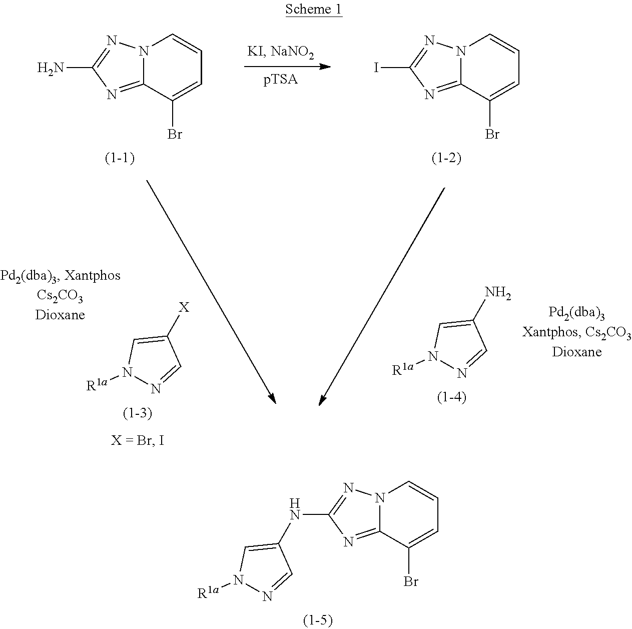

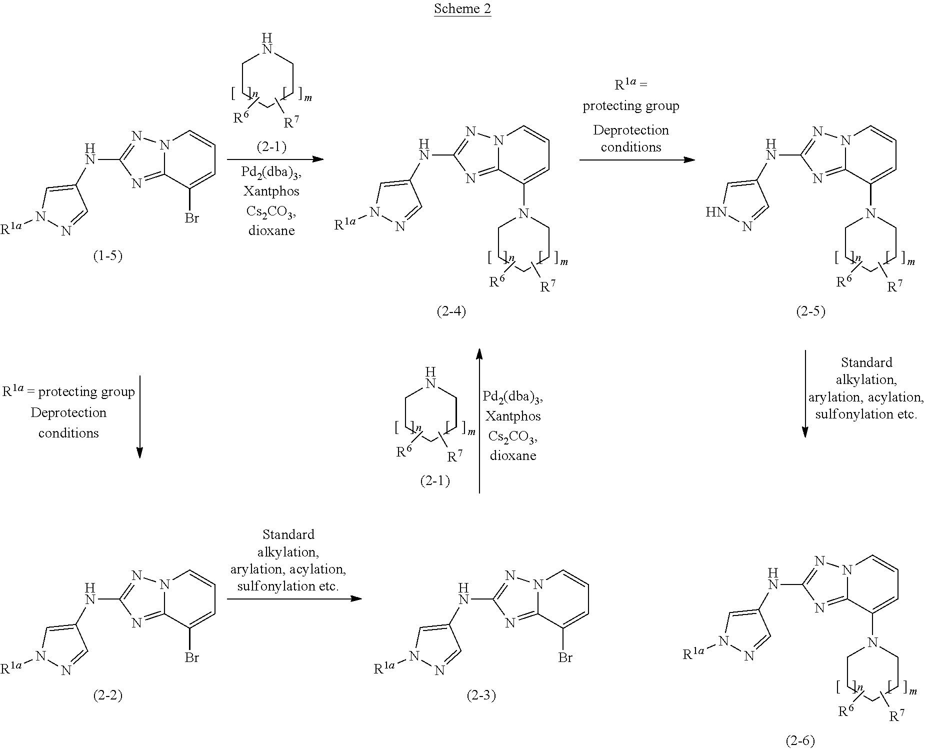

Scheme 2 provides details of the reactions available for the preparation of compounds of the invention wherein R² of Formula (I) is of type (a). Compounds of Formula (1-5) from Scheme 1 and compounds of Formula (2-3) may undergo a palladium catalysed Buchwald-Hartwig cross-coupling with an amine of Formula (2-1) using a catalyst such as tris(dibenzylideneacetone)dipalladium (0) (Pd₂(dba)₃) or palladium (II) acetate (Pd(OAc)₂), a phosphine ligand such as 4,5-bis(diphenylphosphino)-9,9-dimethylxanthene (Xantphos) or BINAP and a base such as caesium carbonate. Where R^{1a} in (1-5), or indeed (2-4), is a protecting group, it may be removed under standard conditions and the resulting amine (2-2) or (2-5) may be further modified using standard chemistries through alkylation, arylation, acylation, sulfonylation etc. to afford compounds of Formula (2-3) and (2-6).

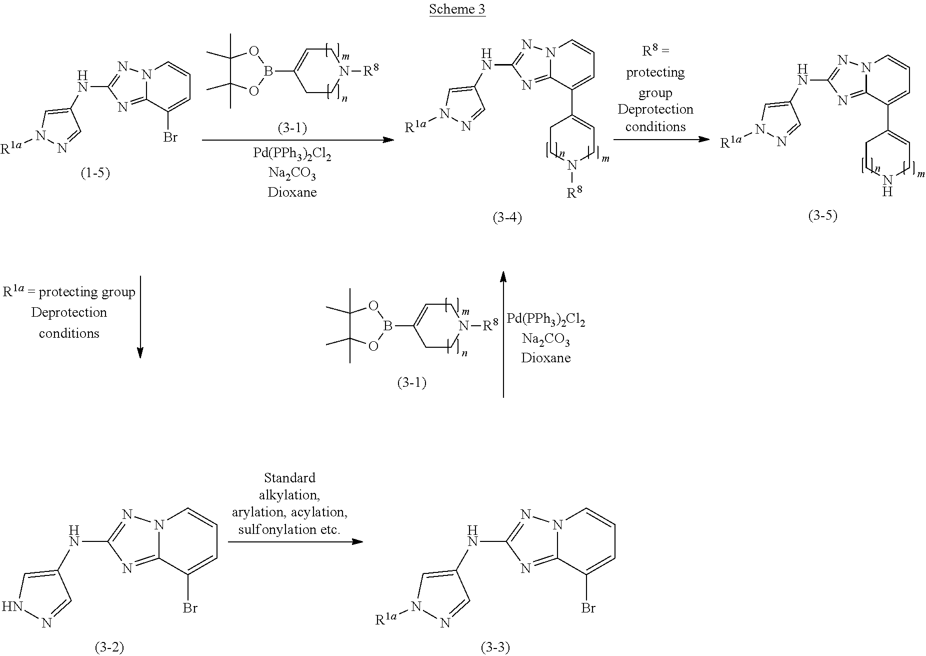

Scheme 3 provides details of the reactions available for the preparation of compounds of the invention wherein R² of Formula (I) is of type (c). Compounds of Formula (1-5) from Scheme 1 and compounds of Formula (3-3) may undergo a palladium catalysed Suzuki cross-coupling with the boronate (3-1) using a catalyst such as palladium bis-triphenylphosphine dichloride (Pd(PPh₃)₂Cl₂) or tetrakistriphenylphosphine palladium (0) (Pd(PPh₃)₄) with a base such as caesium carbonate. Where R^{1a} in (1-5), or indeed R⁸ in (3-4), is a protecting group, it may be removed under standard conditions. The resulting amine (3-2) may be further modified using standard chemistries through alkylation, arylation, acylation, sulfonylation etc. to afford compounds of Formula (3-3).

The amine (3-5) in Scheme 4 may be further modified using standard acylation, alkylation, arylation and sulfonylation chemistries. These specifically include: (i) Reaction with a chloroformate in the presence of a base such as triethylamine to give the corresponding carbamate; (ii) Alkylation with an alkyl halide in the presence of a base or reductive alkylation using an aldehyde or ketone and a reducing agent such as sodium triacetoxyborohydride; (iii) Acylation by reaction with a carboxylic acid and an amide coupling agent such as HATU or by reaction with an acid chloride in the presence of a base; (iv) Hydrogenation using hydrogen gas over a palladium catalyst; (v) Formation of an activated carbamate by reaction with 4-nitrophenyl chloroformate and then further reaction with an amine to form a urea; (vi) Arylation using an arylboronic acid or boronate ester in the presence of copper (II) acetate; (vii) Formation of a sulphonamide by reaction with a sulfonyl chloride in the presence of a base. The product of hydrogenation (4-3) can be subjected to reactions (i), (ii), (iii), (v), (vi) and (vii) in order to form compounds (4-4) wherein R² of is of type (e).

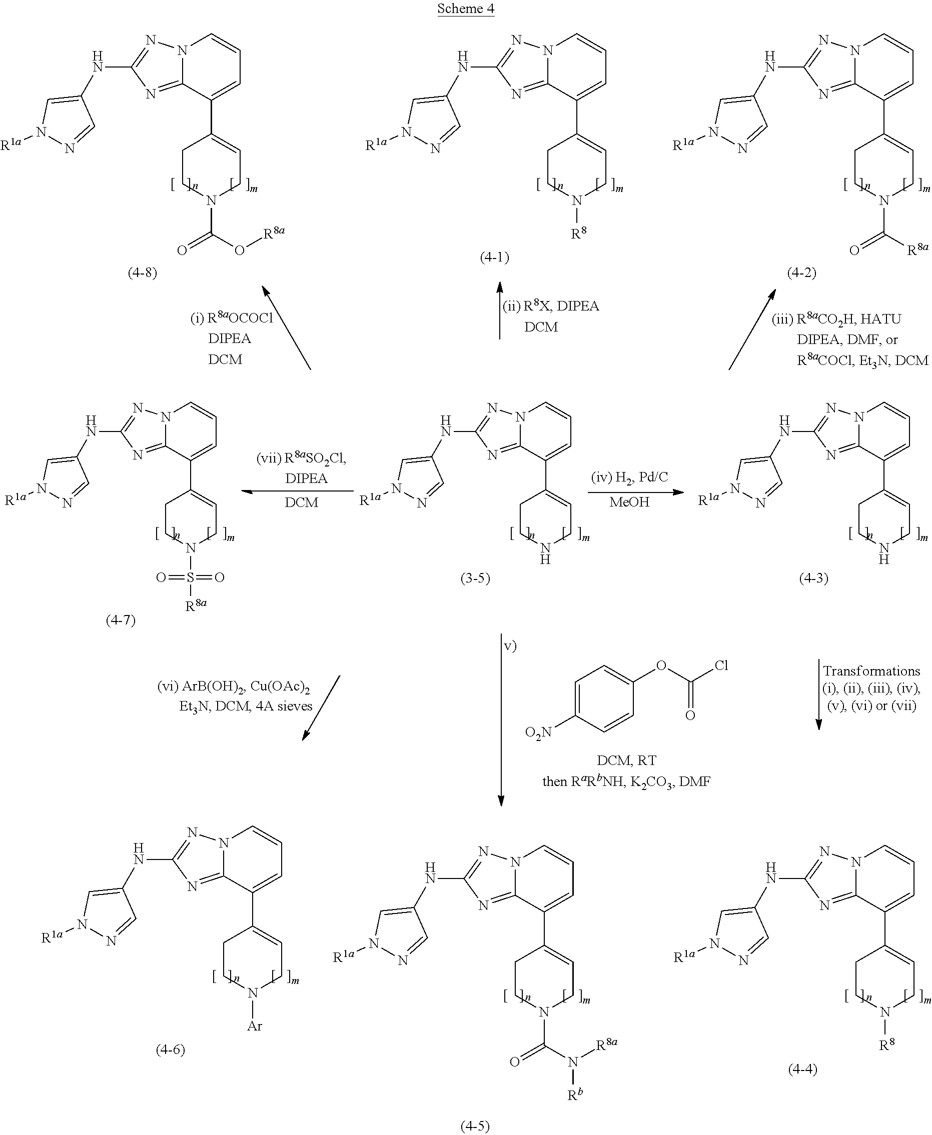

Scheme 5 provides details of the reactions available for the preparation of compounds of the invention wherein R² of Formula (I) is of type (d). Compounds of Formula (1-5) from Scheme 1 may undergo a palladium catalysed Buchwald-Hartwig cross-coupling with a diamine of Formula (5-1) using a catalyst such as tris(dibenzylideneacetone)dipalladium (0) (Pd₂(dba)₃) or palladium (II) acetate (Pd(OAc)₂), a phosphine ligand such as 4,5-bis(diphenylphosphino)-9,9-dimethylxanthene (Xantphos) and a base such as caesium carbonate. Where R^{1a} in (1-5), or indeed R⁸ in (5-4), is a protecting group, it may be removed under standard conditions and the resulting amine (5-2) or (5-5) may be further modified using standard chemistries through alkylation, arylation etc. to afford compounds of Formula (5-3) and (5-6).

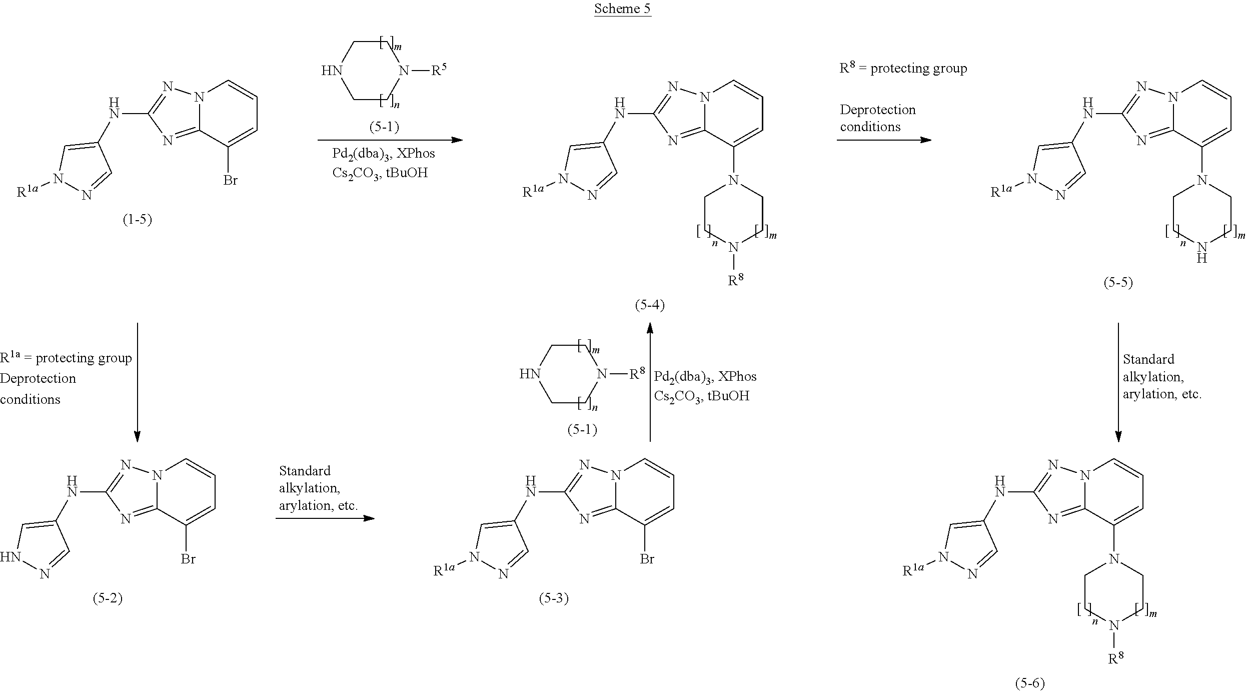

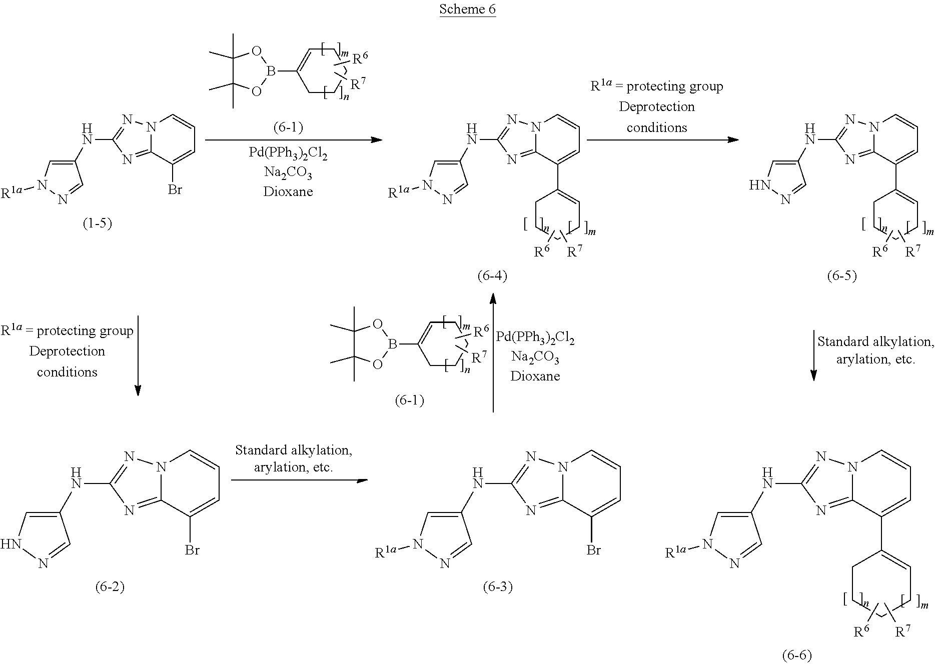

Scheme 6 provides details of the reactions available for the preparation of compounds of the invention wherein R² of Formula (I) is of type (f). Compounds of Formula (1-5) from Scheme 1 and compounds of Formula (6-3) may undergo a palladium catalysed Suzuki cross-coupling with the boronate (6-1) using a catalyst such as palladium bis-triphenylphosphine dichloride (Pd(PPh₃)₂Cl₂) or tetrakistriphenylphosphine palladium (0) (Pd(PPh₃)₄) and a base such as caesium carbonate. Where R^{1a} in (1-5), or indeed (6-4), is a protecting group, it may be removed under standard conditions and the resulting amine (6-2) and (6-5) may be further modified using standard chemistries through alkylation, arylation etc. to afford compounds of Formula (6-3) and (6-6).

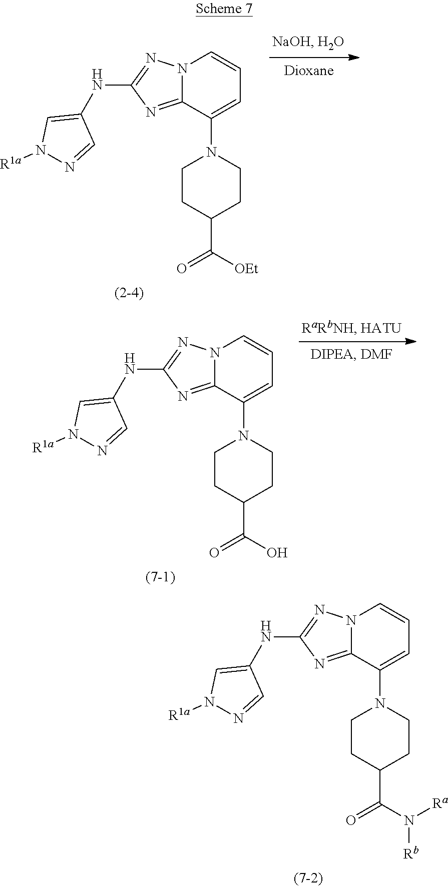

Where R⁶ in the compounds of Scheme 2 is an ester, exemplified by structure (2-4; R⁶=CO₂Et, R⁷═H)), this group may be hydrolyzed under standard conditions to give an acid of type (7-1). Common amidation conditions may then be used to prepare an amide (7-2) from the acid (7-1) and a suitable amine R^{a}R^{b}NH.

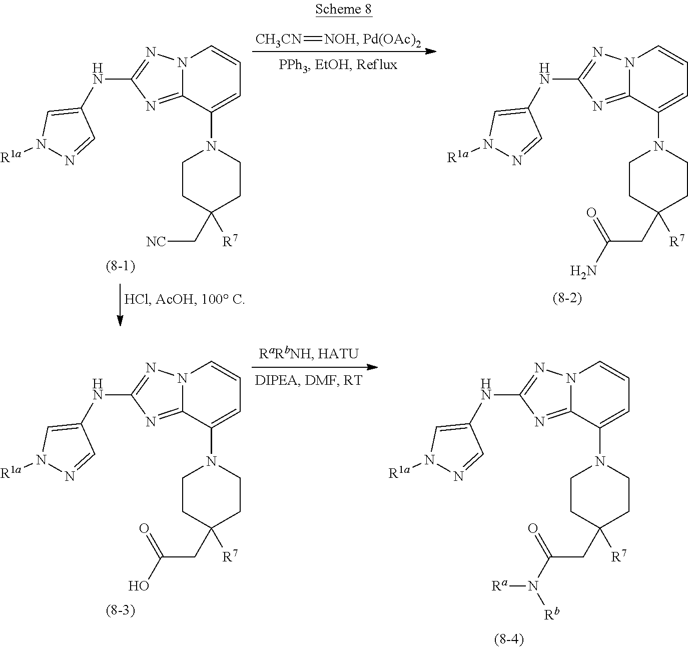

In Scheme 2, R⁶ or R⁷ may be further elaborated. For example, in Scheme 2, where R⁶ is CH₂CN (8-1), the nitrile group may be converted into the corresponding primary amide (8-2). Reagents suitable for this conversion include acetaldoxime in the presence of palladium (II) acetate and triphenylphosphine. The nitrile group in (8-1) may also be hydrolyzed to the corresponding carboxylic acid (8-3) which in turn can be treated with an amine R^{a}R^{b}NH under standard amidation conditions to provide compounds of type (8-4).

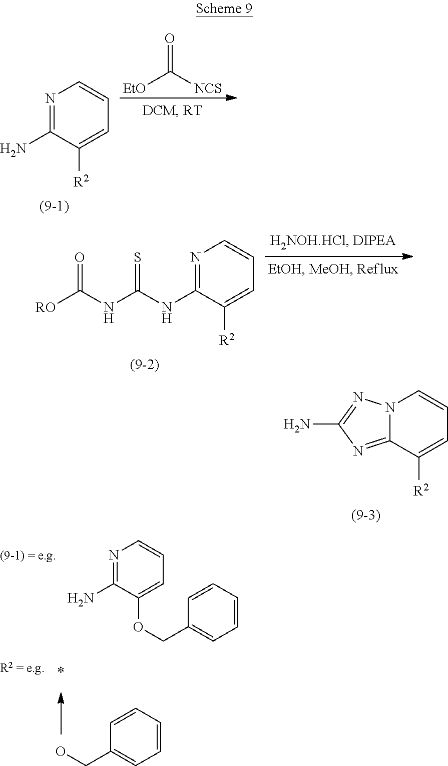

Scheme 9 shows an alternative approach available for the preparation of compounds of the invention. The group R² may be incorporated into a 2-aminopyridine (9-1) prior to formation of the bicycle (9-3) which may then be further modified using the methodologies described herein. This method is of particular use in the synthesis of compounds of Formula (I) wherein R² is of type (g). A method for preparing compounds (9-3) from 3-bromo-2-aminopyridine is available in WO2012/020848, incorporated herein by reference.

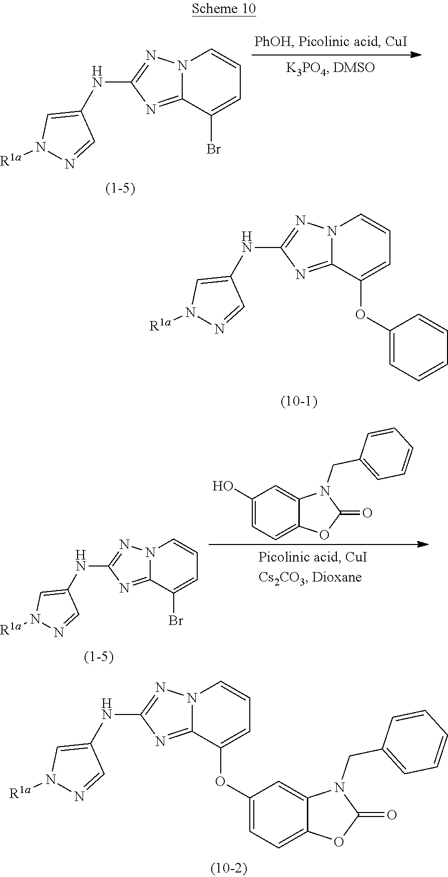

An alternative route to compounds of structure (g) wherein R² is OAr is described in Scheme 10. An intermediate (1-5) can be reacted with a phenol using a copper catalyst. Specifically, (1-5) can be converted into a compound of Formula (10-1) and (10-2) by heating with the appropriate phenol in the presence of picolinic acid, copper (I) iodide and a base such as potassium phosphate tribasic or caesium carbonate.

Scheme 11 shows a method that may be used to prepare a secondary amine as an intermediate towards the synthesis of compounds of Formula (I) type (a) wherein R⁶ is 2-(2-methoxyethoxy)pyridin-4-ylmethoxy (11-4). Treatment of Boc-protected 4-piperidinol (11-2) with a base such as potassium tert-butoxide, and reaction with commercially available 4-(chloromethyl)-2-(2-methoxyethoxy)pyridine (11-1) in the presence of an iodide source such as tetrabutylammonium iodide gives (11-3). Treatment of the latter with acid leads to Boc deprotection and provides amine (11-4).

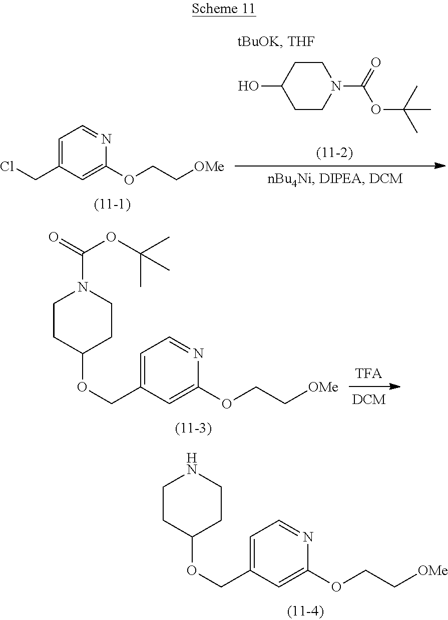 [SMILES: COCCOc1cc(CCl)ccn1 ; CC(C)(C)OC(=O)N1CCC(O)CC1 ; COCCOc1cc(COC2CCN(C(=O)OC(C)(C)C)CC2)ccn1 ; COCCOc1cc(COC2CCNCC2)ccn1]

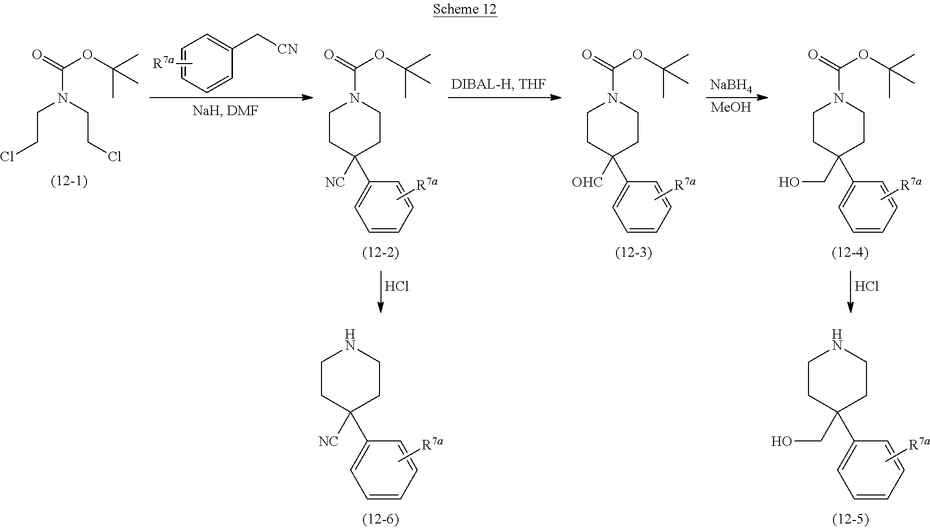

Scheme 12 describes routes to secondary amines (2-1) of Scheme 2 in which R⁷ is phenyl or substituted phenyl and R⁶ is either cyano (12-6) or hydroxymethyl (12-5). Treatment of a cyanomethylbenzene with a base such as sodium hydride and subsequent reaction of the resultant anion with commercially available (12-1) may provide piperidines of the formula (12-2). The deprotection of (12-2) under acidic conditions gives amine (12-6). Reduction of the nitrile in intermediate (12-2), using diisobutylaluminium hydride for example, affords the aldehyde (12-3) which may be further reduced to the alcohol (12-4) by treatment with sodium borohydride. Boc deprotection will give the amine (12-5).

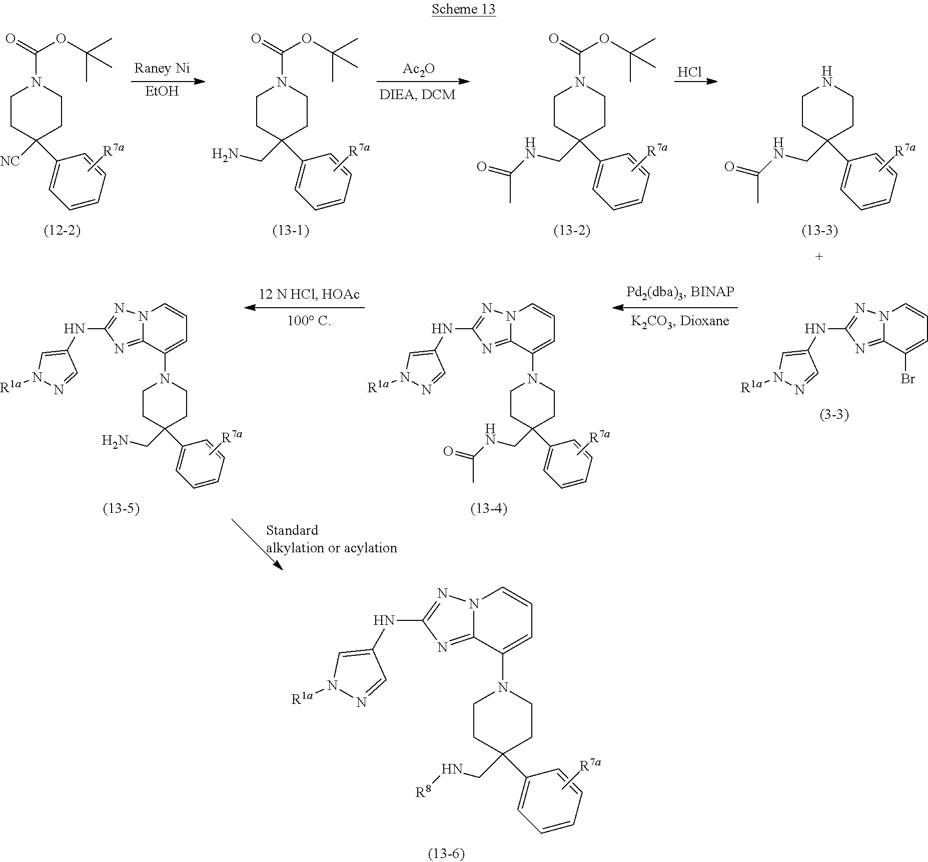

Scheme 13 describes the preparation of compounds of type 13-6. Reduction of the nitrile (12-2) using a metal catalyzed reduction under an atmosphere of hydrogen with a reagent such as Raney Nickel may be used to afford the methylamino intermediate (13-1) which may be protected as the acetamide using acetic anhydride and a base to afford acetamide intermediate (13-2). Boc deprotection under standard acidic conditions may be used to afford the amine (13-3). Coupling 13-3 with 3-3 under palladium catalyzed Buchwald-Hartwig conditions provides compounds of type 13-4. Deprotection of the acetamide group under acidic conditions provides 13-5, which may be then treated with standard alkylating or acylating conditions (e.g. reductive amination conditions, or reaction with an elecrophile and a base) to affort compounds of type 13-6.

Scheme 14 describes routes to amines of Formula (2-1) from Scheme 2 in which R⁷ is a phenyl or substituted phenyl and R⁶ is either cyanomethyl (14-6), hydroxyethyl (14-9) or cyanoethyl (14-12). The synthesis of intermediates of type (14-5) is described in Journal of Medicinal Chemistry, 2011, 54 (11), 3756-3767 and Boc deprotection can be achieved by treatment with acid. Reduction of the nitrile in (14-5) firstly to the aldehyde (14-7) with a reagent such as diisobutylaluminium hydride and then to the alcohol (14-8) with a reagent such as sodium borohydride may be used to provide alcohols of formula (14-8) which may then be Boc-deprotected with acid to afford amine (14-9). Conversion of the alcohol (14-8) into the corresponding methanesulfonate ester (14-10) and subsequent reaction with a cyanide source such as sodium cyanide provides (14-11). Boc-deprotection of (14-11) results in the formation of amine (14-12). Alternatively, 14-10 may be treated with an amine in the presence of a base to afford compounds of type 14-13. Boc deprotection then affords compounds of type 14-14.

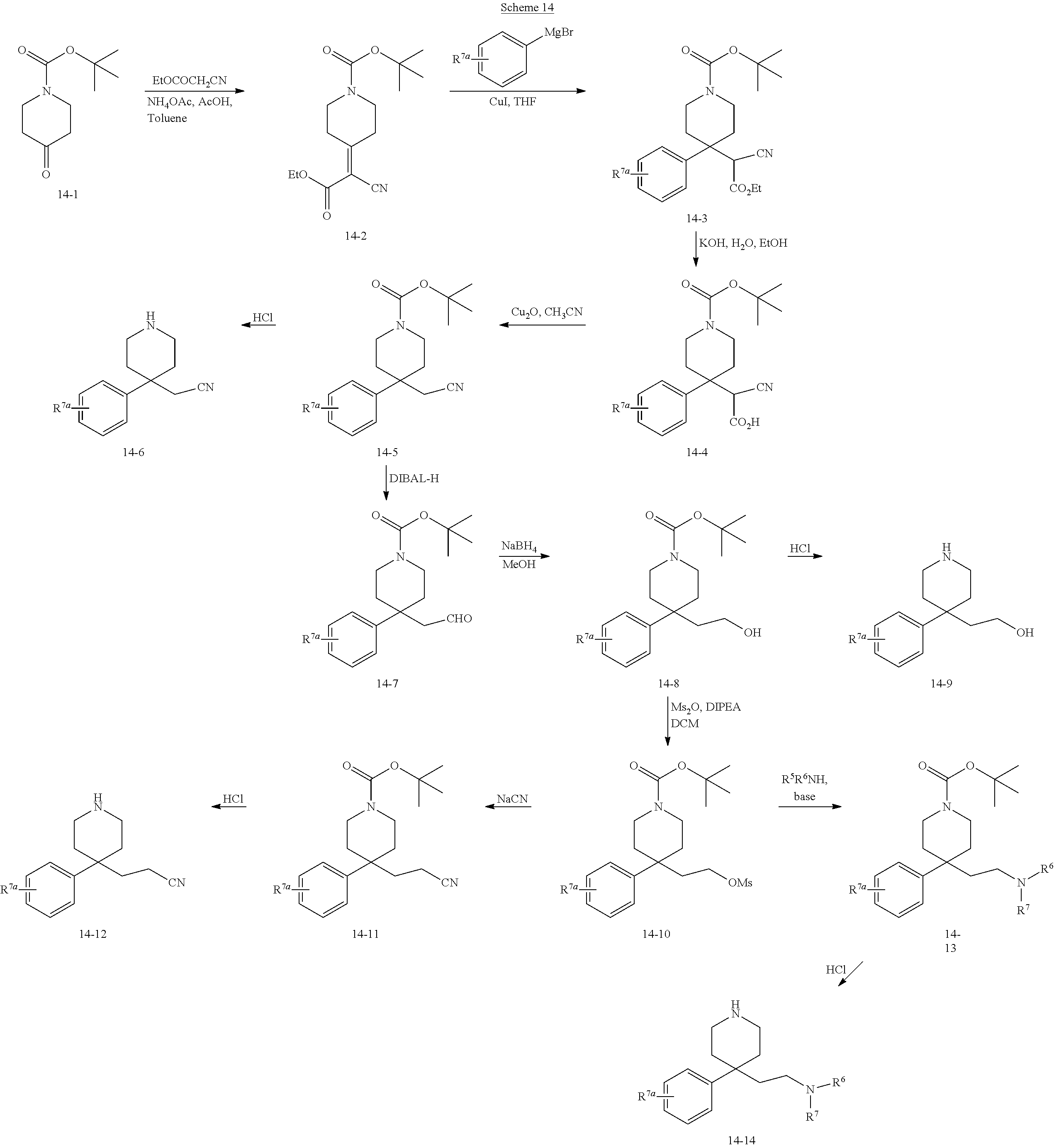

Scheme 15 describes routes to amines of Formula (2-1) from Scheme 2 in which R⁷ is a 4-difluoromethyl substituted phenyl and R⁶ is hydroxymethyl. Treatment of commercially available 4-bromobenzylcyanide with a base such as sodium hydride and reaction of the resultant anion with the commercially available alkylating agent (12-1) may be used to prepare intermediate (15-1). The ester (15-2) may be prepared from Intermediate (15-1) using a carbonylation reaction with a palladium catalyst such as Pd(dppf)Cl₂ under an atmosphere of carbon monoxide. Reduction of the ester in intermediate (15-2) using, for example, DIBAl-H affords the alcohol (15-3) which may then be oxidized to the aldehyde (15-4) using an oxidant such as DMP. The aldehyde of (15-4) may be converted to the difluoromethyl intermediate (15-5) using a reagent such as DAST. Reduction of the nitrile in Intermediate (15-5), using DIBAl-H for example, may afford the aldehyde (15-6) which may be further reduced to the alcohol (15-7) by treatment with sodium borohydride. Boc deprotection under standard conditions may be used to prepare the amine (15-8).

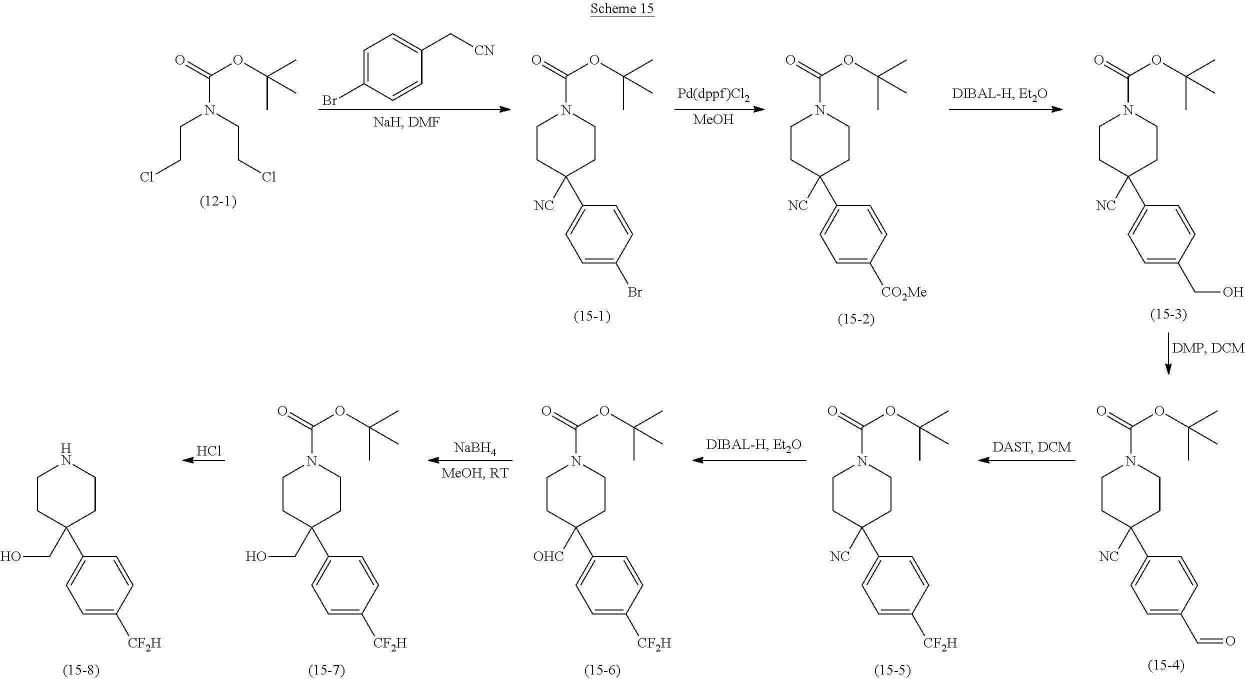

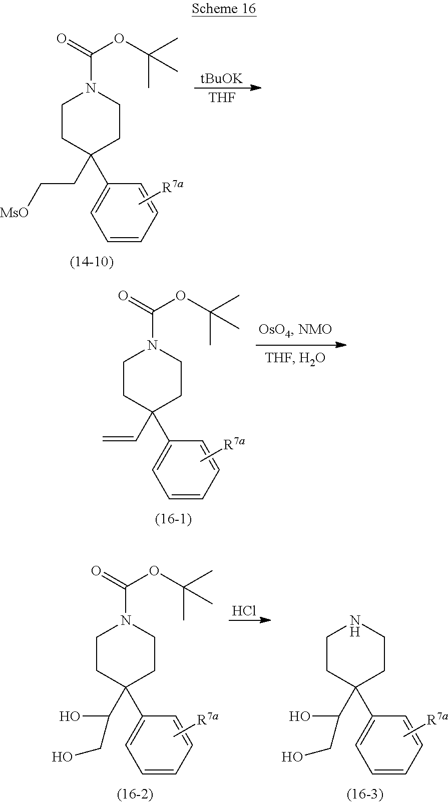

Scheme 16 describes routes to amines of Formula (2-1) from Scheme 2 in which R⁷ is a phenyl or substituted phenyl and R⁶ is a propane-1,2-diol. Treatment of methanesulfonate ester (14-10) with a base such as potassium tert-butoxide may provide the alkene (16-1). Intermediate (16-1) may be treated with an oxidant such as osmium tetroxide to afford diol (16-2). Boc deprotection under standard conditions may be used to give the amine (16-3).

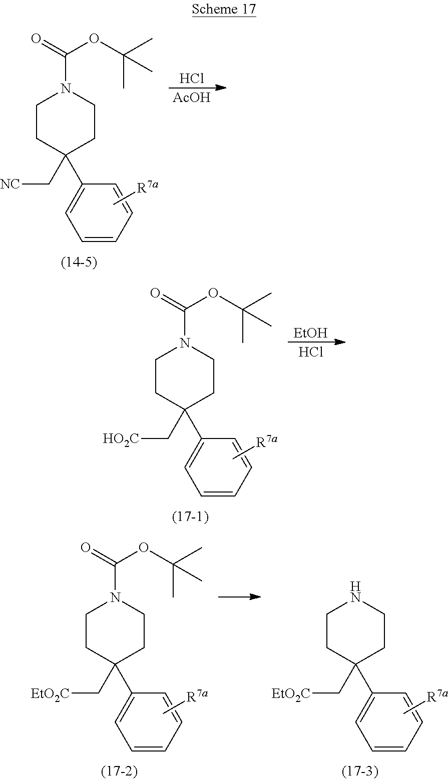

Scheme 17 describes routes to amines of Formula (2-1) from Scheme 2 in which R⁷ is a phenyl or substituted phenyl and R⁶ is a propionic acid ethyl ester. Hydrolysis of the nitrile (14-5) under acidic conditions with a reagent such as HCl in acetic acid may provide the acid (17-1). Esterification of the acid with an alcohol such as ethanol under acidic conditions may provide the ester (17-2). Boc deprotection under standard conditions may be used to give the amine (17-3).

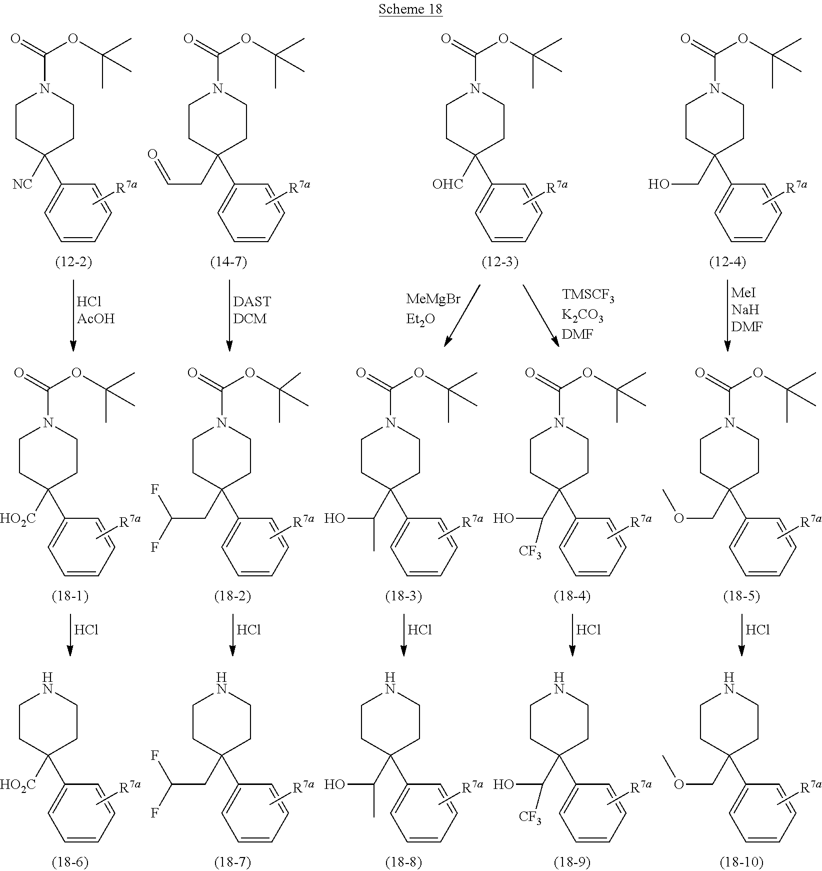

Scheme 18 describes routes to amines of Formula (2-1) from Scheme 2 in which R⁷ is a phenyl or substituted phenyl and R⁶ is carboxylic acid (18-6), difluoroethyl (18-7), 2-hydroxyethyl (18-8), 2-hydroxytrifluoroethyl (18-9), or methoxymethyl (18-10). Hydrolysis of the nitrile (12-2) under acidic conditions with a reagent such as HCl in acetic acid may be used to afford the acid (18-1). Treatment of the aldehyde (14-7) with a reagent such as DAST may be used to afford the difluoroethyl intermediate (18-2). Addition of a methyl Grignard such as methyl magnesium bromide to the aldehyde (12-3) may be used to afford the 2-hydroxyethyl intermediate (18-3). Treatment of the aldehyde (12-3) with a reagent such as CF₃-TMS in the presence of a base such as potassium carbonate may be used to give the trifluorohydroxy intermediate (18-4). Treatment of the alcohol (12-4) with a base such as sodium hydride in the presence of methyl iodide may be used to afford the methyl ether (18-5). Standard conditions may be used to deprotect Intermediates (18-1) to (18-5) to afford the amines (18-6) to (18-10).

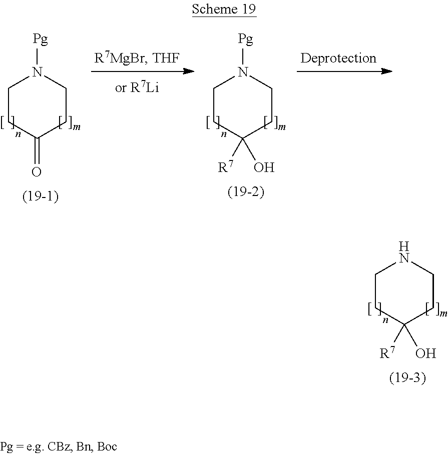

The cyclic secondary amine (2-1) of Scheme 2, in which R⁶ is hydroxyl and R⁷ is an optionally substituted alkyl, aryl or heteroaryl group, may be prepared according to Scheme 19. Reaction of a suitably nitrogen protected aminoketone (19-1) with either a Grignard reagent or an organolithium may provide the alcohol (19-2). Deprotection of the amine nitrogen to give amine (19-3) can then be achieved using conditions designed to remove the protecting group of choice.

Scheme 20 describes routes to amines of Formula (2-1) from Scheme 2 in which R⁷ is ethyl-(2,2,2-trifluoroethyl)amine and R⁶ is hydroxymethyl. Deprotonation adjacent to the nitrile of commercially available (20-1), using a base such as LDA, followed by treatment with BOM-C₁ affords the benzyloxy intermediate (20-2). The nitrile of intermediate (20-2) may be reduced to the aldehyde (20-3) using a suitable reducing agent such as DIBAl-H. The aldehyde may then be converted to the amine (20-4) using trifluoroethylamine and a reducing agent such as sodium cyanoborohydride. Hydrogenation of the benzyloxy intermediate (20-4) using palladium catalysis under an atmosphere of hydrogen may be used to prepare the hydroxymethyl intermediate (20-5). Removal of the Boc protecting group under standard conditions may be used to give the amine (20-6).

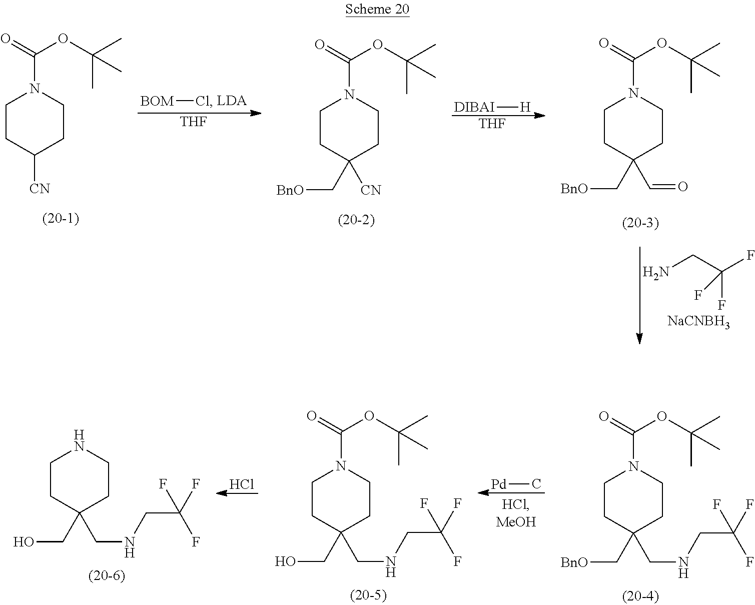

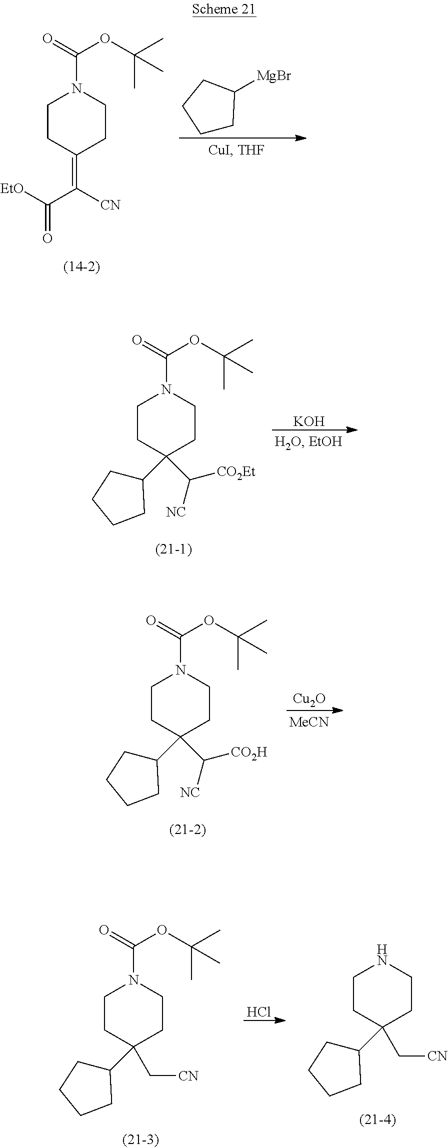

Scheme 21 describes a route to the amine (21-4) in which R⁷ is a cycloalkyl and R⁶ is cyanomethyl. Conjugate addition to a compound of Formula (14-2) using reagents such as a Grignard and copper (I) iodide may be used to prepare intermediate (21-1). Hydrolysis of the ester in compounds of Formula (21-1) with a base such as potassium hydroxide followed by decarboxylation using a reagent such as copper (I) oxide may be used to give compounds of Formula (21-3). Boc deprotection under standard conditions may be used to give the amine (21-4)

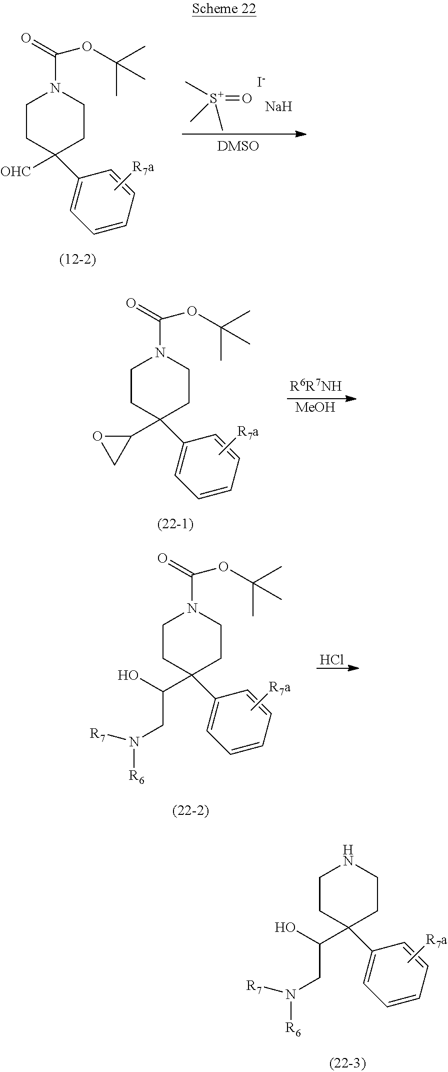

As described in Scheme 22, compounds of type 12-2 may be treated with Corey-Chaykovsky epoxidation conditions to afford compounds of type 22-1. Epoxide opening with an appropriate amine provides compounds of type 22-2, then Boc removal under acidic conditions provides compounds of type 22-3.

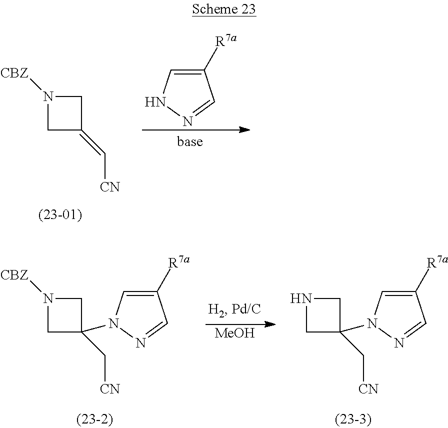

As shown in Scheme 23, compounds of type 23-01 may be treated under Michael-type conditions using a pyrazole as a nucleophile to produce compounds of type 23-2. CBz protecting group removal under standard conditions produces compounds of type 23-3.

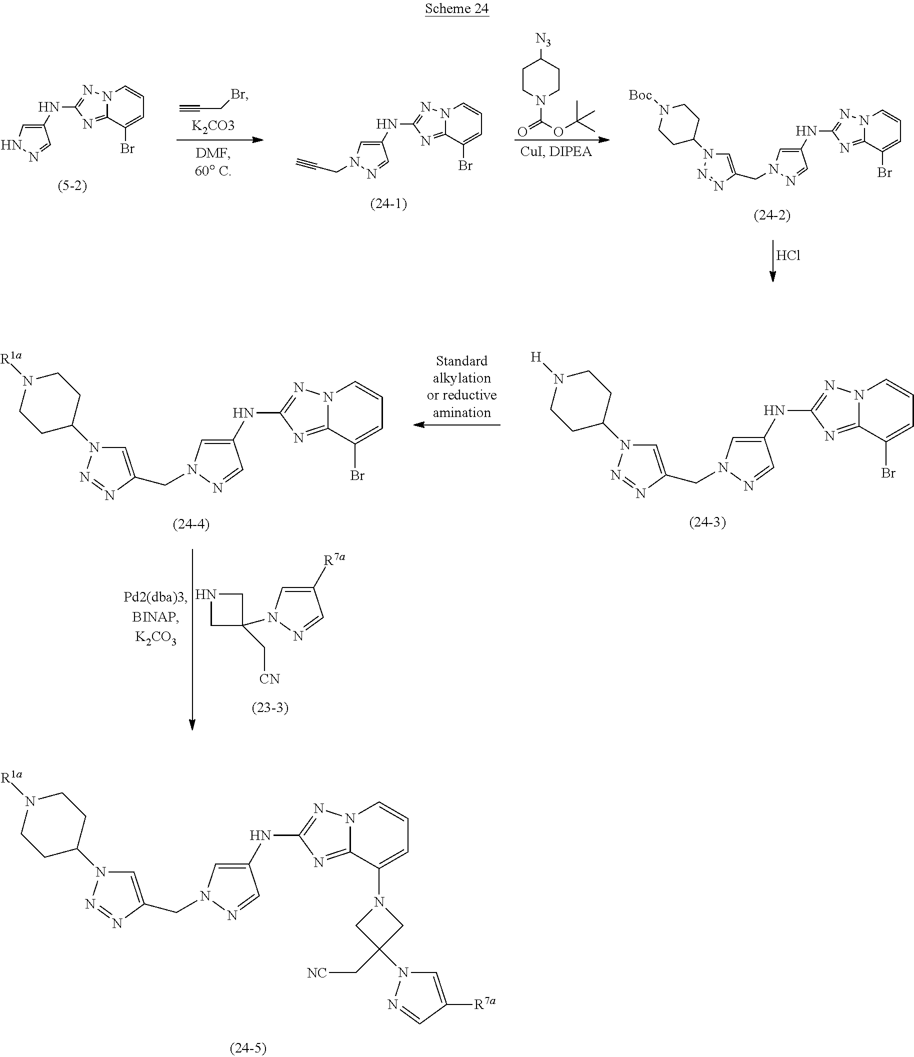

As shown in Scheme 24, compounds of type 5-2 can be propargylated under standard conditions to afford compounds of type 24-1. Copper promoted cycloaddition with an appropriate azide can then produce compounds of type 24-2. Boc deprotection produces compounds of type 24-3, which may then be reacted under standard alkylating or reductive amination conditions to produce compounds of type 24-4. Buchwald-Hartwig cross coupling with an appropriate amine such as 23-3 in the presence of a palladium catalyst then provides compounds of type 24-5.

Schemes 10 to 21 describe the methods that can be used to prepare other cyclic secondary amines (2-1), of Scheme 2, which are required for preparation of examples where the required amine (2-1) is hitherto unknown in the scientific literature. The methods use standard reactions known to those skilled in the art.

Analogous chemistries to those described in Scheme 1 may be used prepare compounds of the invention wherein R² of Formula (I) is of type (b) by replacing the cyclic secondary amine (2-1) with an amine of type (i). Here, R⁶/R⁷ may be modified further using standard chemistries.

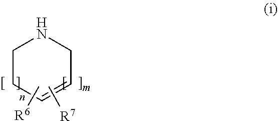

### Methods of Separation

In each of the exemplary Schemes it may be advantageous to separate reaction products from one another or from starting materials. The desired products of each step or series of steps is separated or purified (hereinafter separated) to the desired degree of homogeneity by the techniques common in the art. Typically such separations involve multiphase extraction, crystallization or trituration from a solvent or solvent mixture, distillation, sublimation, or chromatography. Chromatography can involve any number of methods including, for example: reverse-phase and normal phase; size exclusion; ion exchange; supercritical fluid; high, medium, and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. Example separation methods include boiling point, and molecular weight in distillation and sublimation, presence or absence of polar functional groups in chromatography, stability of materials in acidic and basic media in multiphase extraction, and the like. One skilled in the art will apply techniques most likely to achieve the desired separation.

Diastereomeric mixtures can be separated into their individual diastereoisomers on the basis of their physical chemical differences by methods well known to those skilled in the art, such as by chromatography or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column or supercritical fluid chromatography.

A single stereoisomer, e.g., an enantiomer, substantially free of its stereoisomer may be obtained by resolution of the racemic mixture using a method such as formation of diastereomers using optically active resolving agents (Eliel, E. and Wilen, S., Stereochemistry of Organic Compounds, John Wiley & Sons, Inc., New York, 1994; Lochmuller, C. H., J. Chromatogr., 113(3):283-302 (1975)). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Drug Stereochemistry, Analytical Methods and Pharmacology, Irving W. Wainer, Ed., Marcel Dekker, Inc., New York (1993).

Diastereomeric salts can be formed by reaction of enantiomerically pure chiral bases such as brucine, quinine, ephedrine, strychnine, ca-methyl-(3-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, the substrate to be resolved is reacted with one enantiomer of a chiral compound to form a diastereomeric pair (Eliel, E. and Wilen, S., Stereochemistry of Organic Compounds, John Wiley & Sons, Inc., New York, 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob, J. Org. Chem. 47:4165 (1982)), of the racemic mixture, and analyzing the NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111, incorporated herein by reference). By method (3), a racemic mixture of two enantiomers can be separated by chromatography using a chiral stationary phase (Chiral Liquid Chromatography W. J. Lough, Ed., Chapman and Hall, New York, (1989); Okamoto, J. of Chromatogr. 513:375-378 (1990)). Enriched or purified enantiomers can be distinguished by methods used to distinguish other chiral molecules with asymmetric carbon atoms, such as optical rotation and circular dichroism. The absolute stereochemistry of chiral centers and enantiomers can be determined by x-ray crystallography.

Positional isomers, for example E and Z forms, of compounds of Formula (I) or Formula II, and intermediates for their synthesis, may be observed by characterization methods such as NMR and analytical HPLC. For certain compounds where the energy barrier for interconversion is sufficiently high, the E and Z isomers may be separated, for example by preparatory HPLC.

### Pharmaceutical Compositions and Administration

The compounds with which the invention is concerned are JAK kinase inhibitors, such as JAK1 inhibitors, and are useful in the treatment of several diseases, for example, inflammatory diseases, such as asthma.

Accordingly, another embodiment provides pharmaceutical compositions or medicaments containing a compound of the invention, such as a compound of formula (I), (Ia), (Ib), (Ic), (Id), (Ie), (If), (Ig), (Ih), (Ii), (Ij), (Ik) or (II), or any variations described herein (e.g., a compound selected from Compound Nos. 1-1 to 1-15, 2-1, 2-2, 3-1 to 3-5 and Letters A-S), or a stereoisomer, tautomer, solvate or prodrug thereof, or a pharmaceutically acceptable salt thereof, and a pharmaceutically acceptable carrier, diluent or excipient, as well as methods of using the compounds of the invention to prepare such compositions and medicaments.

In one example, a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but typically ranges anywhere from about 3 to about 8. In one example, a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, is formulated in an acetate buffer, at pH 5. In another embodiment, the compounds of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. In general, the daily dose range for oral administration will lie within the range of from about 0.001 mg to about 100 mg per kg body weight of a human, often 0.01 mg to about 50 mg per kg, for example 0.1 to 10 mg per kg, in single or divided doses. In general, the daily dose range for inhaled administration will lie within the range of from about 0.1 μg to about 1 mg per kg body weight of a human, preferably 0.1 μg to 50 μg per kg, in single or divided doses. On the other hand, it may be necessary to use dosages outside these limits in some cases.

The compounds of the invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, may be administered by any suitable means, including oral, topical (including buccal and sublingual), rectal, vaginal, transdermal, parenteral, subcutaneous, intraperitoneal, intrapulmonary, intradermal, intrathecal, inhaled and epidural and intranasal, and, if desired for local treatment, intralesional administration. Parenteral infusions include intramuscular, intravenous, intraarterial, intraperitoneal, or subcutaneous administration. In some embodiments, inhaled administration is employed.

The compounds of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, may be administered in any convenient administrative form, e.g., tablets, powders, capsules, lozenges, granules, solutions, dispersions, suspensions, syrups, sprays, vapors, suppositories, gels, emulsions, patches, etc. Such compositions may contain components conventional in pharmaceutical preparations, e.g., diluents (e.g., glucose, lactose or mannitol), carriers, pH modifiers, buffers, sweeteners, bulking agents, stabilizing agents, surfactants, wetting agents, lubricating agents, emulsifiers, suspending agents, preservatives, antioxidants, opaquing agents, glidants, processing aids, colorants, perfuming agents, flavoring agents, other known additives as well as further active agents.

Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. For example, carriers include solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, pp 1289-1329, 1990). Except insofar as any conventional carrier is incompatible with the active ingredient, its use in the therapeutic or pharmaceutical compositions is contemplated. Exemplary excipients include dicalcium phosphate, mannitol, lactose, starch, magnesium stearate, sodium saccharine, cellulose, magnesium carbonate or combinations thereof. A pharmaceutical composition may comprise different types of carriers or excipients depending on whether it is to be administered in solid, liquid or aerosol form, and whether it need to be sterile for such routes of administration.

For example, tablets and capsules for oral administration may be in unit dose presentation form, and may contain conventional excipients such as binding agents, for example syrup, acacia, gelatin, sorbitol, tragacanth, or polyvinyl-pyrrolidone; fillers, for example, lactose, sugar, maize-starch, calcium phosphate, sorbitol or glycine; tabletting lubricant, for example, magnesium stearate, talc, polyethylene glycol or silica; disintegrants, for example, potato starch, or acceptable wetting agents such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example, sorbitol, syrup, methyl cellulose, glucose syrup, gelatin hydrogenated edible fats; emulsifying agents, for example, lecithin, sorbitan monooleate, or acacia; non-aqueous vehicles (which may include edible oils), for example, almond oil, fractionated coconut oil, oily esters such as glycerine, propylene glycol, or ethyl alcohol; preservatives, for example, methyl or propyl p-hydroxybenzoate or sorbic acid, and if desired conventional flavoring or coloring agents.

For topical application to the skin, a compound may be made up into a cream, lotion or ointment. Cream or ointment formulations which may be used for the drug are conventional formulations well known in the art, for example as described in standard textbooks of pharmaceutics such as the British Pharmacopoeia.

Compounds of the invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, may also be formulated for inhalation, for example, as a nasal spray, or dry powder or aerosol inhalers. For delivery by inhalation, the compound is typically in the form of microparticles, which can be prepared by a variety of techniques, including spray-drying, freeze-drying and micronisation. Aerosol generation can be carried out using, for example, pressure-driven jet atomizers or ultrasonic atomizers, such as by using propellant-driven metered aerosols or propellant-free administration of micronized compounds from, for example, inhalation capsules or other “dry powder” delivery systems.

By way of example, a composition of the invention may be prepared as a suspension for delivery from a nebulizer or as an aerosol in a liquid propellant, for example, for use in a pressurized metered dose inhaler (PMDI). Propellants suitable for use in a PMDI are known to the skilled person, and include CFC-12, HFA-134a, HFA-227, HCFC-22 (CCl2F2) and HFA-152 (CH4F2 and isobutane).

In some embodiments, a composition of the invention is in dry powder form, for delivery using a dry powder inhaler (DPI). Many types of DPI are known.

Microparticles for delivery by administration may be formulated with excipients that aid delivery and release. For example, in a dry powder formulation, microparticles may be formulated with large carrier particles that aid flow from the DPI into the lung. Suitable carrier particles are known, and include lactose particles; they may have a mass median aerodynamic diameter of, for example, greater than 90 am.

In the case of an aerosol-based formulation, an example is:

| | | |
|---|---|---|
| Compound of the invention* | 24 | mg/canister |
| Lecithin, NF Liq. Conc. | 1.2 | mg/canister |
| Trichlorofluoromethane, NF | 4.025 | g/canister |
| Dichlorodifluoromethane, NF | 12.15 | g/canister. |

*Such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S.

A compound, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, may be dosed as described depending on the inhaler system used. In addition to the compound, the administration forms may additionally contain excipients as described above, or, for example, propellants (e.g., Frigen in the case of metered aerosols), surface-active substances, emulsifiers, stabilizers, preservatives, flavorings, fillers (e.g., lactose in the case of powder inhalers) or, if appropriate, further active compounds.

For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g., Nebulator®, Volumatic®), and automatic devices emitting a puffer spray (Autohaler®), for metered aerosols, in the case of powder inhalers in particular, a number of technical solutions are available (e.g., Diskhaler®, Rotadisk®, Turbohaler® or the inhalers, for example, as described in U.S. Pat. No. 5,263,475, incorporated herein by reference). Additionally, compounds of the invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, may be delivered in multi-chamber devices thus allowing for delivery of combination agents.

The compound, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, may also be administered parenterally in a sterile medium. Depending on the vehicle and concentration used, the compound can either be suspended or dissolved in the vehicle. Advantageously, adjuvants such as a local anaesthetic, preservative or buffering agents can be dissolved in the vehicle.

### Methods of Use

Compounds and compositions of the invention, such as a pharmaceutical composition containing a compound of any formula provided herein or a salt thereof and a pharmaceutically acceptable carrier or excipient, may be used in methods of administration and treatment as provided herein.

The compounds of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, inhibit the activity of a Janus kinase, such as JAK1 kinase. For example, a compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, inhibits the phosphorylation of signal transducers and activators of transcription (STATs) by JAK1 kinase as well as STAT mediated cytokine production. Compounds of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, are useful for inhibiting JAK1 kinase activity in cells through cytokine pathways, such as IL-6, IL-15, IL-7, IL-2, IL-4, IL-9, IL-10, IL-13, IL-21, G-CSF, IFNalpha, IFNbeta or IFNgamma pathways. Accordingly, in one embodiment is provided a method of contacting a cell with a compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, to inhibit a Janus kinase activity in the cell (e.g., JAK1 activity).

The compounds of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, can be used for the treatment of immunological disorders driven by aberrant IL-6, IL-15, IL-7, IL-2, IL-4, IL9, IL-10, IL-13, IL-21, G-CSF, IFNalpha, IFNbeta or IFNgamma cytokine signaling.

Accordingly, one embodiment includes compounds of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, for use in therapy.

In some embodiments, there is provided use a compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, in the treatment of an inflammatory disease. Further provided is use of a compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, for the preparation of a medicament for the treatment of an inflammatory disease, such as asthma. Also provided is a compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, for use in the treatment of an inflammatory disease, such as asthma.

Another embodiment includes a method of preventing, treating or lessening the severity of a disease or condition, such as asthma, responsive to the inhibition of a Janus kinase activity, such as JAK1 kinase activity, in a patient. The method can include the step of administering to a patient a therapeutically effective amount of a compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S. In one embodiment, the disease or condition responsive to the inhibition of a Janus kinase, such as JAK1 kinase, is asthma.

In one embodiment, the disease or condition is cancer, stroke, diabetes, hepatomegaly, cardiovascular disease, multiple sclerosis, Alzheimer's disease, cystic fibrosis, viral disease, autoimmune diseases, atherosclerosis, restenosis, psoriasis, rheumatoid arthritis, inflammatory bowel disease, asthma, allergic disorders, inflammation, neurological disorders, a hormone-related disease, conditions associated with organ transplantation (e.g., transplant rejection), immunodeficiency disorders, destructive bone disorders, proliferative disorders, infectious diseases, conditions associated with cell death, thrombin-induced platelet aggregation, liver disease, pathologic immune conditions involving T cell activation, CNS disorders or a myeloproliferative disorder.

In one embodiment, the inflammatory disease is rheumatoid arthritis, psoriasis, asthma, inflammatory bowel disease, contact dermatitis or delayed hypersensitivity reactions. In one embodiment, the autoimmune disease is rheumatoid arthritis, lupus or multiple sclerosis.

In one embodiment, the cancer is breast, ovary, cervix, prostate, testis, penile, genitourinary tract, seminoma, esophagus, larynx, gastric, stomach, gastrointestinal, skin, keratoacanthoma, follicular carcinoma, melanoma, lung, small cell lung carcinoma, non-small cell lung carcinoma (NSCLC), lung adenocarcinoma, squamous carcinoma of the lung, colon, pancreas, thyroid, papillary, bladder, liver, biliary passage, kidney, bone, myeloid disorders, lymphoid disorders, hairy cells, buccal cavity and pharynx (oral), lip, tongue, mouth, salivary gland, pharynx, small intestine, colon, rectum, anal, renal, prostate, vulval, thyroid, large intestine, endometrial, uterine, brain, central nervous system, cancer of the peritoneum, hepatocellular cancer, head cancer, neck cancer, Hodgkin's or leukemia.

In one embodiment, the disease is a myeloproliferative disorder. In one embodiment, the myeloproliferative disorder is polycythemia vera, essential thrombocytosis, myelofibrosis or chronic myelogenous leukemia (CML).

Another embodiment includes the use of a compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, for the manufacture of a medicament for the treatment of a disease described herein (e.g., an inflammatory disorder, an immunological disorder or cancer).

### Combination Therapy

The compounds of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, may be employed alone or in combination with other agents for treatment. The second compound of a pharmaceutical composition or dosing regimen typically has complementary activities to the compound of this invention such that they do not adversely affect each other. Such agents are suitably present in combination in amounts that are effective for the purpose intended. The compounds may be administered together in a unitary pharmaceutical composition or separately and, when administered separately this may occur simultaneously or sequentially. Such sequential administration may be close or remote in time.

For example, other compounds may be combined with compounds with which the invention is concerned for the prevention and treatment of inflammatory diseases, such as asthma. Thus the present invention is also concerned with pharmaceutical compositions comprising a therapeutically effective amount of a compound of the invention and one or more other therapeutic agents. Suitable therapeutic agents for a combination therapy with compounds of the invention include, but are not limited to: an adenosine A2A receptor antagonist; an anti-infective; a non-steroidal Glucocorticoid Receptor (GR Receptor) agonist; an antioxidant; a □2 adrenoceptor agonist; a CCR1 antagonist; a chemokine antagonist (not CCR1); a corticosteroid; a CRTh2 antagonist; a DP1 antagonist; a formyl peptide receptor antagonist; a histone deacetylase activator; a chloride channel hCLCA1 blocker; an epithelial sodium channel blocker (ENAC blocker; an inter-cellular adhesion molecule 1 blocker (ICAM blocker); an IKK2 inhibitor; a JNK inhibitor; a cyclooxygenase inhibitor (COX inhibitor); a lipoxygenase inhibitor; a leukotriene receptor antagonist; a dual □2 adrenoceptor agonist/M3 receptor antagonist (MABA compound); a MEK-1 inhibitor; a myeloperoxidase inhibitor (MPO inhibitor); a muscarinic antagonist; a p38 MAPK inhibitor; a phosphodiesterase PDE4 inhibitor; a phosphatidylinositol 3-kinase □ inhibitor (PI3-kinase □ inhibitor); a peroxisome proliferator activated receptor agonist (PPAR□ agonist); a protease inhibitor; a retinoic acid receptor modulator (RAR □ modulator); a statin; a thromboxane antagonist; or a vasodilator.

In addition, compounds of the invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, may be combined with: (1) corticosteroids, such as alclometasone dipropionate, amelometasone, beclomethasone dipropionate, budesonide, butixocort propionate, biclesonide, blobetasol propionate, desisobutyrylciclesonide, dexamethasone, dtiprednol dicloacetate, fluocinolone acetonide, fluticasone furoate, fluticasone propionate, loteprednol etabonate (topical) or mometasone furoate; (2) β2-adrenoreceptor agonists such as salbutamol, albuterol, terbutaline, fenoterol, and long acting β2-adrenoreceptor agonists such as metaproterenol, isoproterenol, isoprenaline, salmeterol, indacaterol, formoterol (including formoterol fumarate), arformoterol, carmoterol, GSK 642444, GSK 159797, GSK 159802, GSK 597501, GSK 678007, or AZD3199; (3) corticosteroid/long acting J32 agonist combination products such as salmeterol/fluticasone propionate (Advair®, also sold as Seretide®), formoterol/budesonide (Symbicort®), formoterol/fluticasone propionate (Flutiform®), formoterol/ciclesonide, formoterol/mometasone furoate, indacaterol/mometasone furoate, indacaterol/QAE 397, GSK 159797/GSK 685698, GSK 159802/GSK 685698, GSK 642444/GSK 685698, GSK 159797/GSK 870086, GSK 159802/GSK 870086, GSK 642444/GSK 870086, or arformoterol/ciclesonide; (4) anticholinergic agents, for example, muscarinic-3 (M3) receptor antagonists such as ipratropium bromide, tiotropium bromide, aclidinium (LAS-34273), NVA-237, GSK 233705, darotropium, GSK 573719, GSK 961081, QAT 370, or QAX 028; (5) dual pharmacology M3-anticholinergic/β2-adrenoreceptor agonists such as GSK961081; (6) leukotriene modulators, for example, leukotriene antagonists such as montelukast, zafirulast or pranlukast or leukotriene biosynthesis inhibitors such as zileuton or BAY-1005, or LTB4 antagonists such as amelubant, or FLAP inhibitors such as GSK 2190914, AM-103; (7) phosphodiesterase-IV (PDE-IV) inhibitors (oral or inhaled), such as roflumilast, cilomilast, oglemilast, ONO-6126, tetomilast, tofimilast, UK 500,001, or GSK 256066; (8) antihistamines, for example, selective histamine-1 (H1) receptor antagonists such as fexofenadine, citirizine, loratidine or astemizole or dual H1/H3 receptor antagonists such as GSK 835726, or GSK 1004723; (9) antitussive agents, such as codeine or dextramorphan; (10) a mucolytic, for example, N-acetyl cysteine or fudostein; (11) a expectorant/mucokinetic modulator, for example, ambroxol, hypertonic solutions (e.g., saline or mannitol) or surfactant; (12) a peptide mucolytic, for example, recombinant human deoxyribonoclease I (dornase-alpha and rhDNase) or helicidin; (13) antibiotics, for example azithromycin, tobramycin or aztreonam; (14) non-selective COX-1/COX-2 inhibitors, such as ibuprofen or ketoprofen; (15) COX-2 inhibitors, such as celecoxib and rofecoxib; (16) VLA-4 antagonists, such as those described in WO97/03094 and WO97/02289, each incorporated herein by reference; (17) TACE inhibitors and TNF-α inhibitors, for example anti-TNF monoclonal antibodies, such as Remicade® and CDP-870 and TNF receptor immunoglobulin molecules, such as Enbrel®; (18) inhibitors of matrix metalloprotease, for example MMP-12; (19) human neutrophil elastase inhibitors, such as ONO-6818 or those described in WO2005/026124, WO2003/053930 and WO06/082412, each incorporated herein by reference; (20) A2b antagonists such as those described in WO2002/42298, incorporated herein by reference; (21) modulators of chemokine receptor function, for example antagonists of CCR3 and CCR8; (22) compounds which modulate the action of other prostanoid receptors, for example, a thromboxane A2 antagonist; DP1 antagonists such as MK-0524, CRTH2 antagonists such as ODC9101 and AZD1981 and mixed DP1/CRTH2 antagonists such as AMG 009; (23) PPAR agonists including PPAR alpha agonists (such as fenofibrate), PPAR delta agonists, PPAR gamma agonists such as pioglitazone, rosiglitazone and balaglitazone; (24) methylxanthines such as theophylline or aminophylline and methylxanthine/corticosteroid combinations such as theophylline/budesonide, theophylline/fluticasone propionate, theophylline/ciclesonide, theophylline/mometasone furoate and theophylline/beclometasone dipropionate; (25) A2a agonists such as those described in EP1052264 and EP1241176; (26) CXCR2 or IL-8 antagonists such as SCH 527123 or GSK 656933; (27) IL-R signalling modulators such as kineret and ACZ 885; and (28) MCP-1 antagonists such as ABN-912.

In some embodiments, the compounds of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, can be used in combination with one or more additional drugs, for example anti-hyperproliferative, anti-cancer, cytostatic, cytotoxic, anti-inflammatory or chemotherapeutic agents, such as those agents disclosed in U.S. Publ. Appl. No. 2010/0048557, incorporated herein by reference. A compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, can be also used in combination with radiation therapy or surgery, as is known in the art.

### Articles of Manufacture

Another embodiment includes an article of manufacture (e.g., a kit) for treating a disease or disorder responsive to the inhibition of a Janus kinase, such as a JAK1 kinase. The kit can comprise: (a) a first pharmaceutical composition comprising a compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S; and (b) instructions for use. In another embodiment, the kit further comprises: (c) a second pharmaceutical composition, such as a pharmaceutical composition comprising an agent for treatment as described above, such as an agent for treatment of an inflammatory disorder, or a chemotherapeutic agent.

In one embodiment, the instructions describe the simultaneous, sequential or separate administration of said first and second pharmaceutical compositions to a patient in need thereof.

In one embodiment, the first and second compositions are contained in separate containers. In another embodiment, the first and second compositions are contained in the same container.

Containers for use include, for example, bottles, vials, syringes, blister pack, etc. The containers may be formed from a variety of materials such as glass or plastic. The container includes a compound of the present invention, such as a compound of formula (I), (Ia), (Ib), (Ic), (Id), (Ie), (If), (Ig), (Ih), (Ii), (Ij), (Ik) or (II), or a compound selected from Compound Nos. 1-1 to 1-15, 2-1, 2-2, 3-1 to 3-5 and Letters A-S, or composition thereof, which is effective for treating the condition and may have a sterile access port (for example the container may be an intravenous solution bag or a vial having a stopper pierceable by a hypodermic injection needle). The label or package insert indicates that the compound or composition is used for treating the condition of choice, such as asthma or cancer. In one embodiment, the label or package inserts indicates that the compound or composition can be used to treat a disorder. In addition, the label or package insert may indicate that the patient to be treated is one having a disorder characterized by overactive or irregular Janus kinase activity, such as overactive or irregular JAK1 activity. The label or package insert may also indicate that the compound or composition can be used to treat other disorders.

Alternatively, or additionally, the kit may further comprise a second (or third) container comprising a pharmaceutically acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

The following list provides additional embodiments of the present invention:

### Embodiment 1

A compound of formula (I):

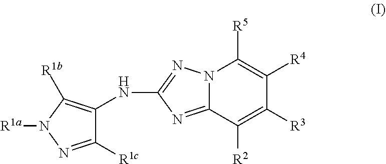

or a stereoisomer, tautomer, solvate, prodrug or salt thereof, wherein:

R^{1a} is hydrogen, C₁-C₆ alkyl, C₃-C₈ cycloalkyl, phenyl, or 3-11 membered heterocyclyl and R^{1a} is optionally substituted by R⁹;

R^{1b} and R^{1c} are each independently hydrogen, C₁-C₆ alkyl, or C₃-C₈ cycloalkyl;

R² is a 3-11 membered heterocyclyl containing at least 1 nitrogen, selected from groups (a)-(e) and (h)-(j); a C₅-C₈ cycloalkenyl ring (f); a —O—(CR^{x}R^{y})_{q}—Ar² group (g); or a Ar¹—O—(CR^{x}R^{y})_{q}—Ar² group (k), where each R^{x} and R^{y} are independently hydrogen or C₁-C₆ alkyl, each q is independently 0 to 3, Ar¹ is 1,4-phenylene and Ar² is optionally substituted C₆-C₁₀ aryl or optionally substituted 5-11 membered heteroaryl:

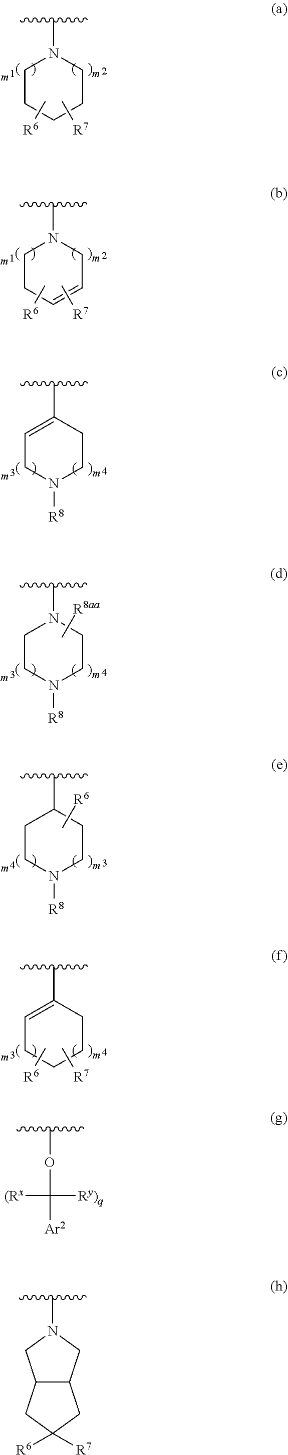

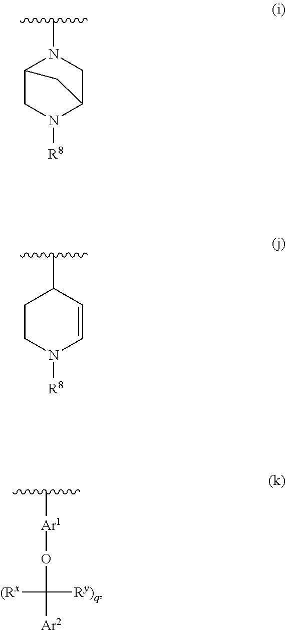

wherein the wavy line represents the point of attachment of R² in formula (I);

R³, R⁴ and R⁵ are each independently selected from the group consisting of hydrogen, CH₃, CH₂CH₃, OCH₃, CF₃, F and C₁;

R⁶ and R⁷ are independently selected from the group consisting of hydrogen, halogen, OH, CN, phenyl, C₁-C₆ alkyl, (C₀-C₆ alkylene)C₃-C₈ cycloalkyl, (C₀-C₆ alkylene)3-11 membered heterocyclyl, (C₀-C₆ alkylene)C(O)NR^{a}R^{b}, (C₀-C₆ alkylene)NR^{a}C(O)(C₁-C₆ alkyl), (C₀-C₆ alkylene)NR^{a}C(O)(phenyl), (C₀-C₆ alkylene)C(O)R^{8a}, (C₀-C₆ alkylene)C(O)OR^{8a}, C₁-C₆ alkoxy, —O—(C₃-C₆ cycloalkyl), —O—(C₀-C₆ alkylene)C(O)NR^{a}R^{b}, —C═N—O—(C₁-C₆ alkyl), —O—(C₁-C₆ alkyl)3-11 membered heterocyclyl, (C₀-C₆ alkylene)NR^{a}SO₂(C₁-C₆ alkyl), (C₀-C₆ alkylene)NR^{a}SO₂(phenyl), and —O-(3-11 membered heterocyclyl); wherein said alkyl, alkylene, alkoxy, cycloalkyl, phenyl and heterocyclyl are each independently optionally substituted,
or R⁶ and R⁷ together form an optionally substituted phenyl or optionally substituted 3-11 membered heterocyclyl;

R⁸ is H, C₁-C₆ alkyl, (C₀-C₆ alkylene)phenyl, (C₀-C₆ alkylene)C₃-C₈ cycloalkyl, (C₀-C₆ alkylene)3-11 membered heterocyclyl, C(O)NR^{a}R^{b}, SO₂NR^{a}R^{b}, (C₁-C₆ alkylene)C(O)OR^{8a} or C(O)R^{8a}, wherein said alkyl, alkylene, heterocyclyl and phenyl are each independently optionally substituted;

R^{8a} is H, NR^{a}R^{b}, C₁-C₆ alkyl, (C₀-C₆ alkylene)C₃-C₈ cycloalkyl, (C₀-C₆ alkylene)phenyl, or (C₀-C₆ alkylene)3-11 membered heterocyclyl, wherein said alkyl, alkylene, cycloalkyl, phenyl and heterocyclyl are each independently optionally substituted;

R^{8aa} is H, C₁-C₆ alkyl optionally substituted by OH, or C(O)NR^{a}R^{b}; or

or R⁸ and R^{8aa} together form an optionally substituted 3-11 membered heterocyclyl;

R⁹, independently at each occurrence, is OH, halogen, CN, C₁-C₆ alkyl, C₃-C₈ cycloalkyl, phenyl, 3-11 membered heterocyclyl, 5-11 membered heteroaryl, —C(O)NR^{a}R^{b}, —NR^{a}R^{b}, (C₁-C₆ alkylene)C₃-C₈ cycloalkyl, (C₁-C₆ alkylene)phenyl, (C₁-C₆ alkylene)3-11 membered heterocyclyl, (C₁-C₆ alkylene)5-11 membered heteroaryl, (C₁-C₆ alkylene)C(O)NR^{a}R^{b}, (C₁-C₆ alkylene)NR^{a}R^{b}, or C(O)(C₁-C₆ alkyl), wherein said alkyl, alkylene, cycloalkyl, phenyl, heterocyclyl and heteroaryl are each independently optionally substituted;

R^{a} and R^{b}, independently at each occurrence, are selected from the group consisting of hydrogen, C₁-C₆ alkyl optionally substituted by halogen or CN, (C₀-C₆ alkylene)C₃-C₈ cycloalkyl, or (C₀-C₆ alkylene)phenyl, and wherein one or more alkylene units of any alkyl group is independently optionally substituted by —O—, or alternatively R^{a} and R^{b} may be joined together with the nitrogen atom to which they are attached to form an optionally substituted 3-11 membered heterocyclyl; and

m¹, m², m³ and m⁴ are each independently 0, 1 or 2.

provided that the compound is other than a compound selected from the group consisting of Compound Nos. 1x to 7x and salts thereof.

### Embodiment 2

The compound of Embodiment 1, or a salt thereof, wherein the compound is of the formula (Ia):

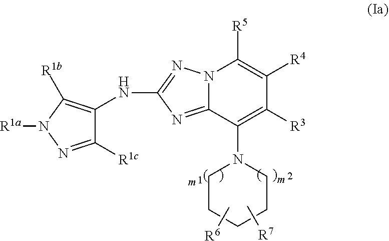

wherein R^{1a}, R^{1b}, R^{1c}, R³, R⁴, R⁵, R⁶ and R⁷, m¹ and m² are as defined in claim 1.

### Embodiment 3

The compound of Embodiment 1 or 2, or a salt thereof, wherein m¹ is 1 and m² is 1, or m¹ is 2 and m² is 1.

### Embodiment 4

The compound of Embodiment 3, or salt thereof, wherein m¹ is 1 and m² is 1.

### Embodiment 5

The compound of any one of Embodiments 1 to 4, or a salt thereof, wherein R⁶ and R⁷ are attached to the ring at the same carbon atom.

### Embodiment 6

The compound of any one of Embodiments 1 to 5, or a salt thereof, wherein R⁶ is optionally substituted C₁-C₆ alkyl.

### Embodiment 7

The compound of Embodiment 6, or a salt thereof, wherein R⁶ is C₁-C₆ alkyl optionally substituted with OH.

### Embodiment 8

The compound of any one of Embodiments 1 to 7, or a salt thereof, wherein R⁷ is optionally substituted phenyl.

### Embodiment 9

The compound of Embodiment 8, or a salt thereof, wherein R′ is phenyl optionally substituted with halo.

### Embodiment 10

The compound of Embodiment 5, or a salt thereof, wherein R⁶ is hydroxymethyl and R⁷ is 4-chlorophenyl.

### Embodiment 11

The compound of Embodiment 1, or salt thereof, wherein the compound is of the formula (Ic):

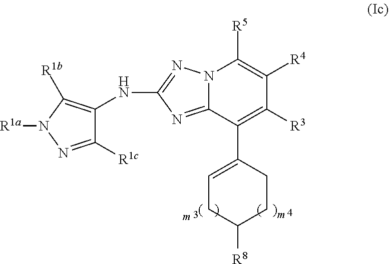

wherein R^{1a}, R^{1b}, R^{1c}, R³, R⁴, R⁵ and R⁸, m³ and m⁴ are as defined in Embodiment 1.

### Embodiment 12

The compound of Embodiment 1 or 11, or a salt thereof, wherein m³ is 1 and m⁴ is 1, or m³ is 1 and m⁴ is 2, or m³ is 1 and m⁴ is 0.

### Embodiment 13

The compound of 12, or a salt thereof, wherein m³ is 1 and m⁴ is 1.

### Embodiment 14

The compound of any one of Embodiments 1 and 11 to 13, or a salt thereof, wherein R⁸ is C(O)R^{a}.

### Embodiment 15

The compound of Embodiment 14, or a salt thereof, wherein R^{8a} is optionally substituted C₁-C₆ alkyl.

### Embodiment 16

The compound of Embodiment 15, or a salt thereof, wherein R^{8a} is C₁-C₆ alkyl optionally substituted with halo.

### Embodiment 17

The compound of Embodiment 14, or a salt thereof, wherein R⁸ is C(O)CH₂CH₂CF₃.

### Embodiment 18

The compound of Embodiment 1, or a stereoisomer, tautomer, solvate, prodrug or salt thereof, wherein the compound is of the formula (Ik):

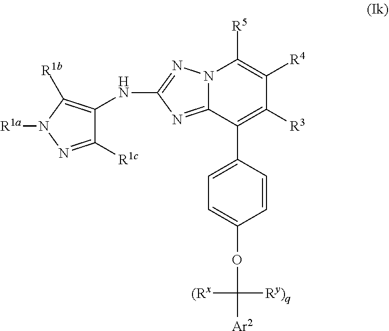

wherein R^{1a}, R³, R⁴, R⁵, R^{x}, R^{y}, Ar² and q are as defined in Embodiment 1.

### Embodiment 19

The compound of Embodiment 1 or 18, or a salt thereof, wherein q is 1.

### Embodiment 20

The compound of Embodiment 19, or a salt thereof, wherein each of R^{x} and R^{y} is hydrogen.

### Embodiment 21

The compound of any one of Embodiments 1 and 18 to 20, or a salt thereof, wherein Ar² is optionally substituted 5-11 membered heteroaryl.

### Embodiment 22

The compound of Embodiment 21, or a salt thereof, wherein Ar² is 6-membered heteroaryl optionally substituted with OR′ where R′ is C₁-C₆ alkyl optionally substituted with C₁-C₆ alkoxy.

### Embodiment 23

The compound of Embodiment 22, or a salt thereof, wherein Ar² is 6-(2-methoxyethoxy)-3-pyridyl.

### Embodiment 24

The compound of any one of Embodiments 1 to 23, or a salt thereof, wherein R³, R⁴ and R⁵ are each hydrogen.

### Embodiment 25

The compound of any one of Embodiments 1 to 24, or a salt thereof, wherein R^{1a} is C₁-C₆ alkyl optionally substituted by R⁹ or 3-11 membered heterocyclyl optionally substituted by R⁹.

### Embodiment 26

The compound of Embodiment 25, or a salt thereof, wherein R^{1a} is other than C₁-C₆ alkyl substituted by —C(O)NR^{a}R^{b}.

### Embodiment 27

The compound of Embodiment 25, or a salt thereof, wherein R^{1a} is C₁-C₆ alkyl optionally substituted by OH, halogen, CN, optionally substituted phenyl, optionally substituted 3-11 membered heterocyclyl, optionally substituted 5-11 membered heteroaryl, or —NR^{a}R^{b}.

### Embodiment 28

The compound of Embodiment 27, or a salt thereof, wherein R^{1a} is unsubstituted C₁-C₆ alkyl.

### Embodiment 29

The compound of Embodiment 27, or a salt thereof, wherein R^{1a} is C₁-C₆ alkyl substituted by 1 to 5 substituents independently selected from OH, halogen and CN.

### Embodiment 30

The compound of Embodiment 27, or a salt thereof, wherein R^{1a} is C₁-C₆ alkyl substituted by phenyl.

### Embodiment 31

The compound of Embodiment 27, or a salt thereof, wherein R^{1a} is C₁-C₆ alkyl substituted by 3-11 membered heterocyclyl optionally substituted by C₁-C₆ alkyl.

### Embodiment 32

The compound of Embodiment 31, or a salt thereof, wherein R^{1a} is C₁-C₆ alkyl substituted by piperidin-4-yl, piperazin-1-yl, 4-methylpiperazin-1-yl, morpholin-1-yl or pyrrolidin-2-yl.

### Embodiment 33

The compound of Embodiment 27, or a salt thereof, wherein R^{1a} is C₁-C₆ alkyl substituted by 5-11 membered heteroaryl.

### Embodiment 34

The compound of Embodiment 27, or a salt thereof, wherein R^{1a} is C₁-C₆ alkyl substituted by —NR^{a}R^{b}, wherein R^{a} and R^{b} are independently hydrogen or methyl.

### Embodiment 35

The compound of Embodiment 25, or a salt thereof, wherein R^{1a} is 3-11 membered heterocyclyl optionally substituted by C₁-C₆ alkyl.

### Embodiment 36

The compound of Embodiment 25, or a salt thereof, wherein R^{1a} is selected from the group consisting of:

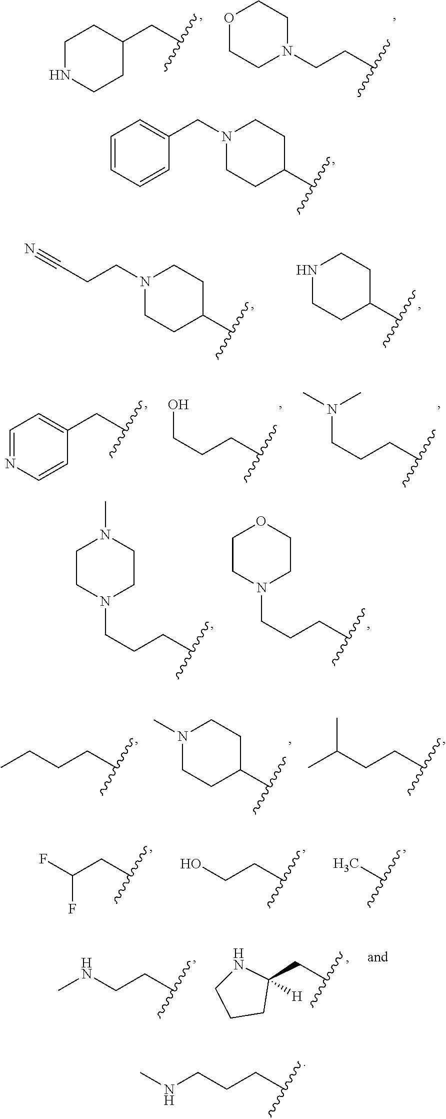

wherein the wavy line represents the point of attachment of R^{1a} in formula (I).

### Embodiment 37

The compound of any one of Embodiments 1 to 36, or a salt thereof, wherein R^{1b} and R^{1c} are each hydrogen.

### Embodiment 38

The compound of Embodiment 1, or a stereoisomer, tautomer, solvate, prodrug or salt thereof, wherein the compound is selected from Compound Nos. 1-1 to 1-15, 2-1, 2-2, 3-1 to 3-5 and Letters A-S:

| Compound No. | Structure |
|---|---|
| 1-1 | 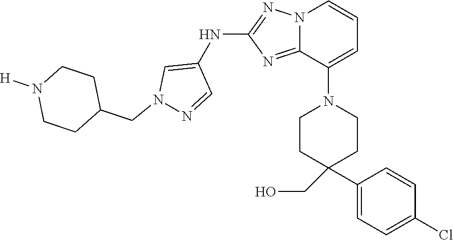 [SMILES: OCC1(c2ccc(Cl)cc2)CCN(c2cccn3nc(Nc4cnn(CC5CCNCC5)c4)nc23)CC1] |
| 1-2 | 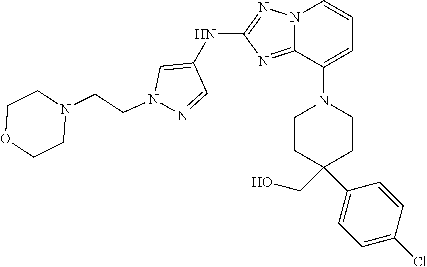 [SMILES: OCC1(c2ccc(Cl)cc2)CCN(c2cccn3nc(Nc4cnn(CCN5CCOCC5)c4)nc23)CC1] |
| 1-3 | 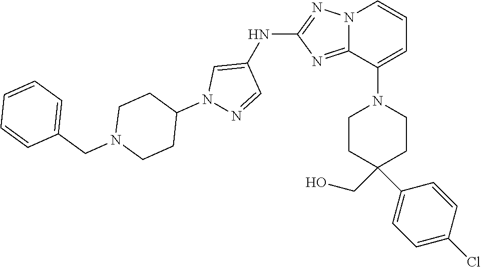 [SMILES: OCC1(c2ccc(Cl)cc2)CCN(c2cccn3nc(Nc4cnn(C5CCN(Cc6ccccc6)CC5)c4)nc23)CC1] |
| 1-4 | 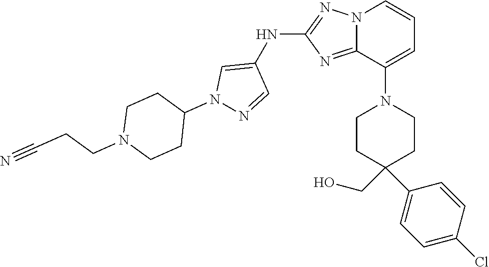 [SMILES: N#CCCN1CCC(n2cc(Nc3nc4c(N5CCC(CO)(c6ccc(Cl)cc6)CC5)cccn4n3)cn2)CC1] |
| 1-5 | 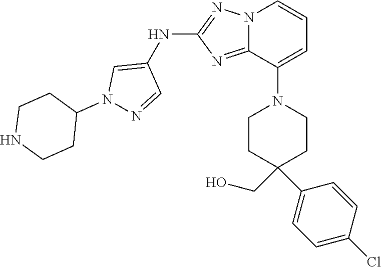 [SMILES: OCC1(c2ccc(Cl)cc2)CCN(c2cccn3nc(Nc4cnn(C5CCNCC5)c4)nc23)CC1] |
| 1-6 | 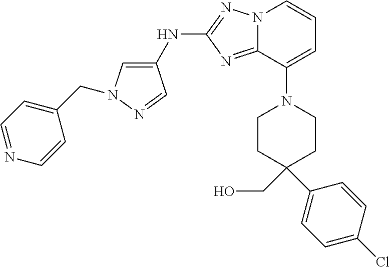 [SMILES: OCC1(c2ccc(Cl)cc2)CCN(c2cccn3nc(Nc4cnn(Cc5ccncc5)c4)nc23)CC1] |
| 1-7 | 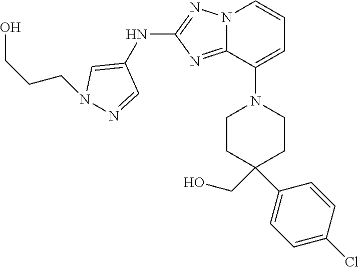 [SMILES: OCCCn1cc(Nc2nc3c(N4CCC(CO)(c5ccc(Cl)cc5)CC4)cccn3n2)cn1] |
| 1-8 | 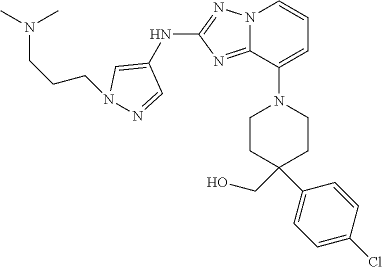 [SMILES: CN(C)CCCn1cc(Nc2nc3c(N4CCC(CO)(c5ccc(Cl)cc5)CC4)cccn3n2)cn1] |
| 1-9 | 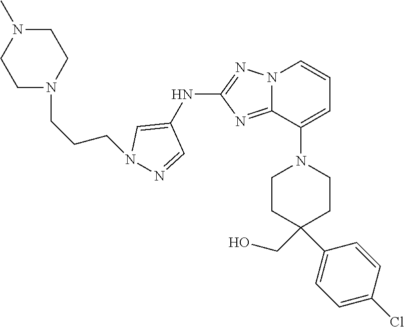 [SMILES: CN1CCN(CCCn2cc(Nc3nc4c(N5CCC(CO)(c6ccc(Cl)cc6)CC5)cccn4n3)cn2)CC1] |
| 1-10 | 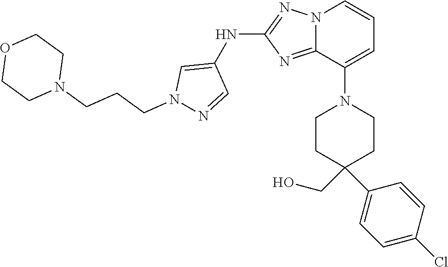 [SMILES: OCC1(c2ccc(Cl)cc2)CCN(c2cccn3nc(Nc4cnn(CCCN5CCOCC5)c4)nc23)CC1] |
| 1-11 | 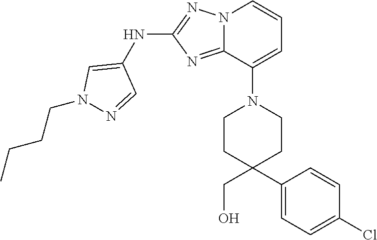 [SMILES: CCCCn1cc(Nc2nc3c(N4CCC(CO)(c5ccc(Cl)cc5)CC4)cccn3n2)cn1] |
| 1-12 | 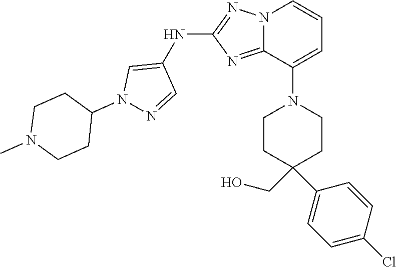 [SMILES: CN1CCC(n2cc(Nc3nc4c(N5CCC(CO)(c6ccc(Cl)cc6)CC5)cccn4n3)cn2)CC1] |
| 1-13 | 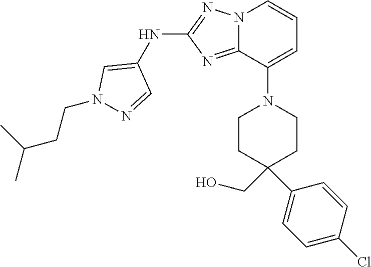 [SMILES: CC(C)CCn1cc(Nc2nc3c(N4CCC(CO)(c5ccc(Cl)cc5)CC4)cccn3n2)cn1] |
| 1-14 | 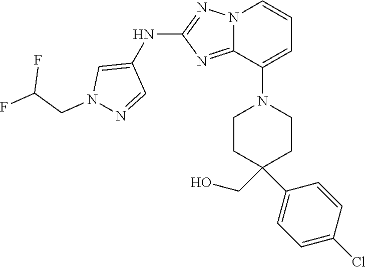 [SMILES: OCC1(c2ccc(Cl)cc2)CCN(c2cccn3nc(Nc4cnn(CC(F)F)c4)nc23)CC1] |
| 1-15 | 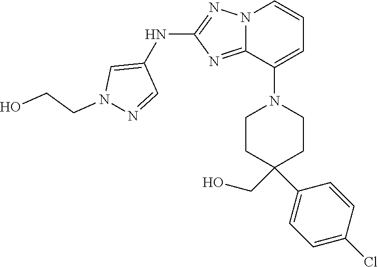 [SMILES: OCCn1cc(Nc2nc3c(N4CCC(CO)(c5ccc(Cl)cc5)CC4)cccn3n2)cn1] |
| 2-1 | 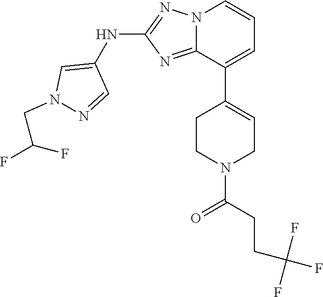 [SMILES: O=C(CCC(F)(F)F)N1CC=C(c2cccn3nc(Nc4cnn(CC(F)F)c4)nc23)CC1] |
| 2-2 | 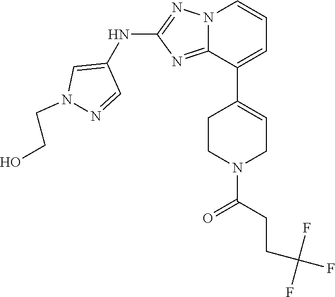 [SMILES: O=C(CCC(F)(F)F)N1CC=C(c2cccn3nc(Nc4cnn(CCO)c4)nc23)CC1] |
| 3-1 | 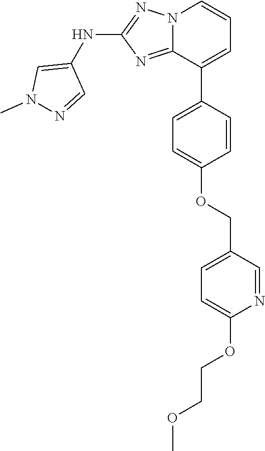 [SMILES: COCCOc1ccc(COc2ccc(-c3cccn4nc(Nc5cnn(C)c5)nc34)cc2)cn1] |
| 3-2 | 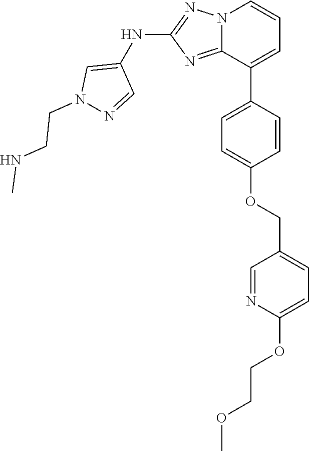 [SMILES: CNCCn1cc(Nc2nc3c(-c4ccc(OCc5ccc(OCCOC)nc5)cc4)cccn3n2)cn1] |
| 3-3 | 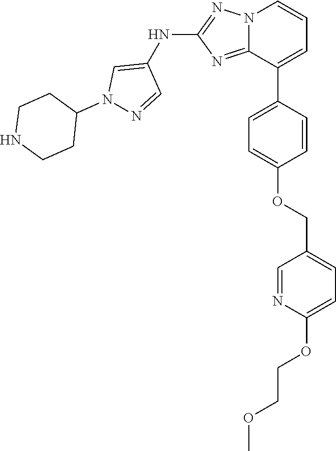 [SMILES: COCCOc1ccc(COc2ccc(-c3cccn4nc(Nc5cnn(C6CCNCC6)c5)nc34)cc2)cn1] |
| 3-4 | 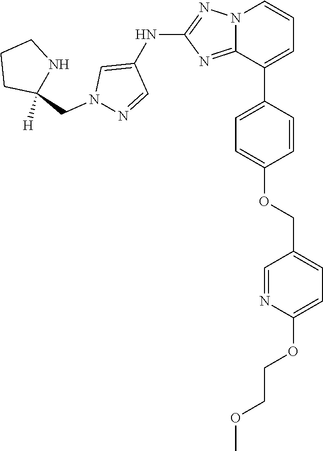 [SMILES: COCCOc1ccc(COc2ccc(-c3cccn4nc(Nc5cnn(C[C@H]6CCCN6)c5)nc34)cc2)cn1] |
| 3-5 | 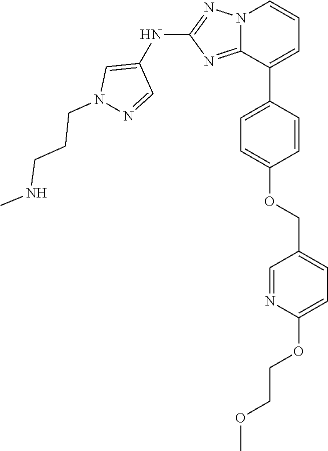 [SMILES: CNCCCn1cc(Nc2nc3c(-c4ccc(OCc5ccc(OCCOC)nc5)cc4)cccn3n2)cn1] |

### Embodiment 38a

The compound of Embodiment 1, or a stereoisomer, tautomer, solvate, prodrug or salt thereof, wherein the compound is selected from Compound Letters A-S:

| | | |
|---|---|---|
| A | 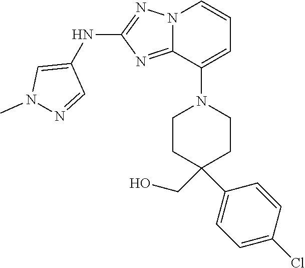 [SMILES: Cn1cc(Nc2nc3c(N4CCC(CO)(c5ccc(Cl)cc5)CC4)cccn3n2)cn1] | [4-(4-chlorophenyl)-1-[2-[(1- methylpyrazol-4-yl)amino]- [1,2,4]triazolo[1,5-a]pyridin-8-yl]-4- piperidyl]methanol |
| B | 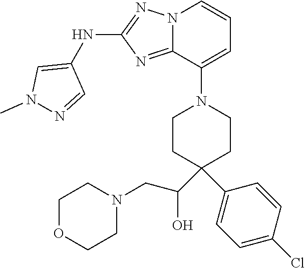 [SMILES: Cn1cc(Nc2nc3c(N4CCC(c5ccc(Cl)cc5)(C(O)CN5CCOCC5)CC4)cccn3n2)cn1] | 1-[4-(4-chlorophenyl)-1-[2-[(1- methylpyrazol-4-yl)amino]- [1,2,4]triazolo[1,5-a]pyridin-8-yl]-4- piperidyl]-2-morpholino-ethanol |
| C | 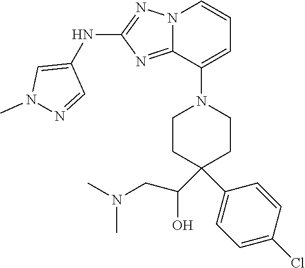 [SMILES: CN(C)CC(O)C1(c2ccc(Cl)cc2)CCN(c2cccn3nc(Nc4cnn(C)c4)nc23)CC1] | 1-[4-(4-chlorophenyl)-1-[2-[(1- methylpyrazol-4-yl)amino]- [1,2,4]triazolo[1,5-a]pyridin-8-yl]-4- piperidyl]-2-(dimethylamino)ethanol |
| D | 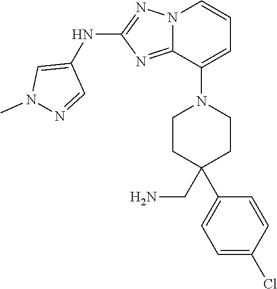 [SMILES: Cn1cc(Nc2nc3c(N4CCC(CN)(c5ccc(Cl)cc5)CC4)cccn3n2)cn1] | 8-[4-(aminomethyl)-4-(4- chlorophenyl)-1-piperidyl]-N-(1- methylpyrazol-4-yl)- [1,2,4]triazolo[1,5-a]pyridin-2-amine |
| E | 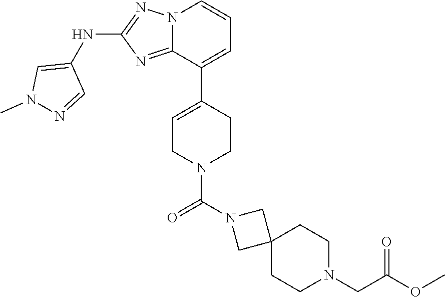 | methyl 2-[2-[4-[2-[(1-methylpyrazol- 4-yl)amino]-[1,2,4]triazolo[1,5- a]pyridin-8-yl]-3,6-dihydro-2H- pyridine-1-carbonyl]-2,7- diazaspiro[3.5]nonan-7-yl]acetate |
| F | 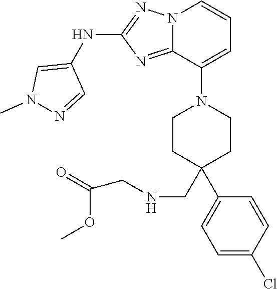 [SMILES: COC(=O)CNCC1(c2ccc(Cl)cc2)CCN(c2cccn3nc(Nc4cnn(C)c4)nc23)CC1] | formic acid; methyl 2-[[4-(4- chlorophenyl)-1-[2-[(1- methylpyrazol-4-yl)amino]- [1,2,4]triazolo[1,5-a]pyridin-8-yl]-4- piperidyl]methylamino] |
| G | 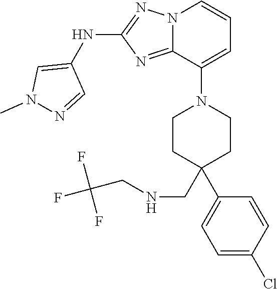 [SMILES: Cn1cc(Nc2nc3c(N4CCC(CNCC(F)(F)F)(c5ccc(Cl)cc5)CC4)cccn3n2)cn1] | 8-[4-(4-chlorophenyl)-4-[(2,2,2- trifluoroethylamino)methyl]-1- piperidyl]-N-(1-methylpyrazol-4-yl)- [1,2,4]triazolo[1,5-a]pyridin-2-amine |
| H | 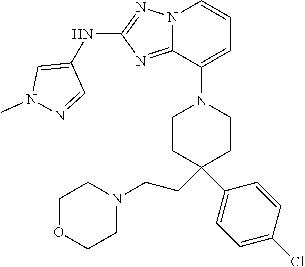 [SMILES: Cn1cc(Nc2nc3c(N4CCC(CCN5CCOCC5)(c5ccc(Cl)cc5)CC4)cccn3n2)cn1] | 8-[4-(4-chlorophenyl)-4-(2- morpholinoethyl)-1-piperidyl]-N-(1- methylpyrazol-4-yl)- [1,2,4]triazolo[1,5-a]pyridin-2-amine |
| I | 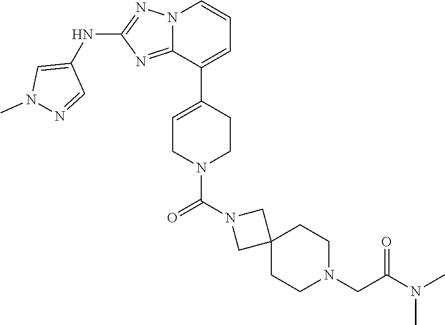 | N,N-dimethyl-2-[2-[4-[2-[(1- methylpyrazol-4-yl)amino]- [1,2,4]triazolo[1,5-a]pyridin-8-yl]- 3.6-dihydro-2H-pyridine-1-carbonyl]- 2,7-diazaspiro[3.5]nonan-7- yl]acetamide |
| J | 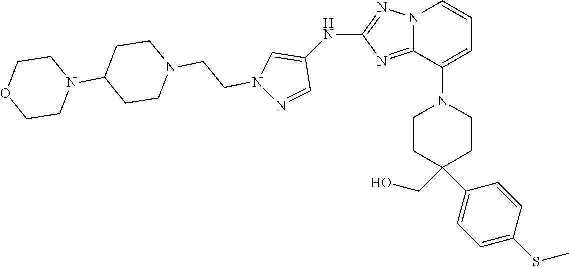 [SMILES: CSc1ccc(C2(CO)CCN(c3cccn4nc(Nc5cnn(CCN6CCC(N7CCOCC7)CC6)c5)nc34)CC2)cc1] | [4-(4-methylsulfanylphenyl)-1-[2-[[1- [2-(4-morpholino-1- piperidyl)ethyl]pyrazol-4-yl]amino]- [1,2,4]triazolo[1,5-a]pyridin-8-yl]-4- piperidyl]methanol |
| K | 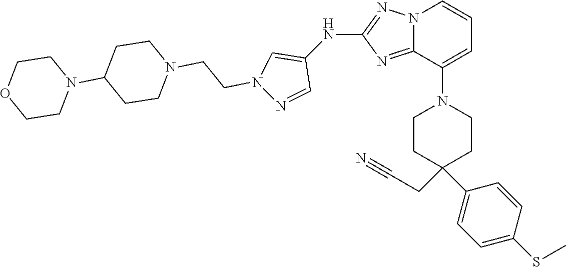 [SMILES: CSc1ccc(C2(CC#N)CCN(c3cccn4nc(Nc5cnn(CCN6CCC(N7CCOCC7)CC6)c5)nc34)CC2)cc1] | 2-[4-(4-methylsulfanylphenyl)-1-[2- [[1-[2-(4-morpholino-1- piperidyl)ethyl]pyrazol-4-yl]amino]- [1,2,4]triazolo[1,5-a]pyridin-8-yl]-4- piperidyl]acetonitrile |
| L | 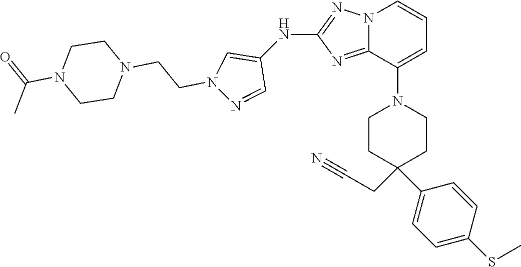 [SMILES: CSc1ccc(C2(CC#N)CCN(c3cccn4nc(Nc5cnn(CCN6CCN(C(C)=O)CC6)c5)nc34)CC2)cc1] | 2-[1-[2-[[1-[2-(4-acetylpiperazin-1- yl)ethyl]pyrazol-4-yl]amino]- [1,2,4]triazolo[1,5-a]pyridin-8-yl]-4- (4-methylsulfanylphenyl)-4- piperidyl]acetonitrile |
| M | 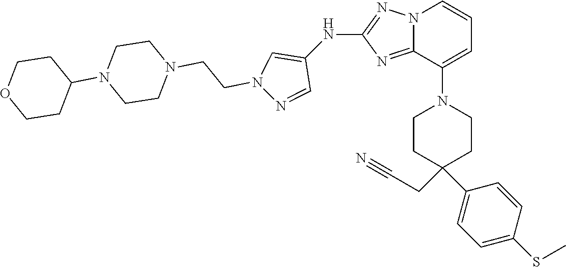 [SMILES: CSc1ccc(C2(CC#N)CCN(c3cccn4nc(Nc5cnn(CCN6CCN(C7CCOCC7)CC6)c5)nc34)CC2)cc1] | 2-[4-(4-methylsulfanylphenyl)-1-[2- [[1-[2-(4-tetrahydropyran-4- ylpiperazin-1-yl)ethyl]pyrazol-4- yl]amino]-[1,2,4]triazolo[1,5- a]pyridin-8-yl]-4- piperidyl]acetonitrile |
| N | 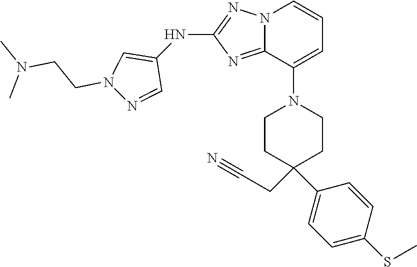 | 2-[1-[2-[[1-[2- (dimethylamino)ethyl]pyrazol-4- yl]amino]-[1,2,4]triazolo[1,5- a]pyridin-8-yl]-4-(4- methylsulfanylphenyl)-4- piperidyl]acetonitrile |
| O | 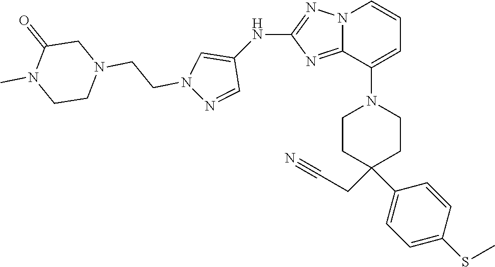 | 2-[1-[2-[[1-[2-(4-methyl-3-oxo- piperazin-1-yl)ethyl]pyrazol-4- yl]amino]-[1,2,4]triazolo[1,5- a]pyridin-8-yl]-4-(4- methylsulfanylphenyl)-4- piperidyl]acetonitrile |
| P | 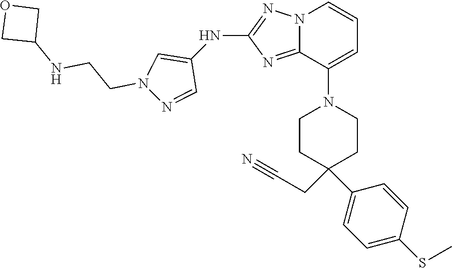 [SMILES: CSc1ccc(C2(CC#N)CCN(c3cccn4nc(Nc5cnn(CCNC6COC6)c5)nc34)CC2)cc1] | 2-[4-(4-methylsulfanylphenyl)-1-[2- [[1-[2-(oxetan-3- ylamino)ethyl]pyrazol-4-yl]amino]- [1,2,4]triazolo[1,5-a]pyridin-8-yl]-4- piperidyl]acetonitrile |
| Q | 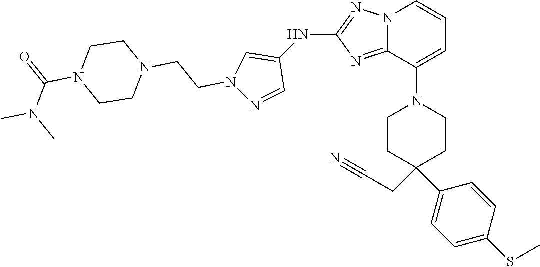 | 4-[2-[4-[[8-[4-(cyanomethyl)-4-(4- methylsulfanylphenyl)-1-piperidyl]- [1,2,4]triazolo[1,5-a]pyridin-2- yl]amino]pyrazol-1-yl]ethyl]-N,N- dimethyl-piperazine-1-carboxamide |
| R and | 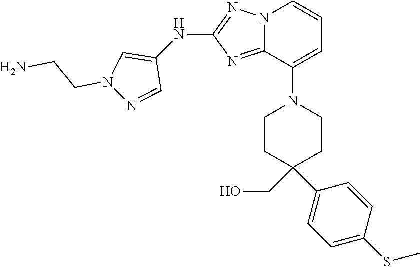 [SMILES: CSc1ccc(C2(CO)CCN(c3cccn4nc(Nc5cnn(CCN)c5)nc34)CC2)cc1] | [1-[2-[[1-(2-aminoethyl)pyrazol-4- yl]amino]-[1,2,4]triazolo[1,5- a]pyridin-8-yl]-4-(4- methylsulfanylphenyl)-4- piperidyl]methanol |
| S | 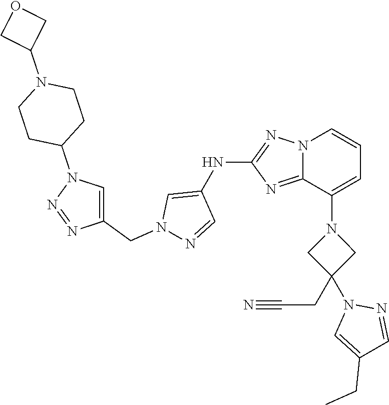 [SMILES: CCc1cnn(C2(CC#N)CN(c3cccn4nc(Nc5cnn(Cc6cn(C7CCN(C8COC8)CC7)nn6)c5)nc34)C2)c1] | 2-[3-(4-ethylpyrazol-1-yl)-1-[2-[[1- [[1-[1-(oxetan-3-yl)-4- piperidyl]triazol-4-yl]methyl]pyrazol- 4-yl]amino]-[1,2,4]triazolo[1,5- a]pyridin-8-yl]azetidin-3- yl]acetonitrile |

### Embodiment 39

A pharmaceutical composition comprising a compound of any one of Embodiments 1 to 38a, or a pharmaceutically acceptable salt thereof.

### Embodiment 40

The composition of Embodiment 39, further comprising a pharmaceutically acceptable carrier, adjuvant or vehicle.

### Embodiment 41

A method of preventing, treating or lessening the severity of a disease or condition responsive to the inhibition of a Janus kinase activity in a patient, comprising administering to the patient a therapeutically effective amount of a compound of any one of Embodiments 1 to 38a, or a pharmaceutically acceptable salt thereof.

### Embodiment 42

The method of Embodiment 41, wherein the Janus kinase is JAK1.

### Embodiment 43

A method of treating an inflammatory disease in a patient, comprising administering to the patient a therapeutically effective amount of a compound of any one of Embodiments 1 to 38a, or a pharmaceutically acceptable salt thereof.

### Embodiment 44

The method of Embodiment 43, wherein the inflammatory disease is asthma.

### Embodiment 45

The method of any one of Embodiments 41 to 44, further comprising administering a second therapeutic agent.

### Embodiment 46

A kit comprising a pharmaceutical composition of Embodiment 39 or 40, or a compound of any one of Embodiments 1 to 38a or a pharmaceutically acceptable salt thereof; and instructions for use.

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare other compounds of the present invention, and alternative methods for preparing the compounds are within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, or by making routine modifications of reaction conditions. Alternatively, other reactions disclosed herein or known in the art will be recognized as having applicability for preparing other compounds of the invention.

### EXAMPLES

Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as defined by the claims.

Abbreviations:
AcOH Acetic acid
BINAP 2,2′-Bis(diphenylphosphino)-1,1′-binaphthalene
n-BuLi n-Butyllithium solution
t-BuOH tert-butanol
t-BuOK Potassium tert-butoxide
t-BuONa Sodium tert-butoxide
CDCl₃ Deuterated chloroform
CD₃OD Deuterated methanol
CO Carbon monoxide
Cs₂CO₃ Cesium carbonate
CuI Copper (I) iodide
Cu₂O Copper (I) oxide
DIAD Diisopropyl azodicarboxylate
DIBAl-H Diisobutylaluminum hydride
DIPEA Diisopropylethylamine
DMF N,N-Dimethylformamide
DMSO Dimethylsulfoxide
DMSO-d6 Deuterated dimethylsulfoxide
EtOAc Ethyl acetate
EtOH Ethanol
g Gram
HATU (O-(7-azabenzotriazol-1-yl)-N,N,N′,N′-tetramethyluronium hexafluorophosphate)
HCl Hydrochloric acid
HCOOH Formic acid
HM-N Isolute HM-N is a modified form of diatomaceous earth
KOAc Potassium acetate
KOH Potassium hydroxide
K₃PO₄ Potassium phosphate tribasic
L Litre
MDAP Mass-directed automated purification
MeCN Acetonitrile
MeOH Methanol
mg Milligram
mL Millilitre
mmol Millimoles
Ms₂O Methanesulfonic anhydride
NaBH₃CN Sodium cyanoborohydride
NaBH₄ Sodium borohydride
NaCN Sodium cyanide
NaHCO₃ Sodium hydrogen carbonate
NaOH Sodium hydroxide
Na₂SO₄ Sodium sulfate
NH₃.H₂O 0.880 ammonia solution
NH₂OH.HCl Hydroxylamine hydrochloride
NH₄HCO₃ Ammonium bicarbonate
NH₄OAc Ammonium acetate
Pd/C Palladium on carbon
Pd₂(dba)₃ Tris(dibenzylidineacetone)palladium(0)
Pd(dppf)Cl₂ [1,1′-Bis(diphenylphosphino)ferrocene]-dichloropalladium-(II), complex with dichloromethane
Pd(OAc)₂ Palladium (II) acetate
Pd(PPh₃)₄ Tetrakis(triphenylphosphine)palladium(0)
PTSA p-Toluene sulfonic acid
SCX-2 ISOLUTE® Si-Propylsulfonic acid
THF Tetrahydrofuran
TFA Trifluoroacetic acid
TLC Thin layer chromatography
XantPhos 4,5-Bis(diphenylphosphino)-9,9-dimethylxanthene
X-phos 2-Dicyclohexylphosphino-2′,4′,6′-triisopropylbiphenyl

NMR Analytical Methods

¹H NMR spectra were recorded at ambient temperature using a Varian Unity Inova (400 MHz) spectrometer with a 400 4NUC 5 mm probe, a Bruker Avance DRX400 (400 MHz) spectrometer with a PABBO 5 mm probe. Chemical shifts are expressed in ppm relative to tetramethylsilane. The following abbreviations have been used: br=broad signal, s=singlet, d=doublet, dd=double doublet, t=triplet, q=quartet, m=multiplet.

LCMS Analytical Methods

High Pressure Liquid Chromatography-Mass Spectrometry (LCMS) experiments to determine retention times (RT) and associated mass ions were performed using one of the following methods with either UV detector monitoring at 220 nm and 254 nm or evaporative light scattering detection, and mass spectrometry scanning 110-800 amu in ESI+ ionization mode.

Method 1 Experiments were performed on a Waters ZMD single quadrupole mass spectrometer with an electrospray source operating in positive and negative ion mode linked to a Waters 1525 LC system. Detection was achieved using a UV diode array detector and a Sedex 85 evaporative light scattering detector. The LC column was a Phenomenex Luna 3 micron C18(2) 30×4.6 mm. The flow rate was 2 mL/min. The initial solvent system was 95% water containing 0.1% formic acid (solvent A) and 5% acetonitrile containing 0.1% formic acid (solvent B) for 0.5 min followed by a gradient up to 5% solvent A and 95% solvent B over the next 4 min. The final solvent system was held constant for a further 1 min.

Method 2 Experiments were performed on a Waters Micromass ZQ2000 single quadrupole mass spectrometer with an electrospray source operating in positive and negative ion mode linked to a Waters Acquity UPLC system. The LC column was an Acquity BEH C18 1.7 um 100×2.1 mm, maintained at 40° C. Detection was achieved using a UV PDA detector. The flow rate was 2 mL/min. The initial solvent system was 95% water containing 0.1% formic acid (solvent A) and 5% acetonitrile containing 0.1% formic acid (solvent B) for 0.4 min followed by a gradient up to 5% solvent A and 95% solvent B over the next 5.6 min. The final solvent system was held constant for a further 0.8 min.

Method 3 Experiments were performed on a Waters Acquity UPLC with a Shim-pack XR-ODS column (50×3.0 mm Acquity BEH C18, 2.2 μm particle size), elution with solvent A: Water/0.05% TFA; solvent B: Acetonitrile/0.05% TFA at 40° C. Gradient:

| Gradient - Time | flow ml/min | % A | % B |
|---|---|---|---|
| 0.00 | 1.0 | 95 | 5 |
| 2.00 | 1.0 | 0 | 100 |
| 3.20 | 1.0 | 0 | 100 |
| 3.30 | 1.0 | 95 | 5 |

### Detection—UV (220 and 254 nm) and ELSD

MS ionization method—ESI+

Method 4 Experiments were performed on a SHIMADZU 20A HPLC with a C18-reverse-phase column (50×3 mm Xtimate TM—C18, 2.2 μm particle size), elution with solvent A: water+0.05% trifluoroacetic acid; solvent B: acetonitrile+0.05% trifluoroacetic acid. Gradient:

| Gradient - Time | flow ml/min | % A | % B |
|---|---|---|---|
| 0.00 | 1.0 | 95 | 5 |
| 2.00 | 1.0 | 0 | 100 |
| 3.20 | 1.0 | 0 | 100 |
| 3.30 | 1.0 | 95 | 5 |

### Detection—UV (220 and 254 nm) and ELSD

Method 5 Experiments were performed on a SHIMADZU 20A HPLC with a C18-reverse-phase column (30×2.1 mm Xtimate TM—C18, 3 μm particle size), elution with solvent A: water+0.05% trifluoroacetic acid; solvent B: acetonitrile+0.05% trifluoroacetic acid. Gradient:

| Gradient - Time | flow ml/min | % A | % B |
|---|---|---|---|
| 0.00 | 1.0 | 95 | 5 |
| 1.10 | 1.0 | 0 | 100 |
| 1.60 | 1.0 | 0 | 100 |
| 1.70 | 1.0 | 95 | 5 |

### Detection—UV (220 and 254 nm) and ELSD

Method 6 Experiments were performed on a SHIMADZU 20A HPLC with a C18-reverse-phase column (50×2.1 mm Xtimate TM-C18, 2.7 μm particle size), elution with solvent A: water+0.05% trifluoroacetic acid; solvent B: acetonitrile+0.05% trifluoroacetic acid. Gradient:

| Gradient - Time | flow ml/min | % A | % B |
|---|---|---|---|
| 0.00 | 1.0 | 95 | 5 |
| 1.10 | 1.0 | 0 | 100 |
| 1.60 | 1.0 | 0 | 100 |
| 1.70 | 1.0 | 95 | 5 |

### Detection—UV (220 and 254 nm) and ELSD

Method 7 Experiments were performed on a Waters Acquity UPLC with a Shim-pack XR-ODS column (50×3.0 mm Acquity BEH C18, 2.2 μm particle size), elution with solvent A: Water/0.05% TFA; solvent B: Acetonitrile/0.05% TFA at 40° C. Gradient:

| Gradient - Time | flow ml/min | % A | % B |
|---|---|---|---|
| 0.00 | 1.0 | 95 | 5 |
| 3.20 | 1.0 | 40 | 60 |
| 3.80 | 1.0 | 0 | 100 |
| 4.60 | 1.0 | 0 | 100 |
| 4.75 | 1.0 | 95 | 5 |

### Detection—UV (220 and 254 nm) and ELSD

### Preparative Mass Directed Automated Purification Conditions

MDAP Method 1 Agilent 1260 infinity purifications system. Agilent 6100 series single Quadrupole LC/MS. XSEELECT CSH Prep C18 5 μm OBD, 30×150 mm, RT. Elution with solvent A: 0.1% aqueous formic acid; solvent B: 0.1% formic acid in acetonitrile 60 ml/min. 10%-95%, 22 min, centered around a specific focused gradient. Injection of a 20-60 mg/ml solution in DMSO (+optional formic acid and water)

MDAP Method 2 Agilent 1260 infinity purifications system. Agilent 6100 series single Quadrupole LC/MS. XBridge Prep C18 5 μm OBD, 30×150 mm, RT. Elution with solvent A: 0.1% aqueous ammonia; solvent B: 0.1% ammonia in acetonitrile 60 ml/min. 10%-95%, 22 min, centered around a specific focused gradient. Injection of a 20-60 mg/ml solution in DMSO (+optional formic acid and water)

### Example 1a

### [1-{2-[1-(1-Benzyl-piperidin-4-yl)-1H-pyrazol-4-ylamino]-[1,2,4]triazolo[1,5-a]pyridin-8-yl}-4-(4-chloro-phenyl)-piperidin-4-yl]-methanol (Compound No. 1-3)

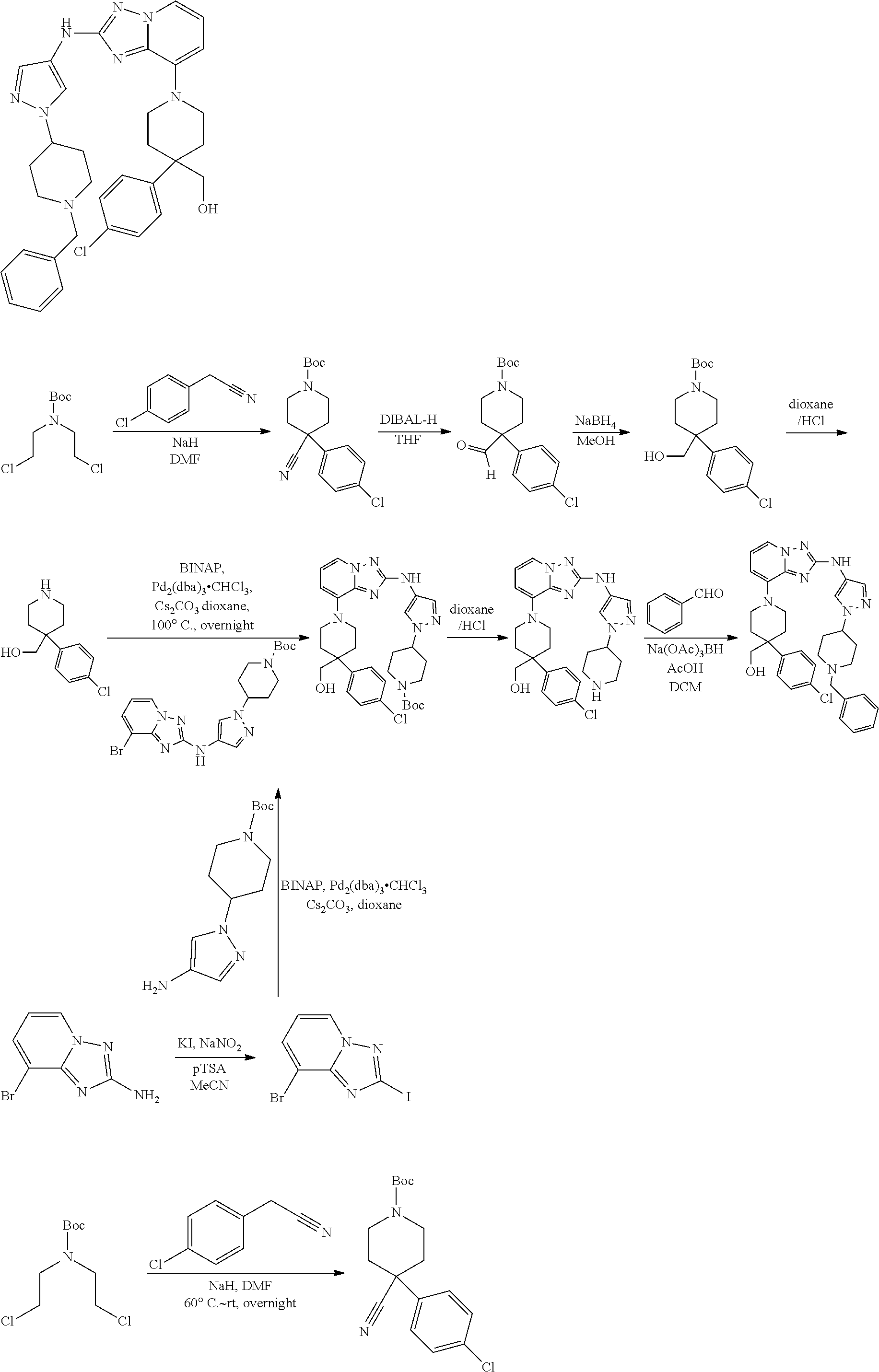

### Step 1:

Sodium hydride (27 g, 60% in mineral oil, 666.73 mmol) was added portionwise at 0° C. under N₂ over 2 hr to a solution of 2-(4-chlorophenyl)acetonitrile (20.1 g, 132.6 mmol) and tert-butyl N,N-bis(2-chloroethyl)carbamate (35.4 g, 146.2 mmol) in anhydrous DMF (200 mL). The resulting solution was stirred at 60° C. for 1.5 h and at ambient temperature overnight. The reaction was quenched by the careful addition of saturated aqueous ammonium chloride solution (250 mL). The resulting solution was extracted with DCM (3×200 mL). The combined organic extract was washed with brine (2×300 mL), dried over anhydrous sodium sulfate and concentrated under vacuum. The residue was purified on a silica gel column with DCM/petroleum ether (1:1). Appropriate fractions were combined and evaporated to afford tert-butyl-4-(4-chlorophenyl)-4-cyanopiperidine-1-carboxylate (30 g, 71%) as a yellow solid. TLC: Rf=0.15; EtOAc/petroleum ether=1/5.

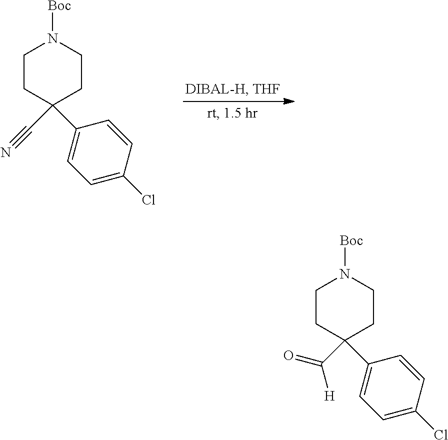 [SMILES: CC(C)(C)OC(=O)N1CCC(C#N)(c2ccc(Cl)cc2)CC1 ; CC(C)(C)OC(=O)N1CCC(C=O)(c2ccc(Cl)cc2)CC1]

### Step 2:

A solution of diisobutyl aluminium hydride (1 M in hexane, 7.8 mL, 7.81 mmol) was added dropwise to a cooled (0° C.) solution of tert-butyl 4-(4-chlorophenyl)-4-cyanopiperidine-1-carboxylate (1 g, 3.12 mmol) in anhydrous tetrahydrofuran (5 mL). On complete addition, the resulting solution was allowed to warm to ambient temperature and stirred for 1.5 h. The reaction mixture was poured onto water/ice (100 mL) and the resulting solution was extracted with EtOAc (200 mL). The organic layer was washed with aqueous 2M hydrogen chloride solution (2×50 mL), saturated aqueous sodium bicarbonate solution (3×50 mL) and brine (1×50 mL), dried over anhydrous sodium sulfate and concentrated under vacuum to afford tert-butyl 4-(4-chlorophenyl)-4-formylpiperidine-1-carboxylate (610 mg, crude) as a yellow solid, which was used in the next step without further purification. TLC: Rf=0.15; EtOAc/petroleum ether=1/5.

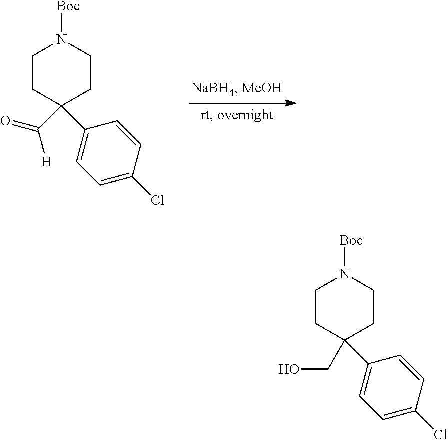 [SMILES: CC(C)(C)OC(=O)N1CCC(C=O)(c2ccc(Cl)cc2)CC1 ; CC(C)(C)OC(=O)N1CCC(CO)(c2ccc(Cl)cc2)CC1]

### Step 3:

NaBH₄ (144 mg, 3.81 mmol) was added to a cooled (0° C.) solution of tert-butyl 4-(4-chlorophenyl)-4-formylpiperidine-1-carboxylate (610 mg, 1.88 mmol) in methanol (5 mL) and the resulting solution was stirred at ambient temperature overnight. The reaction was quenched by the addition of three drops of water. The resulting mixture was concentrated under vacuum and the resultant residue was purified by flash chromatography on a silica gel eluting with EtOAc/petroleum ether (1:10). Appropriate fractions were combined and evaporated to afford tert-butyl 4-(4-chlorophenyl)-4-(hydroxymethyl) piperidine-1-carboxylate (320 mg, 52%) as a white solid. TLC: Rf=0.4; EtOAc/petroleum ether=1/1.

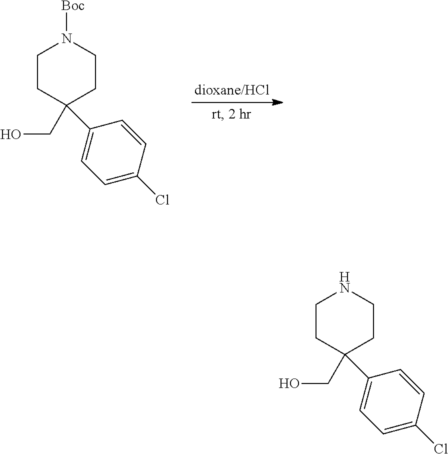 [SMILES: CC(C)(C)OC(=O)N1CCC(CO)(c2ccc(Cl)cc2)CC1 ; OCC1(c2ccc(Cl)cc2)CCNCC1]

### Step 4.

A solution of tert-butyl 4-(4-chlorophenyl)-4-(hydroxymethyl) piperidine-1-carboxylate (300 mg, 0.92 mmol) in HCl/dioxane (1M, 10 mL) was stirred at ambient temperature for 2 h. The resulting mixture was concentrated under vacuum, and the pH of the residue was adjusted to 10 by the addition of saturated aqueous sodium hydrogen carbonate solution. The resulting mixture was concentrated to dryness under vacuum and the residue was purified by flash chromatography on a silica gel column eluting with DCM/MeOH (10:1). Appropriate fractions were combined and evaporated to afford [4-(4-chlorophenyl)piperidin-4-yl]methanol (180 mg, 86%) as a yellow solid. LCMS (Method 3): R_{T}=1.03 min, m/z=226.0 [M+H]⁺.

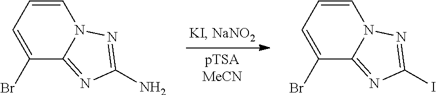 [SMILES: Nc1nc2c(Br)cccn2n1 ; Brc1cccn2nc(I)nc12]

### Step 5.

A solution of potassium iodide (90.9 g, 549 mmol) and sodium nitrite (29.2 g, 420 mmol) in water (180 mL) was added dropwise sub-surface to a cooled 13-15° C. suspension of 8-bromo-[1,2,4]triazolo[1,5-a]pyridin-2-ylamine (30.0 g, 141 mmol) and p-toluenesulfonic acid monohydrate (123.3 g, 648 mmol) in acetonitrile (900 mL) over 60 min. On complete addition the mixture was allowed to warm to ambient temperature and stirred for 30 min. The reaction mixture was heated to 40° C. in a water bath then allowed to cool to ambient temperature. The reaction was quenched by the addition of saturated aqueous sodium bicarbonate solution (600 mL) and extracted with EtOAc (2×500 mL). The combined organic phase was washed with 10% w/w aqueous sodium metabisulfite solution (500 mL), water (500 mL) and brine (500 mL), dried over anhydrous sodium sulfate, filtered and evaporated to afford a yellow solid. The crude solid was purified by flash chromatography on silica eluting with DCM on a gradient of MeOH (0-2%). Appropriate fractions were collected and evaporated to afford 8-bromo-2-iodo-[1,2,4]triazolo[1,5-a]pyridine as a white solid (28.6 g, 63%). LCMS (Method 1) R_{T}=2.56 min, m/z=323.8/325.8 [M+H]⁺.

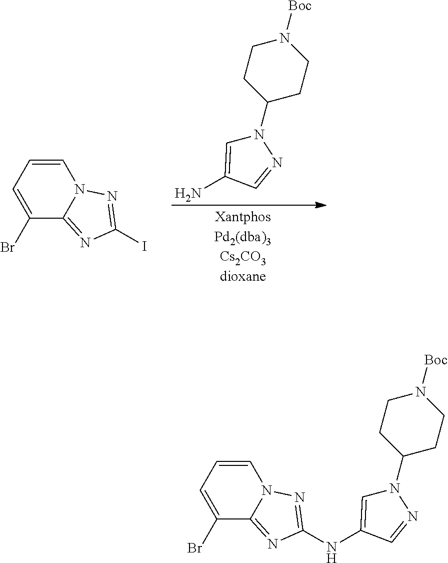 [SMILES: Brc1cccn2nc(I)nc12 ; CC(C)(C)OC(=O)N1CCC(n2cc(N)cn2)CC1 ; CC(C)(C)OC(=O)N1CCC(n2cc(Nc3nc4c(Br)cccn4n3)cn2)CC1]

### Step 6:

A degassed suspension of 8-bromo-2-iodo-[1,2,4]triazolo[1,5-a]pyridine (1.11 g, 3.41 mmol), 4-(4-amino-pyrazol-1-yl)-piperidine-1-carboxylic acid tert-butyl ester (1.00 g, 3.75 mmol), tris(dibenzylidineacetone) palladium (0) (156 mg, 0.17 mmol) and Xantphos (197 mg, 0.34 mmol) in 1,4-dioxane (15 mL) was heated under reflux for 18 h. The reaction mixture was allowed to cool to ambient temperature and the precipitated solid was removed by filtration. The filtrate was concentrated under vacuum and the residue purified by flash chromatography on silica gel eluting with DCM on a gradient of MeOH (0-5%). Appropriate fractions were collected and evaporated to afford 4-[4-(8-bromo-[1,2,4]triazolo[1,5-a]pyridin-2-ylamino)-pyrazol-1-yl]-piperidine-1-carboxylic acid tert-butyl ester (1.36 g, 86%) as a brown solid. LCMS (Method 1) R_{T}=3.19 min, m/z=462.1/464.1 [M+H]⁺.

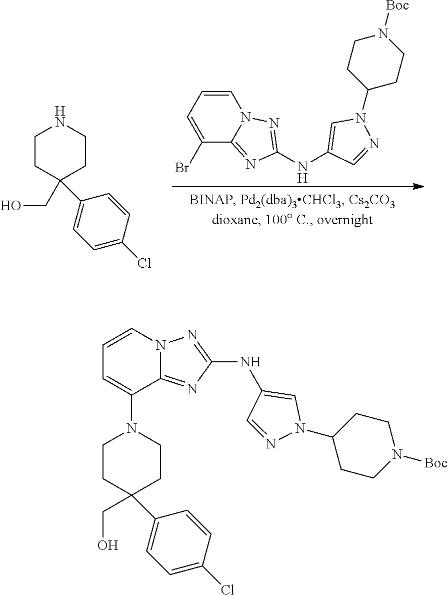 [SMILES: OCC1(c2ccc(Cl)cc2)CCNCC1 ; CC(C)(C)OC(=O)N1CCC(n2cc(Nc3nc4c(Br)cccn4n3)cn2)CC1 ; CC(C)(C)OC(=O)N1CCC(n2cc(Nc3nc4c(N5CCC(CO)(c6ccc(Cl)cc6)CC5)cccn4n3)cn2)CC1]

### Step 7:

A degassed mixture of 4-[4-(8-bromo-[1,2,4]triazolo[1,5-a]pyridin-2-ylamino)-pyrazol-1-yl]-piperidine-1-carboxylic acid tert-butyl ester (1.26 g, 2.72 mmol), [4-(4-chlorophenyl)piperidin-4-yl]methanol (920 mg, 4.08 mmol), Cs₂CO₃ (1.77 g, 5.44 mmol), Pd₂(dba)₃ (125 mg, 0.14 mmol) and BINAP (169 mg, 0.27 mmol) in 1,4-dioxane (15 mL) was heated under reflux for 18 h. The reaction mixture was allowed to cool to ambient temperature and the resultant solid removed by filtration through celite. The filtrate was concentrated under vacuum and the residue was purified by flash chromatography on silica gel eluting with DCM on a gradient of MeOH (0-6%). Appropriate fractions were combined and evaporated to afford 4-(4-{8-[4-(4-chloro-phenyl)-4-hydroxymethyl-piperidin-1-yl]-[1,2,4]triazolo[1,5-a]pyridin-2-ylamino}-pyrazol-1-yl)-piperidine-1-carboxylic acid tert-butyl ester (1.35 g, 82%) as a brown glass. LCMS (Method 1): R_{T}=3.74 min, m/z=607.1 [M+H]⁺.

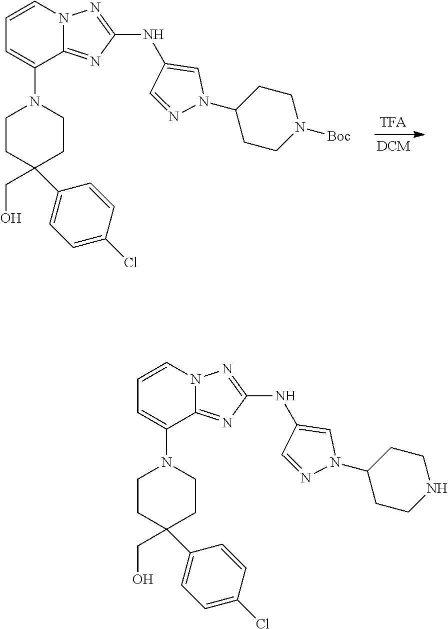 [SMILES: CC(C)(C)OC(=O)N1CCC(n2cc(Nc3nc4c(N5CCC(CO)(c6ccc(Cl)cc6)CC5)cccn4n3)cn2)CC1 ; OCC1(c2ccc(Cl)cc2)CCN(c2cccn3nc(Nc4cnn(C5CCNCC5)c4)nc23)CC1]

### Step 8:

TFA (15 mL) was added to a solution of 4-(4-{8-[4-(4-chloro-phenyl)-4-hydroxymethyl-piperidin-1-yl]-[1,2,4]triazolo[1,5-a]pyridin-2-ylamino}-pyrazol-1-yl)-piperidine-1-carboxylic acid tert-butyl ester in DCM (15 mL) and the mixture left to stir at ambient temperature for 3 h. The mixture was diluted with MeOH and loaded onto an SCX-2 cartridge. The cartridge was washed with MeOH and eluted with 2M ammonia in MeOH. Appropriate basic methanolic fractions were combined and evaporated to afford {4-(4-chloro-phenyl)-1-[2-(1-piperidin-4-yl-1H-pyrazol-4-ylamino)-[1,2,4]triazolo[1,5-a]pyridin-8-yl]-piperidin-4-yl}-methanol (856 mg, 76%) as a brown glass. LCMS (Method 1): R_{T}=2.47 min, m/z=507.2 [M+H]⁺.

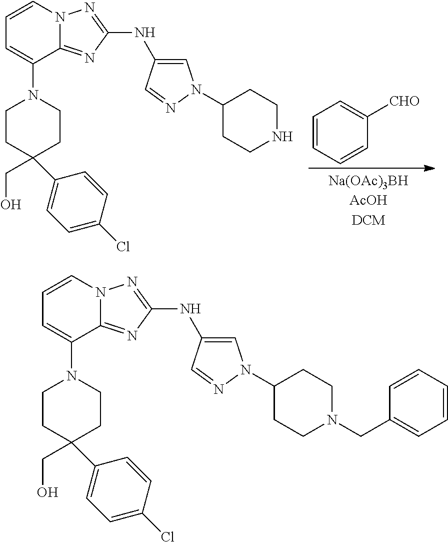

### Step 9:

Sodium triacetoxyborohydride (83 mg, 0.39 mmol) was added to a cooled (0° C.) solution of {4-(4-chloro-phenyl)-1-[2-(1-piperidin-4-yl-1H-pyrazol-4-ylamino)-[1,2,4]triazolo[1,5-a]pyridin-8-yl]-piperidin-4-yl}-methanol (131 mg, 0.26 mmol), benzaldehyde (29 μL, 0.28 mmol) and acetic acid (250 μL) in DCM (3 mL). On complete addition, the mixture was allowed to warm to ambient temperature and stirred for 1.5 h, diluted with MeOH and water and loaded onto an SCX-2 cartridge. The cartridge was washed with MeOH and the product eluted with 2M ammonia in MeOH. Appropriate basic methanolic fractions were combined and evaporated. The resultant residue was purified by flash chromatography on silica eluting with DCM on a gradient of 2M ammonia in MeOH (0-5%). Appropriate fractions were combined and evaporated and the residue purified by prep-HPLC (MDAP, Method 1). Appropriate fractions were collected and evaporated to afford [1-{2-[1-(1-benzyl-piperidin-4-yl)-1H-pyrazol-4-ylamino]-[1,2,4]triazolo[1,5-a]pyridin-8-yl}-4-(4-chloro-phenyl)-piperidin-4-yl]-methanol—formic acid salt (96 mg, 57%), Example 1-3, as a pale yellow glass. LCMS (Method 2): R_{T}=3.55 min, m/z=597.2 [M+H]⁺. ¹H NMR (400 MHz, DMSO-d₆): δ 9.13 (s, 1H), 8.18 (d, 1H, J=6.6 Hz), 8.15 (s, 1H), 7.81 (s, 1H), 7.47-7.43 (m, 2H), 7.39-7.33 (m, 5H), 7.31-7.21 (m, 1H), 6.79-6.74 (m, 1H), 6.68 (d, 1H, J=7.5 Hz), 4.14-4.04 (m, 1H), 3.84-3.74 (m, 2H), 3.52 (s, 3H), 3.44 (s, 2H), 3.36-3.35 (m, 1H), 3.05-2.86 (m, 4H), 2.24-1.88 (m, 9H).

### Example 1b

### 3-[4-(4-{8-[4-(4-Chloro-phenyl)-4-hydroxymethyl-piperidin-1-yl]-[1,2,4]triazolo[1,5-a]pyridin-2-ylamino}-pyrazol-1-yl)-piperidin-1-yl]-propionitrile (Compound No. 1-4)

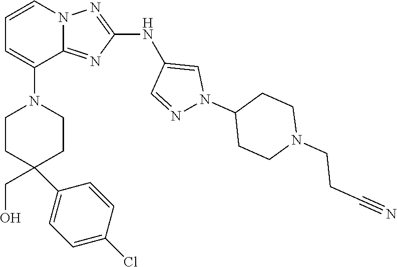 [SMILES: N#CCCN1CCC(n2cc(Nc3nc4c(N5CCC(CO)(c6ccc(Cl)cc6)CC5)cccn4n3)cn2)CC1]

A solution of {4-(4-chloro-phenyl)-1-[2-(1-piperidin-4-yl-1H-pyrazol-4-ylamino)-[1,2,4]triazolo[1,5-a]pyridin-8-yl]-piperidin-4-yl}-methanol (100 mg, 0.20 mmol), 3-chloropropionitrile (31 μL, 0.39 mmol) and triethylamine (165 μL, 1.18 mmol) in DCM (2 mL) was left to stir at ambient temperature for 18 h. The mixture was diluted with MeOH and loaded onto an SCX-2 cartridge. The cartridge was washed with MeOH and eluted with 2M ammonia in MeOH. Appropriate basic methanolic fractions were combined and evaporated. The resultant residue was purified by MDAP (Method 1). Appropriate fractions were combined and evaporated to afford 3-[4-(4-{8-[4-(4-chloro-phenyl)-4-hydroxymethyl-piperidin-1-yl]-[1,2,4]triazolo[1,5-a]pyridin-2-ylamino)}-pyrazol-1-yl)-piperidin-1-yl]-propionitrile—formic acid salt (61 mg, 54%), Example 1-4, as a colourless glass. LCMS (Method 2): R_{T}=3.27 min, m/z=560.2 [M+H]⁺. ¹H NMR (400 MHz, CDCl₃): δ 8.01 (d, 1H, J=6.3 Hz), 7.89 (s, 1H), 7.52 (s, 1H), 7.36 (s, 4H), 6.75-6.61 (m, 2H), 4.17-4.07 (m, 1H), 3.78-3.68 (m, 2H), 3.63 (s, 2H), 3.04 (dd, 4H, J=10.8, 10.8 Hz), 2.75 (dd, 3H, J=6.9, 6.9 Hz), 2.55 (dd, 2H, J=6.8, 6.8 Hz), 2.39-2.24 (m, 4H), 2.22-2.05 (m, 5H).

### Example 1c

### [4-(4-Chloro-phenyl)-1-(2-{1-[3-(4-methyl-piperazin-1-yl)-propyl]-1H-pyrazol-4-ylamino}-[1,2,4]triazolo[1,5-a]pyridin-8-yl)-piperidin-4-yl]-methanol (Compound No. 1-9)

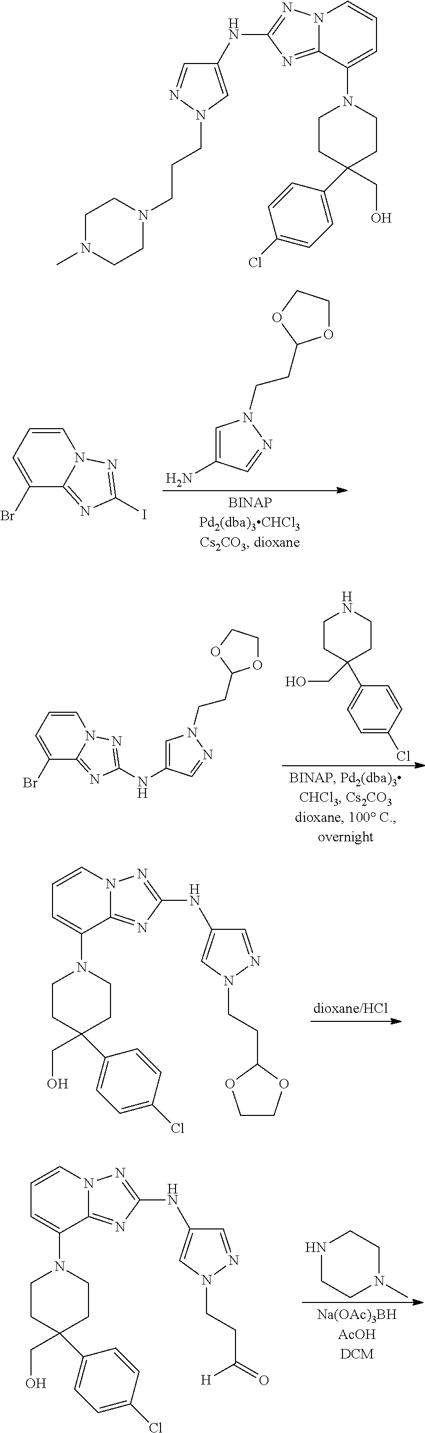

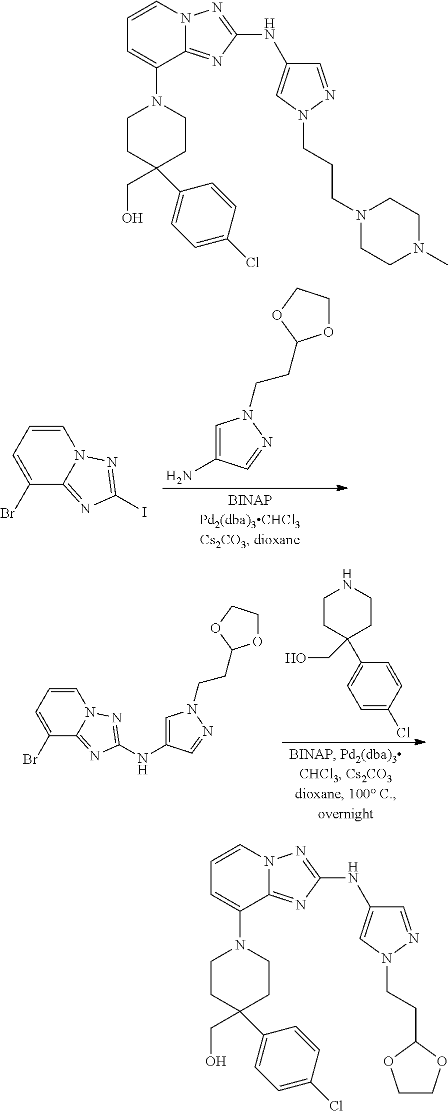

### Steps 1 and 2:

(4-(4-chloro-phenyl)-1-{2-[1-(2-[1,3]dioxolan-2-yl-ethyl)-1H-pyrazol-4-ylamino]-[1,2,4]triazolo[1,5-a]pyridin-8-yl}-piperidin-4-yl)-methanol was prepared in two steps from 8-bromo-2-iodo-[1,2,4]triazolo[1,5-a]pyridine (500 mg, 1.54 mmol) and 1-(2-[1,3]dioxolan-2-yl-ethyl)-1H-pyrazol-4-ylamine (311 mg, 1.70 mmol) following the palladium coupling procedures from step 6 and step 7 in Example 1a in 54% overall yield. LCMS (Method 1): R_{T}=3.14 min, m/z=524.2 [M+H]⁺.

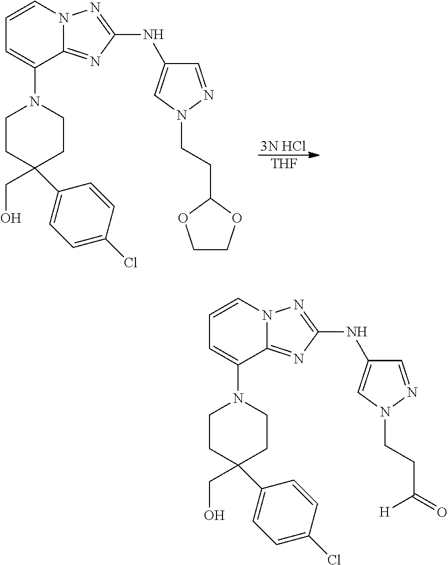

### Step 3:

A mixture of (4-(4-chloro-phenyl)-1-{2-[1-(2-[1,3]dioxolan-2-yl-ethyl)-1H-pyrazol-4-ylamino]-[1,2,4]triazolo[1,5-a]pyridin-8-yl}-piperidin-4-yl)-methanol (300 mg, 0.57 mmol) in 3N hydrochloric acid (20 mL) and THF (15 mL) was left to stir at ambient temperature for 18 h. The mixture was diluted with EtOAc and the pH of the aqueous phase adjusted to 7 by the addition of solid sodium hydrogen carbonate. The layers were separated and the aqueous phase was washed with brine, dried over anhydrous sodium sulphate, filtered evaporated to afford 3-(4-{8-[4-(4-chloro-phenyl)-4-hydroxymethyl-piperidin-1-yl]-[1,2,4]triazolo[1,5-a]pyridin-2-ylamino}-pyrazol-1-yl)-propionaldehyde (284 mg, quant.) as an off white solid. LCMS (Method 1): R_{T}=2.98 min, m/z=480.1 [M+H]⁺.

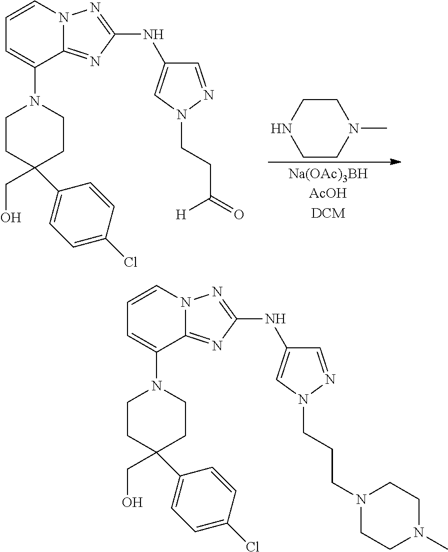

### Step 4:

Sodium triacetoxyborohydride (61 mg, 0.29 mmol) was added to a cooled (0° C.) solution of 3-(4-{8-[4-(4-chloro-phenyl)-4-hydroxymethyl-piperidin-1-yl]-[1,2,4]-triazolo[1,5-a]pyridin-2-ylamino}-pyrazol-1-yl)-propionaldehyde (91 mg, 0.19 mmol), N-methylpiperazine (32 μL, 0.29 mmol) and acetic acid (170 μL) in DCM (2 mL). On complete addition, the mixture was allowed to warm to ambient temperature and stirred for 2 h, diluted with MeOH and water and loaded onto an SCX-2 cartridge. The cartridge was washed with MeOH and the product eluted with 2M ammonia in MeOH. Appropriate basic methanolic fractions were combined and evaporated. The resultant residue was purified by prep-HPLC (MDAP, Method 1). Appropriate fractions were combined and evaporated to afford [4-(4-chloro-phenyl)-1-(2-{1-[3-(4-methyl-piperazin-1-yl)-propyl]-1H-pyrazol-4-ylamino}-[1,2,4]triazolo[1,5-a]pyridin-8-yl)-piperidin-4-yl]-methanol—formic acid salt (66 mg, 57%), Example 1-9, as a colourless glass. LCMS (Method 2): R_{T}=3.03 min, m/z=564.1 [M+H]⁺. ¹H NMR (400 MHz, DMSO-d₆): δ 9.14 (s, 1H), 8.17 (s, 1H), 8.15 (dd, 1H, J=0.8, 6.5 Hz), 7.75 (s, 1H), 7.47-7.42 (m, 2H), 7.40-7.37 (m, 2H), 6.77 (dd, 1H, J=6.6, 7.7 Hz), 6.68 (d, 1H, J=7.3 Hz), 4.06 (dd, 2H, J=6.8, 6.8 Hz), 3.81 (dd, 2H, J=4.8, 7.6 Hz), 3.42 (s, 2H), 2.98 (dd, 2H, J=10.1, 10.1 Hz), 2.53-2.49 (m, 2H), 2.36-2.32 (m, 6H), 2.26-2.18 (m, 4H), 2.17 (s, 3H), 2.08-1.98 (m, 2H), 1.93-1.84 (m, 2H).

### Example 1d

### {4-(4-Chloro-phenyl)-1-[2-(1-piperidin-4-ylmethyl-1H-pyrazol-4-ylamino)-[1,2,4]triazolo[1,5-a]pyridin-8-yl]-piperidin-4-yl}-methanol (Compound No. 1-1)

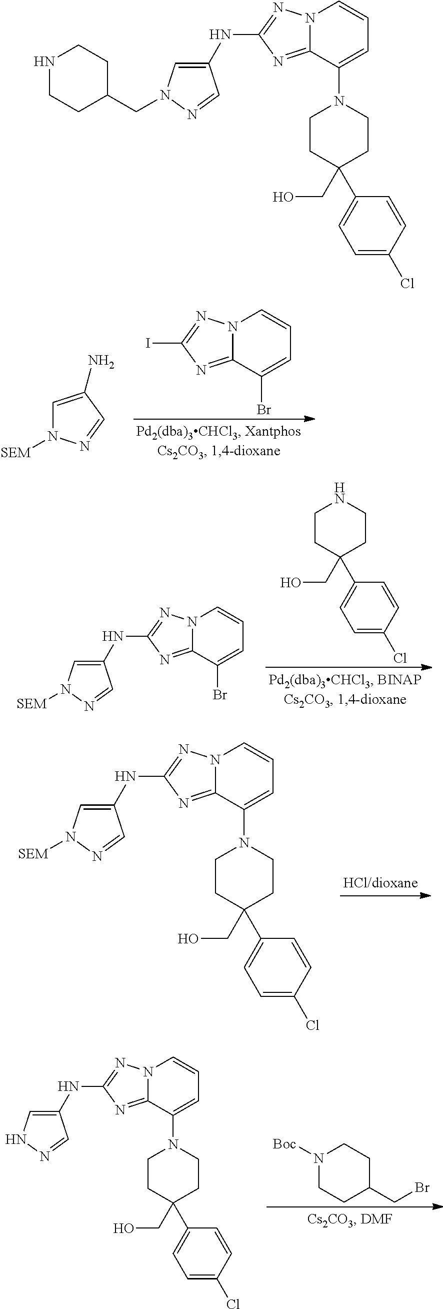

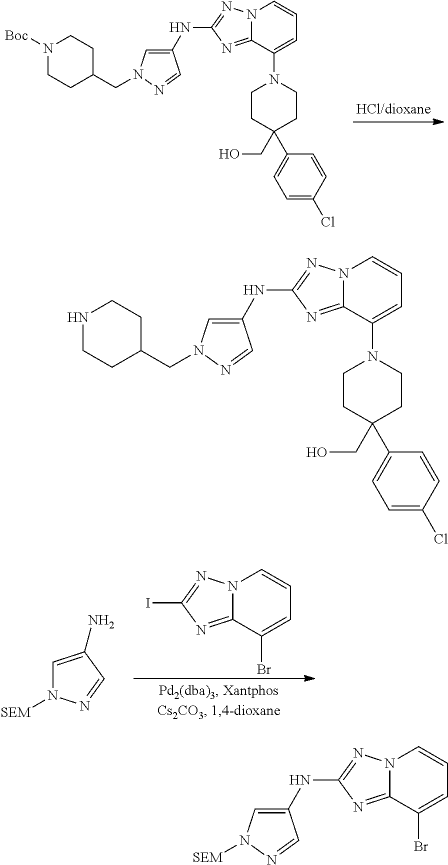

### Step 1:

A degassed suspension of 1-[[2-(trimethylsilyl)ethoxy]methyl]-1H-pyrazol-4-amine (630 mg, 2.95 mmol), 8-bromo-2-iodo-[1,2,4]triazolo[1,5-a]pyridine (960 mg, 2.96 mmol), Pd₂(dba)₃ (150 mg, 0.16 mmol), XantPhos (170 mg, 0.29 mmol) and Cs₂CO₃ (1.9 g, 5.83 mmol) and 1,4-dioxane (15 mL) was heated at 60° C. for 20 h then allowed to cool to ambient temperature. The resulting mixture was concentrated under vacuum and the residue was purified by flash chromatography on silica gel eluting with EtOAc/petroleum ether (1/1) to afford N-[8-bromo-[1,2,4]triazolo[1,5-a]pyridin-2-yl]-1-[[2-(trimethylsilyl)ethoxy]methyl]-1H-pyrazol-4-amine (800 mg, 66%) as a red solid. TLC: R_{f}=0.5; EtOAc/petroleum ether=1/1.

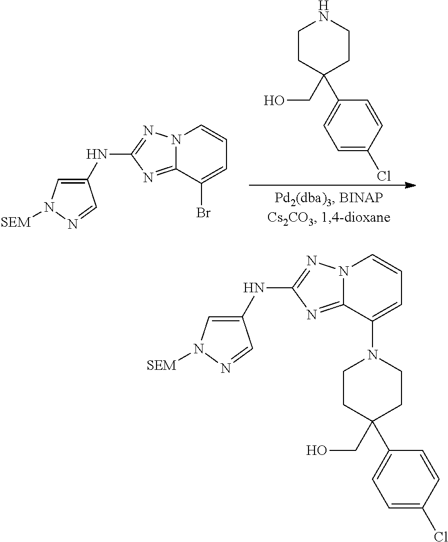 [SMILES: C[Si](C)(C)CCOCn1cc(Nc2nc3c(Br)cccn3n2)cn1 ; OCC1(c2ccc(Cl)cc2)CCNCC1 ; C[Si](C)(C)CCOCn1cc(Nc2nc3c(N4CCC(CO)(c5ccc(Cl)cc5)CC4)cccn3n2)cn1]

### Step 2:

N-[8-bromo-[1,2,4]triazolo[1,5-a]pyridin-2-yl]-1-[[2-(trimethylsilyl)-ethoxy]methyl]-1H-pyrazol-4-amine (400 mg, 0.98 mmol) and [4-(4-chlorophenyl)piperidin-4-yl]methanol (270 mg, 1.20 mmol) were coupled following the procedure detailed in Example 1a, step 7. The reaction mixture was concentrated under vacuum and the residue was purified by flash chromatography on silica gel eluting with EtOAc/petroleum ether (1:1). Appropriate fractions were combined and evaporated to afford [4-(4-chlorophenyl)-1-[2-[(1-[[2-(trimethylsilyl)ethoxy]methyl]-1H-pyrazol-4-yl)amino]-[1,2,4]triazolo[1,5-a]pyridin-8-yl]piperidin-4-yl]methanol (350 mg, 65%) as a yellow solid. LCMS (Method 5): R_{T}=1.12 min, m/z=554.2 [M+H]⁺.

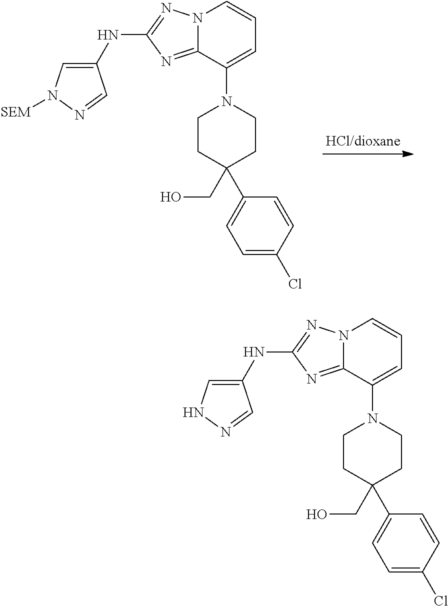

### Step 3:

A mixture of [4-(4-chlorophenyl)-1-[2-[(1-[[2-(trimethylsilyl)ethoxy]methyl]-1H-pyrazol-4-yl)amino]-[1,2,4]triazolo[1,5-a]pyridin-8-yl]piperidin-4-yl]methanol (650 mg, 1.17 mmol) in a saturated solution of HCl in 1,4-dioxane (20 mL) was stirred at ambient temperature for 20 h. The mixture was concentrated under vacuum to afford 500 mg of [4-(4-chlorophenyl)-1-[2-[(1H-pyrazol-4-yl)amino]-[1,2,4]triazolo[1,5-a]pyridine-8-yl]piperidin-4-yl]methanol as a light yellow solid. LCMS (Method 5): R_{T}=0.61 min, m/z=424.0 [M+H]⁺.

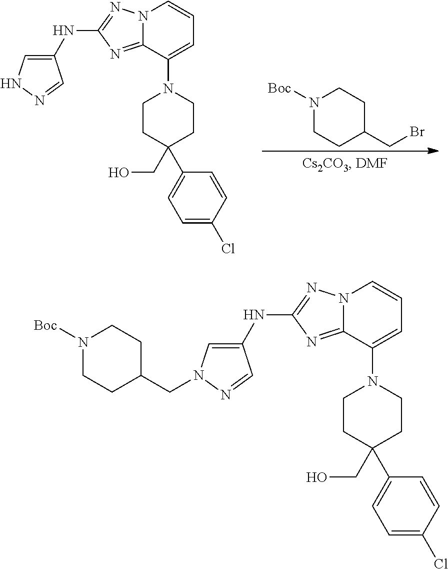 [SMILES: OCC1(c2ccc(Cl)cc2)CCN(c2cccn3nc(Nc4cn[nH]c4)nc23)CC1 ; CC(C)(C)OC(=O)N1CCC(CBr)CC1 ; CC(C)(C)OC(=O)N1CCC(Cn2cc(Nc3nc4c(N5CCC(CO)(c6ccc(Cl)cc6)CC5)cccn4n3)cn2)CC1]

### Step 4:

A mixture of [4-(4-chlorophenyl)-1-[2-[(1H-pyrazol-4-yl)amino]-[1,2,4]triazolo[1,5-a]pyridin-8-yl]piperidin-4-yl]methanol (57 mg, 0.14 mmol), Cs₂CO₃ (88 mg, 3.07 mmol) and 4-bromomethyl-piperidine-1-carboxylic acid tert-butyl ester (38 mg, 0.14 mmol) in N,N-dimethylformamide (3 mL) was heated at 80° C. for 18 h. The reaction mixture was allowed to cool to ambient temperature, diluted with water (30 mL) and extracted with EtOAc (2×15 mL). The combined organic phase was concentrated under vacuum and the residue was purified by prep-HPLC (MDAP, Method 1) to afford 4-(4-{8-[4-(4-chloro-phenyl)-4-hydroxymethyl-piperidin-1-yl]-[1,2,4]triazolo[1,5-a]pyridin-2-ylamino}-pyrazol-1-ylmethyl)-piperidine-1-carboxylic acid tert-butyl ester (21 mg, 25%) as white solid. LCMS (Method 1): R_{T}=3.80 min, m/z=621.1 [M+H]⁺.

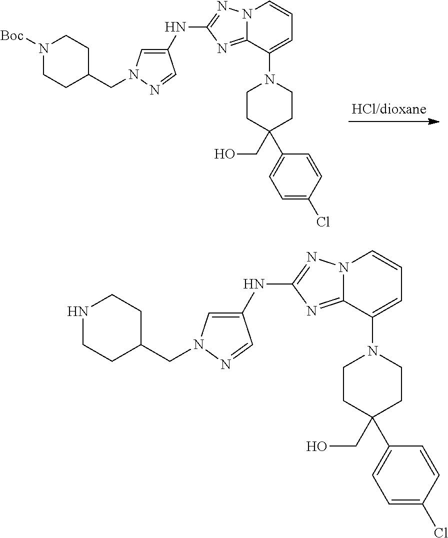 [SMILES: CC(C)(C)OC(=O)N1CCC(Cn2cc(Nc3nc4c(N5CCC(CO)(c6ccc(Cl)cc6)CC5)cccn4n3)cn2)CC1 ; OCC1(c2ccc(Cl)cc2)CCN(c2cccn3nc(Nc4cnn(CC5CCNCC5)c4)nc23)CC1]

### Step 5:

A mixture of 4-(4-{8-[4-(4-chloro-phenyl)-4-hydroxymethyl-piperidin-1-yl]-[1,2,4]triazolo[1,5-a]pyridin-2-ylamino}-pyrazol-1-ylmethyl)-piperidine-1-carboxylic acid tert-butyl ester (20 mg, 0.03 mmol) in a 4M solution of HCl in 1,4-dioxane (2 mL) was stirred at ambient temperature for 1 h. The mother liquors were decanted and the solid triturated with dioxane (5 mL). The solid was collected by filtration to afford {4-(4-chloro-phenyl)-1-[2-(1-piperidin-4-ylmethyl-1H-pyrazol-4-ylamino)-[1,2,4]triazolo[1,5-a]pyridin-8-yl]-piperidin-4-yl}-methanol—hydrochloride salt (15 mg, 89%) as a white solid. LCMS (Method 2) R_{T}=3.20 min, m/z=521.0 [M+H]⁺. ¹H NMR (400 MHz, CDCl₃): δ 8.03-8.00 (m, 1H), 7.84 (s, 1H), 7.70 (s, 1H), 7.49 (s, 1H), 7.34 (s, 4H), 6.72-6.68 (m, 1H), 6.62 (d, 1H, J=6.8 Hz), 3.98 (d, 2H, J=6.6 Hz), 3.87-3.80 (m, 2H), 3.53 (s, 2H), 3.11-3.05 (m, 2H), 3.01-2.94 (m, 2H), 2.63-2.54 (m, 2H), 2.35 (dd, 2H, J=2.8, 11.1 Hz), 2.14-2.04 (m, 2H), 2.03-1.97 (m, 1H), 1.65 (d, 2H, J=11.4 Hz), 1.35-1.22 (m, 2H).

The immediately preceding Examples may be modified via conventionally known chemistries to provide access to other compounds that fall within the scope of the present invention, such as compounds of formula (I), non-limiting examples of which are seen in Table 2.

TABLE 2

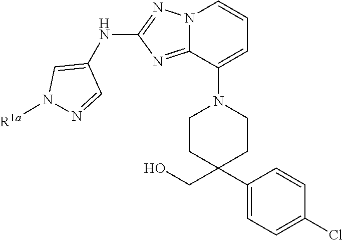

| Compound | | | LCMS(ESI) |
|---|---|---|---|
| No. | R^{1a} | Name | m/z [M + H]⁺ |
| 1-1 | 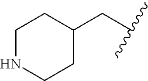 [SMILES: *CC1CCNCC1] | [4-(4-chlorophenyl)-1-[2-[[1-(4- piperidylmethyl)pyrazol-4- yl]amino]-[1,2,4]triazolo[1,5- a]pyridin-8-yl]-4- piperidyl]methanol | 522 |
| 1-2 | 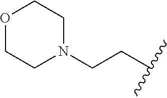 [SMILES: *CCN1CCOCC1] | [4-(4-chlorophenyl)-1-[2-[[1-(2- morpholinoethyl)pyrazol-4- yl]amino]-[1,2,4]triazolo[1,5- a]pyridin-8-yl]-4- piperidyl]methanol | 538 |
| 1-3 | 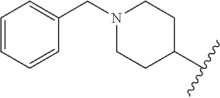 [SMILES: *C1CCN(Cc2ccccc2)CC1] | [1-[2-[[1-(1-benzyl-4- piperidyl)pyrazol-4-yl]amino]- [1,2,4]triazolo[1,5-a]pyridin-8-yl]- 4-(4-chlorophenyl)-4- piperidyl]methanol | 598 |
| 1-4 | 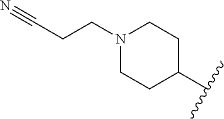 [SMILES: *C1CCN(CCC#N)CC1] | 3-[4-[4-[[8-[4-(4-chlorophenyl)-4- (hydroxymethyl)-1-piperidyl]- [1,2,4]triazolo[1,5-a]pyridin-2- yl]amino]pyrazol-1-yl]-1- piperidyl]propanenitrile | 561 |
| 1-5 | 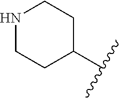 [SMILES: *C1CCNCC1] | [4-(4-chlorophenyl)-1-[2-[[1-(4- piperidyl)pyrazol-4-yl]amino]- [1,2,4]triazolo[1,5-a]pyridin-8-yl]- 4-piperidyl]methanol | 508 |
| 1-6 | 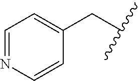 [SMILES: *Cc1ccncc1] | [4-(4-chlorophenyl)-1-[2-[[1-(4- pyridylmethyl)pyrazol-4- yl]amino]-[1,2,4]triazolo[1,5- a]pyridin-8-yl]-4- piperidyl]methanol | 516 |
| 1-7 | 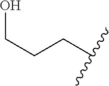 [SMILES: *CCCO] | 3-[4-[[8-[4-(4-chlorophenyl)-4- (hydroxymethyl)-1-piperidyl]- [1,2,4]triazolo[1,5-a]pyridin-2- yl]amino]pyrazol-1-yl]propan-1-ol | 483 |
| 1-8 | 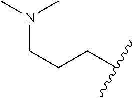 [SMILES: *CCCN(C)C] | [4-(4-chlorophenyl)-1-[2-[[1-[3- (dimethylamino)propyl]pyrazol-4- yl]amino]-[1,2,4]triazolo[1,5- a]pyridin-8-yl]-4- piperidyl]methanol | 510 |
| 1-9 | 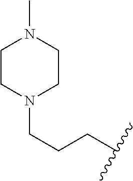 [SMILES: *CCCN1CCN(C)CC1] | [4-(4-chlorophenyl)-1-[2-[[1-[3-(4- methylpiperazin-1- yl)propyl]pyrazol-4-yl]amino]- [1,2,4]triazolo[1,5-a]pyridin-8-yl]- 4-piperidyl]methanol | 565 |
| 1-10 | 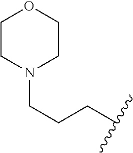 [SMILES: *CCCN1CCOCC1] | [4-(4-chlorophenyl)-1-[2-[[1-(3- morpholinopropyl)pyrazol-4- yl]amino]-[1,2,4]triazolo[1,5- a]pyridin-8-yl]-4- piperidyl]methanol | 552 |
| 1-11 | 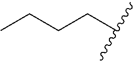 [SMILES: *CCCC] | [1-[2-[(1-butylpyrazol-4-yl)amino]- [1,2,4]triazolo[1,5-a]pyridin-8-yl]- 4-(4-chlorophenyl)-4- piperidyl]methanol | 481 |
| 1-12 | 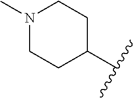 [SMILES: *C1CCN(C)CC1] | [4-(4-chlorophenyl)-1-[2-[[1-(1- methyl-4-piperidyl)pyrazol-4- yl]amino]-[1,2,4]triazolo[1,5- a]pyridin-8-yl]-4- piperidyl]methanol | 522 |
| 1-13 | 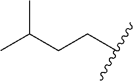 [SMILES: *CCC(C)C] | [4-(4-chlorophenyl)-1-[2-[(1- isopentylpyrazol-4-yl)amino]- [1,2,4]triazolo[1,5-a]pyridin-8-yl]- 4-piperidyl]methanol | 495 |
| 1-14 | 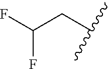 [SMILES: *CC(F)F] | [4-(4-chlorophenyl)-1-[2-[[1-(2,2- difluoroethyl)pyrazol-4-yl]amino]- [1,2,4]triazolo[1,5-a]pyridin-8-yl]- 4-piperidyl]methanol | 489 |
| 1-15 | 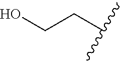 [SMILES: *CCO] | 2-[4-[[8-[4-(4-chlorophenyl)-4- (hydroxymethyl)-1-piperidyl]- [1,2,4]triazolo[1,5-a]pyridin-2- yl]amino]pyrazol-1-yl]ethanol | 469 |

### Example 2a

### 1-(4-{2-[1-(2,2-Difluoro-ethyl)-1H-pyrazol-4-ylamino]-[1,2,4]triazolo[1,5-a]pyridin-8 yl}-3,6-dihydro-2H-pyridin-1 yl)-4,4,4-trifluoro-butan-1-one (Compound No. 2-1)

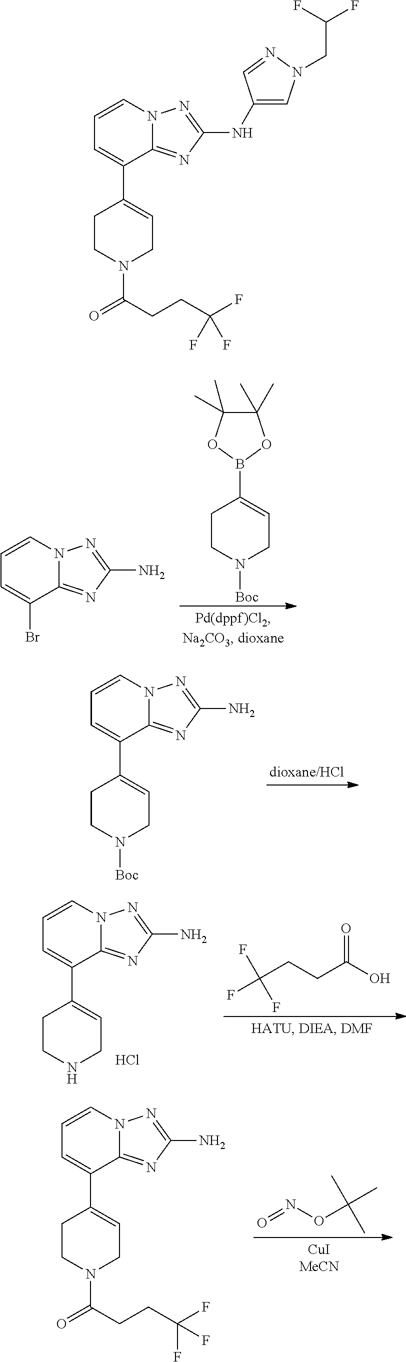

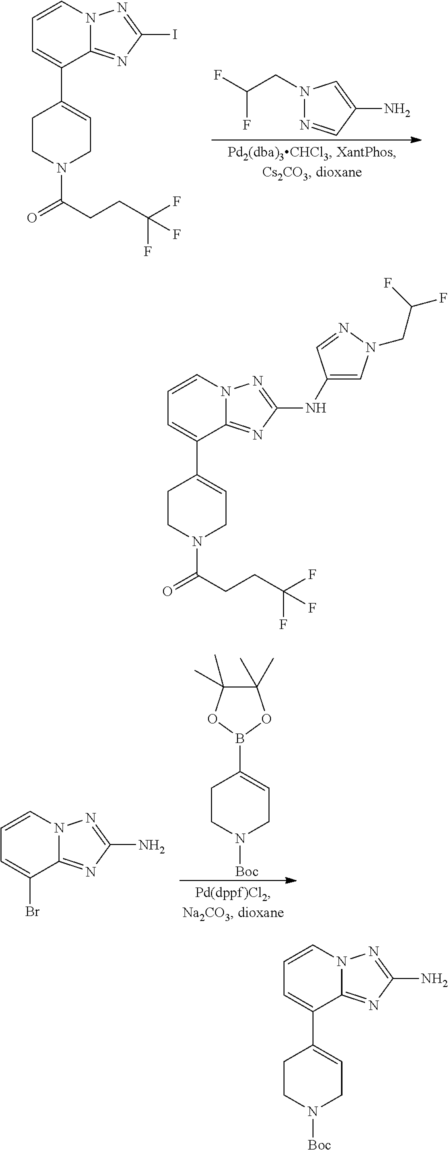

### Step 1:

A degassed mixture of 8-bromo-[1,2,4]triazolo[1,5-a]pyridin-2-amine (50 g, 234.70 mmol), tert-butyl 4-(4,4,5,5-tetramethyl-1,3-dioxolan-2-yl)-1,2,3,6-tetrahydropyridine-1-carboxylate (110 g, 353.2 mmol), Pd(dppf)Cl₂.CH₂Cl₂ (19.6 g, 24.00 mmol) and Cs₂CO₃ (156 g, 478.79 mmol) in dioxane (800 mL) and water (100 mL) was heated at 90° C. for 12 h. The reaction mixture was allowed to cool to ambient temperature and the solid removed by filtration. The filtrate was concentrated under vacuum, and the resultant residue was treated with water (1 L) and DCM (600 mL) and the phases were separated. The aqueous phase was extracted with DCM (2×600 mL). The combined organic layer was washed with brine (1.5 L), dried over anhydrous sodium sulfate and concentrated under vacuum. The residue was purified by flash chromatography on silica gel eluting with EtOAc/petroleum ether (1/1). Appropriate fractions were combined and evaporated to afford tert-butyl 4-[2-amino-[1,2,4]triazolo[1,5-a]pyridin-8-yl]-1,2,3,6-tetrahydropyridine-1-carboxylate (62.4 g, 84%) as a yellow solid. LCMS (Method 4): R_{T}=0.98 min, m/z=316.0 [M+H]⁺.

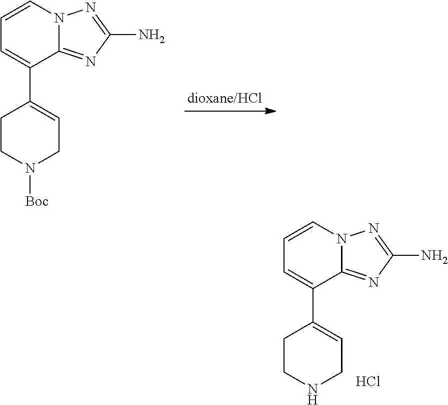 [SMILES: CC(C)(C)OC(=O)N1CC=C(c2cccn3nc(N)nc23)CC1 ; Nc1nc2c(C3=CCNCC3)cccn2n1 ; Cl]

### Step 2:

A mixture of tert-butyl 4-[2-amino-[1,2,4]triazolo[1,5-a]pyridin-8-yl]-1,2,3,6-tetrahydropyridine-1-carboxylate (13 g, 41.22 mmol) in a saturated solution of HCl in 1,4-dioxane (150 mL) was stirred at ambient temperature overnight. The precipitated solid was collected by filtration to afford the hydrochloride salt of 8-(1,2,3,6-tetrahydropyridin-4-yl)-[1,2,4]triazolo[1,5-a]pyridin-2-amine (10 g, crude) as a yellow solid. LCMS (Method 4): R_{T}=0.49 min, m/z=216.0 [M+H]⁺.

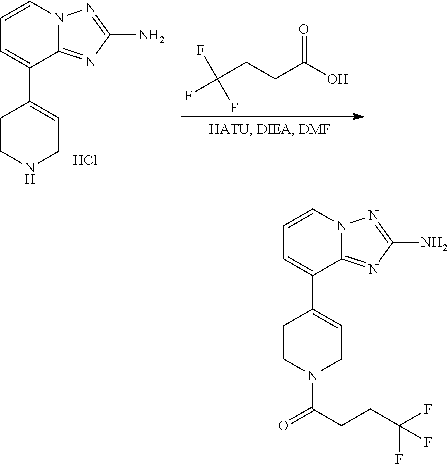 [SMILES: Nc1nc2c(C3=CCNCC3)cccn2n1 ; Cl ; O=C(O)CCC(F)(F)F ; Nc1nc2c(C3=CCN(C(=O)CCC(F)(F)F)CC3)cccn2n1]

### Step 3:

A mixture of 8-(1,2,3,6-tetrahydropyridin-4-yl)-[1,2,4]triazolo[1,5-a]pyridin-2-amine hydrochloride (10 g, 39.73 mmol), DIPEA (14 g, 108.32 mmol), 4,4,4-trifluorobutanoic acid (6 g, 42.23 mmol) and HATU (16 g, 42.08 mmol) in N,N-dimethylformamide (100 mL) was stirred at ambient temperature overnight. The reaction mixture was evaporated and the residue treated with water (250 mL) and EtOAc (200 mL). The phases were separated and the aqueous phase was extracted with EtOAc (200 mL). The combined organic phase was washed with brine, dried over sodium sulfate and concentrated under vacuum. The residue was purified by flash chromatography on silica gel eluting with 65% EtOAc/petroleum ether. Appropriate fractions were combined and evaporated to afford 1-(4-[2-amino-[1,2,4]triazolo[1,5-a]pyridin-8-yl]-1,2,3,6-tetrahydropyridin-1-yl)-4,4,4-trifluorobutan-1-one (10 g, 74%) as a yellow solid. LCMS (Method 4): R_{T}=1.18 min, m/z=340.0 [M+H]⁺.

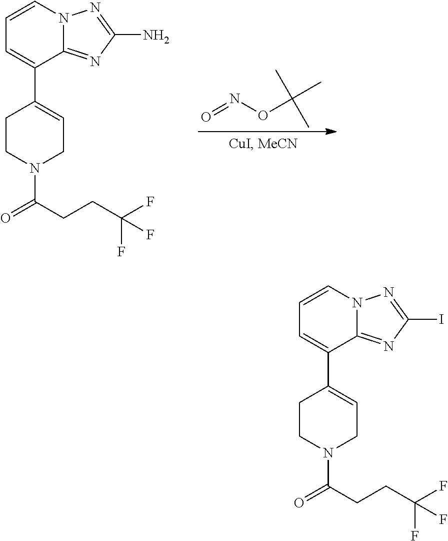

### Step 4:

tert-butyl nitrite (15.20 g, 147.4 mmol) was added to a solution of 1-(4-[2-amino-[1,2,4]triazolo[1,5-a]pyridin-8-yl]-1,2,3,6-tetrahydropyridin-1-yl)-4,4,4-trifluorobutan-1-one (10.0 g, 29.5 mmol) and CuI (11.23 g, 59.0 mmol) in MeCN (150 mL) under nitrogen. The mixture was stirred at ambient temperature for 20 min then heated at 55° C. for 30 min. The reaction was allowed to cool to ambient temperature and the precipitated solid removed by filtration. The filtrate was concentrated under vacuum and the residue was dissolved in water (500 mL). The pH of the aqueous phase was adjusted to 7 by the addition of 2M aqueous sodium hydroxide solution then extracted with DCM (3×200 mL). The combined organic layer was washed with brine (500 mL), dried over anhydrous sodium sulfate and concentrated. The residue was purified by column chromatography eluting with DCM/EtOAc (3/1). Appropriate fractions were combined and evaporated to afford 4,4,4-trifluoro-1-(4-[2-iodo-[1,2,4]triazolo[1,5-a]pyridin-8-yl]-1,2,3,6-tetrahydropyridin-1-yl)butan-1-one (5.3 g, 40%) as a light yellow solid. LCMS (Method 4): R_{T}=1.48 min, m/z=450.9 [M+H]⁺.

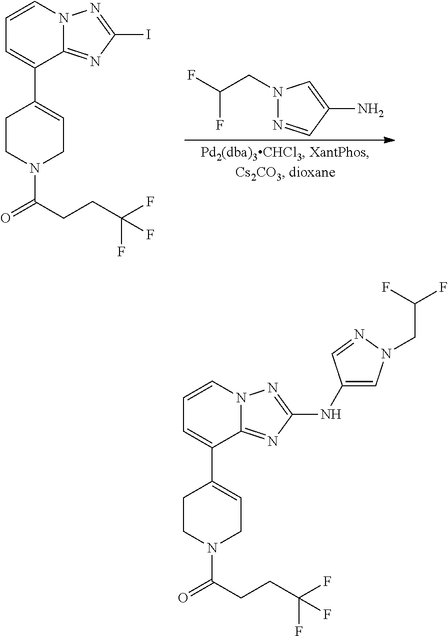 [SMILES: O=C(CCC(F)(F)F)N1CC=C(c2cccn3nc(I)nc23)CC1 ; Nc1cnn(CC(F)F)c1 ; O=C(CCC(F)(F)F)N1CC=C(c2cccn3nc(Nc4cnn(CC(F)F)c4)nc23)CC1]

### Step 5:

A degassed mixture of 4,4,4-trifluoro-1-(4-[2-iodo-[1,2,4]triazolo[1,5-a]pyridin-8-yl]-1,2,3,6-tetrahydropyridin-1-yl)butan-1-one (200 mg, 0.44 mmol), 1-(2,2-difluoroethyl)-1H-pyrazol-4-amine (163 mg, 1.11 mmol), Pd₂(dba)₃.CHCl₃ (46 mg, 0.04 mmol), XantPhos (51.5 mg, 0.09 mmol) and Cs₂CO₃ (290 mg, 0.89 mmol) in dioxane (10 mL) was heated at 100° C. overnight. The mixture was allowed to cool to ambient temperature and the solid removed by filtration. The filtrate was concentrated under vacuum and the residue was purified by flash chromatography on silica gel eluting with EtOAc/petroleum ether (4:1). Appropriate fractions were combined and evaporated and the crude product was purified by Prep-HPLC using the following conditions: Column, XBridge Prep C18 OBD Column, 5 um, 19*150 mm, mobile phase, water with 10 mmol NH₄HCO₃ and MeCN (MeCN (60.0% MeCN up to 73.0% over 10 min, up to 95.0% over 1 min, hold at 95.0% for 1 min, down to 60.0% over 2 min); Detector, UV 254/220 nm. Appropriate fractions were combined and evaporated to afford 1-[4-(2-[[1-(2,2-difluoroethyl)-1H-pyrazol-4-yl]amino]-[1,2,4]triazolo[1,5-a]pyridin-8-yl)-1,2,3,6-tetrahydropyridin-1-yl]-4,4,4-trifluorobutan-1-one (83.2 mg, 40%) as a purple solid. LCMS (Method 4): R_{T}=1.44 min, m/z=470.2 [M+H]⁺. ¹H NMR (400 MHz, DMSO-d6): δ 9.38 (s, 1H), 8.60 (d, 1H, J=6.8 Hz), 7.89 (d, 1H, J=4.4 Hz), 7.57-7.48 (m, 3H), 7.00-6.95 (m, 1H), 6.50-6.10 (m, 1H), 4.65-4.52 (m, 2H), 4.30-4.15 (m, 2H), 3.79-3.70 (m, 2H), 2.85-2.55 (m, 6H).

The immediately preceding Example may be modified via conventionally known chemistries to provide access to other compounds that fall within the scope of the present invention, such as compounds of Formula I, non-limiting examples of which are seen in Table 3.

TABLE 3

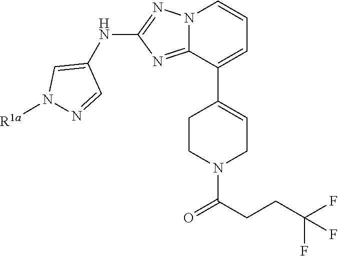

| Compound | | | LCMS(ESI) |
|---|---|---|---|
| No. | R^{1a} | Name | m/z [M + H]⁺ |
| 2-1 | 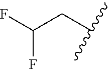 [SMILES: *CC(F)F] | 1-[4-[2-[[1-(2,2-difluoroethyl)pyrazol-4- yl]amino]-[1,2,4]triazolo[1,5-a]pyridin-8-yl]- 3,6-dihydro-2H-pyridin-1-yl]-4,4,4-trifluoro- butan-1-one | 470 |
| 2-2 | 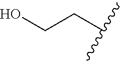 [SMILES: *CCO] | 4,4,4-trifluoro-1-[4-[2-[[1-(2- hydroxyethyl)pyrazol-4-yl]amino]- [1,2,4]triazolo[1,5-a]pyridin-8-yl]-3,6- dihydro-2H-pyridin-1-yl]butan-1-one | 450 |

### Example 3a

### (8-{4-[4-(2-Methoxy-ethoxy)-benzyloxy]-phenyl}-[1,2,4]triazolo[1,5-a]pyridin-2-yl)-(1-methyl-1H-pyrazol-4-yl)-amine (Compound No. 3-1)

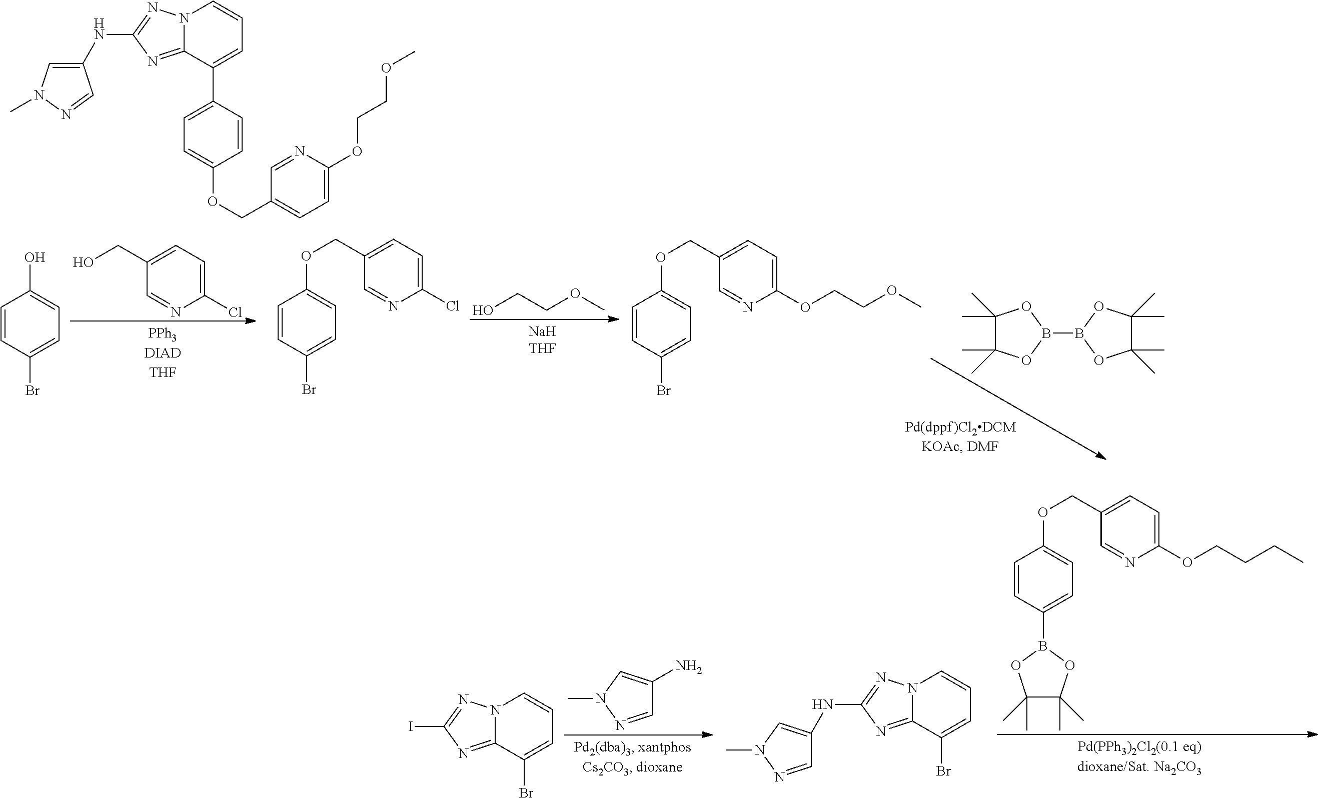

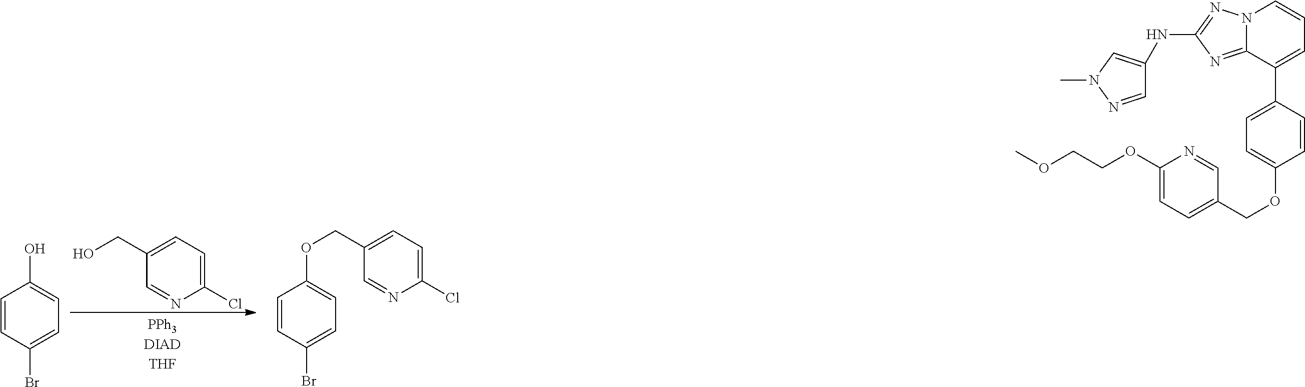

### Step 1

DIAD (4.1 mL, 20.8 mmol) was added dropwise to a cooled (0° C.) solution of 4-bromophenol (3.0 g, 17.3 mmol), 2-chloro-5-hydroxymethylpyridine (3.0 g, 20.8 mmol) and triphenylphosphine (5.5 g, 20.8 mmol) in THF (100 mL). The reaction mixture was allowed to warm to ambient temperature and left to stir for 4 h. The mixture was diluted with EtOAc and washed with water, aqueous saturated sodium hydrogen carbonate solution, water and brine, dried over anhydrous sodium sulfate, filtered and evaporated. The residual solid was triturated with DCM and collected by filtration to afford 5-(4-bromo-phenoxymethyl)-2-chloro-pyridine (3.3 g, 64%) as a white solid. ¹H NMR (400 MHz, DMSO-d₆): δ 8.51 (d, 1H, J=1.8 Hz), 7.94 (dd, 1H, J=2.4, 8.2 Hz), 7.58-7.54 (m, 1H), 7.49-7.46 (m, 2H), 7.03-7.00 (m, 2H), 5.16 (s, 2H).

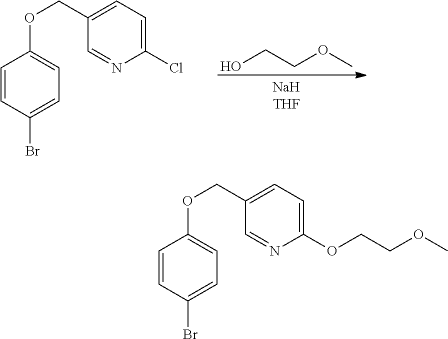

### Step 2:

Sodium hydride (60% dispersion in oil, 1.0 g, 25.1 mmol) was added portionwise over 10 minutes to a cooled (0° C.) solution of 2-methoxyethanol (3.31 mL, 41.9 mmol) in THF (20 mL). The mixture was allowed to stir for 15 min before a solution of 5-(4-bromo-phenoxymethyl)-2-chloro-pyridine (500 mg, 1.67 mmol) in THF (10 mL) was added. On complete addition, the mixture allowed to warm to ambient temperature then heated at 70° C. for 18 h. The reaction mixture was allowed to cool to ambient temperature, diluted with EtOAc and washed with water (×2) and brine, dried over anhydrous sodium sulphate, filtered and evaporated to afford 5-(4-bromo-phenoxymethyl)-2-(2-methoxy-ethoxy)-pyridine (565 mg, quant.) as a white solid. ¹H NMR (400 MHz, CDCl₃): δ 8.16 (d, 1H, J=2.0 Hz), 7.64 (dd, 1H, J=2.4, 8.5 Hz), 7.40-7.37 (m, 2H), 6.85-6.82 (m, 3H), 4.94 (s, 2H), 4.50-4.47 (m, 2H), 3.77-3.73 (m, 2H), 3.44 (s, 3H).

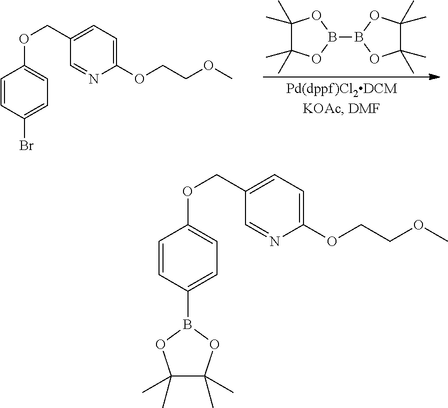

### Step 3:

A degassed solution of 5-(4-bromo-phenoxymethyl)-2-(2-methoxy-ethoxy)-pyridine (2.44 g, 7.21 mmol), bis-pinacolato diboron (2.02 g, 7.94 mmol), KOAc (1.06 g, 10.8 mmol) and PdCl₂(dppf).DCM (294 mg, 0.36 mmol) in DMF (50 mL) was heated at 90° C. for 2.5 h. The reaction mixture was allowed to cool to ambient temperature and partitioned between EtOAc and water. The organic layer was separated, washed with water and brine, dried over anhydrous sodium sulfate, filtered and evaporated. The residue was purified by flash chromatography on silica gel eluting with DCM on a gradient of MeOH (0-10%). Appropriate fractions were combined and evaporated to afford 2-(2-methoxy-ethoxy)-5-[4-(4,4,5,5-tetramethyl-[1,3,2]-dioxaborolan-2-yl)-phenoxymethyl]-pyridine (2.78 g, quant.) as a red solid. ¹H NMR (400 MHz, CDCl₃): δ 8.19-8.16 (m, 1H), 7.76 (d, 2H, J=8.3 Hz), 7.66 (dd, 1H, J=2.3, 8.3 Hz), 6.95 (d, 2H, J=8.3 Hz), 6.85-6.81 (m, 1H), 4.99 (s, 2H), 4.50-4.47 (m, 2H), 3.77-3.73 (m, 2H), 3.44 (s, 3H), 1.33 (s, 12H).

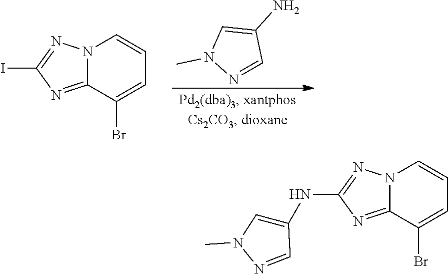

### Step 4:

A degassed mixture of 1-methyl-1H-pyrazol-4-ylamine dihydrochloride (62 mg, 0.46 mmol), 8-bromo-2-iodo-[1,2,4]triazolo[1,5-a]pyridine (100 mg, 0.31 mmol), Pd₂(dba)₃ (28 mg, 0.03 mmol), XantPhos (18 mg, 0.03 mmol) and Cs₂CO₃ (402 mg, 1.24 mmol) in 1,4-dioxane (5 mL) was heated under reflux for 18 h then allowed to cool to ambient temperature. The solvent was evaporated and the residue partitioned between DCM and water. The organic layer was separated, dried over anhydrous sodium sulfate, the solid was removed by filtration and the filtrate evaporated. The resultant residue was purified by flash chromatography on silica gel eluting with DCM on a gradient of MeOH (0-5%). Appropriate fractions were combined and evaporated to afford (8-bromo-[1,2,4]triazolo[1,5-a]pyridin-2-yl)-(1-methyl-1H-pyrazol-4-yl)-amine (50 mg, 66%) as an off white solid. ¹H NMR (400 MHz, CDCl₃): δ 8.36 (d, 1H, J=6.6 Hz), 7.78 (s, 1H), 7.63 (d, 1H, J=7.6 Hz), 7.49 (s, 1H), 6.98 (s, 1H), 6.76-6.70 (m, 1H), 3.91 (s, 3H).

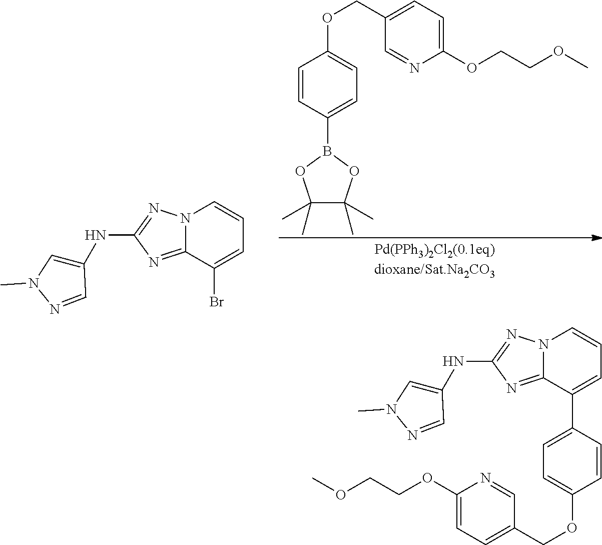

### Step 5:

A degassed solution of (8-bromo-[1,2,4]triazolo[1,5-a]pyridin-2-yl)-(1-methyl-1H-pyrazol-4-yl)-amine (50 mg, 0.17 mmol), 2-(2-methoxy-ethoxy)-5-[4-(4,4,5,5-tetramethyl-[1,3,2]dioxaborolan-2-yl)-phenoxymethyl]-pyridine (99 mg, 0.26 mmol), K₂CO₃ (35 mg, 0.26 mmol) and PdCl₂(dppf).DCM (14 mg, 0.02 mmol) in 1,4-dioxane (2 mL) and water (0.5 mL) was heated under reflux for 0.5 h. The reaction mixture was allowed to cool to ambient temperature and partitioned between EtOAc and water. The organic layer was separated, dried over anhydrous sodium sulfate, filtered and evaporated. The residue was purified by flash chromatography on silica gel eluting with EtOAc on a gradient of MeOH (0-5%). Appropriate fractions were combined and evaporated. The resultant residue was purified by prep-HPLC (MDAP, Method 1) to afford (8-{4-[4-(2-methoxy-ethoxy)-benzyloxy]-phenyl}-[1,2,4]triazolo[1,5-a]pyridin-2-yl)-(1-methyl-1H-pyrazol-4-yl)-amine (30 mg, 37%), Example 3-1, as a white solid. LCMS (Method 2): RT=4.25 min, m/z=472.0 [M+H]⁺. ¹H NMR (400 MHz, DMSO-d₆): δ 9.30 (s, 1H), 8.63 (dd, 1H, J=1.0, 6.6 Hz), 8.29 (d, 1H, J=2.1 Hz), 8.15-8.12 (m, 2H), 7.83 (dd, 1H, J=2.4, 8.6 Hz), 7.77 (s, 1H), 7.75 (dd, 1H, J=1.2, 7.5 Hz), 7.44 (s, 1H), 7.17 (d, 2H, J=8.9 Hz), 7.03 (dd, 1H, J=7.0, 7.0 Hz), 6.88 (d, 1H, J=8.6 Hz), 5.14 (s, 2H), 4.40-4.37 (m, 2H), 3.81 (s, 3H), 3.68-3.64 (m, 2H), 3.30 (s, 3H).

TABLE 4

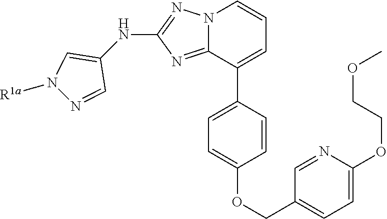

| Compound | | | LCMS(ESI) |
|---|---|---|---|
| No. | R^{1a} | Name | m/z [M + H]⁺ |
| 3-1 | 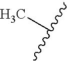 | 8-[4-[[6-(2-methoxyethoxy)-3- pyridyl]methoxy]phenyl]-N-(1- methylpyrazol-4-yl)-[1,2,4]triazolo[1,5- a]pyridin-2-amine | 473 |
| 3-2 | 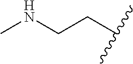 [SMILES: *CCNC] | 8-[4-[[6-(2-methoxyethoxy)-3- pyridyl]methoxy]phenyl]-N-[1-[2- (methylamino)ethyl]pyrazol-4-yl]- [1,2,4]triazolo[1,5-a]pyridin-2-amine | 516 |
| 3-3 | 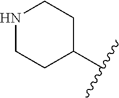 [SMILES: *C1CCNCC1] | 8-[4-[[6-(2-methoxyethoxy)-3- pyridyl]methoxy]phenyl]-N-[1-(4- piperidyl)pyrazol-4-yl]-[1,2,4]triazolo[1,5- a]pyridin-2-amine | 542 |
| 3-4 | 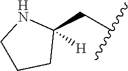 [SMILES: *C[C@H]1CCCN1] | 8-[4-[[6-(2-methoxyethoxy)-3- pyridyl]methoxy]phenyl]-N-[1-[[(2R)- pyrrolidin-2-yl]methyl]pyrazol-4-yl]- [1,2,4]triazolo[1,5-a]pyridin-2-amine | 541 |
| 3-5 | 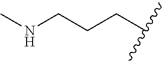 [SMILES: *CCCNC] | 8-[4-[[6-(2-methoxyethoxy)-3- pyridyl]methoxy]phenyl]-N-[1-[3- (methylamino)propyl]pyrazol-4-yl]- [1,2,4]triazolo[1,5-a]pyridin-2-amine | 529 |

### Example 5

### 1-[4-(4-chlorophenyl)-1-[2-[(1-methyl-1H-pyrazol-4-yl)amino]-[1,2,4]triazolo[1,5-a]pyridin-8-yl]piperidin-4-yl]-2-(morpholin-4-yl)ethan-1-ol (Compound B)

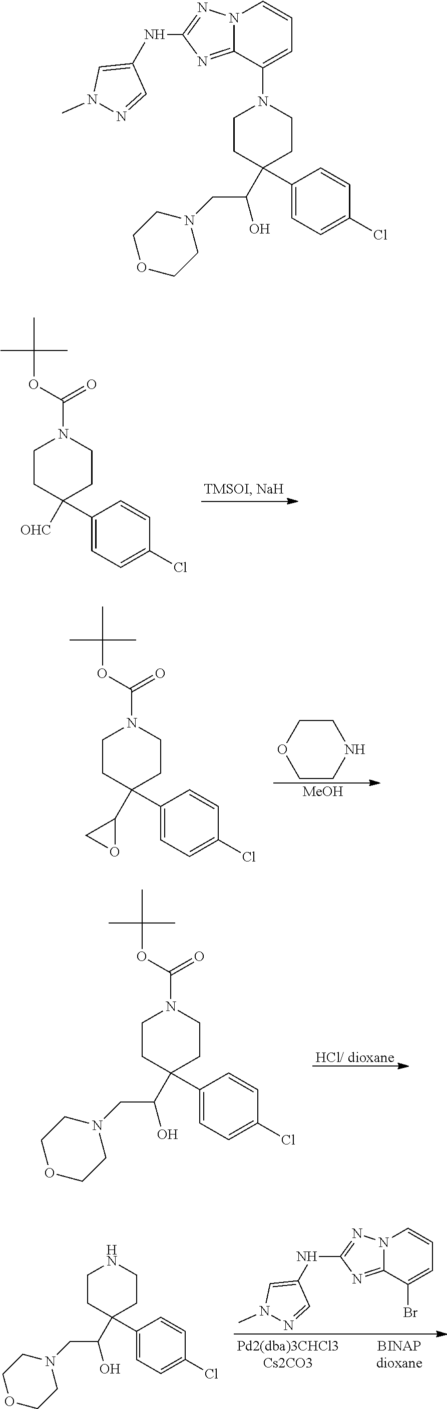

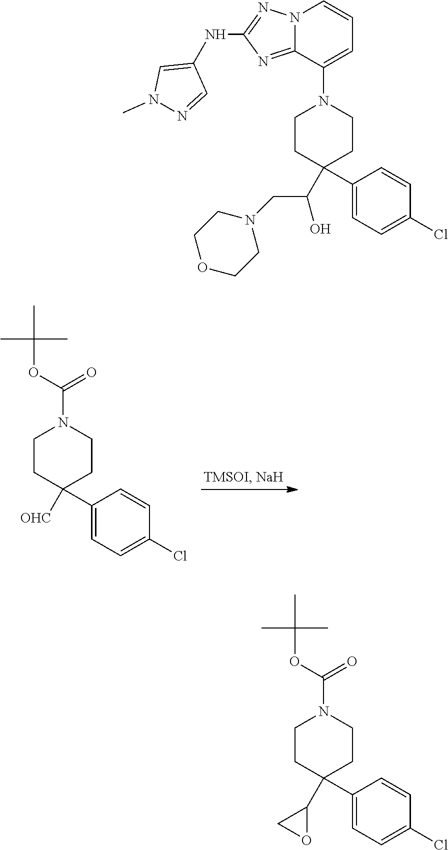 [SMILES: Cn1cc(Nc2nc3c(N4CCC(c5ccc(Cl)cc5)(C(O)CN5CCOCC5)CC4)cccn3n2)cn1 ; CC(C)(C)OC(=O)N1CCC(C=O)(c2ccc(Cl)cc2)CC1 ; CC(C)(C)OC(=O)N1CCC(c2ccc(Cl)cc2)(C2CO2)CC1]

### Step 1:

To a solution of S,S-dimethylmethanesulfinyl iodide (4.00 g, 18.2 mmol) in DMSO (50 mL) was added sodium hydride (1.24 g, 60% dispersion in mineral oil, 30.8 mmol) in small portions under nitrogen. The resulting solution was stirred for 2 h at room temperature under nitrogen. tert-butyl 4-(4-chlorophenyl)-4-formylpiperidine-1-carboxylate (3.00 g, 9.27 mmol) was added portionwise at 0° C. The reaction mixture was stirred at room temperature overnight, and poured into ice water (100 mL). The resulting solution was extracted with 3×100 mL of ethyl acetate and the organic layers combined. The organic extracts were washed with water, brine and dried over anhydrous sodium sulfate. The solids were filtered off and washed with ethyl acetate. The filtrate was concentrated under vacuum. This resulted in 2.51 g of tert-butyl 4-(4-chlorophenyl)-4-(oxiran-2-yl)piperidine-1-carboxylate as colorless oil. TLC: R_{f}=0.5; ethyl acetate/hexane=1/4.

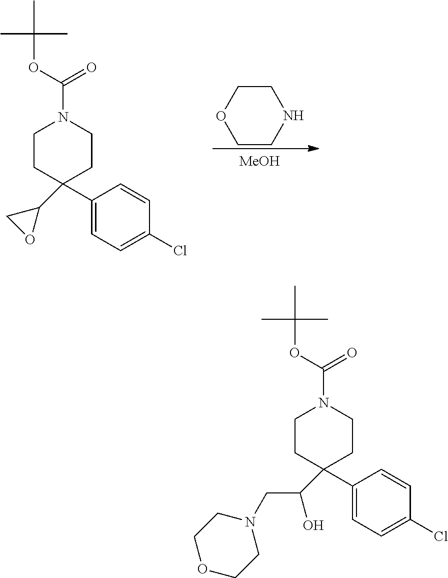 [SMILES: "MeOH" ; CC(C)(C)OC(=O)N1CCC(c2ccc(Cl)cc2)(C2CO2)CC1 ; C1COCCN1 ; CC(C)(C)OC(=O)N1CCC(c2ccc(Cl)cc2)(C(O)CN2CCOCC2)CC1]

### Step 2:

A mixture of tert-butyl 4-(4-chlorophenyl)-4-(oxiran-2-yl)piperidine-1-carboxylate (200 mg, 0.592 mmol) and morpholine (0.50 mL, 5.74 mmol) in MeOH (4 mL) was stirred for 20 h at 60° C. The reaction mixture was allowed to cool to room temperature, and concentrated under vacuum. The residue was purified by flash chromatography on silica gel eluting with dichloromethane/methanol (10/1). The appropriate fractions were combined and concentrated under vacuum. This resulted in 120 mg of tert-butyl 4-(4-chlorophenyl)-4-[1-hydroxy-2-(morpholin-4-yl)ethyl]piperidine-1-carboxylate as light yellow oil. LC/MS (Method 5, ESI): [M+H]⁺=425.2, R_{T}=0.92 min.

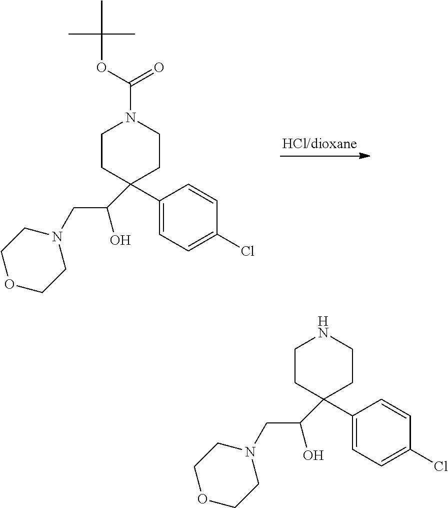 [SMILES: CC(C)(C)OC(=O)N1CCC(c2ccc(Cl)cc2)(C(O)CN2CCOCC2)CC1 ; OC(CN1CCOCC1)C1(c2ccc(Cl)cc2)CCNCC1]

### Step 3:

To a solution of HCl in dioxane (4M, 10 mL) was added tert-butyl 4-(4-chlorophenyl)-4-[1-hydroxy-2-(morpholin-4-yl)ethyl]piperidine-1-carboxylate (130 mg, 0.306 mmol). The resulting solution was stirred for 20 h at room temperature and concentrated under vacuum. Water (1 mL) was added to the residue. K₂CO₃ (100 mg) was then added. The resulting mixture was concentrated under vacuum. The organic residue was taken up in DCM/MeOH (10 mL, 10/1). The solids were filtered out. The filtrate was concentrated under vacuum. This resulted in 80 mg of 1-[4-(4-chlorophenyl)piperidin-4-yl]-2-(morpholin-4-yl)ethan-1-ol as light yellow oil. LC/MS (Method 5, ESI): [M+H]⁺=325.2, R_{T}=0.59 min.

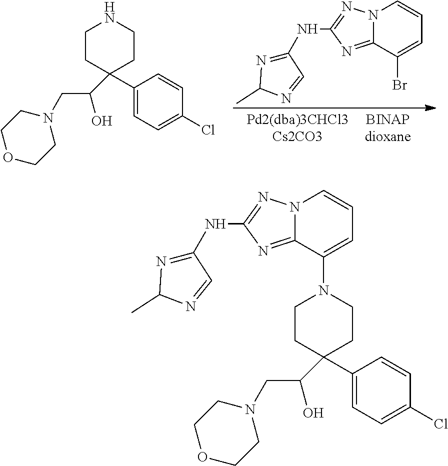

### Step 4.

To a solution of N-[8-bromo-[1,2,4]triazolo[1,5-a]pyridin-2-yl]-1-methyl-1H-pyrazol-4-amine (100 mg, 0.341 mmol) in dioxane (6.0 mL) was added 1-[4-(4-chlorophenyl)piperidin-4-yl]-2-(morpholin-4-yl)ethan-1-ol (333 mg, 1.03 mmol), Pd₂(dba)₃.CHCl₃ (70.9 mg, 0.0685 mmol), BINAP (85.2 mg, 0.137 mmol) and Cs₂CO₃ (223 mg, 0.685 mmol) under nitrogen. The reaction mixture was stirred at 100° C. overnight, and concentrated under vacuum. The residue was filtered through a short pad of silica gel eluting with DCM/MeOH (80/20). Appropriate fractions were combined and concentrated under reduced pressure. The residue (55 mg) was further purified by Prep-HPLC with the following conditions: Column, Gemini-NX C18 AXAI Packed, 21.2*150 mm, 5 um; mobile phase, 10 mM NH₄HCO₃ in water and ACN (13.0% ACN up to 42.0% in 9 min); Detector, UV 254 nm. This resulted in 7.4 mg of 1-[4-(4-chlorophenyl)-1-[2-[(1-methyl-1H-pyrazol-4-yl)amino]-[1,2,4]triazolo[1,5-a]pyridin-8-yl]piperidin-4-yl]-2-(morpholin-4-yl)ethan-1-ol as a light yellow solid. LC/MS (Method 7, ESI): [M+H]⁺=537.2, R_{T}=2.52 min. ¹H NMR (300 MHz, CD₃OD-d₄): δ (ppm) 8.07 (dd, J=6.5, 1.1 Hz, 1H), 7.84 (s, 1H), 7.56 (s, 1H), 7.48 (d, J=8.7 Hz, 2H), 7.38 (d, J=8.7 Hz, 2H), 6.83-6.73 (m, 2H), 4.00-3.96 (m, 2H), 3.88 (s, 3H), 3.80-3.77 (m, 1H), 3.66-3.54 (m, 4H), 2.87-2.74 (m, 2H), 2.56-2.20 (m, 9H), 2.10-1.90 (m, 1H).

### Example 6

### 2-[3-(4-ethyl-1H-pyrazol-1-yl)-1-(2-[[1-([1-[1-(oxetan-3-yl)piperidin-4-yl]-1H-1,2,3-triazol-4-yl]methyl)-1H-pyrazol-4-yl]amino]-[1,2,4]triazolo[1,5-a]pyridin-8-yl)azetidin-3-yl]acetonitrile (Compound S)

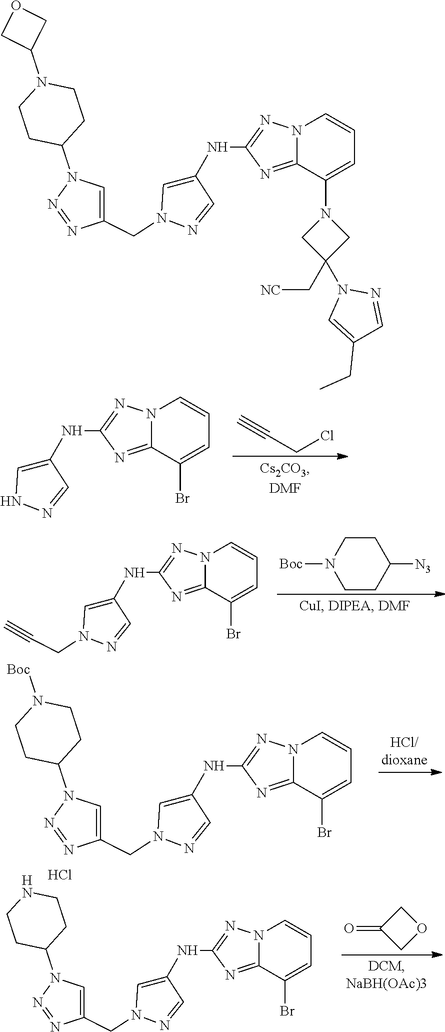

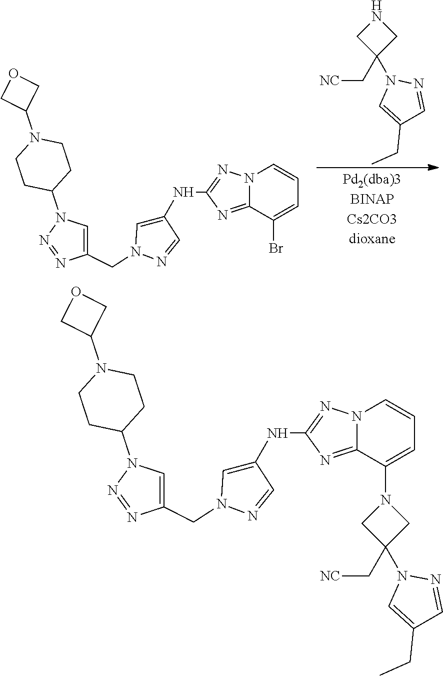

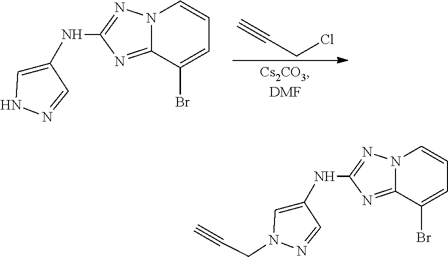

### Step 1:

To a solution of N-[8-bromo-[1,2,4]triazolo[1,5-a]pyridin-2-yl]-1H-pyrazol-4-amine hydrochloric salt (800 mg, 2.54 mmol) in N,N-dimethylformamide (16 mL) was added DIPEA (371 mg, 2.87 mmol) at room temperature. The mixture was stirred at room temperature until all the solids disappeared. Cs₂CO₃ (938 mg, 2.88 mmol) was then added. The reaction mixture was stirred to 60° C. under nitrogen and then 3-chloroprop-1-yne (468 mg, 6.28 mmol) was added dropwise. The resulting mixture was stirred at 60° C. overnight and concentrated under vacuum. The residue was purified on silica gel eluting with ethyl acetate/petroleum ether (1/1) to give 330 mg (36%) of N-[8-bromo-[1,2,4]triazolo[1,5-a]pyridin-2-yl]-1-(prop-2-yn-1-yl)-1H-pyrazol-4-amine as a light yellow solid. LC/MS (Method 6, ESI): [M+H]⁺=317.0, R_{T}=0.72 min.

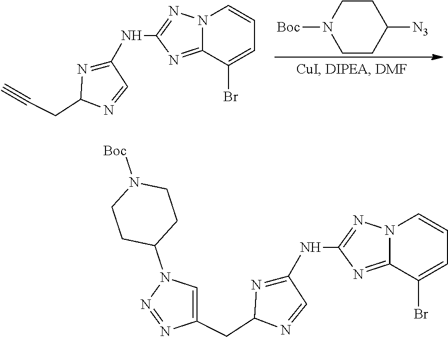

### Step 2.

To a solution of N-[8-bromo-[1,2,4]triazolo[1,5-a]pyridin-2-yl]-1-(prop-2-yn-1-yl)-1H-pyrazol-4-amine (300 mg, 0.946 mmol) in N,N-dimethylformamide (10 mL) was added DIPEA (245 mg, 1.90 mmol), CuI (36.0 mg, 0.189 mmol) and tert-butyl 4-azidopiperidine-1-carboxylate (373 mg, 1.65 mmol) under nitrogen. The resulting solution was stirred at room temperature overnight and concentrated under vacuum. The residue was purified on silica gel eluting with ethyl acetate to obtain 170 mg (33%) of tert-butyl 4-(4-[[4-([8-bromo-[1,2,4]triazolo[1,5-a]pyridin-2-yl]amino)-1H-pyrazol-1-yl]methyl]-1H-1,2,3-triazol-1-yl)piperidine-1-carboxylate as a off-white solid. LC/MS (Method 6, ESI): [M+H]⁺=543.2, R_{T}=1.43 min.

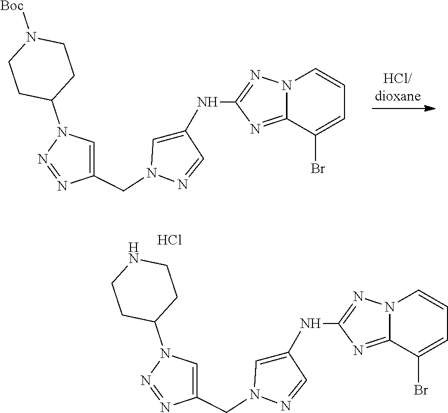 [SMILES: CC(C)(C)OC(=O)N1CCC(n2cc(Cn3cc(Nc4nc5c(Br)cccn5n4)cn3)nn2)CC1 ; Cl ; Brc1cccn2nc(Nc3cnn(Cc4cn(C5CCNCC5)nn4)c3)nc12]

### Step 3:

To a solution of HCl in dioxane (4M, 10 mL) was added tert-butyl 4-(4-[[4-([8-bromo-[1,2,4]triazolo[1,5-a]pyridin-2-yl]amino)-1H-pyrazol-1-yl]methyl]-1H-1,2,3-triazol-1-yl)piperidine-1-carboxylate (120 mg, 0.221 mmol). The resulting solution was stirred at room temperature overnight and concentrated under vacuum. This resulted in 138 mg (crude) of N-[8-bromo-[1,2,4]triazolo[1,5-a]pyridin-2-yl]-1-[[1-(piperidin-4-yl)-1H-1,2,3-triazol-4-yl]methyl]-1H-pyrazol-4-amine hydrochloride as a white solid. LC/MS (Method 6, ESI): [M+H]⁺=443.1, R_{T}=0.64 min.

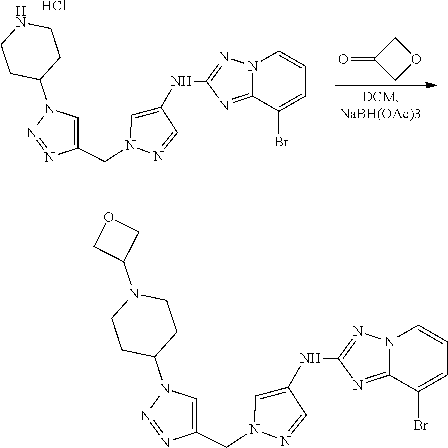

### Step 4:

To a solution of crude N-[8-bromo-[1,2,4]triazolo[1,5-a]pyridin-2-yl]-1-[[1-(piperidin-4-yl)-1H-1,2,3-triazol-4-yl]methyl]-1H-pyrazol-4-amine hydrochloride (138 mg from last step) in dichloromethane (15 mL) was added oxetan-3-one (362 mg, 5.02 mmol). The resulting solution was stirred at room temperature overnight. Then NaBH(OAc)₃ (1.42 g, 6.70 mmol) was added. The resulting solution was stirred at room temperature overnight and concentrated under vacuum. The residue was filtered through a short pad of silica gel eluting with dichloromethane/methanol (80/20) to give crude N-[8-bromo-[1,2,4]triazolo[1,5-a]pyridin-2-yl]-1-([1-[1-(oxetan-3-yl)piperidin-4-yl]-1H-1,2,3-triazol-4-yl]methyl)-1H-pyrazol-4-amine (120 mg) as a yellow solid, which was used for next step without further purification. LC/MS (Method 5, ESI): [M+H]⁺=499.1, R_{T}=1.00 min.

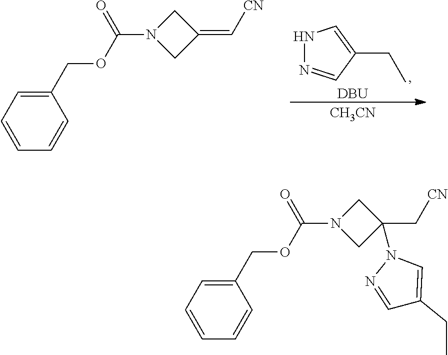

### Step 5:

To a solution of benzyl 3-(cyanomethylidene)azetidine-1-carboxylate (2.4 g, 10.515 mmol) in CH₃CN (20 mL) was added ethyl-1H-pyrazole (1.00 g, 10.40 mmol), DBU (1.08 g, 7.09 mmol). The resulting solution was stirred at 50° C. overnight and concentrated under vacuum. The residue was purified on silica gel eluting with ethyl acetate/petroleum ether (30/70). The appropriate fractions were combined and concentrated under vacuum. This resulted in 3.00 g (88%) of benzyl 3-(cyanomethyl)-3-(4-ethyl-1H-pyrazol-1-yl)azetidine-1-carboxylate as colorless oil. LC/MS (LCMS53, ESI): [M+H]⁺=325.2, R_{T}=1.47 min.

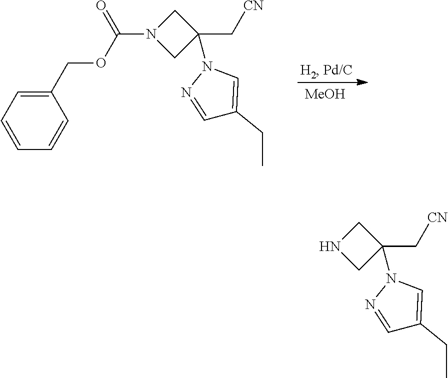 [SMILES: "MeOH" ; CCc1cnn(C2(CC#N)CN(C(=O)OCc3ccccc3)C2)c1 ; "H2, Pd/C" ; CCc1cnn(C2(CC#N)CNC2)c1]

### Step 6:

A mixture of benzyl 3-(cyanomethyl)-3-(4-ethyl-1H-pyrazol-1-yl)azetidine-1-carboxylate (3.00 g, 9.25 mmol), 10% Pd/C (100 mg) in methanol (15 mL) was stirred under an atmosphere of hydrogen balloon at room temperature overnight. The catalyst was filtered off and washed with MeOH. The filtrate was concentrated under vacuum. The residue was purified on silica gel eluting with dichloromethane/methanol (87/13). The appropriate fractions were combined and concentrated under vacuum. This resulted in 1.6 g (91%) of 2-[3-(4-ethyl-1H-pyrazol-1-yl)azetidin-3-yl]acetonitrile as colorless oil. MS (ESI): [M+H]⁺=191.2.

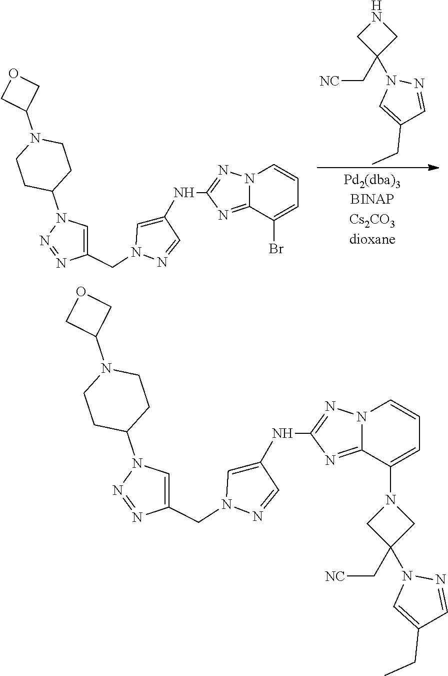

### Step 7:

To a solution of N-[8-bromo-[1,2,4]triazolo[1,5-a]pyridin-2-yl]-1-([1-[1-(oxetan-3-yl)piperidin-4-yl]-1H-1,2,3-triazol-4-yl]methyl)-1H-pyrazol-4-amine (60.0 mg, 0.120 mmol) in dioxane (6.0 mL) was added 2-[3-(4-ethyl-1H-pyrazol-1-yl)azetidin-3-yl]acetonitrile (46.0 mg, 0.242 mmol), Pd₂(dba)₃ (44.0 mg, 0.0481 mmol), BINAP (60.0 mg, 0.0963 mmol) and Cs₂CO₃ (118 mg, 0.362 mmol) under atmosphere of nitrogen. The reaction mixture was stirred at 100° C. overnight. The mixture was allowed to cool to room temperature and concentrated under vacuum. The residue was filtered through a short pad of silica gel eluting with dichloromethane/methanol (85/15). The appropriate fractions were collected and concentrated under reduced pressure. The residue was further purified by Prep-HPLC with the following conditions: Column, Gemini-NX C18 AXAI Packed, 21.2*150 mm, 5 um; mobile phase, 10 mM NH₄HCO₃ in water and ACN (15.0% ACN up to 45.0% in 7 min); Detector, UV 254 nm to obtain 21.2 mg of 2-[3-(4-ethyl-1H-pyrazol-1-yl)-1-(2-[[1-([1-[1-(oxetan-3-yl)piperidin-4-yl]-1H-1,2,3-triazol-4-yl]methyl)-1H-pyrazol-4-yl]amino]-[1,2,4]triazolo[1,5-a]pyridin-8-yl)azetidin-3-yl]acetonitrile as a white solid. LC/MS (Method 3, ESI): [M+H]⁺=609.3, R_{T}=1.46 min. ¹H NMR (300 MHz, CD₃OD-d₄): δ (ppm) 8.01-7.96 (m, 3H), 7.81 (d, J=0.6 Hz, 1H), 7.55 (d, J=0.6 Hz, 1H), 7.48 (s, 1H), 6.83-6.79 (m, 1H), 6.46 (d, J=7.2 Hz, 1H), 5.40 (s, 2H), 4.68 (t, J=6.6 Hz, 2H), 4.61-4.51 (m, 7H), 3.56-3.51 (m, 3H), 2.90-2.87 (m, 2H), 2.54 (q, J=7.5 Hz, 2H), 2.19-2.03 (m, 6H), 1.22 (t, J=7.5 Hz, 3H).

The preceding Examples may be modified via conventionally known chemistries to provide access to other compounds that fall within the scope of the present invention, such as compounds of Formula I, non-limiting examples of which are seen in Table 4a:

TABLE 4a

| | | | LCMS |
|---|---|---|---|
| | | | (ESI) |
| Compound | | | m/z |
| No. | Structure | Name | [M + H]⁺ |
| A | 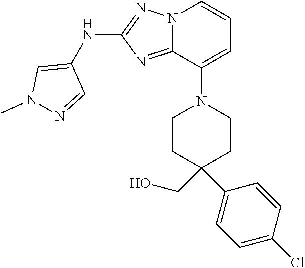 [SMILES: Cn1cc(Nc2nc3c(N4CCC(CO)(c5ccc(Cl)cc5)CC4)cccn3n2)cn1] | [4-(4-chlorophenyl)-1- [2-[(1-methylpyrazol-4- yl)amino]- [1,2,4]triazolo[1,5- a]pyridin-8-yl]-4- piperidyl]methanol | 438.2 |
| B | 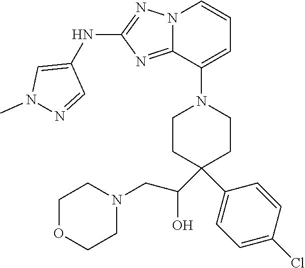 [SMILES: Cn1cc(Nc2nc3c(N4CCC(c5ccc(Cl)cc5)(C(O)CN5CCOCC5)CC4)cccn3n2)cn1] | 1-[4-(4-chlorophenyl)- 1-[2-[(1-methylpyrazol- 4-yl)amino]- [1,2,4]triazolo[1,5- a]pyridin-8-yl]-4- piperidyl]-2- morpholino-ethanol Or 1-[4-(4-chlorophenyl)- 1-[2-[(1-methyl-1H- pyrazol-4-yl)amino]- [1,2,4]triazolo[1,5- a]pyridin-8- yl]piperidin-4-yl]-2- (morpholin-4-yl)ethan- 1-ol | 537.3 |
| C | 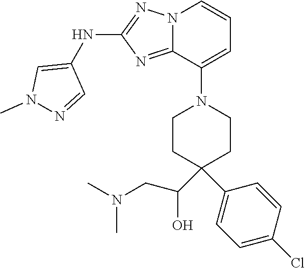 [SMILES: CN(C)CC(O)C1(c2ccc(Cl)cc2)CCN(c2cccn3nc(Nc4cnn(C)c4)nc23)CC1] | 1-[4-(4-chlorophenyl)- 1-[2-[(1-methylpyrazol- 4-yl)amino]- [1,2,4]triazolo[1,5- a]pyridin-8-yl]-4- piperidyl]-2- (dimethylamino)ethanol | 495.3 |
| D | 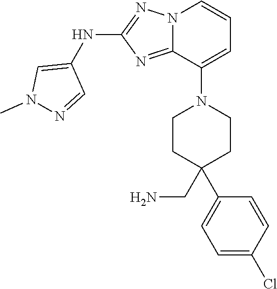 [SMILES: Cn1cc(Nc2nc3c(N4CCC(CN)(c5ccc(Cl)cc5)CC4)cccn3n2)cn1] | 8-[4-(aminomethyl)-4- (4-chlorophenyl)-1- piperidyl]-N-(1- methylpyrazol-4-yl)- [1,2,4]triazolo[1,5- a]pyridin-2-amine | 437.3 |
| E | 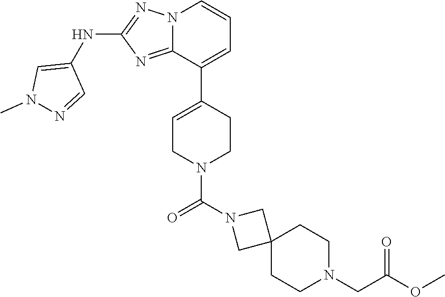 | methyl 2-[2-[4-[2-[(1- methylpyrazol-4- yl)amino]- [1,2,4]triazolo[1,5- a]pyridin-8-yl]-3,6- dihydro-2H-pyridine-1- carbonyl]-2,7- diazaspiro[3.5]nonan-7- yl]acetate | 520.4 |
| F | 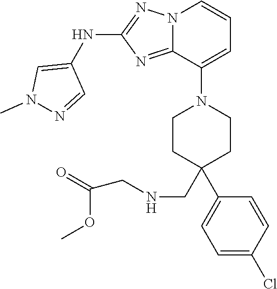 [SMILES: COC(=O)CNCC1(c2ccc(Cl)cc2)CCN(c2cccn3nc(Nc4cnn(C)c4)nc23)CC1] | formic acid; methyl 2- [[4-(4-chlorophenyl)-1- [2-[(1-methylpyrazol-4- yl)amino]- [1,2,4]triazolo[1,5- a]pyridin-8-yl]-4- piperidyl]methylamino] acetate | 509.3 |
| G | 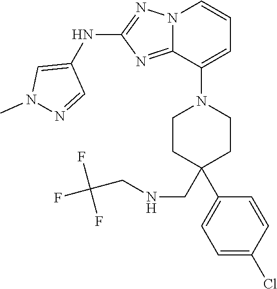 [SMILES: Cn1cc(Nc2nc3c(N4CCC(CNCC(F)(F)F)(c5ccc(Cl)cc5)CC4)cccn3n2)cn1] | 8-[4-(4-chlorophenyl)- 4-[(2,2,2- trifluoroethylamino) methyl]-1-piperidyl]-N-(1- methylpyrazol-4-yl)- [1,2,4]triazolo[1,5- a]pyridin-2-amine | 519.3 |
| H | 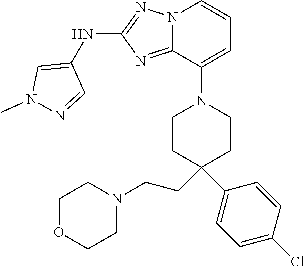 [SMILES: Cn1cc(Nc2nc3c(N4CCC(CCN5CCOCC5)(c5ccc(Cl)cc5)CC4)cccn3n2)cn1] | 8-[4-(4-chlorophenyl)- 4-(2-morpholinoethyl)- 1-piperidyl]-N-(1- methylpyrazol-4-yl)- [1,2,4]triazolo[1,5- a]pyridin-2-amine | 521.3 |
| I | 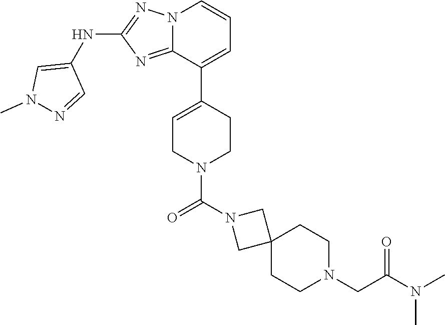 | N,N-dimethyl-2-[2-[4- [2-[(1-methylpyrazol-4- yl)amino]- [1,2,4]triazolo[1,5- a]pyridin-8-yl]-3,6- dihydro-2H-pyridine-1- carbonyl]-2,7- diazaspiro[3.5]nonan-7- yl]acetamide; formic acid | 533.4 |
| J | 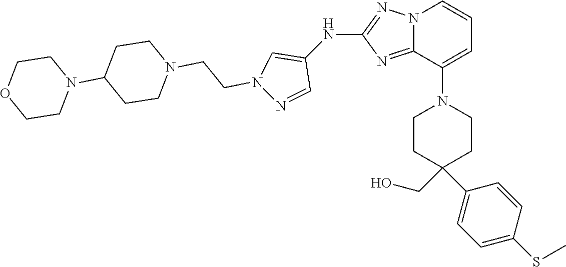 [SMILES: CSc1ccc(C2(CO)CCN(c3cccn4nc(Nc5cnn(CCN6CCC(N7CCOCC7)CC6)c5)nc34)CC2)cc1] | [4-(4- methylsulfanylphenyl)- 1-[2-[[1-[2-(4- morpholino-1- piperidyl)ethyl]pyrazol- 4-yl]amino]- [1,2,4]triazolo[1,5- a]pyridin-8-yl]-4- piperidyl]methanol | 632.5 |
| K | 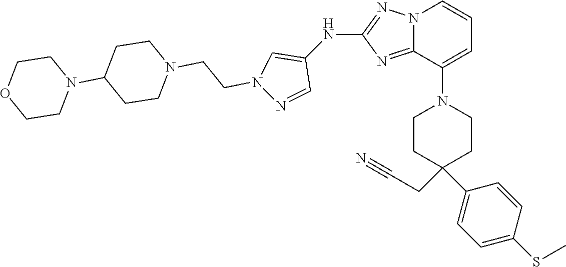 [SMILES: CSc1ccc(C2(CC#N)CCN(c3cccn4nc(Nc5cnn(CCN6CCC(N7CCOCC7)CC6)c5)nc34)CC2)cc1] | 2-[4-(4- methylsulfanylphenyl)- 1-[2-[[1-[2-(4- morpholino-1- piperidyl)ethyl]pyrazol- 4-yl]amino]- [1,2,4]triazolo[1,5- a]pyridin-8-yl]-4- piperidyl]acetonitrile | 641.4 |
| L | 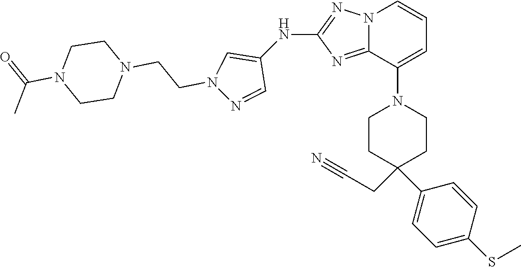 [SMILES: CSc1ccc(C2(CC#N)CCN(c3cccn4nc(Nc5cnn(CCN6CCN(C(C)=O)CC6)c5)nc34)CC2)cc1] | 2-[1-[2-[[1-[2-(4- acetylpiperazin-1- yl)ethyl]pyrazol-4- yl]amino]- [1,2,4]triazolo[1,5- a]pyridin-8-yl]-4-(4- methylsulfanylphenyl)- 4-piperidyl]acetonitrile | 599.4 |
| M | 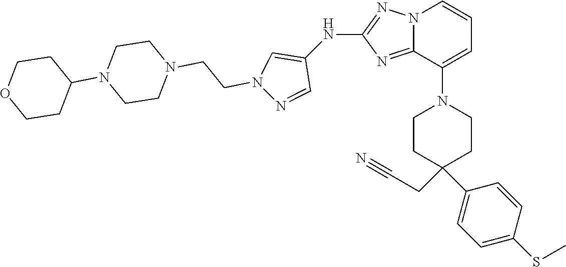 [SMILES: CSc1ccc(C2(CC#N)CCN(c3cccn4nc(Nc5cnn(CCN6CCN(C7CCOCC7)CC6)c5)nc34)CC2)cc1] | 2-[4-(4- methylsulfanylphenyl)- 1-[2-[[1-[2-(4- tetrahydropyran-4- ylpiperazin-1- yl)ethyl]pyrazol-4- yl]amino]- [1,2,4]triazolo[1,5- a]pyridin-8-yl]-4- piperidyl]acetonitrile | 641.5 |
| N | 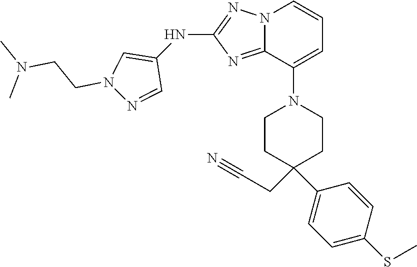 | 2-[1-[2-[[1-[2- (dimethylamino)ethyl] pyrazol-4-yl]amino]- [1,2,4]triazolo[1,5- a]pyridin-8-yl]-4-(4- methylsulfanylphenyl)- 4-piperidyl]acetonitrile | 516.3 |
| O | 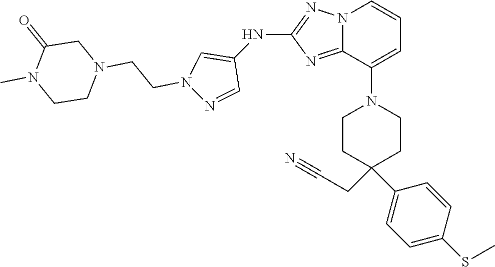 | 2-[1-[2-[[1-[2-(4- methyl-3-oxo- piperazin-1- yl)ethyl]pyrazol-4- yl]amino]- [1,2,4]triazolo[1,5- a]pyridin-8-yl]-4-(4- methylsulfanylphenyl)- 4-piperidyl]acetonitrile | 585.4 |
| P | 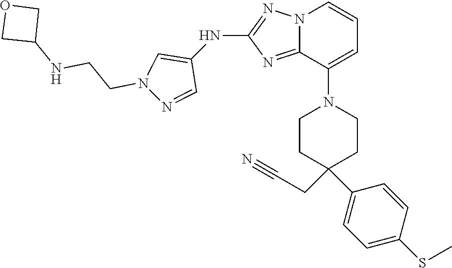 [SMILES: CSc1ccc(C2(CC#N)CCN(c3cccn4nc(Nc5cnn(CCNC6COC6)c5)nc34)CC2)cc1] | 2-[4-(4- methylsulfanylphenyl)- 1-[2-[[1-[2-(oxetan-3- ylamino)ethyl]pyrazol- 4-yl]amino]- [1,2,4]triazolo[1,5- a]pyridin-8-yl]-4- piperidyl]acetonitrile | 544.3 |
| Q | 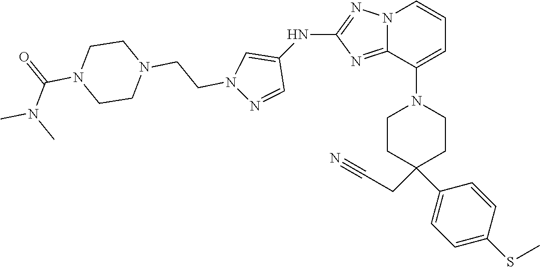 | 4-[2-[4-[[8-[4- (cyanomethyl)-4-(4- methylsulfanylphenyl)- 1-piperidyl]- [1,2,4]triazolo[1,5- a]pyridin-2- yl]amino]pyrazol-1- yl]ethyl]-N,N-dimethyl- piperazine-1- carboxamide | 628.4 |
| R and | 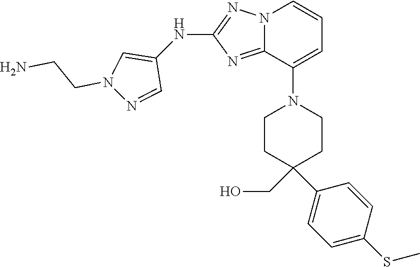 [SMILES: CSc1ccc(C2(CO)CCN(c3cccn4nc(Nc5cnn(CCN)c5)nc34)CC2)cc1] | [1-[2-[[1-(2- aminoethyl)pyrazol-4- yl]amino]- [1,2,4]triazolo[1,5- a]pyridin-8-yl]-4-(4- methylsulfanylphenyl)- 4-piperidyl]methanol | 479.3 |
| S | 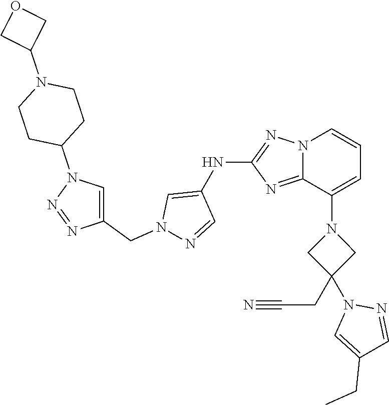 [SMILES: CCc1cnn(C2(CC#N)CN(c3cccn4nc(Nc5cnn(Cc6cn(C7CCN(C8COC8)CC7)nn6)c5)nc34)C2)c1] | 2-[3-(4-ethylpyrazol-1- yl)-1-[2-[[1-[[1-[1- (oxetan-3-yl)-4- piperidyl]triazol-4- yl]methyl]pyrazol-4- yl]amino]- [1,2,4]triazolo[1,5- a]pyridin-8-yl]azetidin- 3-yl]acetonitrile Or 2-[3-(4-ethyl-1H- pyrazol-1-yl)-1-(2-[[1- ([1-[1-(oxetan-3- yl)piperidin-4-yl]-1H- 1,2,3-triazol-4- yl]methyl)-1H-pyrazol- 4-yl]amino]- [1,2,4]triazolo[1,5- a]pyridin-8-yl)azetidin- 3-yl]acetonitrile | 609.3 |

### Example A—JAK Enzyme Assays

The activity of the isolated recombinant JAK1 and JAK2 kinase domain was measured by monitoring phosphorylation of a peptide derived from JAK3 (Val-Ala-Leu-Val-Asp-Gly-Tyr-Phe-Arg-Leu-Thr-Thr, fluorescently labeled on the N-terminus with 5-carboxyfluorescein) using the Caliper LabChip® technology (Caliper Life Sciences, Hopkinton, Mass.). To determine inhibition constants (Kᵢ), compounds were diluted serially in DMSO and added to 50 μL kinase reactions containing purified enzyme (1.5 nM JAK1, or 0.2 nM JAK2), 100 mM HEPES buffer (pH 7.2), 0.015% Brij-35, 1.5 μM peptide substrate, ATP (25 μM), 10 mM MgCl₂, 4 mM DTT at a final DMSO concentration of 2%. Reactions were incubated at 22° C. in 384-well polypropylene microtiter plates for 30 minutes and then stopped by addition of 25 μL of an EDTA containing solution (100 mM HEPES buffer (pH 7.2), 0.015% Brij-35, 150 mM EDTA), resulting in a final EDTA concentration of 50 mM. After termination of the kinase reaction, the proportion of phosphorylated product was determined as a fraction of total peptide substrate using the Caliper LabChip® 3000 according to the manufacturer's specifications. Kᵢ values were then determined using the Morrison tight binding model (Morrison, J. F., Biochim. Biophys. Acta. 185:269-296 (1969); William, J. W. and Morrison, J. F., Meth. Enzymol., 63:437-467 (1979)) modified for ATP-competitive inhibition [Kᵢ=K_{i,app}/(1+[ATP]/ K_{m,app})].

Table 5 provides JAK1 Kᵢ, and JAK2 Kᵢ information for the exemplary compounds. ND=Not Determined.

TABLE 5

| Compound No. | JAK1 Ki (μM) | JAK2 Ki (μM) |
|---|---|---|
| 1-1 | 0.00021 | 0.000045 |
| 1-2 | 0.00052 | 0.00016 |
| 1-3 | 0.00063 | 0.00038 |
| 1-4 | 0.00027 | 0.00011 |
| 1-5 | 0.00023 | 0.00012 |
| 1-6 | 0.00045 | 0.00012 |
| 1-7 | 0.00018 | 0.00011 |
| 1-8 | 0.00056 | 0.00022 |
| 1-9 | 0.00055 | 0.00026 |
| 1-10 | 0.00033 | 0.00016 |
| 1-11 | 0.00042 | 0.00031 |
| 1-12 | 0.00027 | 0.00019 |
| 1-13 | 0.00075 | 0.00044 |
| 1-14 | 0.00016 | 0.00013 |
| 1-15 | 0.00011 | 0.000089 |
| 2-1 | 0.00074 | 0.00088 |
| 2-2 | 0.00087 | 0.00071 |
| 3-1 | 0.00063 | 0.00032 |
| 3-2 | 0.0024 | 0.00086 |
| 3-3 | 0.00082 | 0.0002 |
| 3-4 | 0.0019 | 0.00048 |
| 3-5 | 0.00074 | 0.00028 |
| A | 0.00018 | 0.00013 |
| B | 0.00097 | 0.00058 |
| C | 0.0018 | 0.0029 |
| D | 0.00055 | 0.00094 |
| E | 0.038 | 0.02 |
| F | 0.0014 | 0.00053 |
| G | 0.01 | 0.0029 |
| H | 0.011 | 0.0035 |
| I | 0.19 | 0.086 |
| J | 0.0017 | 0.00047 |
| K | 0.0027 | 0.0011 |
| L | 0.0026 | 0.00065 |
| M | 0.0033 | 0.00096 |
| N | 0.0051 | 0.0012 |
| O | 0.0046 | 0.001 |
| P | 0.0024 | 0.00072 |
| Q | 0.0029 | 0.00067 |
| R | 0.0029 | 0.0014 |
| S | 0.0009 | 0.00057 |

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A compound of formula (I): 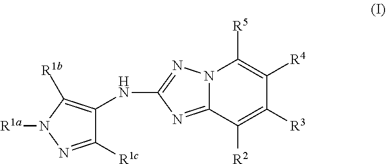
or a stereoisomer, tautomer, solvate, prodrug or salt thereof, wherein:
R^{1a} is hydrogen, C₁-C₆ alkyl, C₃-C₈ cycloalkyl, phenyl, or 3-11 membered heterocyclyl and R^{1a} is optionally substituted by R⁹;
R^{1b} and R^{1c} are each independently hydrogen, C₁-C₆ alkyl, or C₃-C₈ cycloalkyl;
R² is a 3-11 membered heterocyclyl containing at least 1 nitrogen, selected from groups (a)-(e) and (h)-(j); a C₅-C₈ cycloalkenyl ring (f); a —O—(CR^{x}R^{y})_{q}—Ar² group (g); or a Ar¹—O—(CR^{x}R^{y})_{q}—Ar² group (k), where each R^{x} and R^{y} are independently hydrogen or C₁-C₆ alkyl, each q is independently 0 to 3, Ar¹ is 1,4-phenylene and Ar² is optionally substituted C₆-C₁₀ aryl or optionally substituted 5-11 membered heteroaryl: 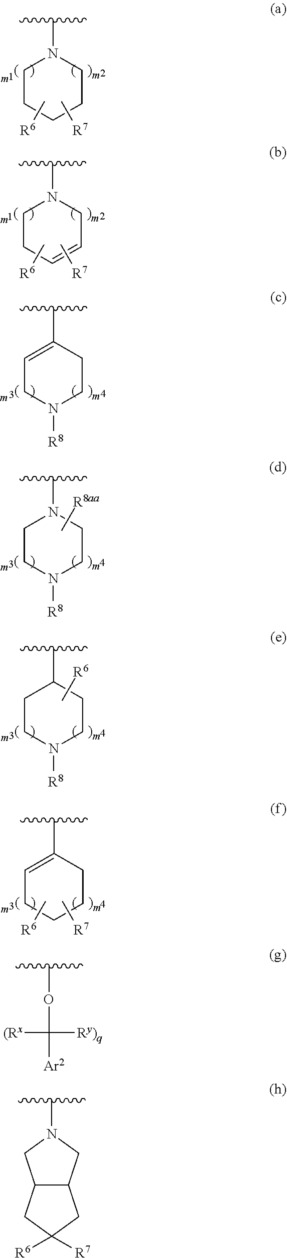 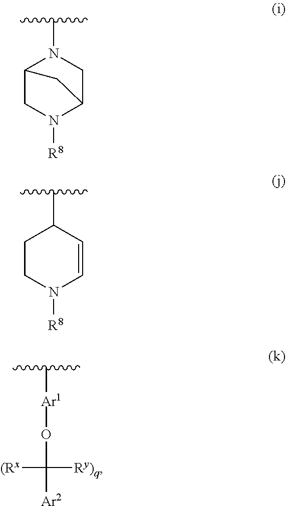
wherein the wavy line represents the point of attachment of R² in formula (I);
R³, R⁴ and R⁵ are each independently selected from the group consisting of hydrogen, CH₃, CH₂CH₃, OCH₃, CF₃, F and Cl;
R⁶ and R⁷ are independently selected from the group consisting of hydrogen, halogen, OH, CN, phenyl, C₁-C₆ alkyl, (C₀-C₆ alkylene)C₃-C₈ cycloalkyl, (C₀-C₆ alkylene)3-11 membered heterocyclyl, (C₀-C₆ alkylene)C(O)NR^{a}R^{b}, (C₀-C₆ alkylene)NR^{a}C(O)(C₁-C₆ alkyl), (C₀-C₆ alkylene)NR^{a}C(O)(phenyl), (C₀-C₆ alkylene)C(O)R^{8a}, (C₀-C₆ alkylene)C(O)OR^{8a}, C₁-C₆ alkoxy, —O—(C₃-C₆ cycloalkyl), —O—(C₀-C₆ alkylene)C(O)NR^{a}R^{b}, —C═N—O—(C₁-C₆ alkyl), —O—(C₁-C₆ alkyl)3-11 membered heterocyclyl, (C₀-C₆ alkylene)NR^{a}SO₂(C₁-C₆ alkyl), (C₀-C₆ alkylene)NR^{a}SO₂(phenyl), and —O-(3-11 membered heterocyclyl); wherein said alkyl, alkylene, alkoxy, cycloalkyl, phenyl and heterocyclyl are each independently optionally substituted,
or R⁶ and R⁷ together form an optionally substituted phenyl or optionally substituted 3-11 membered heterocyclyl;
R⁸ is H, C₁-C₆ alkyl, (C₀-C₆ alkylene)phenyl, (C₀-C₆ alkylene)C₃-C₈ cycloalkyl, (C₀-C₆ alkylene)3-11 membered heterocyclyl, C(O)NR^{a}R^{b}, SO₂NR^{a}R^{b}, (C₁-C₆ alkylene)C(O)OR^{8a} or C(O)R^{8a}, wherein said alkyl, alkylene, heterocyclyl and phenyl are each independently optionally substituted;
R^{8a} is H, NR^{a}R^{b}, C₁-C₆ alkyl, (C₀-C₆ alkylene)C₃-C₈ cycloalkyl, (C₀-C₆ alkylene)phenyl, or (C₀-C₆ alkylene)3-11 membered heterocyclyl, wherein said alkyl, alkylene, cycloalkyl, phenyl and heterocyclyl are each independently optionally substituted;
R^{8aa} is H, C₁-C₆ alkyl optionally substituted by OH, or C(O)NR^{a}R^{b}; or
or R⁸ and R^{8aa} together form an optionally substituted 3-11 membered heterocyclyl;
R⁹, independently at each occurrence, is OH, halogen, CN, C₁-C₆ alkyl, C₃-C₈ cycloalkyl, phenyl, 3-11 membered heterocyclyl, 5-11 membered heteroaryl, —C(O)NR^{a}R^{b}, —NR^{a}R^{b}, (C₁-C₆ alkylene)C₃-C₈ cycloalkyl, (C₁-C₆ alkylene)phenyl, (C₁-C₆ alkylene)3-11 membered heterocyclyl, (C₁-C₆ alkylene)5-11 membered heteroaryl, (C₁-C₆ alkylene)C(O)NR^{a}R^{b}, (C₁-C₆ alkylene)NR^{a}R^{b}, or C(O)(C₁-C₆ alkyl), wherein said alkyl, alkylene, cycloalkyl, phenyl, heterocyclyl and heteroaryl are each independently optionally substituted;
R^{a} and R^{b}, independently at each occurrence, are selected from the group consisting of hydrogen, C₁-C₆ alkyl optionally substituted by halogen or CN, (C₀-C₆ alkylene)C₃-C₈ cycloalkyl, or (C₀-C₆ alkylene)phenyl, and wherein one or more alkylene units of any alkyl group is independently optionally substituted by —O—, or alternatively R^{a} and R^{b} may be joined together with the nitrogen atom to which they are attached to form an optionally substituted 3-11 membered heterocyclyl; and
m¹, m², m³ and m⁴ are each independently 0, 1 or 2.

2. The compound of claim 1, wherein the compound is selected from: 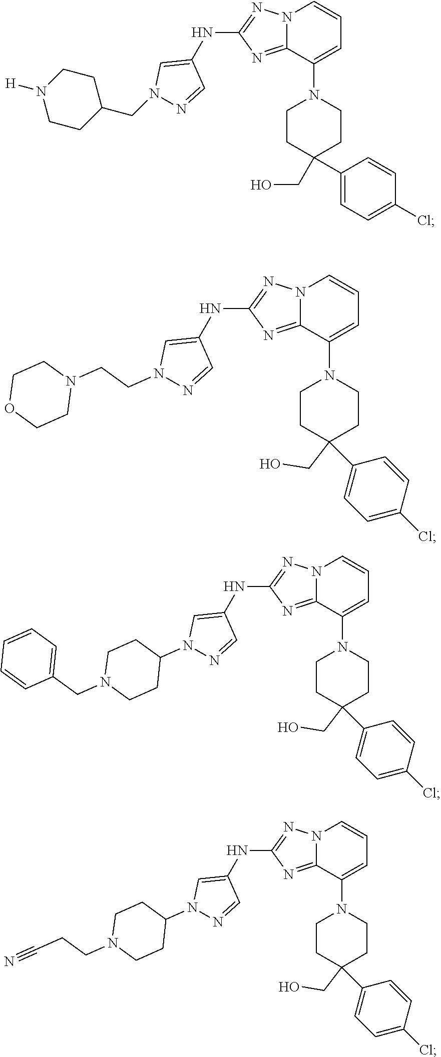 [SMILES: OCC1(c2ccc(Cl)cc2)CCN(c2cccn3nc(Nc4cnn(CC5CCNCC5)c4)nc23)CC1 ; OCC1(c2ccc(Cl)cc2)CCN(c2cccn3nc(Nc4cnn(CCN5CCOCC5)c4)nc23)CC1 ; OCC1(c2ccc(Cl)cc2)CCN(c2cccn3nc(Nc4cnn(C5CCN(Cc6ccccc6)CC5)c4)nc23)CC1 ; N#CCCN1CCC(n2cc(Nc3nc4c(N5CCC(CO)(c6ccc(Cl)cc6)CC5)cccn4n3)cn2)CC1] 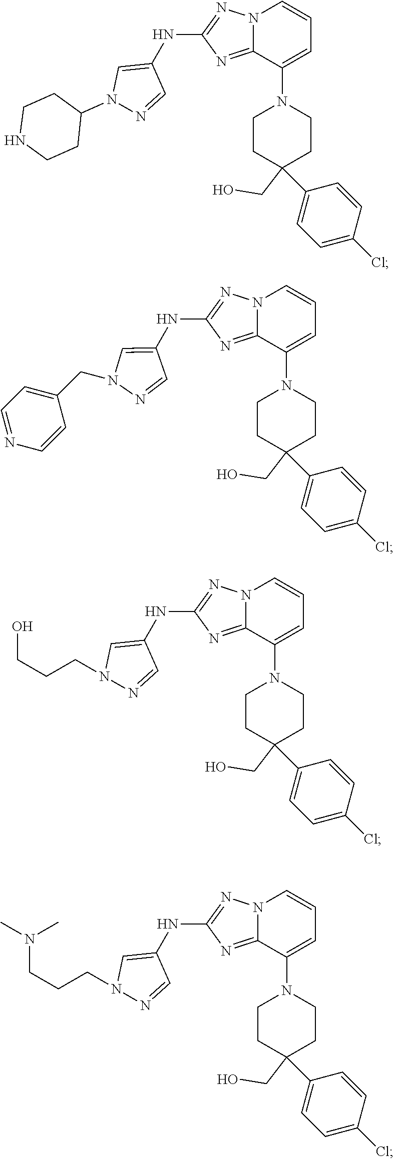 [SMILES: OCC1(c2ccc(Cl)cc2)CCN(c2cccn3nc(Nc4cnn(C5CCNCC5)c4)nc23)CC1 ; OCC1(c2ccc(Cl)cc2)CCN(c2cccn3nc(Nc4cnn(Cc5ccncc5)c4)nc23)CC1 ; OCCCn1cc(Nc2nc3c(N4CCC(CO)(c5ccc(Cl)cc5)CC4)cccn3n2)cn1 ; CN(C)CCCn1cc(Nc2nc3c(N4CCC(CO)(c5ccc(Cl)cc5)CC4)cccn3n2)cn1] 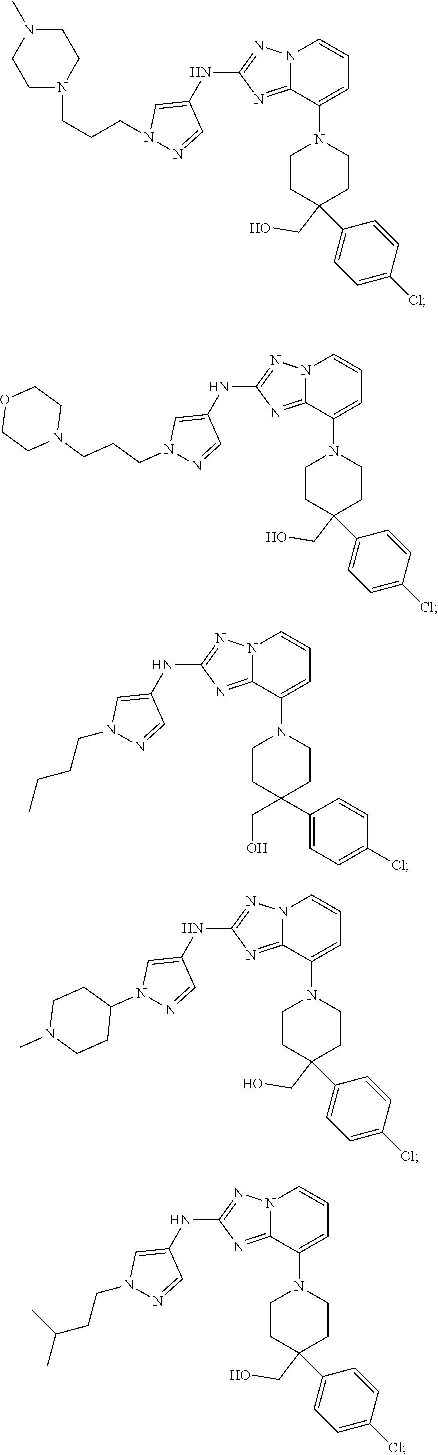 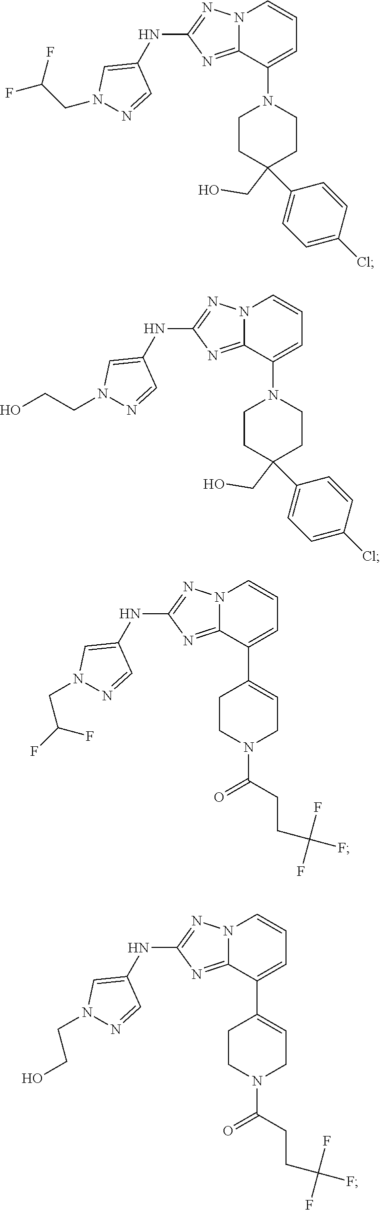 [SMILES: OCC1(c2ccc(Cl)cc2)CCN(c2cccn3nc(Nc4cnn(CC(F)F)c4)nc23)CC1 ; OCCn1cc(Nc2nc3c(N4CCC(CO)(c5ccc(Cl)cc5)CC4)cccn3n2)cn1 ; O=C(CCC(F)(F)F)N1CC=C(c2cccn3nc(Nc4cnn(CC(F)F)c4)nc23)CC1 ; O=C(CCC(F)(F)F)N1CC=C(c2cccn3nc(Nc4cnn(CCO)c4)nc23)CC1] 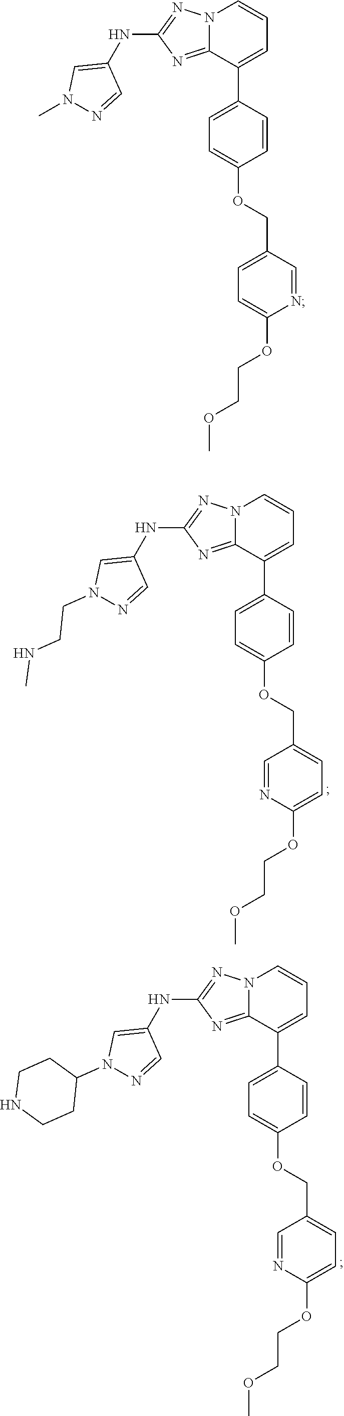 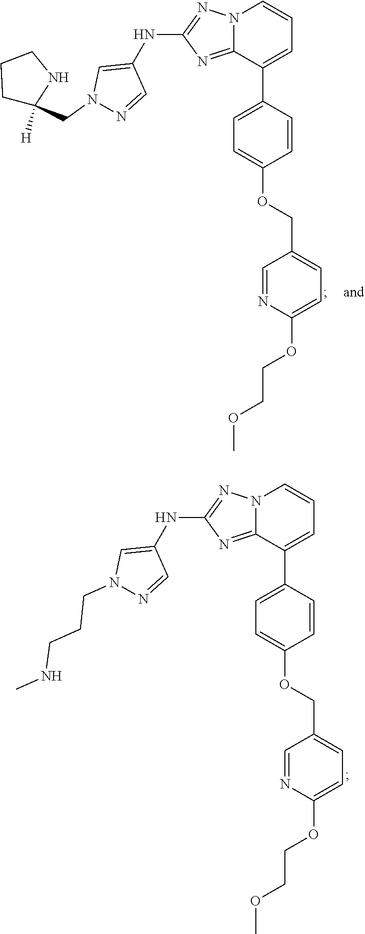
or a pharmaceutically acceptable salt thereof.

3. The compound of claim 1, wherein the compound is selected from: 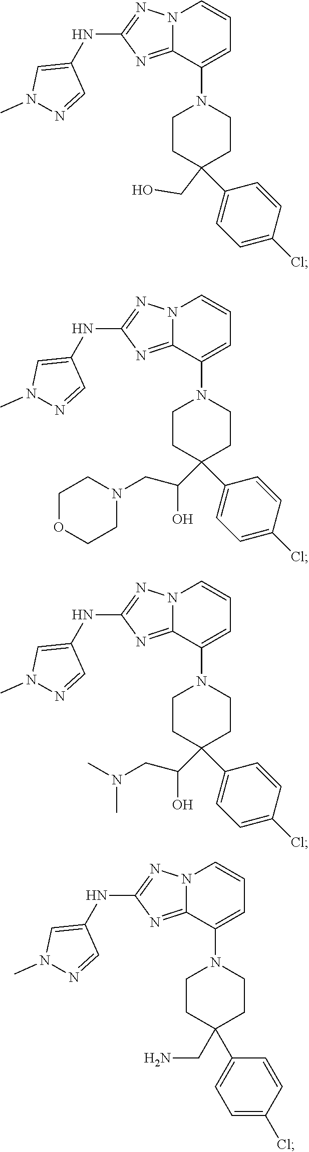 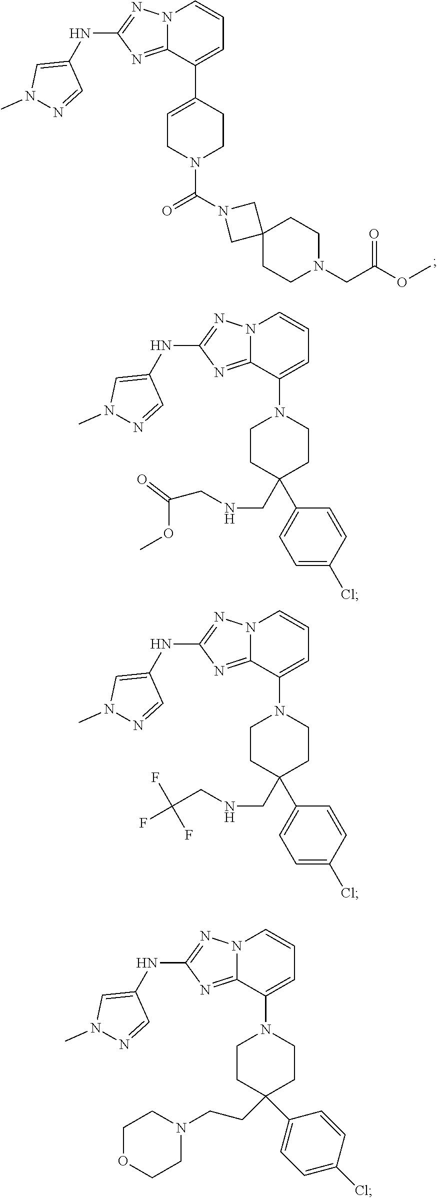 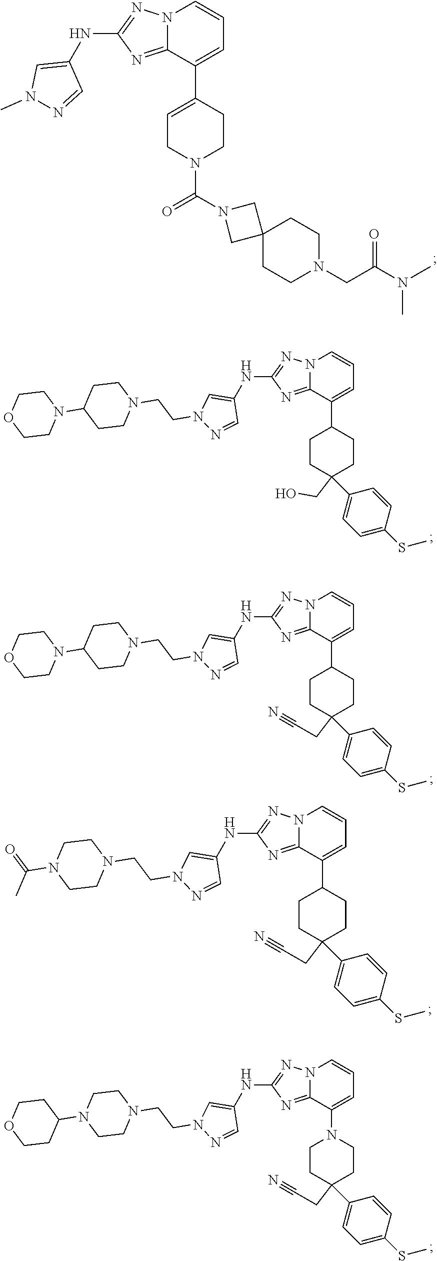 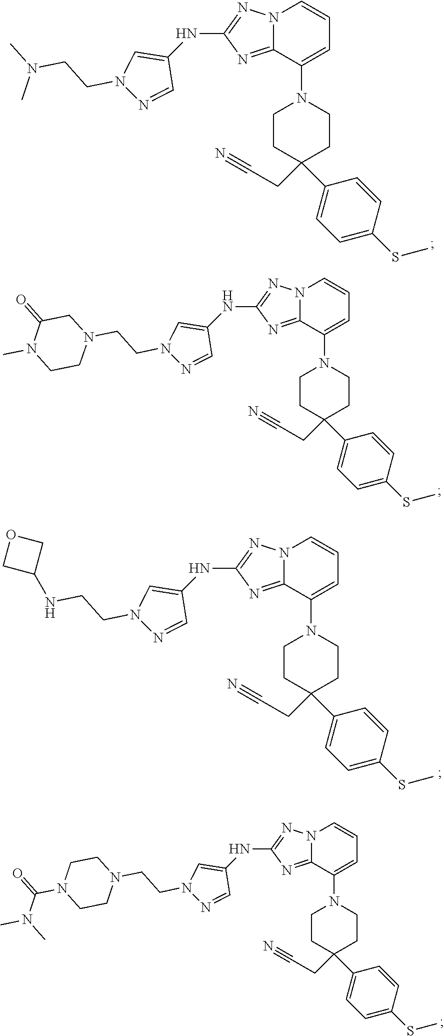 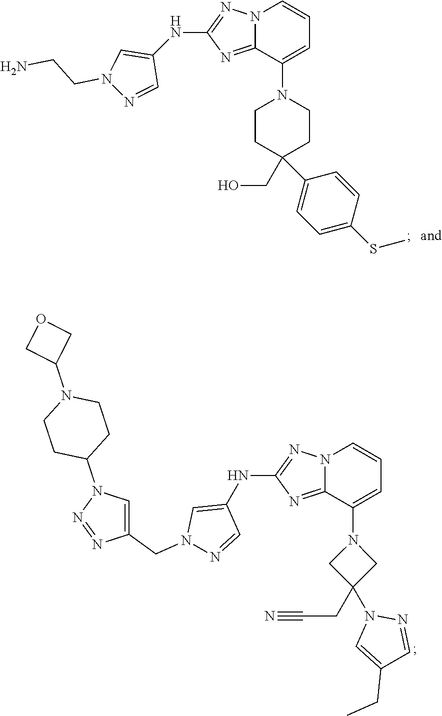 [SMILES: CSc1ccc(C2(CO)CCN(c3cccn4nc(Nc5cnn(CCN)c5)nc34)CC2)cc1 ; CCc1cnn(C2(CC#N)CN(c3cccn4nc(Nc5cnn(Cc6cn(C7CCN(C8COC8)CC7)nn6)c5)nc34)C2)c1]
or a pharmaceutically acceptable salt thereof.

4. A pharmaceutical composition comprising a compound of claim 1, or a pharmaceutically acceptable salt or stereoisomer thereof, and a pharmaceutically acceptable carrier, diluent or excipient.

5. A method of preventing, treating or lessening the severity of a disease or condition responsive to the inhibition of a Janus kinase activity in a patient, comprising administering to the patient a therapeutically effective amount of a compound of claim 1, or a pharmaceutically acceptable salt or stereoisomer thereof.

6. The method of claim 5, wherein the disease or condition is asthma, rhinitis, allergic airway syndrome, atopic dermatitis, bronchitis, rheumatoid arthritis, psoriasis, contact dermatitis, chronic obstructive pulmonary disease or delayed hypersensitivity reaction.

7. The method of claim 5, wherein the Janus kinase is JAK1, JAK2 or JAK3.

8. A lot for treating a disease or disorder responsive to the inhibition of a Janus kinase, comprising: (a) a first pharmaceutical composition comprising a compound of formula (I); and (b) instructions for use.

9. The kit of claim 8, further comprising; (c) a second pharmaceutical composition, the second pharmaceutical composition comprising an agent for treatment of asthma, rhinitis, allergic airway syndrome, atopic dermatitis, bronchitis, rheumatoid arthritis, psoriasis, contact dermatitis, chronic obstructive pulmonary disease or delayed hypersensitivity reaction.
